(19) United States
(12) Patent Application Publication
(10) Pub. No.: US 2022/0401514 A1
(43) Pub. Date: Dec. 22, 2022

(54) ANTIMICROBIAL, BACTERIOPHAGE-DERIVED POLYPEPTIDES AND THEIR USE AGAINST GRAM-NEGATIVE AND ACID-FAST BACTERIA

(71) Applicant: CONTRAFECT CORPORATION, Yonkers, NY (US)

(72) Inventor: Raymond SCHUCH, Mountain Lakes, NJ (US)

(21) Appl. No.: 17/621,357

(22) PCT Filed: Jul. 2, 2020
(86) PCT No.: PCT/US2020/040714

§ 371 Date: Dec. 21, 2021

Related U.S. Application Data
(60) Provisional application No. 62/870,908, filed on Jul. 5, 2019.
(60) Provisional application No. 62/892,783, filed on Aug. 28, 2019.
(60) Provisional application No. 62/911,900, filed on Oct. 7, 2019.
(60) Provisional application No. 62/948,052, filed on Dec. 13, 2019.
(60) Provisional application No. 62/964,743, filed on Jan. 23, 2020.

(51) Int. Cl. A61K 38/16; A61K 45/06; A61P 31/04

(52) U.S. Cl. CPC A61K 38/162; A61K 45/06; A61P 31/04

(57) ABSTRACT

Disclosed herein are pharmaceutical compositions comprising an effective amount of an isolated Chp peptide having an amino acid sequence selected from the group consisting of SEQ ID NOs. 81-91 and 94-102, or a modified Chp peptide having about 80% sequence identity therewith, wherein the modified Chp peptide inhibits growth, reduces the population, or kills at least one species of Gram-negative or acid-fast bacteria; and a pharmaceutically acceptable carrier. Further disclosed herein are isolated Chp peptides, as well as vectors comprising a nucleic acid molecule that encode the Chp peptides and host cells comprising a vector. Also disclosed herein are methods of inhibiting the growth, reducing the population, or killing of at least one species of Gram-negative or acid-fast bacteria, methods of treating a bacterial infection in a subject, and methods for prevention, disruption, or treatment of a biofilm comprising a Gram-negative bacteria.

### CROSS REFERENCE TO RELATED APPLICATIONS

This application claims the benefit of, and relies on the filing date of, U.S. provisional patent application No. 62/870,908, filed 5 Jul. 2019; U.S. provisional patent application No. 62/892,783, filed 28 Aug. 2019; U.S. provisional patent application No. 62/911,900, filed 7 Oct. 2019; U.S. provisional patent application No. 62/948,052, filed 13 Dec. 2019; and U.S. provisional patent application No. 62/964,743, filed 23 Jan. 2020, the entire disclosures of which are incorporated herein by reference.

### SEQUENCE LISTING

The present application contains a Sequence Listing which has been submitted electronically in ASCII format and is hereby incorporated by reference in its entirety. Said ASCII copy, created on Jun. 25, 2020 is named 0341_0019-00-304_SL.txt and is 56,128 bytes in size.

### FIELD OF THE DISCLOSURE

The present disclosure relates to the field of antimicrobial agents and more specifically to phage-derived antimicrobial amurin peptides that infect Gram-negative and/or acid-fast bacteria and the use of these peptides in killing Gram-negative and/or acid-fast bacteria and combatting bacterial infection and contamination.

### BACKGROUND OF THE DISCLOSURE

Gram-negative bacteria, in particular, members of the genus Pseudomonas and the emerging multi-drug resistant pathogen Acinetobacter baumannii, are an important cause of serious and potentially life-threatening invasive infections. Pseudomonas infection presents a major problem in burn wounds, chronic wounds, chronic obstructive pulmonary disorder (COPD), cystic fibrosis, surface growth on implanted biomaterials, and within hospital surface and water supplies where it poses a host of threats to vulnerable patients.

Once established in a patient, P. aeruginosa can be especially difficult to treat. The genome encodes a host of resistance genes, including multidrug efflux pumps and enzymes conferring resistance to beta-lactam and aminoglycoside antibiotics, making therapy against this Gram-negative pathogen particularly challenging due to the lack of novel antimicrobial therapeutics. This challenge is compounded by the ability of P. aeruginosa to grow in a biofilm, which may enhance its ability to cause infections by protecting bacteria from host defenses and chemotherapy.

In the healthcare setting, the incidence of drug-resistant strains of Pseudomonas aeruginosa is increasing. In an observational study of health care-associated bloodstream infections (BSIs) in community hospitals, P. aeruginosa was one of the top four Multiple Drug Resistant (MDR) pathogens, contributing to an overall hospital mortality of 18%. Additionally, outbreaks of MDR P. aeruginosa are well-documented. Poor outcomes are associated with MDR strains of P. aeruginosa that frequently require treatment with drugs of last resort, such as colistin.

Other drug-resistant bacteria that have been identified as significant threats by the World Health Organization (WHO) and Centers for Disease Control (CDC) include the following Gram-negative bacteria: Acinetobacter baumannii, Pseudomonas aeruginosa, Enterobacteriaceae (including Escherichia coli, Klebsiella pneumoniae, and Enterobacter cloacae), Salmonella species, Neisseria gonorrhoeae, and Shigella species (Tillotson G. 2018. A crucial list of pathogens. Lancet Infect Dis 18:234-236).

Acid-fast bacteria, which generally have a high content of mycolic acid in their cell walls, can be identified by measuring the bacteria's resistance to decolorization by acids during laboratory staining such as Ziehl-Neelsen staining. Acid-fast bacteria can resist the decolorization of acid-based stains. Like Gram-negative bacteria, acid-fast bacteria, e.g., actinobacteria, are responsible for life-threatening diseases. For example, mycobacterium is a genus of actinobacteria and includes pathogens known to cause serious diseases including tuberculosis and leprosy. Mycobacterium tuberculosis usually presents by infecting the lungs and may spread through the air when an infected subject coughs, sneezes, or speaks, for example. Like P. aeruginosa, M. tuberculosis also has an increasing incidence of drug-resistant strains, making tuberculosis infections increasingly more difficult to treat.

To address the need for new antimicrobials with novel mechanisms, researchers are investigating a variety of drugs and biologics. One such class of antimicrobial agents includes lysins. Lysins are cell wall peptidoglycan hydrolases, which act as “molecular scissors” to degrade the peptidoglycan meshwork responsible for maintaining cell shape and for withstanding internal osmotic pressure. Degradation of peptidoglycan results in osmotic lysis. However, certain lysins have not been effective against Gram-negative bacteria, at least in part, due to the presence of an outer membrane (OM), which is absent in Gram-positive bacteria and which limits access to subjacent peptidoglycan. Modified lysins (“artilysins”) have also been developed. These agents, which contain lysins fused to specific α-helical domains with polycationic, amphipathic, and hydrophobic features, are capable of translocating across the OM. However, certain artilysins exhibit low in vivo activity. This may be caused by constituents of human serum and specifically by physiologic salt and divalent cations. These constituents compete for lipopolysaccharide binding sites and may interfere with the α-helical translocation domains of lysins, thereby restricting activity in blood and limiting the effectiveness of certain lysins and artilysins for treating invasive infections. A similar lack of activity in blood has been reported for multiple different outer membrane-penetrating and destabilizing antimicrobial peptides.

In addition to lysins and artilysins, other phage-encoded host lysis systems have been identified, including “amurins” (Chamakura K R et al., 2017. Mutational analysis of the MS2 lysis protein L. Microbiology 163:961-969). The term amurin describes a limited set of nonmuralytic (not “wall-destroying,” i.e., not based on peptidoglycan hydrolysis of the cell wall) lysis activities from both ssDNA and ssRNA phages (Microviridae and Leviviridae, respectively). For example, the protein E amurin of phage φX174 (Family Microviridae, genus Microvirus) is a 91 amino acid membrane protein that causes lysis by inhibiting the bacterial translocase MraY, an essential membrane-embedded enzyme that catalyzes the formation of the murein precursor, Lipid I (Zheng Y et al., 2009. Purification and functional characterization of phiX174 lysis protein E. Biochemistry 48:4999-5006). Additionally, the A2 capsid protein of phage Qβ (Family Leviviridae, genus Allolevivirus) is a 420-amino acid structural protein (and amurin) that causes lysis by interfering with MurA activity and dysregulating the process of peptidoglycan biosynthesis (Gorzelnik K V et al., 2016. Proc Natl Acad Sci USA 113:11519-11524). Other non-limiting examples include the LysM amurin of phage M, which is a specific inhibitor of MurJ, the lipid II flippase of E. coli, and the protein L amurin of phage MS2 (Family Levivirdae, genus Levivirus), which is a 75 amino acid integral membrane protein and causes lysis in a manner requiring the activity of host chaperone DnaJ (Chamakura K R et al., 2017. J Bacteriol 199). A putative domain structure for the L-like amurins has been assigned and includes an internal leucylserine dipeptide immediately preceded by a stretch of 10-17 hydrophobic residues. These amurins are integral membrane proteins and have not been purified and used like lysins. Further, their targets are in the cytoplasm. They have not been tested as lytic agents. Some amurins have been described in detail, for example in PCT Published Application No. WO 2001/009382, but at best they constitute a basis for development of therapeutics and have not been developed into antibacterial therapeutics.

Although recent publications have described lysins/artilysins and other host lysis systems (e.g., amurins) that may be used against Gram-negative bacteria with varying levels of efficacy in vivo, there remains a need for additional antibacterial compounds that target MDR P. aeruginosa, M. tuberculosis, and other Gram-negative and acid-fast bacteria for the treatment of invasive infections, and especially antibacterial compounds that are highly soluble, remain active in vivo in the presence of serum and/or pulmonary surfactant, do not have hemolytic activity, and/or have a low propensity for resistance.

### SUMMARY OF THE DISCLOSURE

This application discloses a novel class of phage lytic agents that are derived, for example, from Microviridae genomic sequences and are distinct from other such agents, including known lysins/artilysins and amurins. The phage lytic agents disclosed herein are referred to as Chlamydia phage (Chp) peptides, also referred to as “amurin peptides” (a functional definition not implying sequence similarity with amurins). Disclosed herein are various Chp peptides that have been identified, constituting a family of specific bacteriolytic proteins, as well as non-naturally occurring modified variants of those Chp peptides (corresponding to SEQ ID NOs. 81-91 and 94-102). As used herein, “Chp peptides” refers to both naturally-occurring Chp peptides, non-naturally occurring modified variants thereof, and modified Chp peptides having at least one modification (e.g., substitution) as compared to a wild-type Chp peptide. Several of the Chp peptides disclosed herein exhibit notable sequence similarities to each other but are distinct from other known peptides in the sequence databases. Despite the unique sequences of the Chp peptides, they are all predicted to adopt alpha-helical structures similar to some previously described antimicrobial peptides (AMPs) of vertebrate innate immune systems (E. F. Haney et al, 2017, In Hansen PR (ed), Antimicrobial Peptides: Methods and Protocols, Methods in Molecular Biology, vol. 1548) but with no sequence similarity to such AMPs. Consistent with an antibacterial function for the Chp class, disclosed herein is the potent and broad-spectrum bactericidal activity against Gram-negative and acid-fast pathogens for several different purified Chp peptides. Unlike the previously described amurins of Microviridae, which have cytoplasmic targets in the cell wall biosynthetic apparatus that may not be easily accessed by externally applied proteins, the Chp peptides disclosed herein can be used, in purified forms, to exert bactericidal activity “from without,” i.e., by acting on the outside of the cell wall. The Chp peptides identified here represent a novel class of antimicrobial agents having broad-spectrum activity against Gram-negative and acid-fast pathogens and the ability to persist in the presence of serum and/or pulmonary surfactant.

In one aspect, the present disclosure is directed to a pharmaceutical composition comprising a pharmaceutically acceptable carrier and an effective amount of (i) an isolated Chp peptide having an amino acid sequence selected from the group consisting of SEQ ID NOs. 81-91 and 94-102 or active fragments thereof, or (ii) a modified Chp peptide having at least 80%, such as at least 85%, at least 90%, at least 92.5%, at least 95%, at least 98%, at least 99% sequence identity with at least one of SEQ ID NOs. 81-91 and 94-102, wherein the modified Chp peptide inhibits the growth, reduces the population, and/or kills at least one species of Gram-negative bacteria or acid-fast bacteria, optionally in the presence of human serum and/or pulmonary surfactant. In certain embodiments, the at least one species of Gram-negative bacteria comprises Pseudomonas aeruginosa. In certain embodiments, the at least one species of acid-fast bacteria comprises at least one species of Mycobacterium. In certain embodiments, the at least one species of acid-fast bacteria comprises Mycobacterium tuberculosis. In certain embodiments, the at least one species of acid-fast bacteria comprises nontuberculous mycobacteria (NTM).

In another embodiment disclosed herein, the pharmaceutical composition comprises a pharmaceutically acceptable carrier and an effective amount of an isolated Chp peptide selected from the group consisting of peptides Chp2-M1, Chp2-Cys, Chp2-NC, Chp4::Chp2, Chp2-CAV, and Ecp1-CAV or active fragments thereof.

In some embodiments, the Chp peptide is Chp2-M1, Chp4-M1, Ecp1-M1, Chp6-M1, Chp10-M1, Unp2-M1, Agt1-M1, or Ecp3-M1.

In various embodiments of the disclosure, the pharmaceutical composition comprises a pharmaceutically acceptable carrier and an effective amount of (i) an isolated Chp peptide having an amino acid sequence selected from the group consisting of SEQ ID NO: 81; SEQ ID NO: 82; SEQ ID NO: 83; SEQ ID NO: 84; SEQ ID NO: 85; and SEQ ID NO: 86 or active fragments thereof, or (ii) a modified Chp peptide having at least 80%, such as at least 85%, at least 90%, at least 92.5%, at least 95%, at least 98%, at least 99% sequence identity with at least one of SEQ ID NOs. 81-86, wherein the modified Chp peptide inhibits the growth, reduces the population, and/or kills at least one species of Gram-negative bacteria or acid-fast bacteria, optionally in the presence of human serum and/or pulmonary surfactant.

In certain embodiments, the pharmaceutical composition comprises a pharmaceutically acceptable carrier and an effective amount of (i) an isolated Chp peptide having an amino acid sequence selected from the group consisting of SEQ ID NO: 81; SEQ ID NO: 87, SEQ ID NO: 88; SEQ ID NO: 89; SEQ ID NO: 91; SEQ ID NO: 97; SEQ ID NO: 100; and SEQ ID NO: 101 or active fragments thereof, or (ii) a modified Chp peptide having at least 80%, such as at least 85%, at least 90%, at least 92.5%, at least 95%, at least 98%, at least 99% sequence identity with at least one of SEQ ID NOs. 81, 87, 88, 89, 91, 97, 100, and 101, wherein the modified Chp peptide inhibits the growth, reduces the population, and/or kills at least one species of Gram-negative bacteria or acid-fast bacteria, optionally in the presence of human serum and/or pulmonary surfactant.

In certain embodiments, the Chp peptide as disclosed herein or active fragments thereof contains at least one non-natural modification relative to the amino acid sequence of any one of SEQ ID NOs. 81-91 and 94-102, such as SEQ ID NO: 94 or SEQ ID NO: 102, and in certain embodiments, the non-natural modification is selected from the group consisting of substitution modification, such as a substitution of an amino acid; an N-terminal acetylation modification; and a C-terminal amidation modification. In certain embodiments, the modified Chp peptide comprises at least one amino acid substitution, insertion, or deletion relative to the amino acid sequence of any one of SEQ ID NOs. 81-91 and 94-102, wherein the modified Chp peptide inhibits the growth, reduces the population, and/or kills at least one species of Gram-negative or acid-fast bacteria, optionally in the presence of human serum and/or pulmonary surfactant. In certain embodiments, the at least one species of Gram-negative bacteria comprises Pseudomonas aeruginosa. In certain embodiments, the at least one species of acid-fast bacteria comprises a species of Mycobacterium. In certain embodiments, the at least one species of acid-fast bacteria comprises Mycobacterium tuberculosis. In certain embodiments, the at least one species of acid-fast bacteria comprises nontuberculous mycobacteria (NTM). In certain embodiments, the at least one amino acid substitution is a conservative amino acid substitution. In certain embodiments, the modified Chp peptide comprising at least one amino acid substitution relative to the amino acid sequence of any one of SEQ ID NOs. 81-91 and 94-102 is a cationic peptide having at least one alpha helix domain.

The pharmaceutical composition in some embodiments may be a solution, a suspension, an emulsion, an inhalable powder, an aerosol, or a spray. In some embodiments the pharmaceutical composition may also comprise one or more antibiotics suitable for the treatment of Gram-negative bacteria or acid-fast bacteria. Optionally, the peptide Chp1 is excluded such that the pharmaceutical composition does not comprise Chp1.

In certain embodiments, disclosed herein is a vector comprising a nucleic acid that encodes (i) a Chp peptide having an amino acid sequence selected from the group consisting of SEQ ID NOs. 81-91 and 94-102 or active fragments thereof, or (ii) a Chp peptide having at least 80%, at least 85%, at least 90%, at least 92.5%, at least 95%, at least 98%, or at least 99% sequence identity with at least one of SEQ ID NOs. 81-91 and 94-102, wherein the modified Chp peptide inhibits the growth, reduces the population, and/or kills at least one species of Gram-negative or acid-fast bacteria, optionally in the presence of human serum and/or pulmonary surfactant. In certain embodiments, the at least one species of Gram-negative bacteria comprises Pseudomonas aeruginosa. In certain embodiments, the at least one species of acid-fast bacteria comprises Mycobacterium tuberculosis. In certain embodiments, the at least one species of acid-fast bacteria comprises nontuberculous mycobacteria (NTM).

Also disclosed herein are recombinant expression vectors comprising a nucleic acid encoding (i) a Chp peptide comprising an amino acid sequence selected from the group consisting of SEQ ID NOs. 81-91 and 94-102 or active fragments thereof, or (ii) a modified Chp peptide having at least 80%, at least 85%, at least 90%, at least 92.5%, at least 95%, at least 98%, or at least 99% sequence identity with at least one of SEQ ID NOs. 81-91 and 94-102, wherein the modified Chp peptide inhibits the growth, reduces the population, and/or kills at least one species of Gram-negative bacteria, optionally in the presence of human serum and/or pulmonary surfactant. In certain embodiments, the at least one species of Gram-negative bacteria comprises Pseudomonas aeruginosa. In certain embodiments, the nucleic acid is operatively linked to a heterologous promoter. In certain embodiments, the nucleic acid encodes a Chp peptide comprising an amino acid sequence selected from the group consisting of the group consisting of SEQ ID NOs: 81-86 or active fragments thereof, and in certain embodiments, the nucleic acid encodes a Chp peptide comprising an amino acid sequence selected from the group consisting of SEQ ID NO: 81; SEQ ID NO: 87; SEQ ID NO: 88; SEQ ID NO: 89; SEQ ID NO: 91; SEQ ID NO: 97; SEQ ID NO: 100; and SEQ ID NO: 101 or active fragments thereof.

Further embodiments disclosed herein include an isolated host cell comprising the foregoing vectors. In some embodiments, the nucleic acid sequence is a cDNA sequence.

In yet another aspect, the disclosure is directed to isolated, purified nucleic acid encoding a Chp peptide comprising an amino acid sequence selected from the group consisting of SEQ ID NOs. 81-91 and 94-102 or active fragments thereof. In certain embodiments, the nucleic acid encodes a Chp peptide comprising an amino acid sequence selected from the group consisting of SEQ ID NOs. 81-86 or active fragments thereof. In an alternative embodiment, the isolated, purified DNA comprises a nucleotide sequence selected from the group consisting of SEQ ID NOs. 81, 87, 88, 89, 91, 97, 100 and 101. Optionally, the nucleic acid is cDNA. In certain embodiments, the nucleotide sequence contains at least one non-natural modification, such as a mutation (e.g., substitution, insertion, or deletion) or a nucleic acid sequence encoding an N-terminal modification or a C-terminal modification.

In other aspects, the present disclosure is directed to various methods/uses. One such use is a method for inhibiting the growth, reducing the population, and/or killing of at least one species of Gram-negative bacteria, the method comprising contacting the bacteria with a composition comprising an effective amount of (i) a Chp peptide comprising an amino acid sequence selected from the group consisting of SEQ ID NOs. 81-91 and 94-102 or active fragments thereof, or (ii) a modified Chp peptide having at least 80%, such as at least 85%, at least 90%, at least 92.5%, at least 95%, at least 98%, or at least 99% sequence identity with at least one of SEQ ID NOs. 81-91 and 94-102, wherein the modified Chp peptide inhibits said growth, reduces said population, and/or kills said at least one species of Gram negative bacteria. In certain embodiments, the Chp peptide comprises an amino acid sequence selected from the group consisting of SEQ ID NOs: 81-86 or active fragments thereof.

In other aspects, there is provided a method for inhibiting the growth, reducing the population, and/or killing of at least one species of acid-fast bacteria, the method comprising contacting the bacteria with a composition comprising an effective amount of (i) a Chp peptide comprising an amino acid sequence selected from the group consisting of SEQ ID NOs. 1, 2, 4, 6, 8-16, 18-21, 23-26, 59-61, 63-65, 67, 81-91 and 94-102 or active fragments thereof, or (ii) a modified Chp peptide having at least 80%, such as at least 85%, at least 90%, at least 92.5%, at least 95%, at least 98%, or at least 99% sequence identity with at least one of SEQ ID NOs. 1, 2, 4, 6, 8-16, 18-21, 23-26, 59-61, 63-65, 67, 81-91 and 94-102, wherein the modified Chp peptide inhibits said growth, reduces said population, and/or kills said at least one species of acid-fast bacteria. In certain embodiments, the Chp peptide comprises an amino acid sequence selected from the group consisting of SEQ ID NOs: 81, 87, 88, 89, 91, 97, 100 and 101 or active fragments thereof.

In certain embodiments, the at least one species of Gram-negative bacteria is Pseudomonas aeruginosa, and in certain embodiments, the method further comprises killing at least one other species of Gram-negative bacteria in addition to Pseudomonas aeruginosa.

In certain embodiments, the at least one species of acid-fast bacteria is a species of Mycobacterium, and in certain embodiments, the Mycobacterium is Mycobacterium tuberculosis. In certain embodiments, the at least one species of acid-fast bacteria is a species of non-tuberculosis mycobacterium. In certain embodiments, the non-tuberculosis mycobacterium is selected from at least one of Mycobacterium smegmatis, Mycobacterium avium, Mycobacterium kansasii, Mycobacterium scrofulaceum, Mycobacterium peregrinum, Mycobacterium marinum, Mycobacterium intracellulare, and Mycobacterium fortuitum. In certain embodiments, the non-tuberculosis mycobacterium is M. smegmatis.

Also disclosed herein is a method for treating a bacterial infection caused by a Gram-negative bacteria, comprising administering a pharmaceutical composition comprising a Chp peptide comprising an amino acid sequence selected from the group consisting of SEQ ID NOs. 81-91 and 94-102, active fragments thereof, or a modified Chp peptide thereof, as disclosed herein, to a subject diagnosed with, at risk for, or exhibiting symptoms of a bacterial infection. Further disclosed herein is a method for treating a bacterial infection caused by an acid-fast bacteria, comprising administering a pharmaceutical composition comprising an amino acid sequence selected from the group consisting of SEQ ID NOs. 1, 2, 4, 6, 8-16, 18-21, 23-26, 59-61, 63-65, 67, 81-91 and 94-102, active fragments thereof, or a modified Chp peptide thereof, as disclosed herein, to a subject diagnosed with, at risk for, or exhibiting symptoms of a bacterial infection.

In yet another aspect, there is provided a method for prevention, disruption, or treatment of a biofilm comprising a Gram-negative bacteria, the method comprising contacting a biofilm with a pharmaceutical composition comprising (i) an isolated Chp peptide having an amino acid sequence selected from the group consisting of SEQ ID NOs. 81-91 and 94-102 or active fragment thereof, or (ii) a modified Chp peptide having 80% sequence identity with the amino acid sequence of at least one of SEQ ID NOs. 81-91 and 94-102, wherein the modified Chp peptide inhibits the growth, reduces the population, or kills at least one species of Gram-negative bacteria, and wherein the biofilm is effectively prevented, dispersed, or treated.

In any of the foregoing methods/uses, the Gram-negative bacteria may be at least one Gram-negative bacteria selected from the group consisting of Stenotrophomonas species, Acinetobacter baumannii, Pseudomonas aeruginosa, Escherichia coli, Klebsiella pneumoniae, Enterobacter cloacae, Salmonella species (e.g., Salmonella Senftenberg, Salmonella Enteritidis, Salmonella Typhimurium, and Salmonella Oslo), Neisseria gonorrhoeae, Citrobacter freundii, Serratia marcescens, Morganella morganii, Raoultella ornithinolytica, Kluyvera ascorbata, Klebsiella oxytoca, Proteus mirabilis, Enterbacter aerogenes, Enterococcus faecium, Burkholderia species, Achromobacter species, and Shigella species. In certain embodiments, the Gram-negative bacteria is Pseudomonas aeruginosa. In certain embodiments, the Stenotrophomonas species is Stenotrophomonas maltophilia.

In any of the foregoing methods/uses, the acid-fast bacteria may be at least one actinobacteria selected from the group consisting of Mycobacterium smegmatis, Mycobacterium tuberculosis and non-tuberculosis mycobacteria.

In certain embodiments of the methods/uses disclosed herein, the at least one species of Gram-negative bacteria is resistant to one or more antibiotics typically suitable for the treatment of Gram-negative bacterial infections. In certain embodiments, the at least one species of Gram-negative bacteria is a multi-drug resistant (MDR) pathogen. Also disclosed herein is a method for treating or preventing a topical or systemic pathogenic bacterial infection caused by a Gram-negative bacteria comprising administering a pharmaceutical composition comprising a Chp peptide comprising an amino acid sequence selected from the group consisting of SEQ ID NOs. 81-91 and 94-102, active fragments thereof, or a modified Chp peptide thereof, as disclosed herein, to a subject in need of treatment or prevention. In certain embodiments, disclosed herein a method for treating or preventing a topical or systemic pathogenic bacterial infection caused by an acid-fast bacteria comprising administering a pharmaceutical composition comprising an amino acid sequence selected from the group consisting of SEQ ID NOs. 1, 2, 4, 6, 8-16, 18-21, 23-26, 59-61, 63-65, 67, 81-91 and 94-102, active fragments thereof, or a modified Chp peptide thereof, as disclosed herein, to a subject in need of treatment or prevention.

Further disclosed herein is a method for preventing or treating a bacterial infection comprising co-administering to a subject diagnosed with, at risk for, or exhibiting symptoms of a bacterial infection, a combination of a first amount of a pharmaceutical composition comprising a Chp peptide comprising an amino acid sequence selected from the group consisting of SEQ ID NOs. 81-91 and 94-102, active fragments thereof, or a modified Chp peptide thereof, as disclosed herein, and a second amount of an antibiotic suitable for the treatment of Gram-negative bacterial infection, wherein the first and the second amounts together are effective for preventing or treating the Gram-negative bacterial infection. Also disclosed herein is a method for preventing or treating a bacterial infection comprising co-administering to a subject diagnosed with, at risk for, or exhibiting symptoms of a bacterial infection, a combination of a first amount of a pharmaceutical composition comprising a Chp peptide comprising an amino acid sequence selected from the group consisting of SEQ ID NOs. 1, 2, 4, 6, 8-16, 18-21, 23-26, 59-61, 63-65, 67, 81-91 and 94-102, active fragments thereof, or a modified Chp peptide thereof, as disclosed herein, and a second amount of an antibiotic suitable for the treatment of an acid-fast bacterial infection, wherein the first and the second amounts together are effective for preventing or treating the acid-fast bacterial infection.

In some embodiments, the antibiotic suitable for the treatment of Gram-negative bacterial infection is selected from one or more of ampicillin, cefataxime, ceftriaxone, minocycline, tetracycline, tigecycline, trimethoprim, sulfamethoxazole, ceftazidime, cefepime, cefoperazone, ceftobiprole, ciprofloxacin, levofloxacin, aminoglycosides, imipenem, meropenem, doripenem, gentamicin, tobramycin, amikacin, piperacillin, ticarcillin, penicillin, rifampicin, polymyxin B, and colistin. In certain embodiments, the antibiotic is selected from one or more of amikacin, azithromycin, aztreonam, ciprofloxacin, colistin, fosfomycin, gentamicin, imipenem, piperacillin, rifampicin, and tobramycin. In some embodiments, the antibiotic suitable for the treatment of acid-fast bacterial infection is selected from one or more of isoniazid, rifampin, ethambutol, and pyrazinamide.

In yet another embodiment, there is disclosed a method for augmenting the efficacy of an antibiotic suitable for the treatment of Gram-negative bacterial infection, comprising co-administering the antibiotic in combination with a pharmaceutical composition comprising a Chp peptide comprising an amino acid sequence selected from the group consisting of SEQ ID NOs. 81-91 and 94-102, active fragments thereof, or a modified Chp peptide thereof, as disclosed herein, wherein administration of the combination is more effective in inhibiting the growth, reducing the population, or killing the Gram-negative bacteria than administration of either the antibiotic or the pharmaceutical composition thereof individually. Also disclosed is a method for augmenting the efficacy of an antibiotic suitable for the treatment of acid-fast bacterial infection, comprising co-administering the antibiotic in combination with a pharmaceutical composition comprising a Chp peptide comprising an amino acid sequence selected from the group consisting of SEQ ID NOs. 1, 2, 4, 6, 8-16, 18-21, 23-26, 59-61, 63-65, 67, 81-91 and 94-102, active fragments thereof, or a modified Chp peptide thereof, as disclosed herein, wherein administration of the combination is more effective in inhibiting the growth, reducing the population, or killing the acid-fast bacteria than administration of either the antibiotic or the pharmaceutical composition thereof individually.

### BRIEF DESCRIPTION OF THE DRAWINGS

FIG. 1A are three-dimensional models predicted by I-Tasser for structures of Chlamydia phage peptide (Chp) family members Chp1, Chp2, Chp4, Chp5, Chp6, Chp7, Ecp1, Ecp2, and Osp1. The human innate immune effector peptide LL-37 is included for comparison. Alpha helical structures are evident, and the top terminal is generally the N-terminal.

FIG. 1B shows the consensus secondary structure predictions for Chp2 (SEQ ID NO: 2) using JPRED4. The alpha-helices are indicated by the thick striped bar.

FIG. 1C shows the consensus secondary structure predictions for Chp4 (SEQ ID NO: 4) using JPRED4. The alpha-helices are indicated by the thick striped bar

FIG. 2A is the rooted (UPGMA clustering method) phylogenetic tree of certain Chp family members generated from a ClustalW alignment.

FIG. 2B is the unrooted (neighbor-joining clustering method) phylogenetic tree of certain Chp family members generated from a ClustalW alignment.

FIG. 3 is a series of photomicrographs showing microscopic analysis (×2000 magnification) of Pseudomonas aeruginosa strain 1292 treated for 15 minutes with Chp2 (10 μg/mL) or a buffer control (“untreated”) in 100% human serum. Samples were stained using the Live/Dead Cell Viability Kit (ThermoFisher) and examined by both differential interference contrast (DIC) and fluorescence microscopy. The photomicrographs show an absence of dead bacteria in the untreated row and a reduction of live bacteria in the treated row.

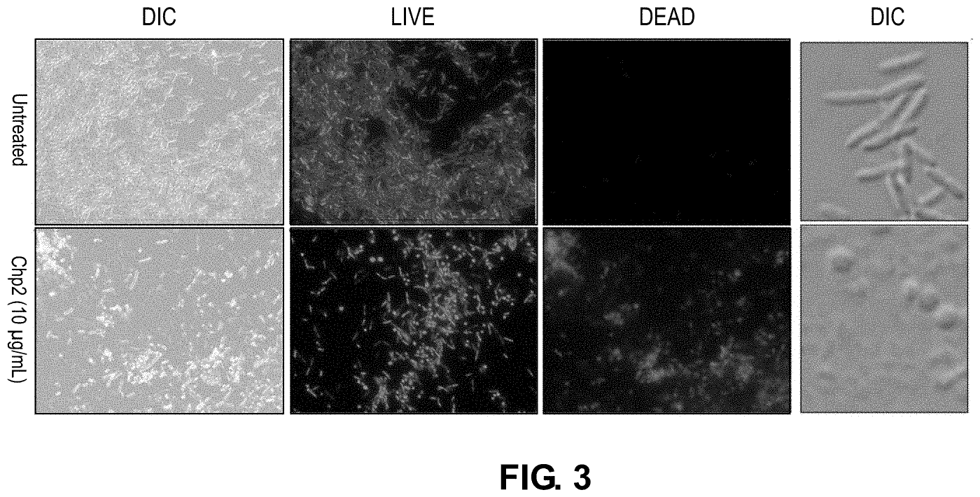

FIG. 4A is a series of photomicrographs showing microscopic analysis (×2000 magnification) of Pseudomonas aeruginosa strain 1292 in Survanta® untreated and 5 minutes after treatment with Chp2-M1 at 1 μg/mL, 10 μg/mL, and 100 μg/mL. Samples were stained using the Live/Dead stains SYTOX® Green (live) and propidium iodide (dead), and examined by both bright field (BF) and fluorescence microscopy. The photomicrographs show an absence of dead bacteria in the untreated row and a reduction of live bacteria in the treated rows, wherein the reduction increases as the concentration of Chp2-M1 increases.

FIG. 4B is a series of photomicrographs showing microscopic analysis (×2000 magnification) of Pseudomonas aeruginosa strain 1292 in Survanta® untreated and 30 minutes after treatment with Chp2-M1 at 1 μg/mL, 10 μg/mL, and 100 μg/mL. Samples were stained using the Live/Dead stains SYTOX® Green (live) and propidium iodide (dead), and examined by both BF and fluorescence microscopy. The photomicrographs show an absence of dead bacteria in the untreated row and a reduction of live bacteria in the treated rows, wherein the reduction increases as the concentration of Chp2-M1 increases.

FIG. 5A is a series of photomicrographs showing microscopic analysis (×2000 magnification) of Pseudomonas aeruginosa strain 1292 in Survanta® untreated and 5 minutes after treatment with Ecp3-M1 at 1 μg/mL, 10 μg/mL, and 100 μg/mL. Samples were stained using the Live/Dead stains SYTOX® Green (live) and propidium iodide (dead), and examined by both BF and fluorescence microscopy. The photomicrographs show an absence of dead bacteria in the untreated row and a reduction of live bacteria in the treated rows, wherein the reduction increases as the concentration of Ecp3-M1 increases.

FIG. 5B is a series of photomicrographs showing microscopic analysis (×2000 magnification) of Pseudomonas aeruginosa strain 1292 in Survanta® untreated and 30 minutes after treatment with Ecp3-M1 at 1 μg/mL, 10 μg/mL, and 100 μg/mL. Samples were stained using the Live/Dead stains SYTOX® Green (live) and propidium iodide (dead), and examined by both BF and fluorescence microscopy. The photomicrographs show an absence of dead bacteria in the untreated row and a reduction of live bacteria in the treated rows, wherein the reduction increases as the concentration of Ecp3-M1 increases.

FIG. 6A is a series of photomicrographs showing microscopic analysis (×2000 magnification) of Pseudomonas aeruginosa strain 1292 in human serum untreated and 5 minutes after treatment with Chp2-M1 at 1 μg/mL, 10 μg/mL, and 100 μg/mL. Samples were stained using the Live/Dead stains SYTOX® Green (live) and propidium iodide (dead), and examined by both BF and fluorescence microscopy. The photomicrographs show an absence of dead bacteria in the untreated row and a reduction of live bacteria in the treated rows, wherein the reduction increases as the concentration of Chp2-M1 increases.

FIG. 6B is a series of photomicrographs showing microscopic analysis (×2000 magnification) of Pseudomonas aeruginosa strain 1292 in human serum untreated and 30 minutes after treatment with Chp2-M1 at 1 μg/mL and 10 μg/mL. Samples were stained using the Live/Dead stains SYTOX® Green (live) and propidium iodide (dead), and examined by both BF and fluorescence microscopy. The photomicrographs show an absence of dead bacteria in the untreated row and a reduction of live bacteria in the treated rows, wherein the reduction increases as the concentration of Chp2-M1 increases.

FIG. 7A is a series of photomicrographs showing microscopic analysis (×2000 magnification) of Pseudomonas aeruginosa strain 1292 in human serum untreated and 5 minutes after treatment with Ecp3-M1 at 1 μg/mL, 10 μg/mL, and 100 μg/mL. Samples were stained using the Live/Dead stains SYTOX® Green (live) and propidium iodide (dead), and examined by both BF and fluorescence microscopy. The photomicrographs show an absence of dead bacteria in the untreated row and a reduction of live bacteria in the treated rows, wherein the reduction increases as the concentration of Ecp3-M1 increases.

FIG. 7B is a series of photomicrographs showing microscopic analysis (×2000 magnification) of Pseudomonas aeruginosa strain 1292 in human serum untreated and 30 minutes after treatment with Ecp3-M1 at 1 μg/mL, 10 μg/mL, and 100 μg/mL. Samples were stained using the Live/Dead stains SYTOX® Green (live) and propidium iodide (dead), and examined by both BF and fluorescence microscopy. The photomicrographs show an absence of dead bacteria in the untreated row and a reduction of live bacteria in the treated rows, wherein the reduction increases as the concentration of Ecp3-M1 increases.

### DETAILED DESCRIPTION

### Definitions

As used herein, the following terms and cognates thereof shall have the following meanings unless the context clearly indicates otherwise:

“Carrier” refers to a solvent, additive, excipient, dispersion medium, solubilizing agent, coating, preservative, isotonic and absorption delaying agent, surfactant, propellant, diluent, vehicle and the like with which an active compound is administered. Such carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil, and the like.

“Pharmaceutically acceptable carrier” refers to any and all solvents, additives, excipients, dispersion media, solubilizing agents, coatings, preservatives, isotonic and absorption delaying agents, surfactants, propellants, diluents, vehicles and the like that are physiologically compatible. The carrier(s) must be “acceptable” in the sense of not being deleterious to the subject to be treated in amounts typically used in medicaments. Pharmaceutically acceptable carriers are compatible with the other ingredients of the composition without rendering the composition unsuitable for its intended purpose. Furthermore, pharmaceutically acceptable carriers are suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers or excipients include any of the standard pharmaceutical carriers such as phosphate buffered saline solutions, water, and emulsions such as oil/water emulsions and microemulsions. Suitable pharmaceutical carriers are described, for example, in Remington's Pharmaceutical Sciences by E. W. Martin, 18th Edition. The pharmaceutically acceptable carrier may be a carrier that does not exist in nature.

“Bactericidal” or “bactericidal activity” refers to the property of causing the death of bacteria or capable of killing bacteria to an extent of at least a 3-log 10 (99.9%) or better reduction among an initial population of bacteria over an 18-24 hour period.

“Bacteriostatic” or “bacteriostatic activity” refers to the property of inhibiting bacterial growth, including inhibiting growing bacterial cells, thus causing a 2-log 10 (99%) or better and up to just under a 3-log reduction among an initial population of bacteria over an 18-24 hour period.

“Antibacterial” refers to both bacteriostatic and bactericidal agents.

“Antibiotic” refers to a compound having properties that have a negative effect on bacteria, such as lethality or reduction of growth. An antibiotic can have a negative effect on any and all combinations of Gram-positive bacteria, Gram-negative bacteria, acid-fast bacteria, and non-acid fast bacteria. By way of example, an antibiotic can affect cell wall peptidoglycan biosynthesis, cell membrane integrity, or DNA or protein synthesis in bacteria. Nonlimiting examples of antibiotics active against Gram-negative bacteria include cephalosporins, such as ceftriaxone-cefotaxime, ceftazidime, cefepime, cefoperazone, and ceftobiprole; fluoroquinolones such as ciprofloxacin and levofloxacin; aminoglycosides such as gentamicin, tobramycin, and amikacin; piperacillin, ticarcillin, imipenem, meropenem, doripenem, broad spectrum penicillins with or without beta-lactamase inhibitors, rifampicin, polymyxin B, and colistin. Non-limiting examples of antibiotics active against acid-fast bacteria include isoniazid, rifampin, ethambutol, and pyrazinamide.

“Drug resistant” generally refers to a bacterium that is resistant to the antibacterial activity of a drug. When used in certain ways, drug resistance may specifically refer to antibiotic resistance. In some cases, a bacterium that is generally susceptible to a particular antibiotic can develop resistance to the antibiotic, thereby becoming a drug resistant microbe or strain. A “multi-drug resistant” (“MDR”) pathogen is one that has developed resistance to at least two classes of antimicrobial drugs, each used as monotherapy. For example, certain strains of S. aureus have been found to be resistant to several antibiotics including methicillin and/or vancomycin (Antibiotic Resistant Threats in the United States, 2013, U.S. Department of Health and Services, Centers for Disease Control and Prevention). One skilled in the art can readily determine if a bacterium is drug resistant using routine laboratory techniques that determine the susceptibility or resistance of a bacterium to a drug or antibiotic.

“Effective amount” refers to an amount which, when applied or administered in an appropriate frequency or dosing regimen, is sufficient to prevent, reduce, inhibit, or eliminate bacterial growth or bacterial burden or to prevent, reduce, or ameliorate the onset, severity, duration, or progression of the disorder being treated (for example, Gram-negative or acid-fast bacterial pathogen growth or infection), prevent the advancement of the disorder being treated, cause the regression of the disorder being treated, or enhance or improve the prophylactic or therapeutic effect(s) of another therapy, such as antibiotic or bacteriostatic therapy.

“Co-administer” refers to the administration of two agents, such as a Chp peptide and an antibiotic or any other antibacterial agent, in a sequential manner, as well as administration of these agents in a substantially simultaneous manner, such as in a single mixture/composition or in doses given separately, but nonetheless administered substantially simultaneously to the subject, for example at different times in the same day or 24-hour period. Such co-administration of Chp peptides with one or more additional antibacterial agents can be provided as a continuous treatment lasting up to days, weeks, or months. Additionally, depending on the use, the co-administration need not be continuous or coextensive. For example, if the use were as a topical antibacterial agent to treat, e.g., a bacterial ulcer or an infected diabetic ulcer, a Chp peptide could be administered only initially within 24 hours of an additional antibiotic, and then the additional antibiotic use may continue without further administration of the Chp peptide.

“Subject” refers to a mammal, a plant, a lower animal, a single cell organism, or a cell culture. For example, the term “subject” is intended to include organisms, e.g., prokaryotes and eukaryotes, which are susceptible to or afflicted with bacterial infections, for example Gram-positive, Gram-negative bacterial infections, or acid-fast bacterial infections. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or susceptible to infection by Gram-negative or acid-fast bacteria, whether such infection be systemic, topical or otherwise concentrated or confined to a particular organ or tissue.

“Polypeptide” is used herein interchangeably with the term “peptide” and refers to a polymer made from amino acid residues and generally having at least about 30 amino acid residues. The term includes not only polypeptides in isolated form, but also active fragments and derivatives thereof, including modified variants. The term “polypeptide” also encompasses fusion proteins or fusion polypeptides comprising a Chp peptide as described herein and maintaining, for example a lytic function. Depending on context, a polypeptide can be a naturally occurring polypeptide or a recombinant, engineered, or synthetically produced polypeptide. A particular Chp peptide can be, for example, derived or removed from a native protein by enzymatic or chemical cleavage, or can be prepared using conventional peptide synthesis techniques (e.g., solid phase synthesis) or molecular biology techniques (such as those disclosed in Sambrook, J. et al., Molecular Cloning: A Laboratory Manual, Cold Spring Harbor Press, Cold Spring Harbor, N.Y. (1989)) or can be strategically truncated or segmented yielding active fragments, maintaining, e.g., lytic activity against the same or at least one common target bacterium.

“Fusion polypeptide” refers to an expression product resulting from the fusion of two or more nucleic acid segments, resulting in a fused expression product typically having two or more domains or segments, which typically have different properties or functionality. In a more particular sense, the term “fusion polypeptide” may also refer to a polypeptide or peptide comprising two or more heterologous polypeptides or peptides covalently linked, either directly or via an amino acid or peptide linker. The polypeptides forming the fusion polypeptide are typically linked C-terminus to N-terminus, although they can also be linked C-terminus to C-terminus, N-terminus to N-terminus, or N-terminus to C-terminus. The term “fusion polypeptide” can be used interchangeably with the term “fusion protein.” The open-ended expression “a polypeptide comprising” a certain structure includes larger molecules than the recited structure, such as fusion polypeptides.

“Heterologous” refers to nucleotide, peptide, or polypeptide sequences that are not naturally contiguous. For example, in the context of the present disclosure, the term “heterologous” can be used to describe a combination or fusion of two or more peptides and/or polypeptides wherein the fusion peptide or polypeptide is not normally found in nature, such as for example a Chp peptide or active fragment thereof and a cationic and/or a polycationic peptide, an amphipathic peptide, a sushi peptide (Ding et al. Cell Mol Life Sci., 65(7-8):1202-19 (2008)), a defensin peptide (Ganz, T. Nature Reviews Immunology 3, 710-720 (2003)), a hydrophobic peptide, and/or an antimicrobial peptide which may have enhanced lytic activity. Included in this definition are two or more Chp peptides or active fragments thereof. These can be used to make a fusion polypeptide with lytic activity.

“Active fragment” refers to a portion of a polypeptide that retains one or more functions or biological activities of the isolated polypeptide from which the fragment was taken, for example bactericidal activity against one or more Gram-negative or acid-fast bacteria.

“Amphipathic peptide” refers to a peptide having both hydrophilic and hydrophobic functional groups. In certain embodiments, secondary structure may place hydrophobic and hydrophilic amino acid residues at opposite sides (e.g., inner side vs outer side when the peptide is in a solvent, such as water) of an amphipathic peptide. These peptides may in certain embodiments adopt a helical secondary structure, such as an alpha-helical secondary structure.

“Cationic peptide” refers to a peptide having a high percentage of positively charged amino acid residues. In certain embodiments, a cationic peptide has a pKa-value of 8.0 or greater. The term “cationic peptide” in the context of the present disclosure also encompasses polycationic peptides that are synthetically produced peptides composed of mostly positively charged amino acid residues, such as lysine (Lys) and/or arginine (Arg) residues. The amino acid residues that are not positively charged can be neutrally charged amino acid residues, negatively charged amino acid residues, and/or hydrophobic amino acid residues.

“Hydrophobic group” refers to a chemical group such as an amino acid side chain that has low or no affinity for water molecules but higher affinity for oil molecules. Hydrophobic substances tend to have low or no solubility in water or aqueous phases and are typically apolar but tend to have higher solubility in oil phases. Examples of hydrophobic amino acids include glycine (Gly), alanine (Ala), valine (Val), Leucine (Leu), isoleucine (Ile), proline (Pro), phenylalanine (Phe), methionine (Met), and tryptophan (Trp).

“Augmenting” refers to a degree of activity of an agent, such as antimicrobial activity, that is higher than it would be otherwise. “Augmenting” encompasses additive as well as synergistic (superadditive) effects.

“Synergistic” or “superadditive” refers to a beneficial effect brought about by two substances in combination that exceeds the sum of the effects of the two agents working independently. In certain embodiments the synergistic or superadditive effect significantly, i.e., statistically significantly, exceeds the sum of the effects of the two agents working independently. One or both active ingredients may be employed at a sub-threshold level, i.e., a level at which if the active substance is employed individually produces no or a very limited effect. The effect can be measured by assays such as the checkerboard assay, described here.

“Treatment” refers to any process, action, application, therapy, or the like, wherein a subject, such as a human being, is subjected to medical aid with the object of curing a disorder, eradicating a pathogen, or improving the subject's condition, directly or indirectly. Treatment also refers to reducing incidence, alleviating symptoms, eliminating recurrence, preventing recurrence, preventing incidence, reducing the risk of incidence, improving symptoms, improving prognosis, or combinations thereof. “Treatment” may further encompass reducing the population, growth rate, or virulence of a bacteria in the subject and thereby controlling or reducing a bacterial infection in a subject or bacterial contamination of an organ, tissue, or environment. Thus “treatment” that reduces incidence may, for example, be effective to inhibit growth of at least one Gram-negative or acid-fast bacterium in a particular milieu, whether it be a subject or an environment. On the other hand, “treatment” of an already established infection refers to inhibiting the growth, reducing the population, killing, including eradicating, a Gram-negative bacteria and/or an acid-fast bacteria responsible for an infection or contamination.

“Preventing” refers to the prevention of the incidence, recurrence, spread, onset or establishment of a disorder such as a bacterial infection. It is not intended that the present disclosure be limited to complete prevention or to prevention of establishment of an infection. In some embodiments, the onset is delayed, or the severity of a subsequently contracted disease or the chance of contracting the disease is reduced, and such constitute examples of prevention.

“Contracted diseases” refers to diseases manifesting with clinical or subclinical symptoms, such as the detection of fever, sepsis, or bacteremia, as well as diseases that may be detected by growth of a bacterial pathogen (e.g., in culture) when symptoms associated with such pathology are not yet manifest.

The term “derivative” in the context of a peptide or polypeptide or active fragments thereof is intended to encompass, for example, a polypeptide modified to contain one or more chemical moieties other than an amino acid that do not substantially adversely impact or destroy the lytic activity. The chemical moiety can be linked covalently to the peptide, e.g., via an amino terminal amino acid residue, a carboxy terminal amino acid residue, or at an internal amino acid residue. Such modifications may be natural or non-natural. In certain embodiments, a non-natural modification may include the addition of a protective or capping group on a reactive moiety, addition of a detectable label, such as antibody and/or fluorescent label, addition or modification of glycosylation, or addition of a bulking group such as PEG (pegylation) and other changes known to those skilled in the art. In certain embodiments, the non-natural modification may be a capping modification, such as N-terminal acetylations and C-terminal amidations. Exemplary protective groups that may be added to Chp peptides include, but are not limited to, t-Boc and Fmoc. Commonly used fluorescent label proteins such as, but not limited to, green fluorescent protein (GFP), red fluorescent protein (RFP), cyan fluorescent protein (CFP), yellow fluorescent protein (YFP), and mCherry, are compact proteins that can be bound covalently or noncovalently to a Chp peptide or fused to a Chp peptide without interfering with normal functions of cellular proteins. In certain embodiments, a polynucleotide encoding a fluorescent protein may be inserted upstream or downstream of the Chp polynucleotide sequence. This will produce a fusion protein (e.g., Chp Peptide::GFP) that does not interfere with cellular function or function of a Chp peptide to which it is attached. Polyethylene glycol (PEG) conjugation to proteins has been used as a method for extending the circulating half-life of many pharmaceutical proteins. Thus, in the context of Chp peptide derivatives, the term “derivative” encompasses Chp peptides chemically modified by covalent attachment of one or more PEG molecules. It is anticipated that pegylated Chp peptides will exhibit prolonged circulation half-life compared to the unpegylated Chp peptides, while retaining biological and therapeutic activity.

“Modified variant” refers to a Chp peptide wherein a non-naturally occurring modification has been made to the amino acid sequence that either enhances the lytic activity or does not substantially adversely impact or destroy the lytic activity of the Chp peptide. Exemplary modifications that may be made to modified variants include modifying an amino acid of the Chp peptide, such as a positively charged amino acid, from an L-form to a D-form; adding an amino acid residue or residues to the C-terminus and/or the N-terminus, forming fusion polypeptides, and forming charge array variants, wherein amino acid charges have been reordered.

“Percent amino acid sequence identity” refers to the percentage of amino acid residues in a candidate sequence that are identical with the amino acid residues in the reference polypeptide sequence, such as a specific Chp peptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for example, using publicly available software such as BLAST or software available commercially, for example from DNASTAR. Two or more polypeptide sequences can be anywhere from 0-100% identical, or any integer value there between. In the context of the present disclosure, two polypeptides are “substantially identical” when at least 80% of the amino acid residues (such as at least about 85%, at least about 90%, at least about 92.5%, at least about 95%, at least about 98%, or at least about 99%) are identical. The term “percent (%) amino acid sequence identity” as described herein applies to Chp peptides as well. Thus, the term “substantially identical” will encompass mutated, truncated, fused, or otherwise sequence-modified forms of isolated Chp polypeptides and peptides described herein, and active fragments thereof, as well as polypeptides with substantial sequence identity (e.g., at least 80%, at least 85%, at least 90%, at least 92.5%, at least 95%, at least 98%, or at least 99% identity as measured for example by one or more methods referenced above) as compared to the reference (wild type or other intact) polypeptide.

As used herein, two amino acid sequences are “substantially homologous” when at least about 80% of the amino acid residues (such as at least about 85%, at least about 90%, at least about 92.5%, at least about 95%, at least about 98%, or at least about 99%) are identical, or represent conservative substitutions. The sequences of the polypeptides of the present disclosure are substantially homologous when one or more, such as up to 10%, up to 15%, or up to 20% of the amino acids of the polypeptide, such as the Chp peptides described herein, are substituted with a similar or conservative amino acid substitution, and wherein the resulting peptides have at least one activity (e.g., antibacterial effect) and/or bacterial specificities of the reference polypeptide, such as the Chp peptides disclosed herein.

As used herein, a “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Inhalable composition” refers to pharmaceutical compositions of the present disclosure that are formulated for direct delivery to the respiratory tract during or in conjunction with routine or assisted respiration (e.g., by intratracheobronchial, pulmonary, and/or nasal administration), including, but not limited to, atomized, nebulized, dry powder, and/or aerosolized formulations.

“Biofilm” refers to bacteria that attach to surfaces and aggregate in a hydrated polymeric matrix that may be comprised of bacterial- and/or host-derived components. A biofilm is an aggregate of microorganisms in which cells adhere to each other on a biotic or abiotic surface. These adherent cells are frequently embedded within a matrix comprised of, but not limited to, extracellular polymeric substance (EPS). Biofilm EPS, which is also referred to as slime (although not everything described as slime is a biofilm) or plaque, is a polymeric conglomeration generally composed of extracellular DNA, proteins, and polysaccharides.

“Preventing biofilm formation” refers to the prevention of the incidence, recurrence, spread, onset or establishment of a biofilm. It is not intended that the present disclosure be limited to complete prevention or to prevention of establishment of biofilm. In some embodiments, the onset of a biofilm is delayed, or the establishment of a biofilm is reduced or the chance of formation of a new biofilm is reduced, and such constitute examples of prevention of a biofilm. Further, prevention of a biofilm may be due to any mechanism including 1) effectively killing planktonic bacteria; 2) killing “persister” bacterial cells in suspensions, i.e., bacteria that are metabolically inactive, tolerant of antibiotics, and highly associated with biofilm formation; and/or 3) preventing “aggregation”, i.e., the ability of bacteria to attach to one another via proteins or polysaccharides.

“Eradication” in reference to a biofilm includes 1) effectively killing bacteria in a biofilm including persister bacterial cells in the biofilm and, optionally 2) effectively destroying and/or damaging the biofilm matrix.

“Disruption” in reference to a biofilm refers to a mechanism that falls between prevention and eradication. A biofilm, which is disrupted, may be “opened”, or otherwise damaged, thus permitting, e.g., an antibiotic, to more readily penetrate the biofilm and kill the bacteria.

“Suitable” in the context of an antibiotic being suitable for use against certain bacteria refers to an antibiotic that was found to be effective against those bacteria even if resistance subsequently developed.

“Outer Membrane” or “OM” refers to a feature of Gram-negative bacteria. The outer membrane is comprised of a lipid bilayer with an internal leaflet of phospholipids and an external amphiphilic leaflet largely consisting of lipopolysaccharide (LPS). The LPS has three main sections: a hexa-acylated glucosamine-based phospholipid called lipid A, a polysaccharide core and an extended, external polysaccharide chain called 0-antigen. The OM presents a non-fluid continuum stabilized by three major interactions, including: i) the avid binding of LPS molecules to each other, especially if cations are present to neutralize phosphate groups; ii) the tight packing of largely saturated acyl chains; and iii) hydrophobic stacking of the lipid A moiety. The resulting structure is a barrier for both hydrophobic and hydrophilic molecules. Below the OM, the peptidoglycan forms a thin layer that is very sensitive to hydrolytic cleavage—unlike the peptidoglycan of Gram-negative bacteria which is 30-100 nanometers (nm) thick and consists of up to 40 layers, the peptidoglycan of Gram-negative bacteria is only 2-3 nm thick and consists of only 1-3 layers.

### Microviridae Phages

Members of the phage family Microviridae may be of particular interest as potential sources of anti-infective agents for several reasons. As disclosed herein, it has been found that a large subset of these phages, including those of the genus Chlamydiamicrovirus (Family Microvirus, subfamily Gokushovirinae), have no conserved amurin sequence and instead encode small, uncharacterized cationic peptides that appear to form the basis of a heretofore uncharacterized lytic system. Additionally, bacteriophages of the family Microviridae infect medically-relevant organisms, including members of the families Enterobacteriaceae, Pseudomonadaceae, and Chlamydiaceae (Doore S M et al, 2016. Virology 491:45-55.). They also lack amurins and instead, as disclosed herein, encode unique uncharacterized antimicrobial-like peptides (called amurin peptides) that have not been previously identified or had a function ascribed to them. It was reasoned that if the putative antimicrobial-like peptides act in a manner similar to previously described antimicrobial peptides (AMPs), they would then be predicted to enable “lysis from without” in a manner not possible with the amurins and their cytoplasmic targets.

Based on a bioinformatics analysis of all annotated Microviridae genomic sequences in GenBank (with a focus on phages that lack amurins), several novel and syntenic open reading frames were identified. They encode small cationic peptides with predicted alpha-helical structures similar to AMPs (but with amino acid sequences dissimilar to AMPs) from the innate immune systems of a variety of vertebrates. These peptides, collectively referred to as “Chp peptides” or “amurin peptides,” are primarily found in the Chlamydiamicrovirus genus and, to a lesser extent, in other related members of the subfamily Gokushovirinae. See, e.g., Tables 1 and 2 below. The Chp peptides from a range of Microviridae phages may exhibit 30-100% identity to each other and may have no or little homology with other peptides in the protein sequence database. See, e.g., Table 3 below. In addition, several modified variants were derived from the identified Chp peptides. Based on the prediction that the Chp peptides possess AMP-like activities, the family members and modified variants were synthesized (Chp2 and Chp3 being identical amino acid sequences) for analysis in different Aspartate Aminotransferase (AST) assays. Based on minimum inhibitory concentration (MIC) values of 0.25-4 μg/mL in the presence of human serum, several Chp peptides have demonstrated superior serum activity compared to a group of up to 17 known AMPs tested (including innate immune effectors and derivatives thereof). Several Chp peptides have additionally demonstrated superior activity in pulmonary surfactant (Survanta®) in concentrations that are inhibitory to other known antibiotics, such as daptomycin. Furthermore, activity against a range of Gram-negative pathogens has been demonstrated, including several on the World Health Organization (WHO) and Centers for Disease Control (CDC) priority lists, including P. aeruginosa, E. coli, E. cloacae, K. pneumoniae, A. baumannii, and S. typhimurium. Likewise, activity against the acid-fast pathogen M. smegmatis has been demonstrated for several Chp peptides, and Chp2-M1 has been demonstrated to have anti-biofilm activity against biofilm comprising Stenotrophomonas species, such as Stenotrophomonas maltophilia.

For several of the Chp peptides, including Chp2, Chp2-M1, Chp4, Chp4-M1, Chp6-M1, Chp10-M1, and Unp2-M1, the ability to synergize in vitro with a range of up to 11 antibiotics against P. aeruginosa, K. pneumoniae, and/or A. baumannii, including antibiotics used in the clinical treatment of Gram-negative infections, has been demonstrated. Additionally, Chp2, Chp2-M1, Chp10-M1, and Chp4 were shown to have potent anti-biofilm activities in the MBEC assay format (MBEC=0.25 μg/mL) and bactericidal activity in the time-kill assay format at concentrations down to 1 μg/mL or lower. See Examples 4 and 5, below.

Overall, these findings are consistent both with a role for the Chp family members in the process of host cell lysis (in the context of the bacteriophage lifecycle) and with the use of purified Chp peptides, modified variants, or derivatives thereof as broad-spectrum antimicrobial agents to target Gram-negative pathogens and/or acid-fast pathogens. One major drawback with the use of previously described AMPs as a treatment for invasive infections concerns toxicity to erythrocytes and a generalized membranolytic activity (i.e., hemolysis) (Oddo A. et al., 2017. Hemolytic Activity of Antimicrobial Peptides. Methods Mol Biol 1548:427-435). Generally, this may be tested in vitro using a standardized assay for detecting the lysis of human red blood cells. Many of the Chp peptides disclosed herein exhibit no hemolytic activity against human red blood cells, in contrast to several AMPs described in the literature (as well as Triton X-100) to have hemolytic activity. In certain embodiments, the Chp peptides disclosed herein may only exhibit minimum hemolytic activity or no hemolytic activity against human red blood cells, as compared to AMPs. Another drawback of AMPs described in the literature concerns a loss of activity in the presence of human blood matrices and physiological salt concentrations (Mohanram H. et al., 2016. Salt-resistant short antimicrobial peptides. Biopolymers 106:345-356); indeed, this effect of known AMPs can be observed as demonstrated in Example 6 and Table 28, below. The data provided herein demonstrate that certain Chp peptides are active in the presence of either human serum or plasma and/or active in growth media, such as Mueller Hinton broth and Casamino Acid medium, containing physiological salt concentrations. Although not wishing to be bound by theory, it is believed that the differences observed in activities of the Chp peptides and AMP peptides (in the literature) may be attributed to the distinct sources of the two types of agents, where the Chp peptides are from phage and the AMPs are based largely on innate immune effectors of vertebrate immune systems. The high activity of Chp peptides, the activity of Chp peptides in blood matrices, and/or the absence of hemolytic activity make them suitable for use in treating invasive diseases. For example, in certain embodiments, the Chp peptides may be active in nanomolar quantities.

In summary, while pathogen-specific targeted lysin therapeutics have the ability to serve as tailored therapy for serious mono-microbial infections caused by known MDR pathogens, there is still an unmet medical need for agents to address serious and life-threatening infections caused by polymicrobial resistant Gram-negative infections (e.g., certain intra-abdominal infections, as well as serious burn, surgical, and other wound infections) and acid-fast bacterial infections (e.g., tuberculosis). The Chp peptides disclosed herein help to meet this need because they have been shown here to exhibit potent activity against all major ESKAPE pathogens (Enterococcus faecium, Staphylococcus aureus, Klebsiella pneumoniae, Acinetobacter baumannii, Pseudomonas aeruginosa, and Enterobacter) commonly associated with MDR, and they are expected to be active against many Gram-negative bacteria, as well as acid-fast bacteria. In certain embodiments, the Chp peptides disclosed herein may be active against other MDR bacterial strains as well, including, for example, MDR bacterial strains from the following species: Citrobacter freundii, Serratia marcescens, Salmonella Senftenberg, Morganella morganii, Raoultella ornithinolytica, Kluyvera ascorbata, Klebsiella oxytoca, Proteus mirabilis, Enterbacter aerogenes, Salmonella Enteritidis, Enterococcus faecium, Salmonella Typhimurium, and Salmonella Oslo. In certain embodiments, the Gram-negative bacteria is a carbapenam-resistant bacterial strain, such as an imipenem-resistant bacterial strain. In certain embodiments, the Gram-negative bacteria is a relebactam-resistant bacterial strain. The Chp peptides disclosed herein may be active at high nanomolar concentrations, comparable to those of active lysins. The Chp peptides disclosed herein may also be responsible for highly potent, rapid, bacteriolytic effects, the ability to clear biofilms, synergy with conventional antibiotics, and synergy with each other, such as synergy between two or more Chp peptides.

Although the Chp peptides of the present disclosure need not be modified by the addition of antimicrobial peptides, in certain embodiments, the Chp peptides disclosed herein may be incorporated into a fusion protein. For example, a fusion protein may comprise a Chp peptide as disclosed herein and a lysin, such as a lysin active against Gram-negative bacteria or may comprise two Chp peptides. In certain embodiments, the Chp peptide may be added to the N-terminus or the C-terminus of a lysin or a second Chp peptide with or without a linker sequence. It is contemplated that fusion polypeptides containing more than one bacteriolytic segment may contribute positively to the bacteriolytic activity of the parent lysin and/or the parent Chp peptide.

### Polypeptides

As demonstrated and explained herein, the Chp peptides described in this section, including wild-type Chp peptides, modified Chp peptides, derivatives, modified variants, or active fragments thereof, can be used in the pharmaceutical compositions and methods described herein.

In some embodiments, the Chp peptide is selected from at least one of Chp1 (SEQ ID NO: 1), Chp2 (SEQ ID NO: 2), CPAR39 (SEQ ID NO: 3), Chp3 (SEQ ID NO: 54); Chp4 (SEQ ID NO: 4), Chp6 (SEQ ID NO: 6), Chp7 (SEQ ID NO: 7), Chp8 (SEQ ID NO: 8), Chp9 (SEQ ID NO: 9), Chp10 (SEQ ID NO: 10), Chp11 (SEQ ID NO: 11), Chp12 (SEQ ID NO: 12), Gkh1 (SEQ ID NO: 13), Gkh2 (SEQ ID NO: 14), Unp1 (SEQ ID NO: 15), Ecp1 (SEQ ID NO: 16), Tma1 (SEQ ID NO: 17), Ecp2 (SEQ ID NO: 18), Osp1 (SEQ ID NO: 19), Unp2 (SEQ ID NO: 20), Unp3 (SEQ ID NO: 21), Gkh3 (SEQ ID NO: 22), Unp5 (SEQ ID NO: 23), Unp6 (SEQ ID NO: 24), Spi1 (SEQ ID NO: 25), Spi2 (SEQ ID NO: 26), Ecp3 (SEQ ID NO: 55), Ecp4 (SEQ ID NO: 56); Lvp1 (SEQ ID NO: 57), Lvp2 (SEQ ID NO: 58), ALCES1 (SEQ ID NO: 59), AVQ206 (SEQ ID NO: 60), AVQ244 (SEQ ID NO: 61), CDL907 (SEQ ID NO: 62), AGT915 (SEQ ID NO: 63), HH3930 (SEQ ID NO: 64), Fen7875 (SEQ ID NO: 65), SBR77 (SEQ ID NO: 66), and Bdp1 (SEQ ID NO: 67) or active fragments thereof having lytic activity.

In some embodiments, the Chp peptide is selected from at least one of Chp2-M1 (SEQ ID NO: 81), Chp2-Cys (SEQ ID NO: 82), Chp2-NC (SEQ ID NO: 83), Chp4::Chp2 (SEQ ID NO: 84), Chp2-CAV (SEQ ID NO: 85), Ecp1-CAV (SEQ ID NO: 86), Ecp1-M1 (SEQ ID NO: 87), Chp6-M1 (SEQ ID NO: 88), Chp10-M1 (SEQ ID NO: 89), Mse-M1 (SEQ ID NO: 90), Chp4-M1 (SEQ ID NO: 91), Chp2-SCR1 (SEQ ID NO: 92), Chp2-SCR1-M1 (SEQ ID NO: 93), Unp4 (SEQ ID NO: 94), Chp7-M1 (SEQ ID NO: 95), Osp1-M1 (SEQ ID NO: 96), Unp2-M1 (SEQ ID NO: 97), Unp3-M1 (SEQ ID NO: 98), Spi2-M1 (SEQ ID NO: 99), Ecp3-M1 (SEQ ID NO: 100), Agt1-M1 (SEQ ID NO: 101), and Myo1 (SEQ ID NO: 102) or active fragments thereof having lytic activity.

The Chp peptide may be a modified Chp peptide or active fragment thereof. In certain embodiments, the Chp peptide or active fragment thereof contains at least one non-naturally occurring modification relative to at least one of SEQ ID NOs. 1-4, 6-26, 54-66, and 81-102, such as at least one amino acid substitution, insertion or deletion.

The modified Chp peptides of the present disclosure are typically designed to retain an α-helix domain, the presence or absence of which can be readily determined using various software programs, such as Jpred4 (compio.dundee.ac.uk/jpred) and Helical Wheel (hael.net/helical.htm).

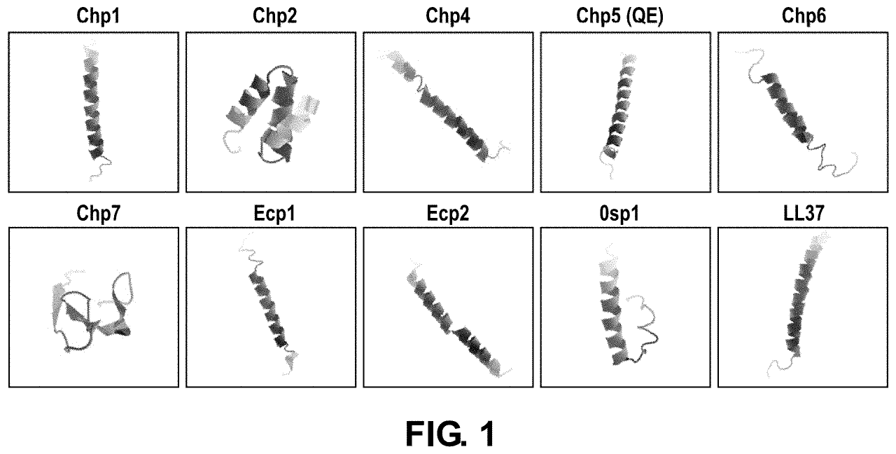

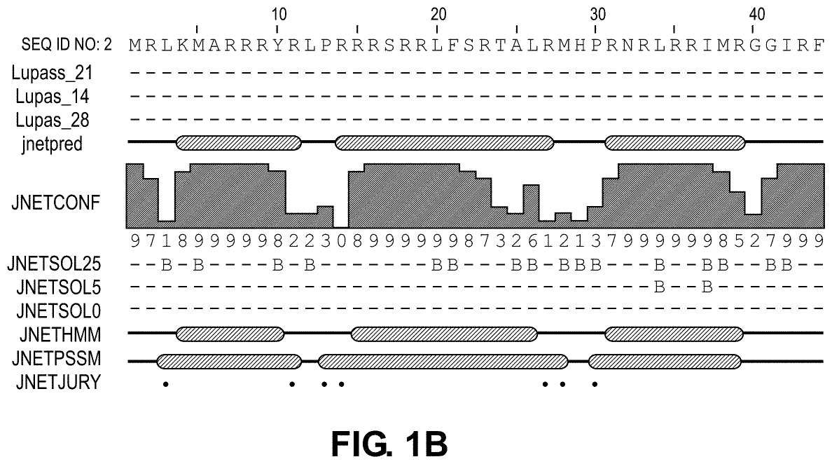

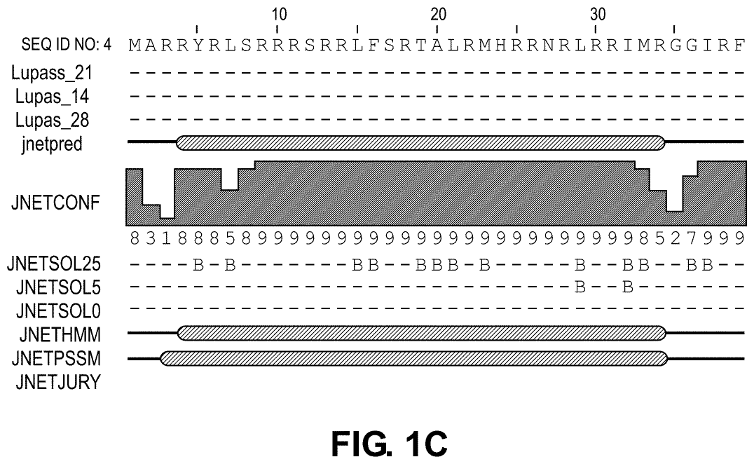

In some embodiments, the α-helix domain spans most of the molecule. See, e.g., Chp1 and Chp4 in FIG. 1. In some embodiments, the α-helix domain is interrupted (see, e.g., Chp2 in FIG. 1), and in some embodiments, the α-helix domain is truncated (see, e.g., Chp6 and Osp1 in FIG. 1). The α-helix domain of the Chp peptides of the present disclosure varies in size between about 3 and 32 amino acids, more typically between about 10 and 25 amino acid residues.

The modified Chp peptides of the present disclosure typically retain one or more functional or biological activities of the reference Chp peptide. In some embodiments, the modification improves the antibacterial activity of the Chp peptide. Typically, the modified Chp peptide has improved in vitro antibacterial activity (e.g., in buffer and/or media) in comparison to the reference Chp peptide. In other embodiments, the modified Chp peptide has improved in vivo antibacterial activity (e.g., in an animal infection model). In some embodiments, the modification improves the antibacterial activity of the Chp peptide in the absence and/or presence of human serum.

In some embodiments, Chp peptides disclosed herein or variants or active fragments thereof are capable of inhibiting the growth of, or reducing the population of, or killing P. aeruginosa and/or at least one species of acid-fast bacteria, such as M. tuberculosis, and, optionally, at least one other species of Gram-negative or acid-fast bacteria in the absence or presence of, or in both the absence and presence of, human serum. In some embodiments, Chp peptides disclosed herein or variants or active fragments thereof are capable of inhibiting the growth of, or reducing the population of, or killing P. aeruginosa and/or at least one species of acid-fast bacteria, such as M. tuberculosis, and, optionally, at least one other species of Gram-negative or acid-fast bacteria in the absence or presence of, or in both the absence and presence of pulmonary surfactant.

In certain embodiments, the modified Chp peptide comprises a polypeptide sequence having at least 80%, such as at least 85%, such as at least 90%, such as at least 92.5%, such as at least 95%, such as at least 98%, or such as at least 99% sequence identity with the amino acid sequence of at least one Chp peptide selected from the group consisting of SEQ ID NOs. 1-4, 6-26, 54-66, 81-91 and 94-102 or an active fragment thereof, wherein the modified Chp peptide inhibits the growth, reduces the population, and/or kills at least one species of Gram-negative bacteria, such as P. aeruginosa, or at least one species of acid-fast bacteria, such as actinobacteria, including mycobacteria, and optionally at least one additional species of Gram-negative or acid-fast bacteria as described herein, optionally in the presence of human serum and/or pulmonary surfactant.

In some embodiments, the Chp peptide is selected from (i) at least one of Chp1 (SEQ ID NO: 1), Chp2 (SEQ ID NO: 2), CPAR39 (SEQ ID NO: 3), Chp3 (SEQ ID NO: 54); Chp4 (SEQ ID NO: 4), Chp6 (SEQ ID NO: 6), Chp7 (SEQ ID NO: 7), Chp8 (SEQ ID NO: 8), Chp10 (SEQ ID NO: 10), Chp11 (SEQ ID NO: 11), Ecp1 (SEQ ID NO: 16), Ecp2 (SEQ ID NO: 18), Ecp3 (SEQ ID NO: 55), Ecp4 (SEQ ID NO: 56), Osp1 (SEQ ID NO: 19), Unp2 (SEQ ID NO: 20), Gkh3 (SEQ ID NO: 22), Unp5 (SEQ ID NO: 23), Unp6 (SEQ ID NO: 24), Spi1 (SEQ ID NO: 25), Lvp1 (SEQ ID NO: 57), ALCES1 (SEQ ID NO: 59), AVQ206 (SEQ ID NO: 60), CDL907 (SEQ ID NO: 62), AGT915 (SEQ ID NO: 63), and SBR77 (SEQ ID NO: 66), or active fragments thereof, or (ii) a modified Chp peptide having at least 80%, such as at least 85%, at least 90%, at least 92.5%, at least 95%, at least 98%, or at least 99% sequence identity with at least one of SEQ ID NOs. 1-4, 6-8, 10, 11, 16, 18, 19, 21-25, 54-57, 59, 60, 62, 63, and 66, wherein the modified Chp peptide inhibits the growth, reduces the population, and/or kills Pseudomonas aeruginosa or at least one species of acid-fast bacteria and optionally at least one additional species of Gram-negative bacteria or acid-fast bacteria, optionally in the presence of human serum and/or pulmonary surfactant.

In some embodiments, the Chp peptide is selected from (i) at least one of Chp2-M1 (SEQ ID NO: 81), Chp2-Cys (SEQ ID NO: 82), Chp2-NC (SEQ ID NO: 83), Chp4::Chp2 (SEQ ID NO: 84), Chp2-CAV (SEQ ID NO: 85), Ecp1-CAV (SEQ ID NO: 86), Ecp1-M1 (SEQ ID NO: 87), Chp6-M1 (SEQ ID NO: 88), Chp10-M1 (SEQ ID NO: 89), Chp4-M1 (SEQ ID NO: 91), Chp7-M1 (SEQ ID NO: 95), Osp1-M1 (SEQ ID NO: 96), Unp2-M1 (SEQ ID NO: 97), Unp3-M1 (SEQ ID NO: 98), Ecp3-M1 (SEQ ID NO: 100), and Agt1-M1 (SEQ ID NO: 101) or active fragments thereof, or (ii) a modified Chp peptide having at least 80%, such as at least 85%, at least 90%, at least 92.5%, at least 95%, at least 98%, or at least 99% sequence identity with at least one of SEQ ID NOs. 81-89, 91, 95-98, 100, and 101, wherein the modified Chp peptide inhibits the growth, reduces the population, and/or kills Pseudomonas aeruginosa or at least one species of acid-fast bacteria and optionally at least one additional species of Gram-negative or acid-fast bacteria, optionally in the presence of human serum and/or pulmonary surfactant.

In some embodiments, the Chp peptide is selected from (i) at least one of Chp2-M1 (SEQ ID NO: 81), Chp2-Cys (SEQ ID NO: 82), Chp2-NC (SEQ ID NO: 83), Chp4::Chp2 (SEQ ID NO: 84), Chp2-CAV (SEQ ID NO: 85), and Ecp1-CAV (SEQ ID NO: 86), or (ii) a modified Chp peptide having at least 80%, such as at least 85%, at least 90%, at least 92.5%, at least 95%, at least 9%, or at least 99% sequence identity with at least one of SEQ ID NOs. 81-86, wherein the modified Chp peptide inhibits the growth, reduces the population, and/or kills Pseudomonas aeruginosa or at least one species of acid-fast bacteria, optionally in the presence of human serum and/or pulmonary surfactant.

In some embodiments, the Chp peptide is selected from (i) at least one of Chp2-M1 (SEQ ID NO: 81), Ecp1-M1 (SEQ ID NO: 87), Chp6-M1 (SEQ ID NO: 88), Chp10-M1 (SEQ ID NO: 89), Chp4-M1 (SEQ ID NO: 91); Unp2-M1 (SEQ ID NO: 97); Ecp3-M1 (SEQ ID NO: 100); and Agt1-M1 (SEQ ID NO: 101), or active fragments thereof, or (ii) a modified Chp peptide having at least 80%, such as at least 85%, at least 90%, at least 92.5%, at least 95%, at least 98%, or at least 99% sequence identity with at least one of SEQ ID NOs. 81, 87, 88, 89, 91, 97, 100, and 101, wherein the modified Chp peptide inhibits the growth, reduces the population, and/or kills at least one species of acid-fast bacteria, optionally in the presence of human serum and/or pulmonary surfactant.

In some embodiments, the Chp peptide is selected from (i) at least one of Chp2 (SEQ ID NO: 2), Chp3 (SEQ ID NO: 54), Chp4 (SEQ ID NO: 4), Chp6 (SEQ ID NO: 6), Ecp1 (SEQ ID NO: 16), and Ecp2 (SEQ ID NO: 18), or active fragments thereof, or (ii) a modified Chp peptide having at least 80%, such as at least 85%, at least 90%, at least 92.5%, at least 95%, at least 98%, or at least 99% sequence identity with at least one of SEQ ID NOs. 2, 4, 6, 16, and 18, wherein the modified Chp peptide inhibits the growth, reduces the population, and/or kills at least one species of Gram-negative bacteria, such as, Pseudomonas aeruginosa and at least one additional species of Gram-negative bacteria, optionally in the presence of human serum and/or pulmonary surfactant.

In certain embodiments, the Chp peptide is selected from (i) at least one Chp peptide having an amino acid sequence selected from the group consisting of SEQ ID NO: 2; SEQ ID NO: 4; and SEQ ID NO: 6 or active fragments thereof, or (ii) a modified Chp peptide having at least 92.5% sequence identity with at least one of SEQ ID NOs. 2, 4, and 6, wherein the modified Chp peptide inhibits the growth, reduces the population, and/or kills at least one species of Gram-negative bacteria, such as Pseudomonas aeruginosa, or at least one species of acid-fast bacteria and at least one additional species of Gram-negative bacteria, optionally in the presence of human serum and/or pulmonary surfactant.

In some embodiments, the Chp peptide of the present disclosure is a derivative of one of the reference Chp peptides that has been chemically modified. A chemical modification includes but is not limited to, adding chemical moieties, creating new bonds, and removing chemical moieties. Chemical modifications can occur anywhere in a Chp peptide, including the amino acid side chains, as well as the amino or carboxyl termini. For example, in certain embodiments, the Chp peptide comprises an N-terminal acetylation modification. In certain embodiments, the Chp peptide or active fragment thereof comprises a C-terminal amidation modification. Such modifications can be present at more than one site in a Chp peptide.

Furthermore, one or more side groups, or terminal groups of a Chp peptide or active fragment thereof may be protected by protective groups known to the person ordinarily-skilled in the art.

In some embodiments, the Chp peptides or active fragments thereof are conjugated to a duration enhancing moiety. In some embodiments, the duration enhancing moiety is polyethylene glycol. Polyethylene glycol (“PEG”) has been used to obtain therapeutic polypeptides of enhanced duration (Zalipsky, S., Bioconjugate Chemistry, 6:150-165 (1995); Mehvar, R., J. Pharm. Pharmaceut. Sci., 3:125-136 (2000), which is herein incorporated by reference in its entirety). The PEG backbone, (CH2CH2-0-)n, wherein n is a number of repeating monomers, is flexible and amphiphilic. When attached to another chemical entity, such as a Chp peptide or active fragment thereof, PEG polymer chains can protect such polypeptides from immune response and other clearance mechanisms. As a result, pegylation can lead to improved efficacy and safety by optimizing pharmacokinetics, increasing bioavailability, and decreasing immunogenicity and dosing amount and/or frequency.

In certain embodiments, the Chp peptide is a modified variant wherein the positive amino acids (arginine, lysine, and histidine), which naturally appear in their L-isoform, have been replaced by the same amino acid in the D-isoform. It has been shown with a different antimicrobial protein derived from sapesin B that variants containing D-isoform amino acids may exhibit higher antimicrobial activity. See, e.g., Manabe et al., Scientific Reports (2017); DOI:10.1038/srep43384. In certain embodiments, the Chp peptide is a modified variant wherein an amino acid residue or residues have been added to the C-terminus, the N-terminus, or both the C-terminus and the N-terminus. For example, in certain embodiments, a cysteine may be added to the C-terminus and/or the N-terminus. In certain embodiments, residues that are known to confer stability to alpha-helices and/or to promote activity in the presence of salt may be added to the C-terminus and/or the N-terminus. See, e.g., Park et al., Helix stability confers salt resistance upon helical antimicrobial peptides, J. Biol. Chem. (2004); 279(14):13896-901. In yet further embodiments, the Chp peptide is a modified variant that is a charge array variant, wherein the amino acids have been reordered based on their charges to maintain amphipathic helical structures. In still further embodiments, the amino acid residues may be scrambled to create the modified variant which may, in certain embodiments, act as a control peptide.

In some embodiments, the Chp peptides disclosed herein and active fragments thereof are capable of penetrating the outer membrane of Gram-negative bacteria. Without being limited by theory, after penetration of the outer membrane, the Chp peptides or active fragments thereof can degrade peptidoglycan, a major structural component of the bacterial cell wall, resulting in cell lysis. In some embodiments, the Chp peptides or active fragments thereof disclosed herein contain positively charged (and amphipathic) N- and/or C-terminal α-helical domains that facilitate binding to the anionic outer membrane of a Gram-negative bacteria to effect translocation into the sub-adjacent peptidoglycan.

The ability of a Chp peptide or active fragment thereof to penetrate an outer membrane of a Gram-negative bacteria may be assessed by any method known in the art, such as described in WO 2017/049233, which is herein incorporated by reference in its entirety. For example, the Chp peptide or active fragment thereof may be incubated with Gram-negative bacteria and a hydrophobic compound. Most Gram-negative bacteria are strongly resistant to hydrophobic compounds, due to the presence of the outer membrane and, thus, do not allow the uptake of hydrophobic agents such as 1-N-phenylnaphthylamine (NPN), crystal violet, or 8-anilino-1-naphthalenesulfonic acid (ANS). NPN is largely excluded by intact Gram-negative bacteria, but enhanced uptake of NPN may occur in cells having a damaged or permeable outer membrane. NPN fluoresces strongly under hydrophobic conditions and weakly under aqueous conditions. Therefore, NPN's interaction with membrane phospholipids in the bacterial envelope increases its fluorescence signal and can be used as an indication of a compromised bacterial membrane and a measurement of the outer membrane permeability.

More particularly, the ability of a Chp peptide or active fragment thereof to penetrate an outer wall may be assessed by incubating, e.g., NPN with a Gram-negative bacteria, e.g., P. aeruginosa strain PA01, in the presence of the Chp peptide or active fragment thereof to be tested for activity. A higher induction of fluorescence in comparison to the fluorescence emitted in the absence of a Chp peptide (negative control) indicates outer membrane penetration. In addition, fluorescence induction can be compared to that of established permeabilizing agents, such as EDTA (ethylene diamine tetraacetate) or an antibiotic such as an antibiotic of last resort used in the treatment of P. aeruginosa, i.e., Polymyxin B (PMB) to assess the level of outer membrane permeabilization.

Multiple protocols throughout the literature detail various method of action studies using NPN and amurin peptides, such as, for example, (1) Mohamed et al., A short D-enantiomeric antimicrobial peptide with potent immunomodulatory and antibiofilm activity against multidrug-resistant Pseudomonas aeruginosa and Acinetobacter baumannii, SCIENTIFIC REPORTS 2017; 6953(7):1-13; (2) Lv et al., Antimicrobial Properties and Membrane-Active Mechanism of a Potential α-Helical Antimicrobial Derived from Cathelicidin PMAP-36, PLoS One 2014; 9:e86364; (3) Wang et al., High specific selectivity and Membrane-Active Mechanism of the synthetic centrosymmetric α-helical peptides with Gly-Gly pairs, SCIENTIFIC REPORTS 2015; 15963(5):1-19; and (4) Shao et al., Symmetrical Modification of Minimized Dermaseptins to Extend the Spectrum of Antimicrobials with Endotoxin Neutralization Potency, INTERNATIONAL J. MOL. SCI. 2019; 1417(20). Accordingly, based on the literature, certain control peptides may be used and compared to the Chp peptides disclosed herein. For example, Lv et al. 2014 discloses melittin, a bee venom peptide that causes rapid (e.g., within minutes) membrane disruption and a fast increase in NPN uptake. Wang et al. 2015 discloses LL-37, a human innate immune molecule that causes membrane disruption and uptake of NPN. Additionally, colistin, a known peptide having potent membrane disruption activity, and charged-reversed variants of the Chp peptides disclosed herein, such as Chp5, may be used as controls, for example in furtherance of the study of the mechanism of action of the Chp peptides disclosed herein.

The ability of a Chp peptide to disrupt the outer membrane of Gram-negative bacteria may be assessed, for example, by measuring the EC₅₀, or the concentration where the bacterial sample had 50% maximal incorporation of NPN into the outer membrane at 10 minutes. When the Chp peptides Chp2, Chp2-M1, and Chp10-M1 were tested for their ability to permeabilize the outer membrane of Gram-negative bacteria (including P. aeruginosa, E. cloacae, K. pneumoniae, E. coli, and A. baumannii), a dose-dependent increase in outer membrane permeability, equivalent to that of colistin, LL37, and melittin, was observed. Thus, in certain embodiments, when Gram-negative bacteria is contacted with a Chp peptide as disclosed herein, the Gram-negative bacteria may exhibit an EC₅₀ comparable to or less than the EC₅₀ of the Gram-negative bacteria exposed to a control peptide, such as colistin, LL-37, or melittin, indicating the Chp peptides disclosed herein allow for increased NPN uptake and percent permeabilization of the outer membrane of Gram-negative bacteria, including P. aeruginosa, E. cloacae, K. pneumoniae, E. coli, and A. baumannii.

The mechanism of action of Chp peptides disclosed herein can also be evaluated by measuring the depolarization of the inner membrane of Gram-negative bacteria. The inner membrane comprises hydroxylated phospholipids such as cardiolipin, phosphatidylglycerol, and phosphatidylserine. This creates a net negative charge at physiological pH, which is believed to enhance the binding of cationic peptides, including the Chp peptides disclosed herein. Upon permeabilization of the outer membrane, the ability of Chp peptides to induce dissipation of the cytoplasmic membrane electrical potential gradient (Δψ) may be examined, for example by following the release of 3,3′-dipropylthiadicarbocyanine (diSC₃-5) as a function of time compared to an untreated control. DiSC₃-5 is a fluorophore that is a caged cation concentrated within the bacterial inner membrane and under the influence of the bacterial membrane electrical potential gradient. At high concentrations, diSC₃-5 self-quenches, leading to the suppression of fluorescence. When the inner membrane deteriorates or becomes leaky for cations, including protons, the A it dissipates, which leads to a release of diSC₃-5 and a subsequent increase in fluorescence. Control peptides such as LL-37 and melittin have been shown to dissipate Δψ, and may be used as a comparison to evaluate the dissipation potential of Chp peptides disclosed herein in various Gram-negative bacteria. When the Chp peptides Chp2, Chp2-M1, and Chp10-M1 were tested for their ability to depolarize the inner membrane of Gram-negative bacteria (including P. aeruginosa, E. cloacae, K. pneumoniae, E. coli, and A. baumannii), a dose-dependent increase in membrane depolarization, equivalent or superior to that of melittin, LL37, RI-18, and PMBN, was observed. Without wishing to be bound by theory, it is believed that the adsorption and binding of Chp peptides to the inner membrane and insertion into the lipid bilayer results in membrane permeabilization and pore/ion channel formation, which is concomitant with the collapse of the membrane's electrical potential.

The damage caused by the Chp peptides disclosed herein to the outer and inner membranes of Gram-negative bacteria can also be assessed with impermeable dyes such as propidium iodide. Protocols for assessing the ability of propidium iodide to cross a bacterial membrane that has been damaged by amurin peptides, intercalate into DNA, and emit a fluorescent signal are known in the art, including, for example, in Mohamed et al. 2017; Wang et al. 2015; Kwon et al., Mechanism of action of antimicrobial peptide P5 truncation against Pseudomonas aeruginosa and Staphylococcus aureus, AMB EXPRESS 2019; 9:122; and Nagant et al., Identification of Peptides Derived from the Human Antimicrobial Peptide LL-37 Active against Biofilms Formed by Pseudomonas aeruginosa Using a Library of Truncated Fragments, ANTIMICROB. AGENTS CHEMO. 2012; 56:5698-5708. As with NPN, the EC₅₀ of propidium iodide may be measured in Gram-negative bacteria contacted with a Chp peptide after a given time and compared to the EC₅₀ of Gram-negative bacteria that is untreated or that has been contacted, for example, with a control peptide such as colistin, LL-37, or melittin. When the Chp peptides Chp2, Chp2-M1, and Chp10-M1 were tested for their ability to enhance the uptake of propidium iodide in Gram-negative bacteria (including P. aeruginosa, E. cloacae, K. pneumoniae, E. coli, and A. baumannii), a dose-dependent increase in cell envelop permeability was equivalent to that of colistin, LL37, and melittin.

Additionally, the mechanism of action of the Chp peptides disclosed herein may be evaluated through the use of Scanning Electron Microscopy (SEM) and Transmission Electron Microscopy (TEM) techniques. Due to the rapid permeabilization and depolarization of bacterial membranes when contacted with Chp peptides, as discussed above, membrane damage may be visualized through SEM and TEM. SEM and TEM images demonstrate that the Chp peptides disclosed herein, as with many known lysins, may be marked by the appearance of “membrane bubbles,” followed by lysis of the membrane. TEM and SEM images taken approximately 2 minutes after Chp peptide (8 μg/mL) contact with P. aeruginosa show the formation of multiple bulges on the bacterial membrane, as well as the appearance of pore formation and degradation of the cellular membrane. TEM and SEM images taken approximately 5 minutes after Chp peptide (8 μg/mL) contact with P. aeruginosa show continued formation of membrane bulges, along with degradation of the cellular membrane, condensation of electron-dense cytoplasmic material, formation of spheroplasts, and pore formation. TEM and SEM images taken approximately 20 minutes after Chp peptide (8 μg/mL) contact with P. aeruginosa show multiple instances of pore formation and the appearance of ghost cells, empty of intracellular content. Taken together, the TEM and SEM images demonstrate that membrane lysis due to contact with Chp peptides disclosed herein may occur through a three-step process comprising membrane bubbling or bulging, pore formation, and cell lysis, resulting in the release of a filamentous material.

In some embodiments, the Chp peptides disclosed herein or active fragments thereof exhibit lytic activity in the presence and/or absence of human serum. Suitable methods for assessing the activity of a Chp peptide or active fragment thereof in human serum are known in the art and described in the examples. Briefly, a MIC value (i.e., the minimum concentration of peptide sufficient to suppress at least 80% of the bacterial growth compared to control) may be determined for a Chp peptide or active fragment thereof and compared to, e.g., a compound inactive in human serum, e.g., T4 phage lysozyme or artilysin GN126. T4 phage lysozyme is commercially available, e.g. from Sigma-Aldrich, Inc. GN126 corresponds to Art-175, which is described in the literature and is obtained by fusing AMP SMAP-29 to GN lysin KZ144. See Briers et al. 2014, Antimicrob, Agents Chemother. 58:3774-3784, which is herein incorporated by reference in its entirety.

In some embodiments, the Chp peptides disclosed herein or active fragments thereof exhibit lytic activity in the presence and/or absence of pulmonary surfactant. Suitable methods for assessing the activity of a Chp peptide or active fragment thereof in pulmonary surfactant are known in the art and described in the examples. As with for assessing the activity in human serum, a MIC value may be determined for a Chp peptide or active fragment thereof in pulmonary surfactant or a suitable substitute (e.g., Survanta®) and optionally compared to a compound exhibiting reduced activity in pulmonary surfactant and/or Survanta®, such as daptomycin.

More particularly MIC values for a Chp peptide or active fragment thereof may be determined against particular bacteria, including e.g., the laboratory P. aeruginosa strains PA01 and CFS-1292, in various standard and non-standard media, e.g., Mueller-Hinton broth (MHB), MHB supplemented with human serum or Survanta®, MHB without starch (MHBns), CAA as described herein, which includes physiological salt concentrations, CAA supplemented with human serum or Survanta®, CAA supplemented with Tween 80®, e.g., 0.002% Tween 80® (CAA_{T}), CAA_{T} supplemented with starch or beef extract, modified RPMI, Dulbecco's Modified Eagle Medium (DMEM), and tryptic soy broth. The use of PA01 enables testing in the presence of elevated serum concentrations since unlike most clinical isolates, PA01 is insensitive to the antibacterial activity of human blood matrices. Other bacteria may also be used to determine MIC values for a Chp peptide or active fragment thereof including, e.g., the laboratory strain Mycobacterium smegmatis MC²155; attenuated Mycobacterium tuberculosis (Zopf) Lehmann and Neumann ATCC® Strains 35818, 25177, 35817, and 35818; and non-tuberculosis mycobacterium strains, including, for example, Mycobacterium avium strain Chester (ATCC® 700898), Mycobacterium kansasii strain Hauduroy (ATCC® 12478), Mycobacterium scrofulaceum strain Prissick and Masson (ATCC® 19981), Mycobacterium peregrinum strain Kusunoki and Ezaki (ATCC® 700686), Mycobacterium marinum strain Aronson (ATCC® 927), Mycobacterium intracellulare strain (Cuttino and McCabe) Runyon (ATCC® 13950), and Mycobacterium fortuitum subspecies fortuitum da Costa Cruz (ATCC® 6841).

In some embodiments, the Chp peptides disclosed herein or active fragments thereof are capable of reducing a biofilm. Methods for assessing the Minimal Biofilm Eradicating Concentration (MBEC) of a Chp peptide or active fragment thereof may be determined using a variation of the broth microdilution MIC method with modifications (See Ceri et al. 1999. J. Clin Microbial. 37:1771-1776, which is herein incorporated by reference in its entirety and Schuch et al., 2017, Antimicrob. Agents Chemother. 61, pages 1-18, which is herein incorporated by reference in its entirety.) In this method, fresh colonies of e.g., a P. aeruginosa strain, such as ATCC 17647, are suspended in medium, e.g., phosphate buffer solution (PBS) diluted e.g., 1:100 in TSBg (tryptic soy broth supplemented with 0.2% glucose), added as e.g., 0.15 ml aliquots, to a Calgary Biofilm Device (96-well plate with a lid bearing 96 polycarbonate pegs; Innovotech Inc.) and incubated e.g., 24 hours at 37° C. Biofilms are then washed and treated with e.g., a 2-fold dilution series of the lysin in TSBg at e.g., 37° C. for 24 hours. After treatment, wells are washed, air-dried at e.g., 37° C. and stained with e.g., 0.05% crystal violet for 10 minutes. After staining, the biofilms are destained in e.g., 33% acetic acid and the OD600 of e.g., extracted crystal violet is determined. The MBEC of each sample is the minimum Chp peptide concentration required to remove at least 95% of the biofilm biomass assessed by crystal violet quantitation.

In some embodiments, the Chp peptides disclosed herein or active fragments thereof reduce the minimum inhibitory concentration (MIC) of an antibiotic in the presence and/or absence of human serum and/or pulmonary surfactant. Any known method to assess MIC may be used. In some embodiments, a checkerboard assay is used to determine the effect of a Chp peptide or active fragment thereof on antibiotic concentration. The checkerboard assay is based on a modification of the CLSI method for MIC determination by broth microdilution (See Clinical and Laboratory Standards Institute (CLSI), CLSI. 2015. Methods for Dilution Antimicrobial Susceptibility Tests for Bacteria That Grow Aerobically; Approved Standard-10th Edition. Clinical and Laboratory Standards Institute, Wayne, Pa., which is herein incorporated by reference in its entirety and Ceri et al. 1999. J. Clin. Microbiol. 37: 1771-1776, which is also herein incorporated by reference in its entirety).

Checkerboards are constructed by first preparing columns of e.g., a 96-well polypropylene microtiter plate, wherein each well has the same amount of antibiotic diluted 2-fold along the horizontal axis. In a separate plate, comparable rows are prepared in which each well has the same amount of Chp peptide or active fragment thereof diluted e.g., 2-fold along the vertical axis. The Chp peptide or active fragment thereof and antibiotic dilutions are then combined, so that each column has a constant amount of antibiotic and doubling dilutions of Chp peptide, while each row has a constant amount of Chp peptide and doubling dilutions of antibiotic. Each well thus has a unique combination of Chp peptide and antibiotic. Bacteria are added to the drug combinations at concentrations of 1×10⁵ GFU/ml in CAA, for example, with or without human serum or pulmonary surfactant. The MIC of each drug, alone and in combination, is then recorded after e.g., 16 hours at 37° C. in ambient air. Summation fractional inhibitory concentrations (ΣFICs) are calculated for each drug and the minimum ΣFIC value (ΣFICmin) is used to determine the effect of the Chp peptide/antibiotic combination.

In some embodiments, the Chp peptides disclosed herein or active fragments thereof show low toxicity against erythrocytes. Any methodology known in the art may be used to assess the potential for hemolytic activity of the present Chp peptides or active fragments thereof.

### Polynucleotides

Chp Peptides and Active Fragments Thereof

In one aspect, the present disclosure is directed to an isolated polynucleotide comprising a nucleic acid molecule encoding a Chp peptide or active fragments thereof having lytic activity. As used herein “lytic activity” encompasses the ability of a Chp peptide to kill bacteria, reduce the population of bacteria or inhibit bacterial growth e.g., by penetrating the outer membrane of a Gram-negative bacteria (e.g., P. aeruginosa) or the cell wall of acid-fast bacteria (e.g., M. tuberculosis) in the presence or absence of human serum. Lytic activity also encompasses the ability to remove or reduce a biofilm and/or the ability to reduce the minimum inhibitory concentration (MIC) of an antibiotic in the presence and/or absence of human serum.

In certain embodiments, the nucleic acid molecule encodes a Chp peptide having an amino acid sequence selected from the group consisting of SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; and SEQ ID NO: 66 or active fragments thereof.

In certain embodiments, the nucleic acid molecule encodes a Chp peptide having an amino acid sequence selected from the group consisting of SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; and SEQ ID NO: 102 or active fragments thereof.

In certain embodiments, the nucleic acid molecule encodes a Chp peptide having an amino acid sequence selected from the group consisting of SEQ ID NO: 81; SEQ ID NO: 82; SEQ ID NO: 83; SEQ ID NO: 84; SEQ ID NO: 85; and SEQ ID NO: 86 or active fragments thereof. In certain embodiments, the nucleic acid molecule encodes a Chp peptide having an amino acid sequence selected from the group consisting of SEQ ID NO: 81; SEQ ID NO: 87; SEQ ID NO: 88; SEQ ID NO: 89; SEQ ID NO: 91; SEQ ID NO: 97; SEQ ID NO: 100; and SEQ ID NO: 101 or active fragments thereof.

In certain embodiments, the nucleic acid molecule encodes a Chp peptide having an amino acid sequence selected from the group consisting of SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 14; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 62; SEQ ID NO: 63; SEQ ID NO: 64; SEQ ID NO: 65; and SEQ ID NO: 66 or active fragments thereof.

In certain embodiments, the nucleic acid molecule encodes a Chp peptide having an amino acid sequence selected from the group consisting of SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 10; SEQ ID NO: 11; SEQ ID NO: 16; SEQ ID NO: 18; SEQ ID NO: 19; SEQ ID NO: 20; SEQ ID NO: 22; SEQ ID NO: 23; SEQ ID NO: 24; SEQ ID NO: 25; SEQ ID NO: 54; SEQ ID NO: 55; SEQ ID NO: 56; SEQ ID NO: 57; SEQ ID NO: 59; SEQ ID NO: 60; SEQ ID NO: 62; SEQ ID NO: 63; and SEQ ID NO: 66 or active fragment thereof, and in certain embodiments, the nucleic acid encodes a Chp peptide having an amino acid sequence selected from the group consisting of SEQ ID NO: 2; SEQ ID NO: 4; SEQ ID NO: 6, SEQ ID NO: 16; SEQ ID NO: 18; and SEQ ID NO: 54 or active fragments thereof.

In some embodiments, the isolated polynucleotides of the present disclosure comprise a nucleic acid molecule that encodes a modified Chp peptide, e.g., a Chp peptide containing one or more insertions, deletions and/or amino acid substitutions in comparison to a reference Chp peptide. Such reference Chp peptides include any one of SEQ ID NOs. 1-4, 6-26, 54-66, and 81-102. In certain embodiments, the modified Chp peptide has at least 80%, at least 85%, at least 90%, at least 95%, at least 98%, or at least 99% sequence identity to a reference Chp polypeptide having the amino acid sequence selected from the group consisting of SEQ ID NOs. 1-4, 6-26, 54-66, and 81-102.

In some embodiments, the nucleic acid molecules of the present disclosure encode an active fragment of the Chp peptides or modified Chp peptides disclosed herein. The term “active fragment” refers to a portion of a full-length Chp peptide, which retains one or more biological activities of the reference peptide. Thus, an active fragment of a Chp peptide or modified Chp peptide, as used herein, inhibits the growth, or reduces the population, or kills P. aeruginosa and/or at least one species of acid-fast bacteria and optionally at least one species of Gram-negative or acid-fast bacteria as described herein in the absence or presence of, or in both the absence and presence of, human serum and/or pulmonary surfactant. Typically, the active fragments retain an α-helix domain. In certain embodiments, the active fragment is a cationic peptide that retains an α-helix domain.

### Vectors and Host Cells

In another aspect, the present disclosure is directed to a vector comprising an isolated polynucleotide comprising a nucleic acid molecule encoding any of the Chp peptides or active fragments thereof disclosed herein or a complementary sequence of the present isolated polynucleotides. In some embodiments, the vector is a plasmid or cosmid. In other embodiments, the vector is a viral vector, wherein additional DNA segments can be ligated into the viral vector. In some embodiments, the vector can autonomously replicate in a host cell into which it is introduced. In some embodiments, the vector can be integrated into the genome of a host cell upon introduction into the host cell and thereby be replicated along with the host genome.

In some embodiments, particular vectors, referred to herein as “recombinant expression vectors” or “expression vectors”, can direct the expression of genes to which they are operatively linked. A polynucleotide sequence is “operatively linked” when it is placed into a functional relationship with another nucleotide sequence. For example, a promoter or regulatory DNA sequence is said to be “operatively linked” to a DNA sequence that codes for an RNA and/or a protein if the two sequences are operatively linked, or situated such that the promoter or regulatory DNA sequence affects the expression level of the coding or structural DNA sequence. Operatively linked DNA sequences are typically, but not necessarily, contiguous.

In some embodiments, the present disclosure is directed to a vector comprising a nucleic acid molecule that encodes a Chp peptide having an amino acid sequence selected from the group consisting of SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; and SEQ ID NO: 66 or active fragments thereof.

In some embodiments, the present disclosure is directed to a vector comprising a nucleic acid molecule that encodes a Chp peptide having an amino acid sequence selected from the group consisting of SEQ ID NO: 81; SEQ ID NO: 82; SEQ ID NO: 83; SEQ ID NO: 84; SEQ ID NO: 85; SEQ ID NO: 86; SEQ ID NO: 87; SEQ ID NO: 88; SEQ ID NO: 89; SEQ ID NO: 90; SEQ ID NO: 91; SEQ ID NO: 92; SEQ ID NO: 93; SEQ ID NO: 94; SEQ ID NO: 95; SEQ ID NO: 96; SEQ ID NO: 97; SEQ ID NO: 98; SEQ ID NO: 99; SEQ ID NO: 100; SEQ ID NO: 101; and SEQ ID NO: 102 or active fragments thereof.

In some embodiments, the present disclosure is directed to a vector comprising a nucleic acid molecule that encodes a Chp peptide having an amino acid sequence selected from the group consisting of SEQ ID NO: 81; SEQ ID NO: 82; SEQ ID NO: 83; SEQ ID NO: 84; SEQ ID NO: 85; and SEQ ID NO: 86 or active fragments thereof. In some embodiments, the present disclosure is directed to a vector comprising a nucleic acid molecule that encodes a Chp peptide having an amino acid sequence selected from the group consisting of SEQ ID NO: 81; SEQ ID NO: 87; SEQ ID NO: 88; SEQ ID NO: 89; SEQ ID NO: 91; SEQ ID NO: 97; SEQ ID NO: 100; and SEQ ID NO: 101 or active fragments thereof.

In certain embodiments, the vector comprises a nucleic acid molecule that encodes a Chp peptide having an amino acid sequence selected from the group consisting of SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 14; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 62; SEQ ID NO: 63; SEQ ID NO: 64; SEQ ID NO: 65; and SEQ ID NO: 66 or active fragments thereof.

In certain embodiments, the vector comprises a nucleic acid molecule that encodes a Chp peptide having an amino acid sequence selected from the group consisting of SEQ ID NO: 1; SEQ ID NO: 2; SEQ ID NO: 3; SEQ ID NO: 4; SEQ ID NO: 6; SEQ ID NO: 7; SEQ ID NO: 8; SEQ ID NO: 10; SEQ ID NO: 11; SEQ ID NO: 16; SEQ ID NO: 18; SEQ ID NO: 19; SEQ ID NO: 20; SEQ ID NO: 22; SEQ ID NO: 23; SEQ ID NO: 24; SEQ ID NO: 25; SEQ ID NO: 54; SEQ ID NO: 55; SEQ ID NO: 56; SEQ ID NO: 57; SEQ ID NO: 59; SEQ ID NO: 60; SEQ ID NO: 62; SEQ ID NO: 63; and SEQ ID NO: 66 or active fragment thereof, and in certain embodiments, the vector comprises a nucleic acid molecule that encodes a Chp peptide having an amino acid sequence selected from the group consisting of SEQ ID NO: 2; SEQ ID NO: 4; SEQ ID NO: 6, SEQ ID NO: 16; SEQ ID NO: 18; and SEQ ID NO: 54 or active fragments thereof.

Generally, any system or vector suitable to maintain, propagate or express a polypeptide in a host may be used for expression of the Chp peptides disclosed herein or active fragments thereof. The appropriate DNA/polynucleotide sequence may be inserted into the expression system by any of a variety of well-known and routine techniques, such as, for example, those set forth in Sambrook et al., eds., Molecular Cloning: A Laboratory Manual (3rd Ed.), Vols. 1-3, Cold Spring Harbor Laboratory (2001). Additionally, tags can also be added to the Chp peptides or active fragments thereof to provide convenient methods of isolation, e.g., c-myc, biotin, poly-His, etc. Kits for such expression systems are commercially available.

A wide variety of host/expression vector combinations may be employed in expressing the polynucleotide sequences encoding the Chp peptides disclosed herein or active fragments thereof. Large numbers of suitable vectors are known to those of skill in the art, and are commercially available. Examples of suitable vectors are provided, e.g., in Sambrook et al, eds., Molecular Cloning: A Laboratory Manual (3rd Ed.), Vols. 1-3, Cold Spring Harbor Laboratory (2001). Such vectors include, among others, chromosomal, episomal and virus derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids.

Furthermore, the vectors may provide for the constitutive or inducible expression of the Chp peptides or active fragments thereof of the present disclosure. Suitable vectors include but are not limited to derivatives of SV40 and known bacterial plasmids, e.g., E. coli plasmids colE1, pCR1, pBR322, pMB9 and their derivatives, plasmids such as RP4, pBAD24 and pBAD-TOPO; phage DNAS, e.g., the numerous derivatives of phage A, e.g., NM989, and other phage DNA, e.g., M13 and filamentous single stranded phage DNA; yeast plasmids such as the 2 D plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. Many of the vectors mentioned above are commercially available from vendors such as New England Biolabs Inc., Addgene, Takara Bio Inc., ThermoFisher Scientific Inc., etc.

Additionally, vectors may comprise various regulatory elements (including promoter, ribosome binding site, terminator, enhancer, various cis-elements for controlling the expression level) wherein the vector is constructed in accordance with the host cell. Any of a wide variety of expression control sequences (sequences that control the expression of a polynucleotide sequence operatively linked to it) may be used in these vectors to express the polynucleotide sequences encoding the Chp peptides or active fragments thereof of the present disclosure. Useful control sequences include, but are not limited to: the early or late promoters of SV40, CMV, vaccinia, polyoma or adenovirus, the lac system, the trp system, the TAC system, the TRC system, the LTR system, the major operator and promoter regions of phage A, the control regions of fd coat protein, the promoter for 3-phosphoglycerate kinase or other glycolytic enzymes, the promoters of acid phosphatase (e.g., Pho5), the promoters of the yeast-mating factors, E. coli promoter for expression in bacteria, and other promoter sequences known to control the expression of genes of prokaryotic or eukaryotic cells or their viruses, and various combinations thereof. Typically, the polynucleotide sequences encoding the Chp peptides or active fragments thereof are operatively linked to a heterologous promoter or regulatory element.

In another aspect, the present disclosure is directed to a host cell comprising any of the vectors disclosed herein including the expression vectors comprising the polynucleotide sequences encoding the Chp peptides or active fragments thereof of the present disclosure. A wide variety of host cells are useful in expressing the present polypeptides. Non-limiting examples of host cells suitable for expression of the present polypeptides include well known eukaryotic and prokaryotic hosts, such as strains of E. coli, Pseudomonas, Bacillus, Streptomyces, fungi such as yeasts, and animal cells, such as CHO, R1.1, B-W and L-M cells, African Green Monkey kidney cells (e.g., COS 1, COS 7, BSC1, BSC40, and BMT10), insect cells (e.g., Sf9), and human cells and plant cells in tissue culture. While the expression host may be any known expression host cell, in a typical embodiment the expression host is one of the strains of E. coli. These include, but are not limited to commercially available E. coli strains such as Top10 (ThermoFisher Scientific, Inc.), DH5a (Thermo Fisher Scientific, Inc.), XLI-Blue (Agilent Technologies, Inc.), SCSllO (Agilent Technologies, Inc.), JM109 (Promega, Inc.), LMG194 (ATCC), and BL21 (Thermo Fisher Scientific, Inc.).

There are several advantages of using E. coli as a host system including: fast growth kinetics, where under the optimal environmental conditions, its doubling time is about 20 min (Sezonov et al., J. Bacterial. 189 8746-8749 (2007)), easily achieved high density cultures, easy and fast transformation with exogenous DNA, etc. Details regarding protein expression in E. coli, including plasmid selection as well as strain selection are discussed in detail by Rosano, G. and Ceccarelli, E., Front Microbial., 5: 172 (2014).

Efficient expression of the present Chp peptides or active fragments thereof depends on a variety of factors such as optimal expression signals (both at the level of transcription and translation), correct protein folding, and cell growth characteristics. Regarding methods for constructing the vector and methods for transducing the constructed recombinant vector into the host cell, conventional methods known in the art can be utilized. While it is understood that not all vectors, expression control sequences, and hosts will function equally well to express the polynucleotide sequences encoding Chp peptides or active fragments thereof of the present disclosure, one skilled in the art will be able to select the proper vectors, expression control sequences, and hosts without undue experimentation to accomplish the desired expression without departing from the scope of this disclosure.

Chp peptides or active fragments thereof of the present disclosure can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. High performance liquid chromatography can also be employed for Chp peptide purification.

Alternatively, the vector system used for the production of Chp peptides or active fragments of the present disclosure may be a cell-free expression system. Various cell-free expression systems are commercially available, including, but are not limited to those available from Promega, LifeTechnologies, Clonetech, etc.

As indicated above, there is an array of choices when it comes to protein production and purification. Examples of suitable methods and strategies to be considered in protein production and purification are provided in WO 2017/049233, which is herein incorporated by reference in its entirety and further provided in Structural Genomics Consortium et al., Nat. Methods., 5(2): 135-146 (2008).

### Pharmaceutical Compositions

The compositions of the present disclosure can take the form of solutions, suspensions, emulsion, tablets, pills, pellets, capsules, capsules containing liquids, powders, sustained-release formulations, suppositories, tampon applications emulsions, aerosols, sprays, suspensions, lozenges, troches, candies, injectants, chewing gums, ointments, smears, time-release patches, liquid absorbed wipes, and combinations thereof.

Administration of the compositions of the present disclosure or pharmaceutically acceptable forms thereof may be topical, i.e., the pharmaceutical composition may be applied directly where its action is desired (for example directly to a wound), or systemic. In turn, systemic administration can be enteral or oral, i.e., the composition may be given via the digestive tract, parenteral, i.e., the composition may be given by other routes than the digestive tract such as by injection or inhalation. Thus, the Chp peptides of the present disclosure and compositions comprising them can be administered to a subject orally, parenterally, by inhalation, topically, rectally, nasally, buccally, via an implanted reservoir, or by any other known method. The Chp peptides of the present disclosure or active fragments thereof can also be administered by means of sustained release dosage forms.

For oral administration, the Chp peptides of the present disclosure or active fragments thereof can be formulated into solid or liquid preparations, for example tablets, capsules, powders, solutions, suspensions, and dispersions. The composition can be formulated with excipients such as, e.g., lactose, sucrose, corn starch, gelatin, potato starch, alginic acid, and/or magnesium stearate.

For preparing solid compositions such as tablets and pills, a Chp peptide of the present disclosure or active fragments thereof may be mixed with a pharmaceutical excipient to form a solid pre-formulation composition. If desired, tablets may be sugar coated or enteric coated by standard techniques. The tablets or pills may be coated or otherwise compounded to provide a dosage form affording the advantage of prolonged action. For example, the tablet or pill can include an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The topical compositions of the present disclosure may further comprise a pharmaceutically or physiologically acceptable carrier, such as a dermatologically or an otically acceptable carrier. Such carriers, in the case of dermatologically acceptable carriers, may be compatible with skin, nails, mucous membranes, tissues, and/or hair, and can include any conventionally-used dermatological carrier meeting these requirements. In the case of otically acceptable carriers, the carrier may be compatible with all parts of the ear. Such carriers can be readily selected by one of ordinary skill in the art. Carriers for topical administration of the compositions of the present disclosure include, but are not limited to, mineral oil, liquid petroleum, white petroleum, propylene glycol, polyoxyethylene and/or polyoxypropylene compounds, emulsifying wax, sorbitan monostearate, polysorbate 60, cetyl esters wax, cetearyl alcohol, 2-octyldodecanol, benzyl alcohol, and water. In formulating skin ointments, the active components of the present disclosure may be formulated, for example, in an oleaginous hydrocarbon base, an anhydrous absorption base, a water-in-oil absorption base, an oil-in-water water-removable base, and/or a water-soluble base. In formulating otic compositions, the active components of the present disclosure may be formulated, for example, in an aqueous polymeric suspension including such carriers as dextrans, polyethylene glycols, polyvinylpyrrolidone, polysaccharide gels, gellan gums such as Gelrite®, cellulosic polymers such as hydroxypropyl methylcellulose, and carboxy-containing polymers such as polymers or copolymers of acrylic acid, as well as other polymeric demulcents. The topical compositions according to the present disclosure may be in any form suitable for topical application, including aqueous, aqueous-alcoholic or oily solutions; lotion or serum dispersions; aqueous, anhydrous or oily gels; emulsions obtained by dispersion of a fatty phase in an aqueous phase (O/W or oil-in-water) or, conversely, (W/O or water-in-oil); microemulsions or alternatively microcapsules, microparticles or lipid vesicle dispersions of ionic and/or nonionic type; creams; lotions; gels; foams (which may use a pressurized canister, a suitable applicator, an emulsifier, and an inert propellant); essences; milks; suspensions; and patches. Topical compositions of the present disclosure may also contain adjuvants such as hydrophilic or lipophilic gelling agents, hydrophilic or lipophilic active agents, preserving agents, antioxidants, solvents, fragrances, fillers, sunscreens, odor-absorbers, and dyestuffs. In a further aspect, the topical compositions disclosed herein may be administered in conjunction with devices such as transdermal patches, dressings, pads, wraps, matrices, and bandages capable of being adhered to or otherwise associated with the skin or other tissue of a subject, being capable of delivering a therapeutically effective amount of one or more Chp peptide or active fragment thereof as disclosed herein.

In one embodiment, the topical compositions of the present disclosure additionally comprise one or more components used to treat topical burns. Such components may include, but are not limited to, a propylene glycol hydrogel; a combination of a glycol, a cellulose derivative, and a water soluble aluminum salt; an antiseptic; an antibiotic; and a corticosteroid. Humectants such as solid or liquid wax esters; absorption promoters such as hydrophilic clays or starches; viscosity building agents; and skin-protecting agents may also be added. Topical formulations may be in the form of rinses such as mouthwash. See, e.g., WO 2004/004650.

The compositions of the present disclosure may also be administered by injection of a therapeutic agent comprising the appropriate amount of a Chp peptide or active fragment thereof and a carrier. For example, the Chp peptide or active fragment thereof can be administered intramuscularly, intrathecally, subdermally, subcutaneously, or intravenously to treat infections by Gram-negative bacteria, such as those caused by P. aeruginosa, and/or infections by acid-fast bacteria, such as those caused by species of actinobacteria, including, for example, M. tuberculosis and non-tuberculosis mycobacteria. The carrier may be comprised of distilled water, a saline solution, albumin, a serum, or any combinations thereof. Additionally, pharmaceutical compositions of parenteral injections can comprise pharmaceutically acceptable aqueous or nonaqueous solutions of Chp peptides as disclosed herein or active fragments thereof in addition to one or more of the following: pH buffered solutions, adjuvants (e.g., preservatives, wetting agents, emulsifying agents, and dispersing agents), liposomal formulations, nanoparticles, dispersions, suspensions or emulsions, as well as sterile powders for reconstitution into sterile injectable solutions or dispersions just prior to use.

In cases where parenteral injection is the chosen mode of administration, an isotonic formulation may be used. Generally, additives for isotonicity can include sodium chloride, dextrose, mannitol, sorbitol, and lactose. In some cases, isotonic solutions such as phosphate buffered saline are preferred. Stabilizers can include gelatin and albumin. A vasoconstriction agent can be added to the formulation. The pharmaceutical preparations according to this type of application may be provided sterile and pyrogen free.

The diluent may further comprise one or more other excipient such as ethanol, propylene glycol, an oil, or a pharmaceutically acceptable emulsifier or surfactant.

In another embodiment, the compositions of the present disclosure are inhalable compositions. The inhalable compositions of the present disclosure can further comprise a pharmaceutically acceptable carrier. In one embodiment, the Chp peptides of the present disclosure or active fragments thereof may be formulated as a dry, inhalable powder. In specific embodiments, an inhalation solution comprising Chp peptides or active fragments thereof may further be formulated with a propellant for aerosol delivery. In certain embodiments, solutions may be nebulized.

A surfactant can be added to an inhalable pharmaceutical composition of the present disclosure in order to lower the surface and interfacial tension between the medicaments and the propellant. Where the medicaments, propellant and excipient are to form a suspension, a surfactant may or may not be used. Where the medicaments, propellant and excipient are to form a solution, a surfactant may or may not be used, depending, for example, on the solubility of the particular medicament and excipient. The surfactant may be any suitable, non-toxic compound which is non-reactive with the medicament and which reduces the surface tension between the medicament, the excipient and the propellant and/or acts as a valve lubricant.

Examples of suitable surfactants include, but are not limited to: oleic acid; sorbitan trioleate; cetyl pyridinium chloride; soya lecithin; polyoxyethylene (20) sorbitan monolaurate; polyoxyethylene (10) stearyl ether; polyoxyethylene (2) oleyl ether; polyoxypropylene-polyoxyethylene ethylene diamine block copolymers; polyoxyethylene (20) sorbitan monostearate; polyoxyethylene(20) sorbitan monooleate; polyoxypropylene-polyoxyethylene block copolymers; castor oil ethoxylate; and combinations thereof.

Examples of suitable propellants include, but are not limited to: dichlorodifluoromethane, trichlorofluoromethane, dichloro-tetrafluoroethane, and carbon dioxide.

Examples of suitable excipients for use in inhalable compositions include, but are not limited to: lactose, starch, propylene glycol diesters of medium chain fatty acids; triglyceride esters of medium chain fatty acids, short chains, or long chains, or any combination thereof; perfluorodimethylcyclobutane; perfluorocyclobutane; polyethylene glycol; menthol; lauroglycol; diethylene glycol monoethylether; polyglycolized glycerides of medium chain fatty acids; alcohols; eucalyptus oil; short chain fatty acids; and combinations thereof.

In some embodiments, the compositions of the present disclosure comprise nasal applications. Nasal applications include applications for direct use, such as nasal sprays, nasal drops, nasal ointments, nasal washes, nasal injections, nasal packings, bronchial sprays and inhalers, as well as applications for indirect use, such as throat lozenges and mouthwashes or gargles, or through the use of ointments applied to the nasal nares or the face, and any combination of these and similar methods of application.

In another embodiment, the pharmaceutical compositions of the present disclosure comprise a complementary agent, including one or more antimicrobial agents and/or one or more conventional antibiotics. In order to accelerate the treatment of the infection, or augment the antibacterial effect, the therapeutic agent containing a Chp peptide of the present disclosure or active fragment thereof may further include at least one complementary agent that can also potentiate the bactericidal activity of the peptide. The complementary agent may be one or more antibiotics used to treat Gram-negative bacteria or one or more antibiotics used to treat acid-fast bacteria. In one embodiment, the complementary agent is an antibiotic or antimicrobial agent used for the treatment of infections caused by P. aeruginosa. In one embodiment, the complementary agent is an antibiotic or antimicrobial agent used for the treatment of infections caused by M. tuberculosis, and in one embodiment, the complementary agent is an antibiotic or antimicrobial agent used for the treatment of infections caused by non-tuberculosis mycobacteria.

The compositions of the present disclosure may be presented in unit dosage form and may be prepared by any methods well known in the art. The amount of active ingredients that can be combined with a carrier material to produce a single dosage form will vary depending, for example, upon the host being treated, the duration of exposure of the recipient to the infectious bacteria, the size and weight of the subject, and the particular mode of administration. The amount of active ingredients that can be combined with a carrier material to produce a single dosage form may, for example, be that amount of each compound which produces a therapeutic effect. In certain embodiments, out of one hundred percent, the total amount may range from about 1 percent to about ninety-nine percent of active ingredients, such as from about 5 percent to about 70 percent, or from about 10 percent to about 30 percent.

### Dosage and Administration

Dosages administered may depend on a number of factors such as the activity of infection being treated; the age, health and general physical condition of the subject to be treated; the activity of a particular Chp peptide or active fragment thereof; the nature and activity of the antibiotic if any with which a Chp peptide or active fragment thereof according to the present disclosure is being paired; and the combined effect of such pairing. In certain embodiments, effective amounts of the Chp peptide or active fragment thereof to be administered may fall within the range of about 1-50 mg/kg (or 1 to 50 mcg/ml). In certain embodiments, effective amounts of the Chp peptide or active fragment thereof to be administered may fall within the range of about 1-50 μg/mL, such as within the range of about 1-10 μg/mL, about 1 μg/mL, or about 10 μg/mL. In certain embodiments, the Chp peptide or active fragment thereof may be administered 1-4 times daily for a period ranging from 1 to 14 days. The antibiotic if one is also used may be administered at standard dosing regimens or in lower amounts in view of any synergism. All such dosages and regimens, however, (whether of the Chp peptide or active fragment thereof or any antibiotic administered in conjunction therewith) are subject to optimization. Optimal dosages can be determined by performing in vitro and in vivo pilot efficacy experiments as is within the skill of the art but taking the present disclosure into account.

It is contemplated that the Chp peptides disclosed herein or active fragments thereof may provide a rapid bactericidal and, when used in sub-MIC amounts, may provide a bacteriostatic effect. It is further contemplated that the Chp peptides disclosed herein or active fragments thereof may be active against a range of antibiotic-resistant bacteria and may not be associated with evolving resistance. Based on the present disclosure, in a clinical setting, the present Chp peptides or active fragments thereof may be a potent alternative (or additive) for treating infections arising from drug- and multidrug-resistant bacteria alone or together with antibiotics (including antibiotics to which resistance has developed). It is believed that existing resistance mechanisms for Gram-negative bacteria do not affect sensitivity to the lytic activity of the present Chp peptides or active fragments thereof.

In some embodiments, time exposure to the Chp peptides disclosed herein or active fragments thereof may influence the desired concentration of active peptide units per ml. Carriers that are classified as “long” or “slow” release carriers (such as, for example, certain nasal sprays or lozenges) may possess or provide a lower concentration of peptide units per ml but over a longer period of time, whereas a “short” or “fast” release carrier (such as, for example, a gargle) may possess or provide a high concentration peptide units (mcg) per ml but over a shorter period of time. There are circumstances where it may be desirable to have a higher unit/ml dosage or a lower unit/ml dosage.

For the Chp peptides or active fragments thereof of the present disclosure, the therapeutically effective dose may be estimated initially either in cell culture assays or in animal models, usually mice, rabbits, dogs, or pigs. The animal model can also be used to achieve a desirable concentration range and route of administration. Obtained information can then be used to determine the effective doses, as well as routes of administration, in humans. Dosage and administration can be further adjusted to provide sufficient levels of the active ingredient or to maintain the desired effect. Additional factors that may be taken into account include the severity of the disease state; age, weight and gender of the patient; diet; desired duration of treatment; method of administration; time and frequency of administration; drug combinations; reaction sensitivities; tolerance/response to therapy; and the judgment of a treating physician.

A treatment regimen can entail administration daily (e.g., once, twice, thrice, etc. daily), every other day (e.g., once, twice, thrice, etc. every other day), semi-weekly, weekly, once every two weeks, once a month, etc. In one embodiment, treatment can be given as a continuous infusion. Unit doses can be administered on multiple occasions. Intervals can also be irregular as indicated by monitoring clinical symptoms. Alternatively, the unit dose can be administered as a sustained release formulation, in which case less frequent administration may be used. Dosage and frequency may vary depending on the patient. It will be understood by one of skill in the art that such guidelines will be adjusted for localized administration, e.g., intranasal, inhalation, rectal, etc., or for systemic administration, e.g., oral, rectal (e.g., via enema), intramuscular (i.m.), intraperitoneal (i.p.), intravenous (i.v.), subcutaneous (s.c.), transurethral, and the like.

### Methods

The Chp peptides and active fragments thereof of the present disclosure can be used in vivo, for example, to treat bacterial infections due to Gram-negative bacteria, such as P. aeruginosa, or due to acid-fast bacteria, such as actinobacteria, in a subject, as well as in vitro, for example to reduce the level of bacterial contamination on, for example, a surface, e.g., of a medical device. In certain embodiments, the Gram-negative bacteria is resistant to at least one antibiotic or is an MDR pathogen.

For example, in some embodiments, the present Chp peptides or active fragments thereof may be used for the prevention, disruption, and/or eradication of bacterial biofilm formed by Gram-negative bacteria or acid-fast bacteria. Biofilm formation occurs when microbial cells adhere to each other and are embedded in a matrix of extracellular polymeric substance (EPS) on a surface. The growth of microbes in such a protected environment that is enriched with biomacromolecules (e.g. polysaccharides, nucleic acids and proteins) and nutrients allow for enhanced microbial cross-talk and increased virulence. Biofilm may develop in any supporting environment including living and nonliving surfaces such as the mucus plugs of the lung (such as the lung of a cystic fibrosis patient), contaminated catheters, contact lenses, etc (Sharma et al. Biologicals, 42(1):1-7 (2014), which is herein incorporated by reference in its entirety). Thus, in one embodiment, the Chp peptides or active fragments thereof of the present disclosure can be used for the prevention, disruption, and/or eradication of bacterial infections due to Gram-negative bacteria or acid-fast bacteria when the bacteria are protected by a bacterial biofilm. In one embodiment, Chp2-M1 or an active fragment thereof can be used for the prevention, disruption, and/or eradication of bacterial infections due to a Gram-negative bacteria when the bacteria are protected by a bacterial biofilm. In one embodiment, Chp2-M1 or an active fragment thereof can be used for the prevention, disruption, and/or eradication of bacterial infections due to a Stenotrophomonas species, such as a Stenotrophomonas maltophilia, when the bacteria are protected by a bacterial biofilm. In certain embodiments, Chp2-M1 or an active fragment thereof can eradicate Gram-negative bacterial biofilm, such as Stenotrophomonas maltophilia bacterial biofilm.

In one aspect, the present disclosure is directed to a method of treating a bacterial infection caused by one or more additional Gram-negative bacteria as described herein, comprising administering to a subject diagnosed with, at risk for, or exhibiting symptoms of a bacterial infection, a pharmaceutical composition as described herein described. In one aspect, the present disclosure is directed to a method of treating a bacterial infection caused by one or more additional acid-fast bacteria as described herein, comprising administering to a subject diagnosed with, at risk for, or exhibiting symptoms of a bacterial infection, a pharmaceutical composition as described herein described.

The terms “infection” and “bacterial infection” are meant to include respiratory tract infections (RTIs), such as respiratory tract infections in patients having cystic fibrosis (CF), lower respiratory tract infections, such as acute exacerbation of chronic bronchitis (ACEB), acute sinusitis, community-acquired pneumonia (CAP), hospital-acquired pneumonia (HAP) and nosocomial respiratory tract infections; sexually transmitted diseases, such as gonococcal cervicitis and gonococcal urethritis; urinary tract infections; acute otitis media; sepsis including neonatal septisemia and catheter-related sepsis; osteomyelitis; tuberculosis, and non-tuberculosis mycobacteria infections. Infections caused by drug-resistant bacteria and multidrug-resistant bacteria are also contemplated.

Non-limiting examples of infections caused by Gram-negative bacteria, such as P. aeruginosa, S. maltophilia, or acid-fast include: A) Nosocomial infections: 1. Respiratory tract infections especially in cystic fibrosis patients and mechanically-ventilated patients; 2. Bacteremia and sepsis; 3. Wound infections, particularly those of burn victims; 4. Urinary tract infections; 5. Post-surgery infections on invasive devises; 6. Endocarditis by intravenous administration of contaminated drug solutions; 7. Infections in patients with acquired immunodeficiency syndrome, cancer chemotherapy, steroid therapy, hematological malignancies, organ transplantation, renal replacement therapy, and other conditions with severe neutropenia. B) Community-acquired infections: 1. Community-acquired respiratory tract infections such as tuberculosis; 2. Meningitis; 3. Folliculitis and infections of the ear canal caused by contaminated water; 4. Malignant otitis externa in the elderly and diabetics; 5. Osteomyelitis of the calcaneus in children; 6. Eye infections commonly associated with contaminated contact lens; 7. Skin infections such as nail infections in people whose hands are frequently exposed to water; 8. Gastrointestinal tract infections; and 9. Musculoskeletal system infections.

The one or more species of Gram-negative bacteria of the present methods may include any of the species of Gram-negative bacteria as described herein. Typically, the additional species of Gram-negative bacteria are selected from one or more of Acinetobacter baumannii, Acinetobacter haemolyticus, Actinobacillus actinomycetemcomitans, Aeromonas hydrophila, Achromobacter spp., such as Achromobacter dolens, Achromobacter ruhlandii, and Achromobacter xylosoxidans, Bacteroides spp., such as, Bacteroides fragilis, Bacteroides theataioatamicron, Bacteroides distasonis, Bacteroides ovatus, and Bacteroides vulgatus, Bartonella Quintana, Bordetella pertussis, Brucella spp., such as, Brucella melitensis, Burkholderia spp, such as, Burkholderia anthina, Burkholderia cepacia, Burkholderia cenacepacia, Burkholderia gladioli, Burkholderia multivorans, Burkholderia pseudomallei, and Burkholderia mallei, Fusobacterium, Prevotella corporis, Prevotella intermedia, Prevotella endodontalis, Porphyromonas asaccharolytica, Campylobacter jejuni, Campylobacter fetus, Campylobacter coli, Chlamydia spp., such as Chlamydia pneumoniae and Chlamydia trachomatis, Citrobacter freundii, Citrobacter koseri, Coxiella burnetii, Edwarsiella spp., such as, Edwarsiella tarda, Eikenella corrodens, Enterobacter spp., such as, Enterobacter cloacae, Enterobacter aerogenes, Enterobacater faecium, and Enterobacter agglomerans, Escherichia coli, Francisella tularensis, Haemophilus influenzae, Haemophilus ducreyi, Helicobacter pylori, Kingella kingae, Klebsiella spp., such as, Klebsiella pneumoniae, Klebsiella oxytoca, Klebsiella rhinoscleromatis, and Klebsiella ozaenae, Kluyvera ascorbata, Legionella penumophila, Moraxella spp., such as, Moraxella catarrhalis, Morganella spp., such as, Morganella morganii, Neisseria gonorrhoeae, Neisseria meningitidis, P. aeruginosa, Pasteurella multocida, Plesiomonas shigelloides, Proteus mirabilis, Proteus vulgaris, Proteus penneri, Proteus myxofaciens, Providencia spp., such as, Providencia stuartii, Providencia rettgeri, Providencia alcalifaciens, Pseudomonas fluorescens, Raoultella ornithinolytica, Salmonella typhi, Salmonella typhimurium, Salmonella paratyphi, Serratia spp., such as, Serratia marcescens, Shigella spp., such as, Shigella flexneri, Shigella boydii, Shigella sonnei, and Shigella dysenteriae, Stenotrophomonas maltophilia, Streptobacillus moniliformis, Vibrio cholerae, Vibrio parahaemolyticus, Vibrio vulnificus, Vibrio alginolyticus, Yersinia enterocolitica, Yersinia pestis, Yersinia pseudotuberculosis, Chlamydia pneumoniae, Chlamydia trachomatis, Ricketsia prowazekii, Coxiella burnetii, Ehrlichia chafeensis and/or Bartonella hensenae.

More typically, the at least one other species of Gram-negative bacteria is selected from one or more of Acinetobacter baumannii, Bordetella pertussis, Burkholderia cepacia, Burkholderia pseudomallei, Burkholderia mallei, Campylobacter jejuni, Campylobacter coli, Enterobacter cloacae, Enterobacter aerogenes, Escherichia coli, Francisella tularensis, Haemophilus influenzae, Haemophilus ducreyi, Helicobacter pylori, Klebsiella pneumoniae, Legionella pneumophila, Moraxella catarrhalis, Morganella morganii, Neisseria gonorrhoeae, Neisseria meningitidis, Pasteurella multocida, Proteus mirabilis, Proteus vulgaris, Salmonella typhi, Serratia marcescens, Shigella flexneri, Shigella boydii, Shigella sonnei, Shigella dysenteriae, Stenotrophomonas maltophilia, Vibrio cholerae, and/or Chlamydia pneumoniae.

Even more typically, the at least one other species of Gram-negative bacteria is selected from one or more of Stenotrophomonas spp. (e.g., Stenotrophomonas maltophilia), Salmonella typhimurium, Salmonella typhi, Shigella spp., Escherichia coli, Acinetobacter baumanii, Klebsiella pneumonia, Neisseria gonorrhoeae, Neisseria meningitides, Serratia spp. Proteus mirabilis, Morganella morganii, Providencia spp., Edwardsiella spp., Yersinia spp., Haemophilus influenza, Bartonella quintana, Brucella spp., Bordetella pertussis, Burkholderia spp., Moraxella spp., Francisella tularensis, Legionella pneumophila, Coxiella burnetii, Bacteroides spp., Enterobacter spp., and/or Chlamydia spp.

Yet even more typically, the at least one other species of Gram-negative bacteria is selected from one or more of Klebsiella spp., Enterobacter spp., Escherichia coli, Citrobacter freundii, Salmonella typhimurium, Yersinia pestis, Stenotrophomonas maltophilia, and/or Franciscella tulerensis.

The one or more species of acid-fast bacteria of the present methods may include any of the species of acid-fast bacteria as described herein. Typically, the additional species of acid-fast bacteria are selected from one or more species of actinobacteria, such as mycobacteria.

Mycobacteria are a family of small, rod-shaped bacilli that can be classified into 3 main groups for the purpose of diagnosis and treatment. The first is Mycobacterium tuberculosis complex which can cause pulmonary tuberculosis and includes M. tuberculosis, M. bovis, M. africanum, M. microti and M. canetti. The second group includes M. leprae and M. lepromatosis, which cause Hansen's disease or leprosy. The third group is nontuberculous mycobacteria (NTM), which include all the other mycobacteria that can cause lung disease resembling tuberculosis, lymphadenitis, skin disease, or disseminated disease. NTM include, but are not limited to, M. avium Complex (MAC), M. avium, M. kansasii, M. abscessus, M. chelonae, M. fortuitum, M. genavense, M. gordonae, M. haemophilum, M. immunogenum, M. intracellulare, M. malmoense, M. marinum, M. mucogenicum, M. nonchromogenicum, M. scrofulaceum, M. simiae, M. smegmatis, M. szulgai, M. terrae, M. terrae complex, M. ulcerans, and M. xenopi. MAC includes at least two mycobacterial species, M. avium and M. intracellulare. These two species cannot be differentiated on the basis of traditional physical or biochemical tests, but there are nucleic acid probes that can be used to identify and differentiate between the two species.

In certain embodiments, the acid-fast bacteria may be selected from one or more of M. smegmatis, M. tuberculosis, M. avium, M. kansasii, M. scrofulaceum, M. peregrinum, M. marinum, M. intracellulare, and/or M. fortuitum.

In some embodiments, infection with Gram-negative bacteria or acid-fast bacteria results in a localized infection, such as a topical bacterial infection, e.g., a skin wound. In other embodiments, the bacterial infection is a systemic pathogenic bacterial infection. Common acid-fast infections include tuberculosis and non-tuberculosis mycobacteria infections. Common Gram-negative pathogens and associated infections are listed in Table A of the present disclosure. These are meant to serve as examples of the bacterial infections that may be treated, mitigated or prevented with the present Chp peptides and active fragments thereof and are not intended to be limiting.

TABLE A
Medically relevant Gram-negative bacteria and associated diseases

| | |
|---|---|
| Salmonella typhimurium | Gastrointestinal (GI) infections—salmonellosis |
| Shigella spp. | shigellosis |
| Escherichia coli | Urinary tract infections (UTIs) |
| Acinetobacter baumanii | Wound infections |
| Pseudomonas aeruginosa | bloodstream infections and pneumonia |
| Klebsiella pneumoniae | UTIs, and bloodstream infections |
| Neisseria gonorrhoeae | Sexually transmitted diseases (STDs)—gonorrhea |
| Neisseria meningitides | Meningitis |
| Serratia spp. | Catheter contaminations, UTIs, and pneumonia |
| Proteus mirabilis | UTIs |
| Morganella spp. | UTIs |
| Providencia spp. | UTIs |
| Edwardsiella spp | UTIs |
| Salmonella typhi | GI infections—typhoid fever |
| Yersinia pestis | Bubonic and pneumonic plague |
| Yersinia enterocolitica | GI infections |
| Yersinia pseudotuberculosis | GI infections |
| Haemophilus influenza | Meningitis |
| Bartonella Quintana | Trench fever |
| Brucella spp. | Brucellosis |
| Bordetella pertussis | Respiratory—Whooping cough |
| Burkholderia spp. | Respiratory |
| Moraxella spp. | Respiratory |
| Francisella tularensis | Tularemia |
| Legionella pneumophila | Respiratory—Legionnaires’ disease |
| Coxiella burnetii | Q fever |
| Bacteroides spp. | Abdominal infections |
| Enterobacter spp. | UTIs and respiratory |
| Chlamydia spp. | STDs, respiratory, and ocular |
| Stenotrophomonas spp. | Medical device contaminations, UTIs, |
| | bloodstream infections, and pneumonia |

In some embodiments, the Chp peptides and active fragments thereof of the present disclosure are used to treat a subject at risk for acquiring an infection due to Gram-negative bacterium or acid-fast bacterium. Subjects at risk for acquiring a Gram-negative or acid-fast bacterial infection include, for example, cystic fibrosis patients, neutropenic patients, patients with necrotising enterocolitis, burn victims, patients with wound infections, and, more generally, patients in a hospital setting, in particular surgical patients and patients being treated using an implantable medical device such as a catheter, for example a central venous catheter, a Hickman device, or electrophysiologic cardiac devices, for example pacemakers and implantable defibrillators. Other patient groups at risk for infection with Gram-negative or acid-fast bacteria include without limitation patients with implanted prostheses such a total joint replacement (for example total knee or hip replacement).

In another aspect, the present disclosure is directed to a method of preventing or treating a bacterial infection comprising co-administering to a subject diagnosed with, at risk for, or exhibiting symptoms of a bacterial infection, a combination of a first effective amount of the composition containing an effective amount of a Chp peptide or active fragment thereof as described herein, and a second effective amount of an antibiotic suitable for the treatment of Gram-negative bacterial infection. In certain aspects, the present disclosure is directed to a method of preventing or treating a bacterial infection comprising co-administering to a subject diagnosed with, at risk for, or exhibiting symptoms of a bacterial infection, a combination of a first effective amount of the composition containing an effective amount of a Chp peptide or active fragment thereof as described herein, and a second effective amount of an antibiotic suitable for the treatment of an acid-fast bacterial infection.

The Chp peptides and active fragments thereof of the present disclosure can be co-administered with standard care antibiotics or with antibiotics of last resort, individually or in various combinations as within the skill of the art. Traditional antibiotics used against mycobacterial infections include, for example, macrolides (clarithromycin, azithromycin), ethambutol, rifamycins (rifampin, rifabutin), isoniazid, pyrazinamide, and aminoglycosides (streptomycin, amikacin). Traditional antibiotics used against P. aeruginosa are described in Table B. Antibiotics for other Gram-negative bacteria, such as Klebsiella spp., Enterobacter spp., Escherichia coli, Citrobacter freundii, Salmonella typhimurium, Yersinia pestis, and Franciscella tulerensis, are similar to that provided in Table B for P. aeruginosa.

TABLE B
Antibiotics used for the treatment of Pseudomonas aeruginosa

| Class | Agent |
|---|---|
| Penicillins | Ticarcillin-clavulanate |
| | Piperacillin-tazobactam |
| Cephalosporins | Ceftazidime |
| | Cefepime |
| | Cefoperazone |
| Monobactams | Aztreonam |
| Fluoroquinolones | Ciprofloxacin |
| | Levofloxacin |
| Carbapenems | Imipenem |
| | Meropenem |
| | Doripenem |
| Aminoglycosides | Gentamicin |
| | Tobramycin |
| | Amikacin |
| Polymixins | Colistin |
| | Polymixin B |

In more specific embodiments, the antibiotic is selected from one or more of ceftazidime, cefepime, cefoperazone, ceftobiprole, ciprofloxacin, levofloxacin, aminoglycosides, imipenem, meropenem, doripenem, gentamicin, tobramycin, amikacin, piperacillin, ticarcillin, penicillin, rifampicin, polymyxin B and colistin. In certain embodiments, the antibiotic is chosen from isoniazid, rifampin, ethambutol, and pyrazinamide.

Combining the Chp peptides or active fragments thereof of the present disclosure with antibiotics provides an efficacious antibacterial regimen. In some embodiments, co-administration of Chp peptides or active fragments thereof of the present disclosure with one or more antibiotics may be carried out at reduced doses and amounts of either the Chp peptides or active fragments thereof or the antibiotic or both, and/or reduced frequency and/or duration of treatment with augmented bactericidal and bacteriostatic activity, reduced risk of antibiotic resistance and with reduced risk of deleterious neurological or renal side effects (such as those associated with colistin or polymyxin B use). Prior studies have shown that total cumulative colistin dose is associated with kidney damage, suggesting that decrease in dosage or shortening of treatment duration using the combination therapy with Chp peptides or active fragments thereof could decrease the incidence of nephrotoxicity (Spapen et al. Ann Intensive Care. 1: 14 (2011), which is herein incorporated by reference in its entirety). As used herein the term “reduced dose” refers to the dose of one active ingredient in the combination compared to monotherapy with the same active ingredient. In some embodiments, the dose of Chp peptides or active fragments thereof or the antibiotic in a combination may be suboptimal or even subthreshold compared to the respective monotherapy.

In some embodiments, the present disclosure provides a method of augmenting antibiotic activity of one or more antibiotics against Gram-negative or acid-fast bacteria compared to the activity of said antibiotics used alone by administering to a subject the Chp peptides or active fragments thereof disclosed herein together with an antibiotic of interest. The combination is effective against the bacteria and permits resistance against the antibiotic to be overcome and/or the antibiotic to be employed at lower doses, decreasing undesirable side effects, such as the nephrotoxic and neurotoxic effects of polymyxin B.

The Chp peptides or active fragments thereof optionally in combination with antibiotics of the present disclosure can be further combined with additional permeabilizing agents of the outer membrane of the Gram-negative bacteria, including, but not limited to metal chelators, such as e.g. EDTA, TRIS, lactic acid, lactoferrin, polymyxins, citric acid (Vaara M. Microbial Rev. 56(3):395-441 (1992), which is herein incorporated by reference in its entirety).

In yet another aspect, the present disclosure is directed to a method of inhibiting the growth, or reducing the population, or killing of at least one species of Gram-negative bacteria or acid-fast bacteria, the method comprising contacting the bacteria with a composition containing an effective amount of a Chp peptide or active fragment thereof as described herein, wherein the Chp peptide or active fragment thereof inhibits the growth, or reduces the population, or kills at least one species of Gram-negative bacteria or acid-fast bacteria.

In some embodiments, inhibiting the growth, or reducing the population, or killing at least one species of Gram-negative bacteria or acid-fast bacteria comprises contacting bacteria with the Chp peptides or active fragments as described herein, wherein the bacteria are present on a surface of e.g., medical devices, floors, stairs, walls and countertops in hospitals and other health related or public use buildings and surfaces of equipment in operating rooms, emergency rooms, hospital rooms, clinics, and bathrooms and the like.

Examples of medical devices that can be protected using the Chp peptides or active fragments thereof described herein include but are not limited to tubing and other surface medical devices, such as urinary catheters, mucous extraction catheters, suction catheters, umbilical cannulae, contact lenses, intrauterine devices, intravaginal and intraintestinal devices, endotracheal tubes, bronchoscopes, dental prostheses and orthodontic devices, surgical instruments, dental instruments, tubings, dental water lines, fabrics, paper, indicator strips (e.g., paper indicator strips or plastic indicator strips), adhesives (e.g., hydrogel adhesives, hot-melt adhesives, or solvent-based adhesives), bandages, tissue dressings or healing devices and occlusive patches, and any other surface devices used in the medical field. The devices may include electrodes, external prostheses, fixation tapes, compression bandages, and monitors of various types. Medical devices can also include any device which can be placed at the insertion or implantation site such as the skin near the insertion or implantation site, and which can include at least one surface which is susceptible to colonization by Gram-negative bacteria and/or acid-fast bacteria.

### EXAMPLES

### Materials and Methods

Bacterial strains and growth conditions. The majority of studies disclosed herein were performed using a carbapenem-resistant P. aeruginosa clinical isolate CFS-1292 obtained from human blood at the Hospital for Special Surgery in New York (provided by Dr. Lars Westblade, Professor of Pathology and Laboratory Medicine), but commercially available antibiotic resistant isolates may also be used. All other isolates were obtained from either the American Type Culture Collection (“ATCC”), the d'Herelle collection (“HER”), BEI Resources (“HM”), or the Hospital for Special Surgery in New York (“HSS”). Isolates were cultured and tested in either lysogeny broth (LB; Sigma-Aldrich), casamino acid (CAA) media (5 g/L casamino acids, Ameresco/VWR; 5.2 mM K₂HPO₄, Sigma-Aldrich; 1 mM MgSO₄, Sigma-Aldrich), CAA supplemented with 100 mM NaCl, or CAA supplemented with 2.5% human serum (Type AB, male, pooled; Sigma-Aldrich). All antibiotics and protein reagents (e.g., T4 lysozyme) were obtained from Sigma-Aldrich unless otherwise indicated.

Bioinformatic studies. All proteins were identified in annotated GenBank database entries for all Microviridae and Leviviridae genomes. The accession number for each Chp group peptide is indicated in Tables 1 and 2 below. Blastp analyses were performed using the UniProt server, available at uniprot.org/blast/. Protein secondary structure predictions were performed using JPRED4, available at www.compbio.dundee.ac.uk/jpred/index, and I-Tasser, available at www.zhanglab.ccmb.med.umich.edu/I-TASSER/. Phylogenetic analyses were performed using ClustalW Multiple Sequence Alignment tools, available at www.genome.jp/tools-bin/clustalw. Predicted molecular weights and isoelectric points were determined using the ExPASy Resource Portal, available at web.expasy.org/compute_pi/.

Determination of Minimal Inhibitory Concentrations (MIC). MIC values were determined using a modification of the standard broth microdilution reference method defined by the Clinical and Laboratory Standards Institute (CLSI) (2015. Methods for Dilution Antimicrobial Susceptibility Tests for Bacteria That Grow Aerobically; Approved Standard-10th Edition. Clinical and Laboratory Standards Institute, Wayne, Pa.). The modification was based on the replacement of Mueller Hinton Broth, in some instances, with either CAA media (with and without NaCl) or CAA supplemented with 2.5% human serum. As used herein, MIC is the minimum concentration of peptide sufficient to suppress at least 80% of the bacterial growth compared to control.

Determination of Minimal Biofilm Eradicating Concentrations (MBEC). MBEC values were determined using a variation of the broth microdilution MIC method with modifications (Ceri H et al., 1999. J Clin Microbiol 37:1771-1776; and Schuch R et al., 2017. Antimicrob Agents Chemother 61). Fresh colonies of P. aeruginosa strain ATCC 17647 were suspended in PBS (0.5 McFarland units), diluted 1:100 in LB with 0.2% glucose, added as 0.15 ml aliquots to each well of a 96-well Calgary Biofilm Device (Innovotech), and incubated for 24 hours at 37° C. for the formation of biofilms on polycarbonate pegs. Biofilms were washed and treated with a 2-fold dilution series of each peptide in TSBg at 37° C. for 16 hours. After treatment, wells were washed, air-dried at 37° C., stained with 0.05% crystal violet for 10 minutes, and destained in 33% acetic acid. The OD₆₀₀ of extracted crystal violet was determined. The MBEC value of each sample was determined as the minimum drug concentration required to remove >95% of biofilm biomass as assessed by crystal violet quantitation (in comparison to untreated controls). T4 phage lysozyme was used as a negative control and does not provide anti-biofilm activity.

Checkerboard assays. The checkerboard assay is based on a modification of the CLSI method for MIC determination by broth microdilution (CLSI 2015; and Moody J. 2010. Synergy testing: broth microdilution checkerboard and broth macrodilution methods, p 5.12.11-15.12.23. In Garcia L S (ed), Clinical Microbiology Procedures Handbook, vol 2). Checkerboards were constructed by first preparing columns of a 96-well polypropylene microtiter plate, in which each well had the same amount of antibiotic diluted 2-fold along the horizontal axis. In a separate plate, comparable rows were prepared in which each well had the same amount of peptide diluted 2-fold along the vertical axis. The peptide and antibiotic dilutions were then combined, so that each column had a constant amount of antibiotic and doubling dilutions of Chp peptide, while each row had a constant amount of Chp peptide and doubling dilutions of antibiotic. Each well thus had a unique combination of peptide and antibiotic. Bacteria were added to each well at a concentration of 1×105 CFU/mL in CAA with 2.5% human serum. The MIC of each agent, alone and in combination, was then recorded after 16 hours at 37° C. in ambient air, unless otherwise indicated. Summation fractional inhibitory concentration index (FICIs) were calculated for each drug and the minimum FICI was used to determine synergy. FICIs were calculated as follows: FICI=FIC A+FIC B, where FIC A is the MIC of each antibiotic in the combination/MIC of the each antibiotic alone, and FIC B is the MIC of each Chp peptide in the combination/MIC of each Chp peptide alone. The combination is considered synergistic when the FICI is ≤0.5, strongly additive when the FICI is >0.5 to <1, additive with the FICI is 1-<2, and antagonistic when the FICI is ≥2. Checkerboard assays were performed using P. aeruginosa strain CFS-1292 in CAA/HuS with combinations of either Chp2 or Chp4 against a range of 11 different antibiotics, including amikacin, azithromycin, aztreonam, ciprofloxacin, colistin, fosfomycin, gentamicin, imipenem, piperacillin, rifamipicin, and tobramycin. FICI values of ≤0.5 were observed for the majority of combinations, indicating the ability of Chp2 and Chp4 to synergize with a broad range of antibiotics (see Table 8 below). These findings suggest that the Chp peptides may provide potent antibacterial activity in the presence of antibiotics.

Assay of Chp Peptide Hemolytic Activity. Hemolytic activity was measured as the amount of hemoglobin released by the lysis of human erythrocytes (Lv Y et al, 2014. PLoS One 9:e86364). Briefly, 3 ml of fresh human blood cells (hRBCs) obtained from pooled healthy donors (BioreclamationIVT) in a polycarbonate tube containing heparin were centrifuged at 1,000×g for 5 min at 4° C. The erythrocytes obtained were washed three times with phosphate-buffered saline (PBS) solution (pH 7.2) and resuspended in 30 ml PBS. A 50 μl volume of the erythrocyte solution was incubated with 50 μl of each Chp peptide (in PBS) in a 2-fold dilution range (from 128 μg/mL to 0.25 μg/mL) for 1 h at 37° C. Intact erythrocytes were pelleted by centrifugation at 1,000×g for 5 min at 4° C., and the supernatant was transferred to a new 96-well plate. The release of hemoglobin was monitored by measuring the absorbance at an optical density (OD) of 570 nm. The minimal hemolytic concentration was determined as lowest peptide concentration exhibiting visual lysis (which corresponds to the minimal concentration resulting in an OD value ≥5% of the untreated control sample). Additional controls were used including hRBCs in PBS treated as above with either 0.1% Triton X-100 or each of a series of antimicrobial peptides with known hemolytic activity, including RR12, RR12polar and RR12hydrophobic (Mohanram H. et al, 2016. Biopolymers 106:345-356), and with little or no hemolytic activity, including RI18 (Lyu Y. et al., 2016. Sci Rep 6:27258) and RR22.

Time-Kill Assay of Chp Peptide Activity. An overnight culture of P. aeruginosa strain CFS-1292 was diluted 1:100 into fresh CAA media with 2.5% human serum (CAA/HuS) and grown for 2.5 hours at 37° C. with agitation. Exponential phase bacteria were then diluted 1:100 into CAA/HuS and peptide was added at a final concentration of either 1 or 10 μg/mL. Control cultures were included with no peptide added (i.e., buffer control). Cultures were incubated at 37° C. with aeration and at 1 hr, 3 hr, and 24 hr time-points, samples were removed for quantitative plating on CAA agar plates.

Microscopy. Aliquots of P. aeruginosa strain CFS-1292, grown for 2.5 hours in LB, were washed with PBS and resuspended in either PBS or 100% human serum and treated for 15 minutes at room temperature with and without peptide Chp2 at a final concentration of 10 μg/mL. Sample subsets were stained using the Live/Dead Cell Viability Kit (ThermoFisher) according to the manufacturer's protocol and examined by differential interference contrast (DIC) microscopy and fluorescence microscopy.

### Example 1: Identification of Chp Peptides

Having knowledge of certain poorly described bacteriophage (Chlamydiamicroviridae) that specifically infect and kill the Gram-negative bacteria Chlamydia, published genomes of these organisms were studied, initially looking to identify novel lysins, although no lysin-like sequences nor any sequences similar to previously described amurins were observed. Chlamydia do not utilize peptidoglycans (a known target of lysins) in their structures as abundantly as other bacteria, but rather Chlamydia generally only use peptidoglycans during division. Therefore, the question arose as to what the target of Chlamydia phage was. It was postulated that the mechanism by which Chlamydia phage invade their target may be different from the ones previously known, and their target may be different and focused on lipopolysaccharide (LPS), a main constituent of the outer membrane of Gram-negative bacteria and an obstacle to penetration by lysins of the outer membrane.

The published genomes of Chlamydiamicrovirus were studied with a view to identifying syntenic loci, i.e., similar genes in the same position in a genome of a group of genetically related phages, which suggested similar function. Small highly cationic peptides were identified that had a very similar molecular charge profile to previously identified antimicrobial peptides (AMPs). While the Chlamydia phage sequences had no protein sequence similarity to AMPs, lysins, or to known amurin proteins (such as Protein A2, protein E and others), the overall positive charge was a prominent feature. Using bioinformatic techniques as described above (JPRED and iTASSAR), structural predictions were conducted that revealed the presence of alpha helices, a hallmark feature of many AMPs. The alpha helices, the overall charge, the conservation among Chlamydia, and the related Gram-negative bacteria phage genomes all suggested that these proteins may represent a family of previously uncharacterized phage lytic polypeptides and that they may define a previously undescribed phage lytic mechanism. The fact that they were predicted to be small in size and soluble (based on their charge profile) also meant that, once synthesized, they would likely be readily amenable to testing by simply adding them to susceptible bacteria cultures.

Based on the foregoing, 12 conserved sequences within syntenic loci were extracted from the Microviridae genomes in the GenBank database and specifically from the Chlamydiamicroviruses genomes (as well as some other viruses described below). The 12 conserved sequences were annotated only as hypothetical, uncharacterized or non-structural proteins and encoded small (putatively) cationic proteins predicted to adopt alpha-helical structures. These 12 sequences are set forth in Table 1. One of the peptides in Table 1, Chp5 was synthesized to have a molecular charge different from Chp4 by replacing arginines and lysines, which are positively charged, with negatively charges amino acid residues. Chp5 was predicted to be inactive. While these peptides exhibit no sequence similarity to other lytic or antimicrobial proteins, they are predicted to adopt alpha-helical structures (for examples, see FIG. 1) similar to subsets of the large family of antibacterial agents AMPs. It was postulated that Chp peptides perform the host lysis function for the phages from which they are derived.

Based on the foregoing considerations, further study of genomes of other phages (related to the Chlamydiamicroviruses, in the same family, Microviridae) that infect Gram-negative bacteria, as well as other uncharacterized sources that presented with the same synteny and charge profile, yielded 29 additional peptides listed in Table 2. Together, all 41 peptides (excluding Chp5) form a related family of novel phage lytic agents. They are all from Microviridae sources, with the exception of Myo1 (SEQ ID NO: 102), which is from Microbacterium.

Furthermore, certain of these peptides were modified to synthesize novel variants. Notably, for Chp2, the L-form of each of the positively charged amino acids (arginine and lysine) was substituted for the D-form of that amino acid, to see if the D-form may enhance antibacterial activity.

This modification resulted in Chp2-M1 (SEQ ID. NO: 81). Similar D-form variants were created from the native Chp peptides or modified variants of Chp peptides to arrive at Ecp1-M1 (SEQ ID NO: 87), Chp6-M1 (SEQ ID NO: 88), Chp10-M1 (SEQ ID NO: 89), Mse-M1 (SEQ ID NO: 90), Chp4-M1 (SEQ ID NO: 91), Chp2-SCR-M1 (SEQ ID NO: 93), Chp7-M1 (SEQ ID NO: 95), Osp-M1 (SEQ ID NO: 96), Unp2-M1 (SEQ ID NO: 97), Unp3-M1 (SEQ ID NO: 98), Spi2-M1 (SEQ ID NO: 99), Ecp3-M1 (SEQ ID NO: 100), and Agt1-M1 (SEQ ID NO: 101).

Likewise for Chp2, a cysteine residue was added to the C-terminus to arrive at Chp2-Cys (SEQ ID NO: 82), and additional residues previously shown to confer alpha-helix stability and promote activity in the presence of salt were added to both the C-terminus and the N-terminus to arrive at Chp2-NC (SEQ ID NO: 83). Park et al., Helix stability confers salt resistance upon helical antimicrobial peptides, J. Biol. Chem. (2004); 279(14):13896-901.

Chp4::Chp2 (SEQ ID NO: 84) is a fusion peptide comprising alpha helices from Chp4 (SEQ ID NO: 4) and Chp2 (SEQ ID NO: 2). Chp2-CAV (SEQ ID NO: 85) and Ecp1-CAV (SEQ ID NO: 86) are charge array variants, wherein various amino acid charges were reordered to maintain amphipathic helices. Chp2-SCR1 (SEQ ID NO: 92) is a modified variant of Chp2 (SEQ ID NO: 2), wherein the amino acid residues have been scrambled to create a control peptide.

Thus, a complete list of all Chp family members (including certain features of each peptide) is provided in Table 1, Table 2, and Table C. Included in this group are peptides Chp1-4 and 6-12 and CPAR39, which are derived from 11 different Chlamydiamicroviruses and are described in Table 1; peptides Chp2 and Chp3 are two identical peptides from two different phages. As stated above, Chp5 is a modified derivative of Chp4 generated by the replacement of all positively charged amino acids, including arginines and lysines, with negatively charged amino acids, including glutamine and glutamic acid. The additional members of the Chp family were identified by homology with the Chlamydiamicrovirus proteins and are described in Table 2 (“Additional Chp family members”). The additional Chp family members are not from Chlamydiamicrovirus sources but from putative Microviridae and Microbacterium phage sources. Table C provides several modified variants of Chp peptides, including D-form variants and charge array variants as discussed above. In Table C, amino acids that are italicized and in bold indicate amino acid residues that have been changed from the L-form to the D-form.

TABLE 1
Chlamydia phage (Chp)-derived lytic agents

| Protein | Identifier | Protein | pI/kDa | |
|---|---|---|---|---|
| name | Information | Sequence | (amino acids) | DNA Sequence |
| Chp1 | Phage Chp1 | MVRRRRLRR | 13.23/4669.64 | ATGGTTCGTAGAAGA |
| | Gene: Chp1p08 | RISRRIFRR | (36) | CGTTTGAGAAGAAGA |
| | GenBank: | TVARVGRRR | | ATAAGTAGAAGAATT |
| | NP_044319.1 | RSFRGGIRF | | TTTAGAAGAACAGTA |
| | Family: | (SEQ ID | | GCTAGAGTTGGTAGA |
| | Microviridae | NO: 1) | | AGGCGAAGGTCTTTT |
| | | | | CGTGGTGGTATTAGA |
| | | | | TTTTAA (SEQ ID |
| | | | | NO: 27) |
| Chp2 | Phage 2 | MRLKMARRR | 12.90/5708.98 | ATGAGGTTAAAAATG |
| | Gene: Ch-2p5 | YRLPRRRSR | (44) | GCACGAAGAAGATAC |
| | GenBank: | RLFSRTALR | | AGACTTCCGCGACGT |
| | NP_054652.1 | MHPRNRLRR | | AGAAGTCGAAGACTT |
| | Family: | IMRGGIRF | | TTTTCAAGAACTGCA |
| | Microviridae | (SEQ ID | | TTGAGGATGCATCCA |
| | | NO: 2) | | AGAAATAGGCTTCGA |
| | | | | AGAATTATGCGTGGC |
| | | | | GGCATTAGGTTCTAG |
| | | | | (SEQ ID NO: 28) |
| CPAR39 | Phage CPAR39 | MCKKVCKKC | 10.26/3993.91 | TTGTGCAAAAAAGTG |
| | Gene: | PKKGPKNAP | (35) | TGCAAAAAATGCCCA |
| | CPA000S | KIGAFYERK | | AAAAAAGGGCCAAAA |
| | GenBank: | TPRLKQST | | AATGCCCCCAAAATC |
| | NP_063898.1 | (SEQ ID | | GGAGCATTTTACGAG |
| | Family: | NO: 3) | | AGAAAAACACCTAGA |
| | Microviridae | | | CTTAAACAGTCTACT |
| | | | | TGA (SEQ ID NO: |
| | | | | 29) |
| Chp3 | Phage 3 | MRLKMARRR | 12.90/5708.98 | ATGAGGTTAAAAATG |
| | Gene: CP3p6 | YRLPRRRSR | (44) | GCACGAAGAAGATAC |
| | GenBank: | RLFSRTALR | | AGACTTCCGCGACGT |
| | YP_022484.1 | MHPRNRLRR | | AGAAGTCGAAGACTT |
| | Family: | IMRGGIRF | | TTTTCAAGAACTGCA |
| | Microviridae | (same | | TTAAGGATGCATCCA |
| | | sequence | | AGAAATAGGCTTCGA |
| | | as Chp2) | | AGAATTATGCGTGGC |
| | | (SEQ ID | | GGCATTAGGTTCTAG |
| | | NO: 54) | | (SEQ ID NO: 53) |
| Chp4 | Phage 4 | MARRYRLSR | 12.88/5073.11 | ATGGCACGAAGATAC |
| | Gene: Chp4p6 | RRSRRLFSR | (39) | AGACTTTCGCGACGC |
| | GenBank: | TALRMHRRN | | AGAAGTCGACGACTT |
| | YP_338243.1 | RLRRIMRGG | | TTTTCAAGAACTGCA |
| | Family: | IRF (SEQ | | TTAAGAATGCATCGA |
| | Microviridae | ID NO: 4) | | AGAAATAGACTTCGA |
| | | | | AGAATTATGCGTGGC |
| | | | | GGCATTAGGTTTTAG |
| | | | | (SEQ ID NO: 30) |
| Chp5 | Phage ChpQE | MAEQYELSQ | 3.73/4605.01 | ATGGCGGAACAGTAT |
| | Derivative of | EQSEQLFSE | (39) | GAACTGAGCCAGGAA |
| | Phage 4 | TALQMHEQN | | CAGAGCGAACAGCTG |
| | | ELQEIMQGG | | TTTAGCGAAACCGCG |
| | | IEF (SEQ | | CTGCAGATGCATGAA |
| | | ID NO: 5) | | CAGAACGAACTGCAG |
| | | | | GAAATTATGCAGGGC |
| | | | | GGCATTGAATTTTAA |
| | | | | (SEQ ID NO: 31) |
| Chp6 | Guinea pig | MARRRYRLP | 12.88/5180.27 | ATGGCACGAAGAAGA |
| | Chlamydia | RRRSRRLFS | (40) | TACAGACTTCCGCGA |
| | phage | RTALRMHPR | | CGTAGAAGTCGAAGA |
| | GenBank: | NRLRRIMRG | | CTTTTTTCAAGAACT |
| | NP_510878.1 | GIRF (SEQ | | GCATTAAGGATGCAT |
| | Family: | ID NO: 6) | | CCAAGAAATAGGCTT |
| | Microviridae | | | CGAAGAATTATGCGT |
| | | | | GGCGGCATTAGGTTC |
| | | | | TAG (SEQ ID NO: |
| | | | | 32) |
| Chp7 | Uncharacterized | MKRRKMTRK | 12.31/4302.19 | ATGAAACGTAGAAAA |
| | protein | GSKRLFTAT | (38) | ATGACAAGAAAAGGT |
| | [Chlamydia | ADKTKSINT | | TCTAAGCGTCTTTTT |
| | trachomatis] | APPPMRGGI | | ACTGCAACTGCTGAT |
| | GenBank: | RL (SEQ | | AAAACTAAATCTATC |
| | CRH73061.1 | ID NO: 7) | | AATACTGCCCCGCCG |
| | Family: | | | TCCAAGCGTGGCGGT |
| | Microviridae | | | ATCCGGTTGTAA |
| | | | | (SEQ ID NO: 33) |
| Chp8 | Uncharacterized | MSKKRSRMS | 12.91/4672.61 | ATGTCTAAAAAGCGTT |
| | protein (C. | RRRSKKLFS | (39) | CTCGCATGTCTCGCCG |
| | trachomatis) | KTALRTKSV | | CCGTTCTAAGAAGTTG |
| | GenBank: | NTRPPMRGG | | TTCTCGAAAACGGCTC |
| | CRH64983.1 | FRF (SEQ | | TCCGCACGAAGAGTGT |
| | Family: | ID NO: 8) | | CAACACCCGTCCGCCT |
| | Microviridae | | | ATGCGCGGAGGGTTCC |
| | | | | GGTTCTGA (SEQ ID |
| | | | | NO: 34) |
| Chp9 | Uncharacterized | MSLRRHKLS | 12.91/4672.60 | ATGTCTCTTCGTCGTC |
| | protein (C. | RKASKRIFR | (40) | ATAAGCTTTCTCGTAA |
| | trachomatis) | KGASRTKTL | | GGCGTCTAAGCGTATT |
| | GenBank: | NTRATPMRG | | TTTCGTAAAGGTGCAT |
| | CRH84960.1 | GFRI (SEQ | | CACGCACGAAGACTTT |
| | Family: | ID NO: 9) | | GAATACTCGTGCTACG |
| | Microviridae | | | CCTATGCGCGGCGGTT |
| | | | | TCCGTATTTAA (SEQ |
| | | | | ID NO: 35) |
| Chp10 | Uncharacterized | MKRRKLSKK | 12.91/4570.64 | ATGAAACGTCGTAAAC |
| | protein (C. | KSRKIFTRG | (38) | TGTCCAAAAAGAAATC |
| | trachomatis) | AVNVKKRNL | | TCGCAAGATTTTCACT |
| | GenBank: | RARPMRGGF | | CGCGGTGCTGTAAATG |
| | CRH93270.1 | RI (SEQ | | TGAAAAAGCGTAACCT |
| | Family: | ID NO: | | TCGCGCTCGCCCAATG |
| | Microviridae | 10) | | CGCGGCGGTTTCCGGA |
| | | | | TCTAA (SEQ ID |
| | | | | NO: 36) |
| Chp11 | Uncharacterized | MAKKMTKGK | 11.74/4375.32 | ATGGCTAAAAAAATGA |
| | protein (C. | DRQVFRKTA | (37) | CTAAAGGCAAGGATCG |
| | trachomatis) | DRTKKLNVR | | TCAGGTTTTTCGTAAA |
| | GenBank: | PLLYRGGIR | | ACCGCTGATCGTACTA |
| | CRH59954.1 | L (SEQ ID | | AGAAACTCAATGTTAG |
| | Family: | NO: 11) | | ACCGTTGTTATATCGA |
| | Microviridae | | | GGAGGTATCAGATTAT |
| | | | | GA (SEQ ID NO: |
| | | | | 37) |
| Chp12 | Uncharacterized | MAGKKMVSK | 11.74/4549.53 | ATGGCAGGAAAAAAAA |
| | protein (C. | GKDRQIFRK | (39) | TGGTATCAAAAGGAAA |
| | trachomatis) | TADRTKKMN | | AGATAGACAGATTTTC |
| | GenBank: | VRPLLYRGG | | CGAAAAACTGCTGATC |
| | CRH59965.1 | IRL (SEQ | | GCACTAAAAAAATGAA |
| | Family: | ID NO: | | TGTGCGCCCGCTATTA |
| | Microviridae | 12) | | TATCGTGGAGGTATTA |
| | | | | GATTATGA (SEQ ID |
| | | | | NO: 38) |

TABLE 2
Additional Chp family members

| Protein | Identifier | Protein | pI/kDa | |
|---|---|---|---|---|
| name | Information | Sequence | (amino acids) | DNA Sequence |
| Gkhl | Marine | MRRPRKMNY | 12.66/4974.97 | ATGAGAAGACCAAGA |
| | gokushovirus | KKSKRMFSR | (41) | AAAATGAACTATAAA |
| | Gene: | TAARTHRKN | | AAATCAAAAAGAATG |
| | V508_gp1 | SLRGSRPMR | | TTTTCACGCACAGCA |
| | GenBank: | GGIRL | | GCGAGAACACACAGA |
| | YP_008798245.1 | (SEQ ID | | AAAAACTCTCTAAGA |
| | Unclassified | NO: 13) | | GGTAGCCGACCTATG |
| | Gokushovirinae | | | AGAGGCGGAATACGT |
| | | | | CTTTAA (SEQ ID |
| | | | | NO: 39) |
| Gkh2 | Gokushovirinae | MSKKASRKS | 12.49/3794.63 | ATGTCGAAGAAGGCG |
| | Fen672_31 | FTKGAVKVH | (34) | TCGAGGAAGAGTTTT |
| | Gene: | KKNVPTRVP | | ACTAAGGGTGCCGTT |
| | AFL78_gp4 | MRGGIRL | | AAGGTTCATAAGAAA |
| | GenBank: | (SEQ ID | | AATGTTCCTACTCGT |
| | YP_009160382.1 | NO: 14) | | GTTCCTATGCGTGGC |
| | Unclassified | | | GGTATTAGGCTTTAG |
| | Gokushovirinae | | | (SEQ ID NO: 40) |
| Unp1 | Unnamed | MKMRKRTDK | 12.31/4104.04 | ATGAAAATGCGTAAG |
| | protein product | RVFTRTAAK | (35) | CGGACGGACAAGCGA |
| | (uncultured | SKKVNIAPK | | GTGTTTACCCGCACC |
| | bacterium) | IFRGGIRL | | GCTGCTAAGTCCAAG |
| | GenBank: | (SEQ ID | | AAAGTGAACATTGCC |
| | CDL66944.1 | NO: 15) | | CCGAAAATTTTTAGA |
| | Circular | | | GGAGGTATCCGTCTG |
| | plasmid, rat | | | TGA (SEQ ID NO: |
| | cecum | | | 41) |
| Ecp1 | Nonstructural | MARSRRRMS | 12.70/4812.72 | ATGGCTCGTTCTCGC |
| | protein | KRSSRRSFR | (39) | CGTCGTATGTCCAAG |
| | (Escherichia | KYAKTHKRN | | CGTTCTTCCCGTCGT |
| | coli) | FKARSMRGG | | TCGTTCCGTAAGTAC |
| | GenBank: | IRL (SEQ | | GCAAAGACGCATAAA |
| | WP_100756432.1 | ID NO: | | CGTAACTTTAAAGCC |
| | sEPEC Feces | 16) | | CGCTCTATGCGTGGT |
| | strain | | | GGAATTCGTCTTTGA |
| | | | | (SEQ ID NO: 42) |
| Tma1 | Hypothetical | MESPNSRSQL | 7.80/5433.39 | ATGGAAAGCCCGAAC |
| | protein (T. | GITLYLLSTI | (47) | AGCCGCAGCCAGCTG |
| | maritimus) | FPDACFRYRR | | GGCATTACCCTGTAT |
| | SAMN04488044_0855 | ELPYPLVIWG | | CTGCTGAGCACCATT |
| | GenBank: | VATLCLQ | | TTTCCGGATGCGTGC |
| | SHG47122.1 | (SEQ ID | | TTTCGCTATCGCCGC |
| | | NO: 17) | | GAACTGCCGTATCCG |
| | | | | CTGGTGATTTGGGGC |
| | | | | GTGGCGACCCTGTGC |
| | | | | CTGCAGTAA (SEQ |
| | | | | ID NO: 43) |
| Ecp2 | Hypothetical | MARSRRRMSK | 12.66/4770.68 | ATGGCTCGTTCCCGT |
| | protein | RSSRRSFRKY | (39) | AGACGTATGTCTAAG |
| | EC13107_44c0 | AKSHKKNFKA | | CGTTCTTCCCGCCGT |
| | 0010 (E. coli) | RSMRGGIRL | | TCGTTCCGCAAGTAT |
| | GenBank: | (SEQ ID | | GCGAAGTCGCATAAG |
| | OAC14041.1 | NO: 18) | | AAGAACTTTAAAGCC |
| | Udder, acute | | | CGCTCAATGCGTGGC |
| | mastitis | | | GGTATCCGTTTATAA |
| | | | | (SEQ ID NO: 44) |
| Osp1 | Hypothetical | MRKRMSKRVD | 11.90/4389.35 | ATGAGAAAGCGAATG |
| | protein | KKVFRRTAAS | (37) | TCTAAGCGTGTTGAC |
| | SAMN05216343_103150 | AKKINIDPKI | | AAGAAGGTGTTCCGT |
| | (Oscillibacter | YRGGIRL | | CGTACTGCCGCATCT |
| | sp. PC13) | (SEQ ID | | GCCAAGAAGATTAAC |
| | GenBank: | NO: 19) | | ATTGACCCCAAGATT |
| | SFP13761.1 | | | TACCGTGGAGGTATT |
| | | | | CGCCTATGA (SEQ |
| | | | | ID NO: 45) |
| Unp2 | Unnamed | MRRRRLSRRT | 13.18/4757.77 | ATGAGACGTCGTCGT |
| | protein product | SRRFFRKGLK | (37) | CTATCCCGCAGAACT |
| | GenBank: | VRRRNLRARP | | TCCCGCCGTTTTTTC |
| | CDL65918.1 | MRGGFRI | | CGTAAAGGACTTAAG |
| | Extrachromosomal | (SEQ ID | | GTTCGCCGTCGTAAC |
| | DNA RG100327 | NO: 20) | | CTCCGCGCGAGACCC |
| | | | | ATGAGAGGCGGATTC |
| | | | | AGAATTTGA (SEQ |
| | | | | ID NO: 46) |
| Unp3 | Unnamed | MARRKKMKGK | 12.32/4545.51 | ATGGCACGACGCAAG |
| | protein product | RDKRVFKQTA | (39) | AAGATGAAAGGCAAG |
| | GenBank: | NKTKAINISP | | CGGGATAAACGGGTG |
| | CDL65808.1 | KNMRGGTRL | | TTTAAGCAGACAGCC |
| | Extrachromosomal | (SEQ ID | | AACAAAACCAAGGCT |
| | DNA RG100234 | NO: 21) | | ATCAACATCAGCCCA |
| | | | | AAAAACATGAGAGGG |
| | | | | GGTACGAGACTGTGA |
| | | | | (SEQ ID NO: 47) |
| Gkh3 | Hypothetical | MLTVWSDTPT | 11.2/6440.82 | ATGTTAACTGTGTGG |
| | protein (Marine | IKRRKDMYRK | (53) | AGTGACACCCCTACC |
| | gokushovirus) | RMSRKKSKKV | | ATAAAAAGGAGAAAA |
| | GenBank: | FAKTAMKVNK | | GACATGTATAGAAAG |
| | AGT39941.1 | RNHVKPMRGG | | AGAATGTCAAGAAAG |
| | | YRI (SEQ | | AAAAGTAAAAAGGTT |
| | | ID NO: 22) | | TTTGCAAAAACCGCA |
| | | | | ATGAAAGTAAATAAA |
| | | | | AGAAACCACGTTAAA |
| | | | | CCTATGCGTGGTGGA |
| | | | | TATAGAATATAA |
| | | | | (SEQ ID NO: 48) |
| Unp5 | Hypothetical | MMKYRKKMSA | 12.04/4536.61 | ATGATGAAGTACAGA |
| | protein (Marine | KSSRKQFTKG | (39) | AAAAAAATGAGCGCT |
| | gokushovirus) | AMKVKGKNFT | | AAAAGTAGCCGAAAG |
| | GenBank: | KPMRGGIRL | | CAATTTACAAAAGGC |
| | AGT39924.1 | (SEQ ID | | GCCATGAAAGTGAAG |
| | | NO: 23) | | GGTAAAAACTTCACA |
| | | | | AAACCAATGCGCGGA |
| | | | | GGCATCCGTCTATAG |
| | | | | (SEQ ID NO: 49) |
| Unp6 | Hypothetical | MRRYNVNKGK | 12.31/4492.34 | ATGCGACGTTACAAT |
| | protein (Marine | SAKKFRKQVS | (38) | GTAAATAAAGGTAAA |
| | gokushovirus) | KTKVANLRSN | | TCTGCTAAGAAGTTT |
| | GenBank: | PMRGGWRL | | CGAAAGCAGGTAAGT |
| | AGT39915.1 | (SEQ ID | | AAGACGAAGGTTGCA |
| | | NO: 24) | | AACCTACGTTCTAAT |
| | | | | CCAATGCGAGGTGGT |
| | | | | TGGAGACTCTAA |
| | | | | (SEQ ID NO: 50) |
| Spi1 | Hypothetical | MAYRGFKTSR | 12.37/3776.45 | ATGGCTTATCGTGGT |
| | protein Sp-4p3 | VVKHRVRRRW | (28) | TTTAAAACGAGTCGT |
| | (Spiroplasma | FNHRRRYR | | GTTGTAAAACATAGA |
| | virus SpV4] | (SEQ ID | | GTACGTAGAAGATGG |
| | Orf9 | NO: 25) | | TTTAATCATAGAAGA |
| | NCBI Ref. Seq: | | | CGTTATAGATAG |
| | NP_598337.1 | | | (SEQ ID NO: 51) |
| Spi2 | Hypothetical | MRRKVKNTKR | 12.91/4629.45 | ATGCGTCGTAAAGTT |
| | protein Sp-4p2 | HQWRLTHSAR | (38) | AAAAACACCAAACGT |
| | (Spiroplasma | SIKRANIMPS | | CACCAGTGGCGTCTG |
| | virus SpV4) | NPRGGRRF | | ACCCACTCTGCTCGT |
| | Orf8 | (SEQ ID: | | TCTATCAAACGTGCT |
| | NCBI Ref. Seq: | 26) | | AACATCATGCCGTCT |
| | NP_598336.1 | | | AACCCGCGTGGTGGT |
| | | | | CGTCGTTTC (SEQ |
| | | | | ID NO: 52) |
| Ecp3 | Nonstructural | MARSRRRMSK | 12.76/4784.69 | ATGGCTCGTTCTCGT |
| | protein | RSSRRSFRKY | (39) | CGTCGTATGTCTAAA |
| | (Escherichia) | AKTHKKNFKA | | CGTTCTTCTCGTCGT |
| | NCBI Ref. Seq: | RSMRGGIRL | | TCTTTTCGTAAATAT |
| | WP_105269219.1 | (SEQ ID | | GCTAAAACTCATAAA |
| | | NO: 55) | | AAAAATTTTAAAGCT |
| | | | | CGTTCTATGCGTGGA |
| | | | | GGAATTCGTTTATAA |
| | | | | (SEQ ID NO: 68) |
| Ecp4 | Nonstructural | MARSRRRMSK | 12.66/4770.68 | ATGGCGCGCAGCCGC |
| | protein | RSSRRSFRKY | (39) | CGCCGCATGAGCAAA |
| | (Escherichia) | AKSHKKNFKA | | CGCAGCAGCCGCCGC |
| | NCBI Ref. Seq: | RSMRGGIRL | | AGCTTTCGCAAATAT |
| | WP_105466506.1 | (SEQ ID | | GCGAAAAGCCATAAA |
| | | NO: 56) | | AAAAACTTTAAAGCG |
| | | | | CGCAGCATGCGCGGC |
| | | | | GGCATTCGCCTG |
| | | | | (SEQ ID NO: 69) |
| Lvp1 | Lysis protein | MSSTLCRWAV | 9.7/6346.6 | ATGTCTTCTACCCTG |
| | (Pseudomonas | KALRCTRVYK | (55) | TGCCGTTGGGCTGTT |
| | phage PP7) | EFIWKPLVAL | | AAAGCTCTGCGTTGC |
| | NCBI Ref. Seq: | SYVTLYLLSS | | ACCCGTGTTTACAAA |
| | NP_042306.1 | VFLSQLSYPI | | GAATTCATCTGGAAA |
| | | GSWAV (SEQ | | CCGCTGGTTGCTCTG |
| | | ID NO: 57) | | TCTTACGTTACCCTG |
| | | | | TACCTGCTGTCTTCT |
| | | | | GTTTTCCTGTCTCAG |
| | | | | CTGTCTTACCCGATC |
| | | | | GGTTCTTGGGCTGTT |
| | | | | (SEQ ID NO: 70) |
| (ABP1) | Lysis protein | MKKRTKALLP | 9.93/4247.21 | ATGAAGAAAAGGACA |
| Lvp2 | (Acinetobacter | YAVFIILSFQ | (35) | AAAGCCTTGCTTCCC |
| | phage AP205) | LTLLTALFMY | | TATGCGGTTTTCATC |
| | NCBI Ref. Seq: | YHYTF (SEQ | | ATACTCAGCTTTCAA |
| | NP_085469.1 | ID NO: 58) | | CTAACATTGTTGACT |
| | | | | GCCTTGTTTATGTAT |
| | | | | TACCATTATACCTTT |
| | | | | TAG (SEQ ID NO: |
| | | | | 71) |
| ALCES1 | Hypothetical | MAKKIRNKAR | 12.70/4599.52 | ATGGCAAAGAAAATT |
| | protein (Alces | DRRIFTRTAS | (38) | AGAAACAAAGCACGT |
| | alces faeces | RMHKANRTPR | | GATAGACGTATCTTC |
| | associated | FMRGGIRL | | ACAAGAACAGCTTCA |
| | micro virus | (SEQ ID | | CGCATGCACAAGGCA |
| | MP12 5423) | NO: 59) | | AACCGCACACCAAGA |
| | NCBI Ref. Seq: | | | TTTATGAGAGGCGGT |
| | AXB22573.1 | | | ATTAGGTTATGA |
| | | | | (SEQ ID NO: 72) |
| AVQ206 | Hypothetical | MRRKKMSRGK | 13.10/4680.78 | ATGCGTCGTAAAAAA |
| | protein | SKKLFRRTAK | (38) | ATGTCTCGTGGTAAA |
| | (Gokushovirinae | RVHRKNLRAR | | TCTAAAAAACTGTTC |
| | environmental | PMRGGIRM | | CGTCGTACCGCTAAA |
| | samples) | (SEQ ID | | CGTGTTCACCGTAAA |
| | NCBI Ref. Seq: | NO: 60) | | AACCTGCGTGCTCGT |
| | AVQ10236.1 | | | CCGATGCGTGGTGGT |
| | | | | ATCCGTATG (SEQ |
| | | | | ID NO: 73) |
| AVQ244 | Hypothetical | MAKRHKIPQR | 12.8/4566.43 | ATGGCTAAACGTCAC |
| | protein | ASQHSFTRHA | (39) | AAAATCCCGCAGCGT |
| | (Gokushovirinae | QKVHPKNVPR | | GCTTCTCAGCACTCT |
| | environmental | LPMRGGIRL | | TTCACCCGTCACGCT |
| | samples) | (SEQ ID | | CAGAAAGTTCACCCG |
| | NCBI Ref. Seq: | NO: 61) | | AAAAACGTTCCGCGT |
| | AVQ10244.1 | | | CTGCCGATGCGTGGT |
| | | | | GGTATCCGTCTG |
| | | | | (SEQ ID NO: 74) |
| CDL907 | Unnamed | MRKKMHKSLD | 11.96/4398.22 | ATGCGTAAAAAAATG |
| | protein product | KRVFNRTAKK | (37) | CACAAATCTCTGGAC |
| | (uncultured | SKKINVNPVV | | AAACGTGTTTTCAAC |
| | bacterium) | YRGGIRL | | CGTACCGCTAAAAAA |
| | NCBI Ref. Seq: | (SEQ ID | | TCTAAAAAAATCAAC |
| | CDL65907.1 | NO: 62) | | GTTAACCCGGTTGTT |
| | | | | TACCGTGGTGGTATC |
| | | | | CGTCTG (SEQ ID |
| | | | | NO: 75) |
| AGT915 | Hypothetical | MRRYNVNKGK | 12.41/4492.32 | ATGCGACGTTACAAT |
| | protein (Marine | SAKKFRKQVS | (38) | GTAAATAAAGGTAAA |
| | gokushovirus) | KTKVANLRSN | | TCTGCTAAGAAGTTT |
| | NCBI Ref. Seq: | PMRGGWRL | | CGAAAGCAGGTAAGT |
| | AGT39915.1 | (SEQ ID | | AAGACGAAGGTTGCA |
| | | NO: 63) | | AACCTACGTTCTAAT |
| | | | | CCAATGCGAGGTGGT |
| | | | | TGGAGACTCTAA |
| | | | | (SEQ ID NO: 76) |
| HH3930 | Hypothetical | MRPVKRSRVN | 12.95/4755.69 | ATGCGTCCAGTTAAA |
| | protein | KARSAGKFRK | (41) | AGATCAAGAGTAAAT |
| | RINTHH_3930 | QVGKTKMANL | | AAGGCCCGATCTGCA |
| | (Richelia | RSNPMRGGWR | | GGCAAGTTTCGTAAG |
| | intracellularis | L (SEQ ID | | CAGGTCGGTAAAACA |
| | HH01) | NO: 64) | | AAGATGGCAAATCTG |
| | NCBI Ref. Seq: | | | CGTAGTAATCCGATG |
| | CCH66548.1 | | | CGCGGCGGATGGCGG |
| | | | | CTGTGA (SEQ ID |
| | | | | NO: 77) |
| Fen7875 | Hypothetical | MKPLKRKPVQ | 12.81/4699.7 | ATGAAGCCATTGAAG |
| | protein | KARSAAKFRR | (41) | CGTAAGCCGGTTCAG |
| | (Gokushovirinae | NVSTVKAANM | | AAGGCGCGGTCAGCA |
| | Fen7875_21) | AVKPMRGGWR | | GCCAAGTTCCGTCGA |
| | NCBI Ref. Seq: | F (SEQ ID | | AATGTGTCTACCGTT |
| | YP_009160399.1 | NO: 65) | | AAGGCTGCCAATATG |
| | | | | GCGGTGAAGCCGATG |
| | | | | CGCGGCGGTTGGCGG |
| | | | | TTCTGA (SEQ ID |
| | | | | NO: 78) |
| SBR77 | Hypothetical | MTKRDIEYRK | 11.48/4882.78 | ATGACCAAGAGAGAC |
| | protein | ALGLNPSEPL | (44) | ATCGAGTACCGGAAA |
| | SEA_BABYRAY_77 | PKIVGAVTRH | | GCTTTGGGGCTCAAC |
| | (Mycobacterium | GATLKRPRVT | | CCATCTGAGCCGCTC |
| | phage BabyRay) | ALAR (SEQ | | CCGAAGATTGTGGGT |
| | NCBI Ref. Seq: | ID NO: 66) | | GCCGTCACCCGCCAC |
| | AOT25441 | | | GGGGCCACTCTGAAA |
| | | | | CGCCCACGGGTCACC |
| | | | | GCACTGGCCCGATAG |
| | | | | (SEQ ID NO: 79) |
| Bdpl | Putative DNA | MKRKPMSRKA | 12.9/5708.98 | ATGAAAAGAAAACCA |
| | binding protein | SQKTFKKNTG | (38) | ATGAGCCGCAAGGCC |
| | (B dello vibrio | VQRMNHLNPR | | TCTCAAAAAACCTTC |
| | phage phiMH2K) | AMRGGIRL | | AAAAAGAACACAGGC |
| | NCBI Ref. Seq: | (SEQ ID | | GTTCAACGCATGAAC |
| | NP_073546.1 | NO: 67) | | CATCTCAACCCACGC |
| | | | | GCCATGCGTGGTGGC |
| | | | | ATTAGACTATAA |
| | | | | (SEQ ID NO: 80) |
| Unp4 | Hypothetical | MIVRRHKMSR | 12.8/4918.88 | ATGATCGTTCGTCGT |
| | protein (Marine | RRSRKLFSKT | (40) | CACAAAATGTCTCGT |
| | gokushovirus) | ASRTRSKNLR | | CGTCGTTCTCGTAAA |
| | NCBI Ref. Seq: | SRPMRGGYRI | | CTGTTCTCTAAAACC |
| | WP_113076974.1 | (SEQ ID | | GCTTCTCGTACCCGT |
| | | NO: 94) | | TCTAAAAACCTGCGT |
| | | | | TCTCGTCCGATGCGT |
| | | | | GGTGGTTACCGTATC |
| | | | | (SEQ ID NO: |
| | | | | 103) |
| Myo1 | Hypothetical | MKLTKSDIAY | 11.7/5287.23 | ATGAAACTGACCAAA |
| | protein Peaches 72 | REALGLSTTD | (46) | TCTGACATCGCTTAC |
| | (Mycobacterium | PLPAEIGMVT | | CGTGAAGCTCTGGGT |
| | virus Peaches) | RRANRLKRPR | | CTGTCTACCACCGAC |
| | NCBI Ref. Seq: | KTARFR | | CCGCTGCCGGCTGAA |
| | YP_003358775.1 | (SEQ ID | | ATCGGTATGGTTACC |
| | | NO: 102) | | CGTCGTGCTAACCGT |
| | | | | CTGAAACGTCCGCGT |
| | | | | AAAACCGCTCGTTTC |
| | | | | CGT (SEQ ID NO: |
| | | | | 104) |

TABLE C
Modified Chp peptide family members

| Protein | Identifier | Protein | pI/kDa | |
|---|---|---|---|---|
| name | Information | Sequence | (amino acids) | DNA Sequence |
| Chp2-M1 | Modified | M [symbol] L [symbol] MA [symbol] [symbol] [symbol] | 12.9/5708.98 | ATGAGGTTAAAAATGG |
| | variant of | Y [symbol] LP [symbol] [symbol] [symbol] S [symbol] | (44) | CACGAAGAAGATACAG |
| | Chp2 | [symbol] LFS [symbol] TAL [symbol] | | ACTTCCGCGACGTAGA |
| | | MHP [symbol] N [symbol] L [symbol] [symbol] | | AGTCGAAGACTTTTTT |
| | | IM [symbol] GGI [symbol] F | | CAAGAACTGCATTGAG |
| | | (SEQ ID | | GATGCATCCAAGAAAT |
| | | NO: 81) | | AGGCTTCGAAGAATTA |
| | | | | TGCGTGGCGGCATTAG |
| | | | | GTTC (SEQ ID NO: |
| | | | | 105) |
| Chp2-Cys | Modified | MRLKMARRR | 12.8/5812.15 | ATGAGGTTAAAAATGG |
| | variant of | YRLPRRRSR | (45) | CACGAAGAAGATACAG |
| | Chp2 | RLFSRTALR | | ACTTCCGCGACGTAGA |
| | | MHPRNRLRR | | AGTCGAAGACTTTTTT |
| | | IMRGGIRFC | | CAAGAACTGCATTGAG |
| | | (SEQ ID | | GATGCATCCAAGAAAT |
| | | NO: 82) | | AGGCTTCGAAGAATTA |
| | | | | TGCGTGGCGGCATTAG |
| | | | | GTTCTGT (SEQ ID |
| | | | | NO: 106) |
| Chp2-NC | Modified | APKAMRLKM | 13.00/6744.29 | GCTCCTAAAGCTAGGT |
| | variant of | ARRRYRLPR | (54) | TAAAAATGGCACGAAG |
| | Chp2 | RRSRRLFSR | | AAGATACAGACTTCCG |
| | | TALRMHPRN | | CGACGTAGAAGTCGAA |
| | | RLRRIMRGG | | GACTTTTTTCAAGAAC |
| | | IRFLQKKGI | | TGCATTGAGGATGCAT |
| | | (SEQ ID | | CCAAGAAATAGGCTTC |
| | | NO: 83) | | GAAGAATTATGCGTGG |
| | | | | CGGCATTAGGTTCTTA |
| | | | | CAAAAAAAAGGAATT |
| | | | | (SEQ ID NO: 107) |
| Chp4::Chp2 | Fusion | MARRYRLSR | 13.18/7851.54 | ATGGCTCGTCGTTATC |
| | peptide of | RRSRRLFSR | (60) | GTTTATCTCGTCGTCG |
| | Chp4 and | TALRMHRRN | | TTCTCGTCGTTTATTT |
| | Chp2 | RLRRIMRRL | | TCTCGTACTGCTTTAC |
| | | RFSTALRMH | | GTATGCATCGTCGTAA |
| | | RRNRLRRIM | | TCGTTTACGTCGTATT |
| | | RGGIRF | | ATGCGTCGTTTATTTT |
| | | (SEQ ID | | CTCGTACTGCTTTACG |
| | | NO: 84) | | TATGCATCGTCGTAAT |
| | | | | CGTTTACGTCGTATTA |
| | | | | TGCGTGGAGGAATTCG |
| | | | | TTTT (SEQ ID NO: |
| | | | | 108) |
| Chp2-CAV | Modified | GRLYRFHRP | 13.00/5709 | GGACGTTTATATCGTT |
| | variant of | RRRNAIGMS | (44) | TTCATCGTCCTCGTCG |
| | Chp2 | RMRRKMFLR | | TCGTAATGCTATTGGA |
| | | RMLRLISRR | | ATGTCTCGTATGCGTC |
| | | TRRPRLRA | | GTAAAATGTTTTTACG |
| | | (SEQ ID | | TCGTATGTTACGTTTA |
| | | NO: 85) | | ATTTCTCGTCGTACTC |
| | | | | GTCGTCCTCGTTTACG |
| | | | | TGCT (SEQ ID NO: |
| | | | | 109) |
| Ecp1-CAV | Modified | RTRNFRIRR | 12.80/4812.71 | CGTACTCGTAATTTTC |
| | variant of | AKARRKMML | (39) | GTATTCGTCGTGCTAA |
| | Ecpl | SHFKYGMAR | | AGCTCGTCGTAAAATG |
| | | KGSKSRSSR | | ATGTTATCTCATTTTA |
| | | RSR (SEQ | | AATATGGAATGGCTCG |
| | | ID NO: | | TAAAGGATCTAAATCT |
| | | 86) | | CGTTCTTCTCGTCGTT |
| | | | | CTCGT (SEQ ID |
| | | | | NO: 110) |
| Ecp1-M1 | Modified | MA [symbol] S [symbol] [symbol] [symbol] MS | 12.7/4812.72 | ATGGCTCGTTCTCGTC |
| | variant of | [symbol] [symbol] SS [symbol] [symbol] SF [symbol] | (39) | GTCGTATGTCTAAACG |
| | Ecpl | [symbol] YA [symbol] TH [symbol] [symbol] N | | TTCTTCTCGTCGTTCT |
| | | F [symbol] A [symbol] SM [symbol] GG | | TTTCGTAAATATGCTA |
| | | I [symbol] L (SEQ | | AAACTCATAAACGTAA |
| | | ID NO: | | TTTTAAAGCTCGTTCT |
| | | 87) | | ATGCGTGGAGGAATTC |
| | | | | GTTTATAA (SEQ ID |
| | | | | NO: 111) |
| Chp6-M1 | Modified | MA [symbol] [symbol] [symbol] Y [symbol] LP | 12.31/4492.34 | ATGGCACGAAGAAGAT |
| | variant of | [symbol] [symbol] [symbol] S [symbol] [symbol] LFS | (38) | ACAGACTTCCGCGACG |
| | Chp6 | [symbol] TAL [symbol] MHP [symbol] | | TAGAAGTCGAAGACTT |
| | | N [symbol] L [symbol] [symbol] IMRG | | TTTTCAAGAACTGCAT |
| | | GI [symbol] F (SEQ | | TAAGGATGCATCCAAG |
| | | ID NO: | | AAATAGGCTTCGAAGA |
| | | 88) | | ATTATGCGTGGCGGCA |
| | | | | TTAGGTTCTAG (SEQ |
| | | | | ID NO: 112) |
| Chp10-M1 | Modified | M [symbol] [symbol] [symbol] [symbol] LS [symbol] [symbol] | 12.91/4570.64 | GTGAAACGTCGTAAAC |
| | variant of | [symbol] S [symbol] [symbol] IFT [symbol] G | (38) | TGTCCAAAAAGAAATC |
| | Chp10 | AVNV [symbol] [symbol] [symbol] NL | | TCGCAAGATTTTCACT |
| | | [symbol] A [symbol] PM [symbol] GGF | | CGCGGTGCTGTAAATG |
| | | [symbol] I (SEQ | | TGAAAAAGCGTAACCT |
| | | ID NO: | | TCGCGCTCGCCCAATG |
| | | 89) | | CGCGGCGGTTTCCGGA |
| | | | | TCTAA (SEQ ID |
| | | | | NO: 113) |
| Mse-M1 | Modified | MA [symbol] [symbol] I [symbol] N [symbol] A | 12.70/4599.52 | ATGGCTAAAAAAATCC |
| | variant of | [symbol] D [symbol] [symbol] IFT [symbol] T | (38) | GTAACAAAGCTCGTGA |
| | Mse | AS [symbol] MH [symbol] AN [symbol] | | CCGTCGTATCTTCACC |
| | | TP [symbol] FM [symbol] GGI | | CGTACCGCTTCTCGTA |
| | | [symbol] L (SEQ | | TGCACAAAGCTAACCG |
| | | ID NO: | | TACCCCGCGTTTCATG |
| | | 90) | | CGTGGTGGTATCCGTC |
| | | | | TG (SEQ ID NO: |
| | | | | 114) |
| Chp4-M1 | Modified | MA [symbol] [symbol] Y [symbol] LS [symbol] | 12.88/5073.11 | ATGGCACGAAGATACA |
| | variant of | [symbol] [symbol] S [symbol] [symbol] LFS [symbol] | (39) | GACTTTCGCGACGCAG |
| | Chp4 | TAL [symbol] MH [symbol] [symbol] N | | AAGTCGACGACTTTTT |
| | | [symbol] L [symbol] [symbol] IM [symbol] GG | | TCAAGAACTGCATTAA |
| | | I [symbol] F (SEQ | | GAATGCATCGAAGAAA |
| | | ID NO: | | TAGACTTCGAAGAATT |
| | | 91) | | ATGCGTGGCGGCATTA |
| | | | | GGTTTTAG (SEQ ID |
| | | | | NO: 115) |
| Chp2-SCR1 | Modified | MRLRYGHRR | 13.00/5709 | ATGCGTCTGCGTTACG |
| | variant of | MTAGRIRMR | (44) | GTCACCGTCGTATGAC |
| | Chp2 | SRRKFMLPR | | CGCTGGTCGTATCCGT |
| | | FRLLRIPRR | | ATGCGTTCTCGTCGTA |
| | | SNRRRLRA | | AATTCATGCTGCCGCG |
| | | (SEQ ID | | TTTCCGTCTGCTGCGT |
| | | NO: 92) | | ATCCCGCGTCGTTCTA |
| | | | | ACCGTCGTCGTCTGCG |
| | | | | TGCT (SEQ ID NO: |
| | | | | 116) |
| Chp2-SCR- | Modified | M [symbol] L [symbol] YGH [symbol] [symbol] | 13.00/5709 | ATGCGTCTGCGTTACG |
| M1 | variant of | MTAG [symbol] I [symbol] M [symbol] | (44) | GTCACCGTCGTATGAC |
| | Chp2 | S [symbol] [symbol] KFMLP [symbol] | | CGCTGGTCGTATCCGT |
| | | F [symbol] LL [symbol] IP [symbol] [symbol] | | ATGCGTTCTCGTCGTA |
| | | SN [symbol] [symbol] [symbol] L [symbol] A | | AATTCATGCTGCCGCG |
| | | (SEQ ID | | TTTCCGTCTGCTGCGT |
| | | NO: 93) | | ATCCCGCGTCGTTCTA |
| | | | | ACCGTCGTCGTCTGCG |
| | | | | TGCT (SEQ ID NO: |
| | | | | 117) |
| Chp7-M1 | Modified | MK [symbol] [symbol] [symbol] MT [symbol] [symbol] | 12.31/4302.19 | ATGAAACGTAGAAAAA |
| | variant of | GS [symbol] [symbol] LFTAT | (38) | TGACAAGAAAAGGTTC |
| | Chp7 | AD [symbol] T [symbol] SINT | | TAAGCGTCTTTTTACT |
| | | APPPM [symbol] GGI | | GCAACTGCTGATAAAA |
| | | [symbol] L (SEQ | | CTAAATCTATCAATAC |
| | | ID NO: | | TGCCCCGCCGCCAATG |
| | | 95) | | CGTGGCGGTATCCGGT |
| | | | | TGTAA (SEQ ID |
| | | | | NO: 118) |
| Osp1-M1 | Modified | M [symbol] [symbol] [symbol] MS [symbol] [symbol] N | 11.9/4389.35 | ATGAGAAAGCGAATGT |
| | variant of | D [symbol] [symbol] VF [symbol] [symbol] FA | (37) | CTAAGCGTGTTGACAA |
| | Osp1 | ASA [symbol] [symbol] INID | | GAAGGTGTTCCGTCGT |
| | | P [symbol] IYRGGI [symbol] | | ACTGCCGCATCTGCCA |
| | | L (SEQ ID | | AGAAGATTAACATTGA |
| | | NO: 96) | | CCCCAAGATTTACCGT |
| | | | | GGAGGTATTCGCCTAT |
| | | | | GA (SEQ ID NO: |
| | | | | 119) |
| Unp2-M1 | Modified | M [symbol] [symbol] [symbol] [symbol] LS [symbol] [symbol] | 13.18/4757.77 | ATGAGACGTCGTCGTC |
| | variant of | TS [symbol] [symbol] FF [symbol] [symbol] G | (37) | TATCCCGCAGAACTTC |
| | Unp2 | L [symbol] V [symbol] [symbol] [symbol] NL [symbol] | | CCGCCGTTTTTTCCGT |
| | | A [symbol] PM [symbol] GGF [symbol] | | AAAGGACTTAAGGTTC |
| | | I (SEQ ID | | GCCGTCGTAACCTCCG |
| | | NO: 97) | | CGCGAGACCCATGAGA |
| | | | | GGCGGATTCAGAATTT |
| | | | | GA (SEQ ID NO: |
| | | | | 120) |
| Unp3-M1 | Modified | MA [symbol] [symbol] [symbol] [symbol] M [symbol] G | 12.32/4545.51 | ATGGCACGACGCAAGA |
| | variant of | [symbol] [symbol] D [symbol] [symbol] VF [symbol] Q | (39) | AGATGAAAGGCAAGCG |
| | Unp3 | TAN [symbol] T [symbol] AIN | | GGATAAACGGGTGTTT |
| | | ISP [symbol] NM [symbol] GG | | AAGCAGACAGCCAACA |
| | | T [symbol] L (SEQ | | AAACCAAGGCTATCAA |
| | | ID NO: | | CATCAGCCCAAAAAAC |
| | | 98) | | ATGAGAGGGGGTACGA |
| | | | | GACTGTGA (SEQ ID |
| | | | | NO: 121) |
| Spi2-M1 | Modified | M [symbol] [symbol] KV [symbol] NT [symbol] | 12.91/4629.45 | ATGCGTCGTAAAGTTA |
| | variant of | [symbol] HQW [symbol] LTHS | (38) | AAAACACCAAACGTCA |
| | Spi2 | A [symbol] SI [symbol] [symbol] ANI | | CCAGTGGCGTCTGACC |
| | | MPSNP [symbol] GG [symbol] | | CACTCTGCTCGTTCTA |
| | | [symbol] (SEQ ID | | TCAAACGTGCTAACAT |
| | | NO: 99) | | CATGCCGTCTAACCCG |
| | | | | CGTGGTGGTCGTCGTT |
| | | | | TC (SEQ ID NO: |
| | | | | 122) |
| Ecp3-M1 | Modified | MA [symbol] S [symbol] [symbol] [symbol] MS | 12.76/4784.69 | ATGGCTCGTTCTCGTC |
| | variant of | [symbol] [symbol] SS [symbol] [symbol] SF [symbol] | (39) | GTCGTATGTCTAAACG |
| | Ecp3 | [symbol] YA [symbol] TH [symbol] [symbol] N | | TTCTTCTCGTCGTTCT |
| | | F [symbol] A [symbol] SM [symbol] GG | | TTTCGTAAATATGCTA |
| | | I [symbol] L (SEQ | | AAACTCATAAAAAAAA |
| | | ID NO: | | TTTTAAAGCTCGTTCT |
| | | 100) | | ATGCGTGGAGGAATTC |
| | | | | GTTTATAA (SEQ ID |
| | | | | NO: 123) |
| Agt1-M1 | Modified | M [symbol] [symbol] YNVN [symbol] G | 12.41/5287.23 | ATGCGACGTTACAATG |
| | variant of | [symbol] SA [symbol] [symbol] F [symbol] [symbol] Q | (46) | TAAATAAAGGTAAATC |
| | Agt1 | VS [symbol] T [symbol] VANL | | TGCTAAGAAGTTTCGA |
| | | [symbol] SNPM [symbol] GGW | | AAGCAGGTAAGTAAGA |
| | | [symbol] L (SEQ | | CGAAGGTTGCAAACCT |
| | | ID NO: | | ACGTTCTAATCCAATG |
| | | 101) | | CGAGGTGGTTGGAGAC |
| | | | | TCTAA (SEQ ID |
| | | | | NO: 124) |

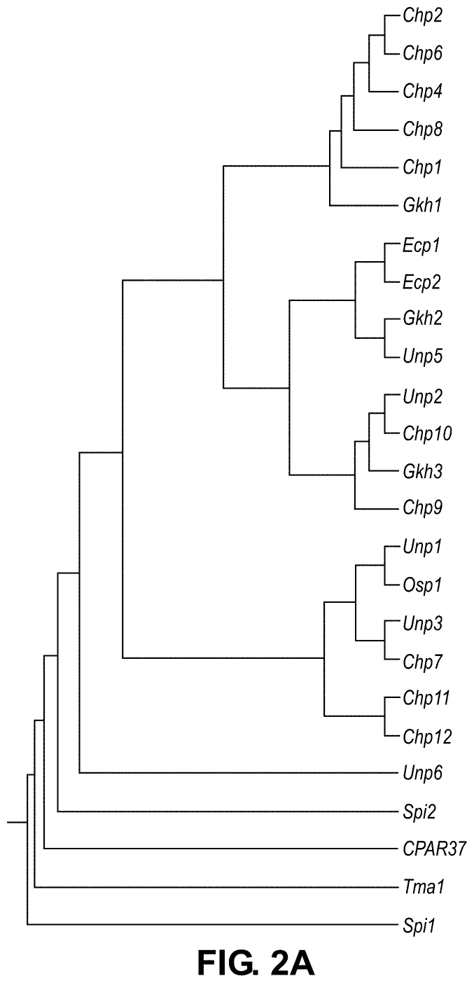

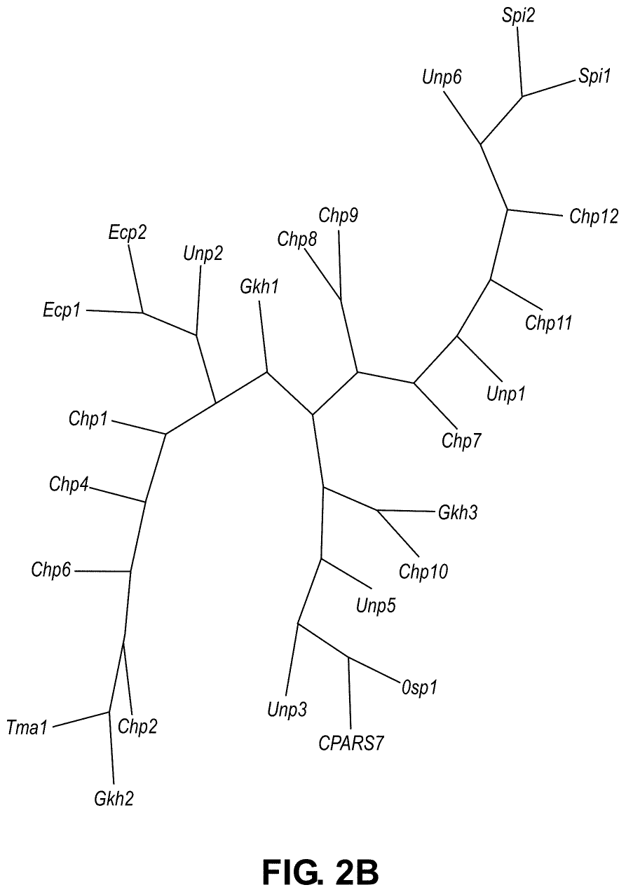

Additional information regarding the protein sequence homologies of several Chp family members is provided in Table 3. Chp1, Bdp1, Lvp1, and Lvp2 are the only Chp family members for which a predicted activity is indicated in the GenBank annotation. Chp1 (GenBank sequence NP_044319.1) is annotated as a DNA binding protein, although no data are provided to support this, and the annotation is inconsistent with a putative role in host lysis. Overall, the Chp proteins are 39-100% identical to each other and are not homologous to other peptides in the protein sequence database. Rooted and unrooted phylogenetic trees showing certain members of the Chp family are indicated in FIGS. 2A and 2B, respectively.

TABLE 3
Annotations and similarities of Chp family proteins

| Protein | Annotation (function) | Noted similarities |
|---|---|---|
| Chp1 | DNA binding protein Orf8; | 61.5% identical to Chp4 |
| | Mediates ssDNA packaging into virion; | 60% identical to Chp2 |
| | locates to the internal surface of the | 60% identical to Chp3 |
| | capsid; | Shared identity to others as well |
| | Plays role in viral attachment to the host | |
| | cell (by similarity) | |
| Chp2 | Nonstructural protein | 60% identical to Chp1 |
| | | 100% identical to Chp3 |
| | | 92.5% identical to Chp4 |
| | | 55% identical to Chp8 |
| | | 54.8% identical to Gkh1 |
| | | 60.5% identical to Unp2 |
| | | Shared identity to others as well |
| CPAR39 | Uncharacterized protein | 60% identical to Chp6 |
| Chp3 | Nonstructural protein | 60% identical to Chp1 |
| | | 100% identical to Chp2 |
| | | 92.5% identical to Chp4 |
| | | 55% identical to Chp8 |
| | | 54.8% identical to Gkh1 |
| | | 60.5% identical to Unp2 |
| | | Shared identity to others as well |
| Chp4 | Putative structural protein | 61.5% identical to Chp1 |
| | | 92.5% identical to Chp2 |
| | | 92.5% identical to Chp3 |
| | | 55% identical to Gkh1 |
| | | 64.1% identical to Unp2 |
| | | 59.5% identical to Chp8 |
| | | Shared identity to others as well |
| Chp5 | Charge reversed variant of Phage Chp4 | RK residues from Chp4 changed to |
| | Generated as a negative control protein | QE residues |
| Chp6 | Nonstructural protein | 60% identical to Chp1 |
| | | 100% identical to Chp2 |
| | | (4 residue truncation) |
| | | 100% identical to Chp3 |
| | | (4 residue truncation) |
| | | 92.5% identical to Chp4 |
| | | 55% identical to Chp8 |
| | | 54.8% identical to Gkh1 |
| | | 60.5% identical to Unp2 |
| | | Shared identity to others as well |
| Chp7 | Uncharacterized protein | 61.1% identical to Chp8 |
| | | 56.% identical to Unp3 |
| | | 50% identical to Chp9 |
| | | 53.7% identical to Gkh1 |
| | | 57.9% identical to Unp4 |
| | | Shared identity to others as well |
| Chp8 | Uncharacterized protein | 59% identical to Chp9 |
| | | 55% identical to Chp2 |
| | | 55% identical to Chp3 |
| | | 61.1% identical to Chp7 |
| | | 56.8% identical to Gkh3 |
| | | 59.5% identical to Chp4 |
| | | 47.2% identical to Chp10 |
| | | 50% identical to Gkh2 |
| | | 47.4% identical to Unp5 |
| | | 46.2% identical to Gkh1 |
| | | Shared identity to others as well |
| Chp9 | Uncharacterized protein | 59% identical to Chp8 |
| | | 59% identical to Unp2 |
| | | 57.9% identical to Chp10 |
| | | 50% identical to Chp7 |
| | | 46.2% identical to Unp6 |
| | | Shared identity to others as well |
| Chp10 | Uncharacterized protein | 63.% identical to Unp2 |
| | | 52.6% identical to Gkh2 |
| | | 57.9% identical to Chp9 |
| | | 61.8% identical to Gkh2 |
| | | 56.4% identical to Unp5 |
| | | 51.3% identical to Chp4 |
| | | 47.5% identical to Chp2 |
| | | 47.5% identical to Chp3 |
| | | 47.4% identical to Chp7 |
| | | 47.2% identical to Chp8 |
| | | 44.4% identical to Chp1 |
| | | Shared identity to others as well |
| Chp11 | Uncharacterized protein | Similar to above |
| Chp12 | Uncharacterized protein | Similar to above |
| Gkh1 | Uncharacterized protein | 55% identical to Chp4 |
| | | 54.8% identical to Chp2 |
| | | 54.8% identical to Chp3 |
| | | 53.7% identical to Chp7 |
| | | 48.8% identical to Chp10 |
| | | 46.2% identical to Chp8 |
| | | 40.5% identical to Gkh3 |
| | | 42.5% identical to Chp1 |
| | | Shared identity to others as well |
| Gkh2 | Uncharacterized protein | 70.6% identical to Unp5 |
| | | 63.6% identical to Chp10 |
| Unp1 | Unnamed protein product | 70.6% identical to Osp1 |
| | | 57.9% identical to Chp7 |
| | | 42.4% identical to Chp1 |
| | | 39.5% identical to Chp10 |
| | | 45.2% identical to Chp4 |
| | | 41.2% identical to Gkh2 |
| | | 45.2% identical to Chp2 |
| | | Shared identity to others as well |
| Ecp1 | Nonstructural protein | 60% identical to Unp2 |
| | | 56.4% identical to Chp4 |
| | | 53.8% identical to Chp2 |
| | | 53.8% identical to Chp3 |
| | | 61.8% identical to Gkh2 |
| | | 50% identical to Chp10 |
| | | 50% identical to Unp5 |
| | | 51.3% identical to Chp1 |
| | | Shared identity to others as well |
| Ecp2 | Hypothetical protein | 57.1% identical to Unp2 |
| | | 64.7% identical to Gkh2 |
| | | 53.8% identical to Chp4 |
| | | 51.3% identical to Chp2 |
| | | 51.3% identical to Chp3 |
| | | 52.8% identical to Osp1 |
| | | 47.2% identical to Chp10 |
| | | 47.5% identical to Chp1 |
| | | Shared identity to others as well |
| Tma1 | Uncharacterized protein | None |
| Osp1 | Hypothetical protein | 70.6% identical to Unp1 |
| | | 37.1% identical to Chp1 |
| | | 48.6% identical to Chp8 |
| | | 48.6% identical to Gkh3 |
| | | Shared identity to others as well |
| Unp2 | Unnamed protein product | 63.2% identical to Chp10 |
| | | 59% identical to Chp9 |
| | | 64.1% identical to Chp4 |
| | | 56.8% identical to Chp1 |
| | | 60.5% identical to Chp2 |
| | | 60.5% identical to Chp3 |
| | | Shared identity to others as well |
| Unp3 | Unnamed protein product | 56.8% identical to Chp7 |
| | | 58.8% identical to Unp1 |
| | | 59.5% identical to Osp1 |
| | | 43.2% identical to Chp9 |
| | | 45.9% identical to Gkh3 |
| | | Shared identity to others as well |
| Gkh3 | Uncharacterized protein | 52.6% identical to Chp10 |
| | | 55.9% identical to Chp8 |
| | | 50% identical to Unp2 |
| | | 42.9% identical to Chp4 |
| | | 47.2% identical to Chp9 |
| | | 40% identical to Chp2 |
| | | 40% identical to Chp3 |
| | | Shared identity to others as well |
| Unp5 | Uncharacterized protein | 61.1% identical to Gkh3 |
| | | 56.4% identical to Chp10 |
| | | 70.6% identical to Gkh2 |
| | | 53.8% identical to Chp7 |
| | | 43.6% identical to Unp2 |
| | | 48.6% identical to Chp9 |
| | | Shared identity to others as well |
| Unp6 | Uncharacterized protein | 46.2% identical to Chp9 |
| | | 44.7% identical to Chp10 |
| | | Shared identity to others as well |
| Spi1 | Hypothetical protein | No homology |
| Spi2 | Hypothetical protein | No homology |

### Example 2: Synthesis of the Chp Peptides

All Chp peptides were synthesized by GenScript, NJ, USA with capping [N-terminal acetylation (Ac) and C-terminal amidation (NH₂)] on a fee-for-service basis. GenScript assessed the purity of each peptide by high performance liquid chromatography (HPLC) and mass spectrometry (MS). GenScript also performed a solubility test for all peptides and determined the net peptide content (NPC %) using a Vario MICRO Organic Elemental Analyzer. With the exception of Chp5, Lvp1, and Lvp2, all peptides were soluble in water and were suspended at a concentration of either 1 mg/mL, 5 mg/mL or 10 mg/mL. Chp5 and Lvp1 were suspended in DMSO at a concentration of 10 mg/mL; Lvp2 was suspended in DMSO at a concentration of 2 mg/mL. The solubility of Ecp1-CAV was not determined. Control peptides RI18, RP-1, WLBU2, BAC3, GN-2 amp, GN-3 amp, GN-4 amp, GN-6 amp, and Bac8c were also synthesized at GenScript as above. All additional peptides were commercial products purchased from either GenScript or Anaspec.

### Example 3: Activity of Chp Peptides—Minimum Inhibitory Concentration (MIC) Against Gram-Negative Bacteria

The Chp peptides (excluding Chp3, which has an identical peptide sequence to Chp2) were synthesized and examined in antimicrobial susceptibility testing (AST) formats. MIC values were determined against the carbapenem-resistant P. aeruginosa clinical isolate CFS-1292 in 100% CAA medium; CAA medium supplemented with 2.5% human serum; and CAA medium supplemented with 12.5% human serum (Table 4). Several peptides, including Chp1, Chp2, Chp4, Chp6, CPAR39 (with dithiothreitol (DTT)), Chp7, Chp8, Chp10, Chp11, Ecp1, Ecp2, Osp1, Spi1, Gkh3, Unp2, Unp5, Unp6, Ecp3, Ecp4, Lvp1, ALCES1, AVQ206, CDL907, AGT915, SBR77, Chp2-M1, Chp2-Cys, Chp4::Chp2, and Chp2-CAV, exhibited superior MIC values ranging from 0.25-4 μg/mL in CAA medium supplemented with 2.5% human serum. Peptides Chp5, CPAR39 (without DTT), Gkh1, Unp1, Spi2, and Bdp1 were only poorly active and exhibited MIC values of ≥32 μg/mL in CAA medium supplemented with 2.5% human serum. Moreover, several peptides also exhibited superior MIC values ranging from 0.25-4 μg/mL in CAA medium supplemented with 12.5% human serum, as described below in Example 14.

CPAR39 is unique in this group as it contains internal cysteine residues and requires the presence of 0.5 mM DTT for activity. Chp5 was designed as a derivative of Chp4 in which all positively charged residues were changed to negative charges; it is predicted, based on studies of cationic AMPs, that cationic residues are required for the antibacterial activity and removal of the cationic residues with anionic residues will ablate activity. Accordingly, Chp5 (MIC>64 μg/mL) is an inactive variant of Chp4 (MIC=0.5 μg/mL). Both CPAR39 (without DTT) and Chp5 are used as negative controls.

TABLE 4

| | MIC (μg/mL) | MIC (μg/mL) | MIC (μg/mL) |
|---|---|---|---|
| | against CFS-1292 | against CFS-1292 | against CFS-1292 |
| | in 100% CAA | in CAA + 2.5% | in CAA + 12.5% |
| Peptide | (1^{st} run/2^{nd} run) | human serum | human serum |
| Chp1 | 1 | 2 | 1 |
| Chp2 | 0.5 | 0.5 | 0.5 |
| CPAR39 + DTT | 4 | 4 | 1 |
| CPAR39 − DTT | n.d. | 64 | n.d. |
| Chp4 | 0.5 | 0.5 | 0.5 |
| Chp5 | >32 | >64 | >32 |
| Chp6 | 0.5 | 0.25 | 0.5 |
| Chp7 | 16 | 4 | 1 |
| Chp8 | 0.5 | 2 | 2 |
| Chp9 | 8 | 8 | 2 |
| Chp10 | 0.5 | 2 | 0.5 |
| Chp11 | 16 | 4 | 4 |
| Chp12 | 8 | 8 | 4 |
| Gkh1 | 1 | 128 | 1 |
| Gkh2 | 16 | 8 | 4 |
| Gkh3 | 0.5 | 2 | 1 |
| Unp1 | 4 | 32 | 4 |
| Unp2 | 0.5 | 1 | 0.5 |
| Unp3 | 8 | 8 | 0.25 |
| Unp4 | 2 | n.d. | >16 |
| Unp5 | 2 | 2 | 0.5 |
| Unp6 | n.d. | 4 | n.d. |
| Ecp1 | 0.5 | 0.5 | 0.25 |
| Ecp2 | 0.25 | 1 | 0.25 |
| Osp1 | 16 | 0.5 | 1 |
| Spi1 | 2 | 2 | 0.25 |
| Spi2 | 16 | 64 | 0.25 |
| Ecp3 | 0.5 | 4 | 0.5 |
| Ecp4 | n.d. | 2 | n.d. |
| Bdp1 | 16 | >128 | >32 |
| Lvp1 + DTT | >64 | 2 | 0.5 |
| Lvp2 (Abp1) | 32 | 8 | n.d. |
| ALCES1 | n.d. | 2 | n.d. |
| AVQ206 | 0.5 | 2 | 0.25 |
| AVG244 | 64 | >16 | >32 |
| CDL907 | 8 | 2 | 0.5 |
| AGT915 | 2 | 1 | 0.25 |
| HH3930 | 2 | n.d. | 4 |
| Fen7875 | 2 | n.d. | 2 |
| SBR77 (Sbr1) | 4 | 0.5 | >16 |
| Chp2-M1 | 0.125 | 1 | 0.125 |
| Chp2-Cys | 8 | 2 | n.d. |
| Chp2-NC | >8 | >8 | n.d. |
| Chp4::Chp2 | 4 | 2 | n.d. |
| Chp2-CAV | n.d. | 2 | n.d. |
| Ecp1-CAV | 1 | n.d. | n.d. |
| Ecp1-M1 | 0.125/0.125 | n.d. | 0.125 |
| Chp6-M1 | 0.5/0.5 | n.d. | 0.25 |
| Chp10-M1 | 0.25/0.25 | n.d. | 0.25 |
| Chp4-M1 | <1/0.25 | n.d. | 0.25 |
| Chp7-M1 | <1 | n.d. | n.d. |
| Osp1-M1 | <l/>32 | n.d. | 16 |
| Unp2-M1 | <1/0.25 | n.d. | 0.5 |
| Unp3-M1 | <1/4 | n.d. | 4 |
| Ecp3-M1 | 0.25/0.25 | n.d. | 0.25 |
| Myo1 | >64 | n.d. | n.d. |
| Spi2-M1 | 8 | n.d. | 8 |
| Agt1-M1 | 1 | n.d. | 2 |

Control peptides

| | | | |
|---|---|---|---|
| WLBU2 | 1 | n.d | >64 |
| BAC3 | 4 | n.d | 32 |
| PRR | 32 | n.d | >128 |
| RR12 | 4 | n.d | 8 |
| RR12 polar | 4 | n.d | 32 |
| RI18 | 2 | n.d | 1 |
| RR12 hydro | 16 | n.d. | 128 |

Additional MIC testing was performed using peptides Chp1, Chp2, Chp4, CPAR39 (without DTT), Chp6, Ecp1 and Ecp2 against a range of Gram-negative organisms including Pseudomonas aeruginosa, Escherichia coli, Enterobacter cloacae, Klebsiella pneumoniae, and Acinetobacter baumannii, which includes certain major ESKAPE pathogens (Table 5). Testing was performed in CAA (containing physiological salt concentrations) that was not supplemented with 2.5% human serum, owing to the differential susceptibilities of target organisms to the presence of human serum. Superior MIC values of 1-4 μg/mL were observed against all strains tested for Chp2, Chp4, Chp6, Ecp1, and Ecp2, indicating broad spectrum activity for the present Chp peptides in the context of physiological salt concentrations. Chp2 and Ecp1 were additionally tested against Salmonella typhimurium and demonstrated to have an MIC of 2 μg/mL.

TABLE 5

| | | MIC (μg/mL) | | | | | | |
|---|---|---|---|---|---|---|---|---|
| | Organism | | CPAR | | | | | |
| | (strain | | 39 | | | | | |
| RSC | number) | Chp2 | -DTT | Chp4 | Ecp1 | Chp6 | Chp1 | Ecp2 |
| 489 | P. aeruginosa | 4 | 128 | 4 | 2 | 2 | 4 | 2 |
| | (ATCC 15692, | | | | | | | |
| | infected wound) | | | | | | | |
| 490 | P. aeruginosa | 4 | 128 | 4 | 2 | 1 | 8 | 2 |
| | (PAO1, alter- | | | | | | | |
| | nate source, | | | | | | | |
| | HER1018) | | | | | | | |
| 815 | P. aeruginosa | 4 | >128 | 4 | 2 | 1 | 8 | 2 |
| | (ATCC 27853, | | | | | | | |
| | MIC control | | | | | | | |
| | strain) | | | | | | | |
| 1108 | P. aeruginosa | 2 | 16 | 4 | 1 | 2 | 8 | 4 |
| | (ATCC 19142, | | | | | | | |
| | tracheobronchi- | | | | | | | |
| | al secretion) | | | | | | | |
| 1109 | P. aeruginosa | 4 | >128 | 4 | 2 | 4 | 8 | 4 |
| | (ATCC 17646, | | | | | | | |
| | human liver | | | | | | | |
| | abscess) | | | | | | | |
| 1110 | P. aeruginosa | 4 | 128 | 4 | 1 | 2 | 4 | 1 |
| | (ATCC 15152, | | | | | | | |
| | abscess in | | | | | | | |
| | middle ear) | | | | | | | |
| 1111 | P. aeruginosa | 4 | >128 | 4 | 2 | 4 | 8 | 4 |
| | (ATCC 14213, | | | | | | | |
| | human hip | | | | | | | |
| | wound) | | | | | | | |
| 1113 | P. aeruginosa | 4 | >128 | 4 | 4 | 2 | 8 | 2 |
| | (ATCC BAA- | | | | | | | |
| | 27, lab strain) | | | | | | | |
| 1114 | P. aeruginosa | 4 | >128 | 4 | 2 | 2 | 8 | 2 |
| | (ATCC 25102, | | | | | | | |
| | bacteriophage | | | | | | | |
| | host) | | | | | | | |
| 1115 | P. aeruginosa | 4 | 128 | 4 | 2 | 4 | 8 | 8 |
| | (ATCC 15692, | | | | | | | |
| | infected wound) | | | | | | | |
| 1292 | P. aeruginosa | 4 | 16 | 4 | 2 | 2 | 8 | 8 |
| | 453 (Human | | | | | | | |
| | clinical isolate, | | | | | | | |
| | HSS) | | | | | | | |
| 813 | E. coli (ATCC | 2 | 32 | 2 | 2 | 2 | 8 | 2 |
| | 25922, MIC | | | | | | | |
| | control strain) | | | | | | | |
| 1212 | E. coli (HM346, | 2 | 16 | 4 | 2 | 2 | 8 | 4 |
| | colon, Crohn's | | | | | | | |
| | disease) | | | | | | | |
| 1240 | Enterobacter | 4 | >128 | 4 | 2 | 4 | 8 | 4 |
| | cloacae | | | | | | | |
| | (ATCC 13047, | | | | | | | |
| | MIC control | | | | | | | |
| | strain) | | | | | | | |
| 814 | Klebsiella | 4 | 64 | 4 | 2 | 4 | 16 | 4 |
| | pneumoniae | | | | | | | |
| | (ATCC 10031 | | | | | | | |
| | (MIC control | | | | | | | |
| | strain) | | | | | | | |
| 1131 | Klebsiella spp. | 4 | >128 | 4 | 2 | 4 | 8 | 2 |
| | (HM-223; gut | | | | | | | |
| | of Crohn's | | | | | | | |
| | disease patient) | | | | | | | |
| 1138 | Klebsiella | 4 | >128 | 4 | 2 | 4 | 16 | 2 |
| | pneumoniae | | | | | | | |
| | (g2-3 HM35K) | | | | | | | |
| 1139 | Klebsiella sp. | 2 | 64 | 4 | 2 | 1 | 8 | 4 |
| | (HM-44; colon, | | | | | | | |
| | Crohn's disease) | | | | | | | |
| 30 | Acinetobacter | 2 | 64 | 4 | 2 | 2 | 8 | 2 |
| | baumannii | | | | | | | |
| | (clinical isolate | | | | | | | |
| | HSS) | | | | | | | |
| 32 | Acinetobacter | 2 | 64 | 4 | 2 | 2 | 8 | 2 |
| | baumannii | | | | | | | |
| | (clinical isolate | | | | | | | |
| | HSS) | | | | | | | |
| 27 | Salmonella | 2 | n.d. | n.d. | 2 | n.d. | n.d. | n.d. |
| | typhimurium | | | | | | | |
| | LT2 (lab | | | | | | | |
| | isolate) | | | | | | | |

n.d. = not determined

Additional MIC testing was performed for several peptides against the ESKAPE pathogens Escherichia coli, Enterobacter cloacae, Klebsiella pneumoniae, and Acinetobacter baumannii, in CAA supplemented with human serum (Table D). Superior MIC values of 1-4 μg/mL were observed against all strains tested for several peptides, as shown in Table D.

TABLE D

| | Klebsiella | Enterobacter | | Acinetobacter |
|---|---|---|---|---|
| | pneumondiae | cloacae | Escherichia coli | baumannii |
| Chp peptide | HM-44 | ATCC 13047 | ATCC 25922 | ATCC BAA 747 |
| Chp1 | 1 | 4 | 2 | 2 |
| Chp2 | 0.125 | 1 | 1 | 0.125 |
| Chp4 | 0.25 | 1 | 0.5 | 0.125 |
| Chp6 | 0.125 | 2 | 1 | 1 |
| Chp7 | 1 | >32 | 8 | >32 |
| Chp8 | 4 | 16 | 0.5 | 2 |
| Chp9 | 1 | 32 | 1 | 4 |
| Chp10 | 0.5 | 1 | 0.5 | 2 |
| Chp11 | 16 | 32 | 2 | 32 |
| Chp12 | 16 | 32 | 8 | 1 |
| Gkh1 | 0.5 | 32 | 1 | 2 |
| Gkh2 | 16 | 8 | 1 | 8 |
| Gkh3 | 4 | 4 | 2 | 4 |
| Ecp1 | 0.5 | 16 | 0.25 | 1 |
| Ecp2 | 0.25 | 16 | 0.5 | 1 |
| Ecp3 | 0.5 | 4 | 1 | 2 |
| Osp1 | 0.125 | 32 | 0.5 | 32 |
| Unp1 | 8 | >32 | 0.5 | 8 |
| Unp2 | 0.125 | 16 | 0.25 | 1 |
| Unp3 | 4 | 8 | 0.5 | 2 |
| Unp5 | 4 | 4 | 1 | 8 |
| Spi2 | 16 | 4 | 0.25 | 16 |
| Bdp1 | >32 | >32 | >32 | >32 |
| Mse1 | 8 | 8 | 0.5 | 4 |
| Avq1 | 0.25 | 4 | 0.5 | 2 |
| Avq2 | >32 | >32 | 2 | 32 |
| Cd11 | 4 | >32 | 2 | >32 |
| Agt1 | 0.5 | 4 | 1 | 4 |
| Hh1 | 4 | 32 | 2 | 32 |
| Fen1 | 4 | >32 | 4 | 32 |
| Chp2-M1 | 0.125 | 0.25 | 0.125 | 0.125 |
| Ecp1-M1 | 0.5 | 0.125 | 1 | 0.5 |
| Chp4-M1 | 0.125 | 0.25 | 0.5 | 1 |
| Chp6-M1 | 0.25 | 0.25 | 1 | 0.125 |
| Chp10-M1 | 0.25 | 0.125 | 1 | 0.5 |
| Osp1-M1 | 4 | 4 | 16 | 4 |
| Unp2-M1 | 0.25 | 0.125 | 1 | 0.125 |
| Unp3-M1 | 2 | 0.5 | 1 | 2 |
| Spi2-M1 | 1 | 1 | 2 | 4 |
| Ecp3-M1 | 1 | 1 | 1 | 4 |
| Agt1-M1 | 1 | 1 | 8 | 0.125 |
| Chp5 | 8 | >32 | 4 | >32 |
| Chp2 (amino | 0.25 | >32 | 0.5 | >32 |
| acids scrambled) | | | | |
| Chp2-M1 | 0.125 | 0.5 | 0.5 | 0.25 |
| (amino acids | | | | |
| scrambled) | | | | |
| Ecp1 (amino | 32 | >32 | >32 | >32 |
| acids scrambled) | | | | |

While not wishing to be bound by theory, it is postulated that the presence of arginine at certain positions of a Chp peptide in place of lysine may contributed to enhanced activity against Gram-negative ESKAPE pathogens.

The MIC values for both Chp2 and Chp4 were also determined and compared to that of a range of AMPs from the literature (including innate immune effectors and derivatives thereof), against the laboratory P. aeruginosa strain PAO1 in Mueller-Hinton broth supplemented with either 50% human plasma or human serum (Table 6). Here, the use of PAO1 (a laboratory isolate) enables testing in the presence of elevated serum or plasma concentrations; PAO1, unlike most clinical isolates, is insensitive to the antibacterial activity of human blood matrices. In Table 6, the MIC values for Chp2 and Chp4 were 2 μg/mL; in comparison, only RI18 and protegrin were similarly active (MIC=1-4 μg/mL), and the 18 additional peptides tested were either inactive or poorly active.

TABLE 6

| | Minimal inhibitory | |
|---|---|---|
| | concentration | |
| | (μg/mL) | |
| | Human | Human |
| Agent | Plasma | Serum |
| Protegrin 1 | 1 | 4 |
| Indolicidin | >64 | >64 |
| LL-37 | >64 | >64 |
| LL-37 (18-37) | >64 | >64 |
| LL-37 (17-29) | >64 | >64 |
| GN-2 amp | >64 | >64 |
| GN-3 amp | >64 | >64 |
| GN-4 amp | >64 | >64 |
| Pediocin | >64 | >64 |
| Parasin | >64 | >64 |
| PGLa | >64 | >64 |
| OV-1 | 32 | 32 |
| Dermaseptin | >64 | >64 |
| WLBU2 | >64 | >64 |
| RP-1 | 32 | 64 |
| T9W | 16 | 32 |
| BAC3 | >64 | >64 |
| GN-6 amp | >64 | >64 |
| Bac 8c | >64 | >64 |
| RI18 | 2 | 1 |
| Chp2 | 2 | 2 |
| Chp4 | 2 | 2 |

### Example 4: Activity of Chp Peptides—Eradication of Biofilm of Gram-Negative Bacteria

To evaluate anti-biofilm activity, MBEC (minimum biofilm eradication concentration) values were determined for peptides Chp2 and Chp4 against mature biofilms formed by P. aeruginosa strain ATCC 17647 in tryptic soy broth medium supplemented with 2% glucose. MBEC values of 0.25 μg/mL were observed for both Chp2 and Chp4 (Table 7), which are consistent with a potent ability to eradicate mature biofilms. In comparison, the activity of RI18, a highly active AMP (15), was observed to be substantially lower, 4 μg/mL, and the activity of T4 lysozyme, a poorly active lysin, was observed to be >64 μg/mL.

TABLE 7

| Agent | MBEC (μg/mL) |
|---|---|
| RI18 | 4 |
| Chp2 | 0.25 |
| Chp4 | 0.25 |
| T4LYZ | >64 |

MBEC values were likewise determined for Chp2, Chp2-M1, and Chp10-M1 in eight different strains of five species of Gram-negative bacteria. Biofilms were formed over 24 hours in LB media, washed with phosphate-buffered saline, and then treated for 16 hours with either the Chp peptide or a control (LL-37 antimicrobial peptide or tobramycin antibiotic). The films were then washed and stained with crystal violet to visualize the MBEC. The results, shown below in Table 8, demonstrate that all of Chp2, Chp2-M1, and Chp10-M1 exhibited potent antibiofilm activity.

TABLE 8

| | MBEC (μg/mL) | | | | | | |
|---|---|---|---|---|---|---|---|
| | | | | LL-37 | | Tobramycin | |
| Bacterial strain | Chp2 | Chp2-M1 | Chp10-M1 | (MIC) | | (MIC) | |
| P. aeruginosa | 0.25 | 0.125 | 0.125 | 8 | (16) | 0.03 | (<0.5) |
| (ATCC 27853) | | | | | | | |
| A. baumanni | 0.5 | 0.25 | 0.25 | 0.25 | (16) | 0.03 | (<0.5) |
| (ATCC BAA-747) | | | | | | | |
| K. pneumoniae | 4 | 4 | 2 | 32 | (16) | 1 | (<0.5) |
| (ATCC 700603) | | | | | | | |
| K. pneumoniae (KP1) | 2 | 2 | 1 | 16 | (16) | 1 | (<0.5) |
| E. cloacae | 2 | 2 | 1 | 8 | (16) | <0.03 | (<0.5) |
| (CCUG 63317) | | | | | | | |
| E. coli (ATCC 25922) | 2 | 1 | 1 | 16 | (16) | <0.03 | (<0.5) |
| E. coli (ATCC 35329) | 4 | 4 | 2 | 16 | (16) | <0.03 | (<0.5) |
| E. coli (EC2) | 2 | 2 | 1 | 8 | (16) | <0.03 | (<0.5) |

### Example 5: Combination of Chp Peptides and Antibiotics

To evaluate synergy between either Chp2 or Chp4 and a range of 11 antibiotics, each combination of Chp2 with the 11 antibiotics and Chp4 with the 11 antibiotics was tested in a standard checkerboard assay format using P. aeruginosa strain CFS-1292 in CAA media supplemented with 2.5% human serum. In the checkerboard assay, fractional inhibitory concentration index (FICI) values are calculated. FICI values ≤0.5 are consistent with synergy, values >0.5-1 are consistent with strongly additive activity, values of 1-2 are consistent with additive activity, and values >2 are considered antagonistic. As shown below in Table 9, for both Chp2 and Chp4, the values were consistent with either synergy (i.e., ≤0.5) or strongly additive (i.e., >0.5-1) interactions between the Chp peptide and the antibiotic.

TABLE 9

| Antibiotic | Chp2 | Chp4 |
|---|---|---|
| Amikacin | 0.500 | 0.500 |
| Azithromycin | 0.156 | 0.156 |
| Aztreonam | 0.500 | 0.375 |
| Ciprofloxacin | 0.500 | 0.375 |
| Colistin | 0.375 | 0.375 |
| Fosfomycin | 0.250 | 0.250 |
| Gentamicin | 0.281 | 0.250 |
| Imipenem | 0.188 | 0.375 |
| Piperacillin | 0.188 | 0.188 |
| Rifampicin | 0.563 | 0.750 |
| Tobramycin | 0.266 | 0.266 |

Additional FICI values were calculated for Chp2, Chp2-M1, Chp4-M1, Chp6-M1, Chp10-M1, and Unp2-M1 using the same method with P. aeruginosa strain CFS-1292. The results are shown below in Tables 10-15. For all of the tested Chp peptides, the FICI values were consistent with either synergy (i.e., ≤0.5) or strongly additive (i.e., >0.5-1) interactions between the Chp peptide and the antibiotic, with the exception of Chp4-M1 and imipenem.

TABLE 10
Chp 2 and P. aeruginosa FICI values

| | Chp2 | | Antibiotic | | |
|---|---|---|---|---|---|
| | MIC | Chp2 | MIC | Antibiotic | FIC |
| Antibiotic | (μg/mL) | FIC | (μg/mL) | FIC | Index |
| Azithromycin | 0.0625 | 0.125 | 16 | 0.25 | 0.375 |
| Aztreonam | 0.0625 | 0.125 | 16 | 0.25 | 0.375 |
| Colistin | 0.0625 | 0.125 | 0.015625 | 0.25 | 0.375 |
| Imipenem | 0.125 | 0.25 | 4 | 0.125 | 0.375 |
| Levofloxacin | 0.0625 | 0.25 | 2 | 0.25 | 0.5 |
| Meropenem | 0.0625 | 0.125 | 4 | 0.25 | 0.375 |
| Tobramycin | 0.0625 | 0.125 | 0.125 | 0.25 | 0.375 |
| Amikacin | 0.25 | 0.25 | 0.5 | 0.25 | 0.5 |
| Ciprofloxacin | 0.125 | 0.25 | 0.5 | 0.5 | 0.75 |
| Gentamicin | 0.03125 | 0.0625 | 0.5 | 0.5 | 0.5625 |
| Piperacillin | 0.25 | 0.5 | 4 | 0.5 | 1 |

TABLE 11
Chp2-Ml and P. aeruginosa FICI values

| | Chp2-M1 | | Antibiotic | | |
|---|---|---|---|---|---|
| | MIC | Chp2-M1 | MIC | Antibiotic | FIC |
| Antibiotic | (μg/mL) | FIC | (μg/mL) | FIC | Index |
| Azithromycin | 0.0625 | 0.25 | 8 | 0.125 | 0.375 |
| Aztreonam | 0.015625 | 0.0625 | 4 | 0.5 | 0.5625 |
| Colistin | 0.03125 | 0.125 | 0.015625 | 0.25 | 0.375 |
| Imipenem | 0.0625 | 0.25 | 8 | 0.25 | 0.5 |
| Levofloxacin | 0.0625 | 0.25 | 2 | 0.25 | 0.5 |
| Meropenem | 0.0625 | 0.25 | 4 | 0.25 | 0.5 |
| Tobramycin | 0.015625 | 0.0625 | 0.125 | 0.25 | 0.3125 |
| Amikacin | 0.0625 | 0.125 | 0.5 | 0.25 | 0.375 |
| Ciprofloxacin | 0.25 | 0.5 | 0.0625 | 0.0625 | 0.5625 |
| Gentamicin | 0.25 | 0.5 | 0.03125 | 0.0625 | 0.5625 |
| Piperacillin | 0.25 | 0.5 | 1 | 0.125 | 0.625 |

TABLE 12
Chp4-M1 and P. aeruginosa FICI values

| | Chp4-M1 | | Antibiotic | | |
|---|---|---|---|---|---|
| | MIC | Chp4-M1 | MIC | Antibiotic | FIC |
| Antibiotic | (μg/mL) | FIC | (μg/mL) | FIC | Index |
| Azithromycin | 0.125 | 0.25 | 16 | 0.25 | 0.5 |
| Aztreonam | 0.0625 | 0.125 | 2 | 0.25 | 0.375 |
| Colistin | 0.03125 | 0.0625 | 0.03125 | 0.25 | 0.3125 |
| Imipenem | 0.0625 | 0.125 | 32 | 1 | 1.125 |
| Levofloxacin | 0.125 | 0.25 | 4 | 0.5 | 0.75 |
| Meropenem | 0.125 | 0.25 | 4 | 0.25 | 0.5 |
| Tobramycin | 0.125 | 0.25 | 0.125 | 0.25 | 0.5 |
| Amikacin | 0.25 | 0.5 | 0.125 | 0.0625 | 0.5625 |
| Ciprofloxacin | 0.015625 | 0.03125 | 1 | 0.5 | 0.53125 |
| Gentamicin | 0.25 | 0.5 | 0.0625 | 0.125 | 0.625 |
| Piperacillin | 0.125 | 0.25 | 8 | 0.5 | 0.75 |

TABLE 13
Chp6-M1 and P. aeruginosa FICI values

| | Chp6-M1 | | Antibiotic | | |
|---|---|---|---|---|---|
| | MIC | Chp6-Ml | MIC | Antibiotic | FIC |
| Antibiotic | (μg/mL) | FIC | (μg/mL) | FIC | Index |
| Azithromycin | 0.0625 | 0.0625 | 16 | 0.25 | 0.313 |
| Aztreonam | 0.125 | 0.125 | 2 | 0.25 | 0.375 |
| Colistin | 0.125 | 0.125 | 0.015625 | 0.25 | 0.375 |
| Imipenem | 0.25 | 0.25 | 8 | 0.25 | 0.5 |
| Levofloxacin | 0.25 | 0.25 | 2 | 0.25 | 0.5 |
| Meropenem | 0.25 | 0.25 | 4 | 0.25 | 0.5 |
| Tobramycin | 0.0625 | 0.0625 | 0.125 | 0.25 | 0.313 |
| Amikacin | 0.125 | 0.25 | 0.5 | 0.25 | 0.5 |
| Ciprofloxacin | 0.25 | 0.5 | 0.125 | 0.125 | 0.625 |
| Gentamicin | 0.25 | 0.5 | 0.03125 | 0.0625 | 0.563 |
| Piperacillin | 0.25 | 0.5 | 2 | 0.25 | 0.75 |

TABLE 14
Chp10-M1 and P. aeruginosa FICI values

| | Chp10-M1 | | Antibiotic | | |
|---|---|---|---|---|---|
| | MIC | Chp10-M1 | MIC | Antibiotic | FIC |
| Antibiotic | (μg/mL) | FIC | (μg/mL) | FIC | Index |
| Azithromycin | 0.125 | 0.25 | 8 | 0.125 | 0.375 |
| Aztreonam | 0.125 | 0.25 | 2 | 0.25 | 0.5 |
| Colistin | 0.125 | 0.25 | 0.00780625 | 0.1249 | 0.375 |
| Imipenem | 0.125 | 0.125 | 4 | 0.125 | 0.25 |
| Levofloxacin | 0.25 | 0.25 | 2 | 0.25 | 0.5 |
| Meropenem | 0.125 | 0.125 | 2 | 0.25 | 0.375 |
| Tobramycin | 0.25 | 0.25 | 0.0625 | 0.25 | 0.5 |
| Amikacin | 0.03125 | 0.125 | 0.25 | 0.125 | 0.25 |
| Ciprofloxacin | 0.125 | 0.5 | 0.125 | 0.125 | 0.625 |
| Gentamicin | 0.0625 | 0.25 | 0.0625 | 0.125 | 0.375 |
| Piperacillin | 0.125 | 0.5 | 2 | 0.25 | 0.75 |

TABLE 15
Unp2-M1 and P. aeruginosa FICI values

| | Unp2-M1 | | Antibiotic | | |
|---|---|---|---|---|---|
| | MIC | Unp2-M1 | MIC | Antibiotic | FIC |
| Antibiotic | (μg/mL) | FIC | (μg/mL) | FIC | Index |
| Azithromycin | 0.125 | 0.25 | 8 | 0.125 | 0.375 |
| Aztreonam | 0.25 | 0.25 | 1 | 0.125 | 0.375 |
| Colistin | 0.125 | 0.25 | 0.015625 | 0.25 | 0.5 |
| Imipenem | 0.0625 | 0.125 | 8 | 0.25 | 0.375 |
| Levofloxacin | 0.125 | 0.25 | 2 | 0.25 | 0.5 |
| Meropenem | 0.03125 | 0.0625 | 4 | 0.25 | 0.3125 |
| Tobramycin | 0.03125 | 0.03125 | 0.125 | 0.25 | 0.281 |
| Amikacin | 0.125 | 0.25 | 0.5 | 0.25 | 0.5 |
| Ciprofloxacin | 0.25 | 0.5 | 0.25 | 0.25 | 0.75 |
| Gentamicin | 0.125 | 0.25 | 0.125 | 0.25 | 0.5 |
| Piperacillin | 0.25 | 0.5 | 2 | 0.25 | 0.75 |

FICI values were also calculated for Chp2, Chp2-M1, Chp4-M1, Chp6-M1, Chp10-M1, and Unp2-M1 using the same method with Klebsiella pneumoniae strain 1139. The results are shown below in Tables 16-21. For all of the tested Chp peptides, the values were consistent with either synergy (i.e., ≤0.5) or strongly additive (i.e., >0.5-1) interactions between the Chp peptide and the antibiotic.

TABLE 16
Chp2 and Klebsiella pneumoniae FICI values

| | | | Antibiotic | | |
|---|---|---|---|---|---|
| | Chp2 MIC | | MIC | Antibiotic | FIC |
| Antibiotic | (μg/mL) | Chp2 FIC | (μg/mL) | FIC | Index |
| Azithromycin | 0.0625 | 0.125 | 1 | 0.125 | 0.25 |
| Aztreonam | 0.3125 | 0.0625 | 0.0078125 | 0.5 | 0.563 |
| Colistin | 0.25 | 0.5 | 0.0078125 | 0.125 | 0.625 |
| Imipenem | 0.125 | 0.25 | 0.0625 | 0.25 | 0.5 |
| Levofloxacin | 0.0625 | 0.25 | 0.0078125 | 0.25 | 0.5 |
| Meropenem | 0.0625 | 0.0625 | 0.015625 | 0.25 | 0.313 |
| Tobramycin | 0.125 | 0.25 | 0.0078125 | 0.25 | 0.5 |

TABLE 17
Chp2-M1 and Klebsiella pneumoniae FICI values

| | Chp2-M1 | | Antibiotic | | |
|---|---|---|---|---|---|
| | MIC | Chp2-M1 | MIC | Antibiotic | FIC |
| Antibiotic | (μg/mL) | FIC | (μg/mL) | FIC | Index |
| Azithromycin | 0.0625 | 0.25 | 1 | 0.25 | 0.5 |
| Aztreonam | 0.03125 | 0.125 | 0.0078125 | 0.5 | 0.625 |
| Colistin | 0.0625 | 0.25 | 0.015625 | 0.125 | 0.375 |
| Imipenem | 0.0625 | 0.25 | 0.0625 | 0.25 | 0.5 |
| Levofloxacin | 0.0625 | 0.25 | 0.0078125 | 0.25 | 0.5 |
| Meropenem | 0.0625 | 0.25 | 0.015625 | 0.25 | 0.5 |
| Tobramycin | 0.125 | 0.5 | 0.0039063 | 0.25 | 0.75 |

TABLE 18
Chp4-M1 and Klebsiella pneumoniae FICI values

| | Chp4-M1 | | Antibiotic | | |
|---|---|---|---|---|---|
| | MIC | Chp4-M1 | MIC | Antibiotic | FIC |
| Antibiotic | (μg/mL) | FIC | (μg/mL) | FIC | Index |
| Azithromycin | 0.125 | 0.25 | 1 | 0.25 | 0.5 |
| Aztreonam | 0.0625 | 0.125 | 0.0078125 | 0.5 | 0.625 |
| Colistin | 0.125 | 0.25 | 0.03125 | 0.25 | 0.5 |
| Imipenem | 0.0625 | 0.125 | 0.0625 | 0.25 | 0.375 |
| Levofloxacin | 0.125 | 0.25 | 0.0078125 | 0.25 | 0.5 |
| Meropenem | 0.25 | 0.25 | 0.0078125 | 0.125 | 0.375 |
| Tobramycin | 0.125 | 0.25 | 0.00390625 | 0.125 | 0.375 |

TABLE 19
Chp6-M1 and Klebsiella pneumoniae FICI values

| | Chp6-M1 | | Antibiotic | | |
|---|---|---|---|---|---|
| | MIC | Chp6-Ml | MIC | Antibiotic | FIC |
| Antibiotic | (μg/mL) | FIC | (μg/mL) | FIC | Index |
| Azithromycin | 0.0625 | 0.0625 | 1 | 0.5 | 0.563 |
| Aztreonam | 0.125 | 0.125 | 0.0078125 | 0.25 | 0.375 |
| Colistin | 0.25 | 0.25 | 0.015625 | 0.125 | 0.375 |
| Imipenem | 0.25 | 0.25 | 0.0625 | 0.25 | 0.5 |
| Levofloxacin | 0.25 | 0.25 | 0.0078125 | 0.25 | 0.5 |
| Meropenem | 0.125 | 0.125 | 0.015625 | 0.25 | 0.375 |
| Tobramycin | 0.25 | 0.25 | 0.0039063 | 0.125 | 0.375 |

TABLE 20
Chp10-M1 and Klebsiella pneumoniae FICI values

| | Chp10M1 | | Antibiotic | | |
|---|---|---|---|---|---|
| | MIC | Chp10-M1 | MIC | Antibiotic | FIC |
| Antibiotic | (μg/mL) | FIC | (μg/mL) | FIC | Index |
| Azithromycin | 0.125 | 0.25 | 1 | 0.125 | 0.375 |
| Aztreonam | 0.125 | 0.25 | 0.0078125 | 0.25 | 0.5 |
| Colistin | 0.125 | 0.25 | 0.03125 | 0.25 | 0.5 |
| Imipenem | 0.25 | 0.25 | 0.0625 | 0.25 | 0.5 |
| Levofloxacin | 0.125 | 0.125 | 0.0078125 | 0.125 | 0.25 |
| Meropenem | 0.125 | 0.125 | 0.015625 | 0.125 | 0.25 |
| Tobramycin | 0.25 | 0.25 | 0.00390625 | 0.125 | 0.375 |

TABLE 21
Unp2-M1 and Klebsiella pneumoniae FICI values

| | Unp2-M1 | | Antibiotic | | |
|---|---|---|---|---|---|
| | MIC | Unp2-M1 | MIC | Antibiotic | FIC |
| Antibiotic | (μg/mL) | FIC | (μg/mL) | FIC | Index |
| Azithromycin | 0.0625 | 0.125 | 1 | 0.125 | 0.25 |
| Aztreonam | 0.03125 | 0.0625 | 0.0078125 | 0.0078125 | 0.313 |
| Colistin | 0.125 | 0.25 | 0.03125 | 0.03125 | 0.375 |
| Imipenem | 0.03125 | 0.0625 | 0.0625 | 0.0625 | 0.313 |
| Levofloxacin | 0.03125 | 0.0625 | 0.015625 | 0.015625 | 0.313 |
| Meropenem | 0.015625 | 0.03125 | 0.03125 | 0.03125 | 0.281 |
| Tobramycin | 0.125 | 0.25 | 0.0039063 | 0.125 | 0.375 |

FICI values were also calculated for Chp2, Chp2-M1, Chp4-M1, Chp6-M1, Chp10-Ml, and Unp2-M1 using the same method with Acinetobacter baumannii strain 30. The results are shown below in Tables 22-27. For all of the tested Chp peptides, the values were consistent with either synergy (i.e., ≤0.5) or strongly additive (i.e., >0.5-1) interactions between the Chp peptide and the antibiotic.

TABLE 22
Chp2 and Acinetobacter baumannii FICI values

| | | | Antibiotic | | |
|---|---|---|---|---|---|
| | Chp2 MIC | | MIC | Antibiotic | FIC |
| Antibiotic | (μg/mL) | Chp2 FIC | (μg/mL) | FIC | Index |
| Azithromycin | 0.03125 | 0.0625 | 0.5 | 0.5 | 0.563 |
| Aztreonam | 0.0625 | 0.0625 | 8 | 0.25 | 0.313 |
| Colistin | 0.25 | 0.25 | 0.000976563 | 0.125 | 0.375 |
| Imipenem | 0.015625 | 0.03125 | 0.5 | 0.5 | 0.531 |
| Levofloxacin | 0.25 | 0.25 | 0.03125 | 0.25 | 0.5 |
| Meropenem | 0.125 | 0.125 | 0.0625 | 0.25 | 0.375 |
| Tobramycin | 0.0625 | 0.0625 | 0.015625 | 0.125 | 0.188 |
| Amikacin | 0.03125 | 0.03125 | 0.0625 | 0.25 | 0.281 |
| Ciprofloxacin | 0.5 | 0.5 | 0.001953125 | 0.03125 | 0.531 |
| Gentamicin | 0.0625 | 0.0625 | 0.03125 | 0.25 | 0.313 |
| Piperacillin | 0.125 | 0.125 | 8 | 0.25 | 0.375 |

TABLE 23
Chp2-M1 and Acinetobacter baumannii FICI values

| | Chp2-M1 | | Antibiotic | | |
|---|---|---|---|---|---|
| | MIC | Chp2-M1 | MIC | Antibiotic | FIC |
| Antibiotic | (μg/mL) | FIC | (μg/mL) | FIC | Index |
| Azithromycin | 0.03125 | 0.125 | 0.5 | 0.25 | 0.375 |
| Aztreonam | 0.0625 | 0.25 | 8 | 0.25 | 0.5 |
| Colistin | 0.0625 | 0.25 | 0.001953125 | 0.5 | 0.75 |
| Imipenem | 0.03125 | 0.125 | 0.125 | 0.125 | 0.25 |
| Levofloxacin | 0.03125 | 0.125 | 0.03125 | 0.25 | 0.375 |
| Meropenem | 0.0625 | 0.25 | 0.0625 | 0.25 | 0.5 |
| Tobramycin | 0.03125 | 0.125 | 0.015625 | 0.25 | 0.375 |
| Amikacin | 0.015625 | 0. .03125 | 0.125 | 0.5 | 0.531 |
| Ciprofloxacin | 0.0625 | 0.0625 | 0.0625 | 0.5 | 0.563 |
| Gentamicin | 0.125 | 0.125 | 0.03125 | 0.5 | 0.625 |
| Piperacillin | 0.125 | 0.25 | 8 | 0.25 | 0.5 |

TABLE 24
Chp4-M1 and Acinetobacter baumannii FICI values

| | Chp4- | Chp4- | Antibiotic | | |
|---|---|---|---|---|---|
| | M1 MIC | M1 | MIC | Antibiotic | FIC |
| Antibiotic | (μg/mL) | FIC | (μg/mL) | FIC | Index |
| Azithromycin | 0.0625 | 0.125 | 0.5 | 0.25 | 0.375 |
| Aztreonam | 0.03125 | 0.0625 | 16 | 0.25 | 0.313 |
| Colistin | 0.015625 | 0.03125 | 0.00390625 | 0.5 | 0.531 |
| Imipenem | 0.0625 | 0.125 | 0.125 | 0.125 | 0.25 |
| Levofloxacin | 0.0625 | 0.25 | 0.03125 | 0.25 | 0.5 |
| Meropenem | 0.03125 | 0.0625 | 0.125 | 0.25 | 0.313 |
| Tobramycin | 0.0625 | 0.125 | 0.03125 | 0.5 | 0.625 |
| Amikacin | 0.25 | 0.5 | 0.0625 | 0.5 | 1 |
| Ciprofloxacin | 0.03125 | 0.0625 | 0.0625 | 0.5 | 0.563 |
| Gentamicin | 0.0625 | 0.0625 | 0.03125 | 0.5 | 0.563 |
| Piperacillin | 0.25 | 0.25 | 8 | 0.25 | 0.5 |

TABLE 25
Chp6-M1 and Acinetobacter baumannii FICI values

| | Chp6-M1 | Chp6- | Antibiotic | | |
|---|---|---|---|---|---|
| | MIC | M1 | MIC | Antibiotic | FIC |
| Antibiotic | (μg/mL) | FIC | (μg/mL) | FIC | Index |
| Azithromycin | 0.0625 | 0.25 | 0.5 | 0.25 | 0.5 |
| Aztreonam | 0.125 | 0.25 | 8 | 0.25 | 0.5 |
| Colistin | 0.0625 | 0.25 | 0.001953125 | 0.25 | 0.5 |
| Imipenem | 0.125 | 0.25 | 0.125 | 0.125 | 0.375 |
| Levofloxacin | 0.0625 | 0.125 | 0.0625 | 0.25 | 0.375 |
| Meropenem | 0.03125 | 0.0625 | 0.125 | 0.25 | 0.313 |
| Tobramycin | 0.0625 | 0.125 | 0.015625 | 0.25 | 0.375 |
| Amikacin | 0.0625 | 0.0625 | 0.125 | 0.5 | 0.563 |
| Ciprofloxacin | 0.0625 | 0.125 | 0.0625 | 0.5 | 0.625 |
| Gentamicin | 0.0625 | 0.0625 | 0.03125 | 0.5 | 0.563 |
| Piperacillin | 0.03125 | 0.0625 | 16 | 0.5 | 0.563 |

TABLE 26
Chp10-M1 and Acinetobacter baumannii FICI values

| | Chp10-M1 | Chp10- | Antibiotic | | |
|---|---|---|---|---|---|
| | MIC | M1 | MIC | Antibiotic | FIC |
| Antibiotic | (μg/mL) | FIC | (μg/mL) | FIC | Index |
| Azithromycin | 0.015625 | 0.03125 | 0.5 | 0.5 | 0.531 |
| Aztreonam | 0.125 | 0.25 | 8 | 0.25 | 0.5 |
| Colistin | 0.125 | 0.25 | 0.000976563 | 0.25 | 0.5 |
| Imipenem | 0.125 | 0.25 | 0.125 | 0.125 | 0.375 |
| Levofloxacin | 0.015625 | 0.03125 | 0.0625 | 0.25 | 0.281 |
| Meropenem | 0.125 | 0.25 | 0.0625 | 0.125 | 0.375 |
| Tobramycin | 0.0625 | 0.125 | 0.015625 | 0.25 | 0.375 |
| Amikacin | 0.0625 | 0.0625 | 0.0625 | 0.5 | 0.563 |
| Ciprofloxacin | 0.03125 | 0.0625 | 0.125 | 0.5 | 0.563 |
| Gentamicin | 0.5 | 0.5 | 0.000976563 | 0.015625 | 0.516 |
| Piperacillin | 0.125 | 0.25 | 4 | 0.25 | 0.5 |

TABLE 27
Unp2-M1 and Acinetobacter baumannii FICI values

| | Unp2-M1 | Unp2- | Antibiotic | | |
|---|---|---|---|---|---|
| | MIC | M1 | MIC | Antibiotic | FIC |
| Antibiotic | (μg/mL) | FIC | (μg/mL) | FIC | Index |
| Azithromycin | 0.03125 | 0.03125 | 0.5 | 0.5 | 0.531 |
| Aztreonam | 0.0625 | 0.0625 | 16 | 0.25 | 0.313 |
| Colistin | 0.125 | 0.125 | 0.001953125 | 0.5 | 0.625 |
| Imipenem | 0.125 | 0.125 | 0.25 | 0.25 | 0.375 |
| Levofloxacin | 0.0625 | 0.0625 | 0.0625 | 0.25 | 0.313 |
| Meropenem | 0.125 | 0.25 | 0.125 | 0.25 | 0.5 |
| Tobramycin | 0.125 | 0.125 | 0.015625 | 0.25 | 0.375 |
| Amikacin | 0.0625 | 0.0625 | 0.125 | 0.5 | 0.563 |
| Ciprofloxacin | 0.0625 | 0.125 | 0.0625 | 0.25 | 0.375 |
| Gentamicin | 0.0625 | 0.0625 | 0.03125 | 0.5 | 0.563 |
| Piperacillin | 0.125 | 0.25 | 8 | 0.25 | 0.5 |

### Example 6: Assessment of Hemolytic Activity of Chp Peptides

Antimicrobial peptides amenable for use in treating invasive infections should show low toxicity against erythrocytes (Oddo A. et al, 2017. Methods Mol Biol 1548:427-435). To examine the potential for hemolytic activity, a common methodology (described in Materials and Methods above) was used for measuring the ability of AMPs to lyse red blood cells based on the determination of minimal hemolytic concentrations (MHCs) against human red blood cells. For the majority of Chp peptides tested, no evidence of hemolysis was observed, with MHC values of >128 μg/mL (Table 28). Triton X100 control was tested at a starting concentration of 2%, with MHC being the minimum amount of peptide resulting in more than 5% lysis observed. In comparison, five AMPs with known hemolytic activity, including WLBU2, RI18, R12, RR12p, and RR12h, were observed with MHC values ranging from 1-128 μg/mL. Triton X-100, a membranolytic detergent commonly used as a positive control in hemolytic assays, was hemolytic over a range of concentrations from 2% to 0.007%. These findings suggest that Chp peptides do not have the in vitro toxicity (i.e., hemolytic activity) commonly observed for AMPs. This property is expected of the remaining Chp peptides of Tables 1, 2, and C based not only on percent sequence identity, 3D structural similarity, and charge profile, but also on the anticipation that, as lytic agents, the present peptides will most likely be very highly specific for the Gram-negative cell envelope.

TABLE 28
Minimal hemolytic concentration (MHC) values
determined against human red blood cells

| | |
|---|---|
| Agent | MHC (μg/mL) |

Control Peptides (Run #1/Run #2)

| | |
|---|---|
| WLBU2 | 8 |
| RI18 | 128 |
| RR12 | 8/1 |
| RR12p | 4/4 |
| RR12h | 32/1 |
| Triton control* | 1 |

Chp Peptides

| | |
|---|---|
| Chp1 | >128 |
| Chp2 | >128 |
| CPAR39 | >128 |
| Chp4 | >128 |
| Chp5 | >128 |
| Chp6 | >128 |
| Chp7 | >128 |
| Chp8 | >128 |
| Chp9 | >128 |
| Chp10 | >128 |
| Chp11 | >128 |
| Chp12 | >128 |
| Gkh1 | >128 |
| Gkh2 | >128 |
| Gkh3 | >128 |
| Ecp1 | >128 |
| Ecp2 | >128 |
| Ecp3 | >128 |
| Ecp4 | >128 |
| Osp1 | >128 |
| Unpl | >128 |
| Unp2 | >128 |
| Unp3 | >128 |
| Unp4 | >128 |
| Unp5 | >128 |
| Unp6 | >128 |
| Spi1 | >128 |
| Spi2 | >128 |
| Mse1 | >128 |
| Bdp1 | >128 |
| Lvp1 | n.d. |
| Lvp2 | 8 |
| ALCES1 | >128 |
| AVQ206 | >128 |
| AVQ244 | >128 |
| CDL907 | >128 |
| AGT915 | >128 |
| HH3930 | n.d. |
| Fen7875 | n.d. |
| SBR77 | >128 |
| Chp2-M1 | >128 |
| Chp2-Cys | 8 |
| Chp2-NC | 8 |
| Chp4::Chp2 | 2 |
| Chp2-CAV | 4 |
| Ecp1-CAV | n.d. |
| Ecp1-M1 | >128 |
| Chp4-M1 | >128 |
| Chp6-M1 | >128 |
| Chp10-M1 | >128 |
| Osp1-M1 | >128 |
| Unp2-M1 | >128 |
| Unp3-M1 | >128 |
| Spi2-M1 | >128 |
| Ecp3-M1 | >128 |
| Agt1-M1 | >128 |

### Example 7: Duration of Lytic Activity Against Gram Negative Bacteria

The activity of Chp2 and Chp4 was examined against P. aeruginosa strain CFS-1292 in the time-kill format using CAA with 2.5% human serum as described in Materials and Methods. Assessments of bacterial viability at 1, 3, and 24 hours after treatment with 1 μg/mL and 10 μg/mL concentrations of either Chp2 or Chp4 resulted in multi-log fold decreases consistent with potent bactericidal activity in all cases (Table 29). Table 29 sets forth the log reduction of colony forming units (compared to untreated controls) determined using the time-kill format for P. aeruginosa strain CFS-1292 after treatment in CAA supplemented with 2.5% human serum.

TABLE 29

| | Loss of bacterial viability | | |
|---|---|---|---|
| | (log10 CFU/mL) | | |
| Treatment | 1 hour | 3 hours | 24 hours |
| Chp2 (1 μg/mL) | >3.5 | >4 | >4.8 |
| Chp2 (10 μg/mL) | >3.5 | >4 | >4.8 |
| Chp4 (1 μg/mL) | >3.5 | >4 | >4.8 |
| Chp4 (10 μg/mL) | >3.5 | >4 | >4.8 |

Additional 24-hour time-kill assays were conducted using 13 different Chp peptides at 0.2×, 1×, and 5×MIC (as well as a buffer control) against P. aeruginosa, K. pneumoniae, and A. baumannii. The time-kill assays were performed in CAA media at 37° C. with aeration. Quantitative plating was performed at time periods of 0 hours, 1 hour, 3 hours, and 24 hours. The following Chp peptides were evaluated: Chp2, Chp4, Chp2-M1, Chp4-M1, Chp6, Chp6-M1, Chp10, Chp10-M1, Ecp3, Ecp3-M1, Unp2, Unp2-M1, and Ecp1-M1.

All 13 of the tested Chp peptides significantly reduced Log₁₀ CFU/mL of P. aeruginosa as compared to the untreated control buffer, with all but Chp10 maintaining strong efficacy up to 24 hours. Likewise, all 13 of the tested Chp peptides significantly reduced Log₁₀ CFU/mL of K. pneumoniae as compared to the untreated control buffer, maintaining strong efficacy up to 24 hours. All 13 of the tested Chp peptides significantly reduced Log₁₀ CFU/mL of A. Baumannii as compared to the untreated control buffer, with all but Chp10-M1 maintaining strong efficacy up to 24 hours.

Additionally, a stability assessment was conducted to detect the fold change in MIC after incubation of peptides prepared as described above in Example 2. Stability was assessed after incubation in 100% human serum at 37° C. after 10 minutes, 1 hour, and 2 hours. The results are shown below in Table 30.

TABLE 30

| | Fold change in MIC | | |
|---|---|---|---|
| Peptide | 10 minutes | 1 hour | 2 hours |
| Chp1 | 1 | 1 | 1 |
| Chp2 | 1 | 1 | 2 |
| CPAR39 | 1 | 0.5 | 0.5 |
| Chp4 | 1 | 1 | 0.5 |
| Chp5 | 1 | 2 | 2 |
| Chp6 | 1 | 1 | 1 |
| Chp7 | 1 | 1 | 1 |
| Chp8 | 1 | 1 | 1 |
| Chp9 | 1 | 0.5 | 0.5 |
| Chp10 | 1 | 2 | 2 |
| Chp1l | 1 | 2 | 2 |
| Chp12 | 1 | 1 | 1 |
| Gkh1 | 1 | 0.5 | 1 |
| Gkh2 | 1 | 0.5 | 2 |
| Gkh3 | 1 | 1 | 1 |
| Ecp1 | 1 | 4 | 1 |
| Ecp2 | 1 | 2 | 2 |
| Ecp3 | 1 | 1 | 1 |
| Ecp4 | n.d. | n.d. | n.d. |
| Osp1 | 1 | 0.5 | 1 |
| Unp1 | 1 | 0.5 | 2 |
| Unp2 | 1 | 2 | 1 |
| Unp3 | 1 | 1 | 1 |
| Unp5 | 1 | 1 | 4 |
| Unp6 | 1 | 1 | 1 |
| Spi1 | 1 | 2 | 2 |
| Spi2 | 1 | 1 | 1 |
| Bdp1 | 1 | 1 | 0.25 |
| Lvp1 | n.d. | n.d. | n.d. |
| Lvp2 | 1 | 1 | 0.25 |
| ALCES1 | 1 | 1 | 1 |
| AVQ206 | 1 | 1 | 1 |
| AVQ244 | 1 | 1 | 0.5 |
| CDL907 | 1 | 1 | 1 |
| AGT915 | 1 | 1 | 0.5 |
| HH3930 | n.d. | n.d. | n.d. |
| Fen7875 | n.d | n.d. | n.d |
| SBR77 | 1 | 4 | 1 |
| Chp2-M1 | 1 | 1 | 1 |
| Chp2-Cys | 1 | 2 | 4 |
| Chp2-NC | 1 | 1 | 4 |
| Chp4::Chp2 | 1 | 1 | 2 |
| Chp2-CAV | 1 | 1 | 0.5 |
| Ecp1-CAV | n.d | n.d | n.d. |

As shown in Table 30, all of Chp1, Chp2, CPAR39, Chp4, Chp5, Chp6, Chp7, Chp8, Chp9, Chp10, Chp11, Chp12, Gkh1, Gkh2, Gkh3, Ecp1, Ecp2, Ecp3, Osp1, Unp1, Unp2, Unp3, Unp5, Unp6, Spi1, Spi2, Bdp1, Lvp2, ALCES1, AVQ206, AVQ244, CDL907, AGT915, SBR77, Chp2-M1, Chp2-Cys, Chp2-NC, Chp4::Chp2, and Chp2-CAV were adequately stable after 10 minutes, 1 hour, and 2 hours.

Stability assessment was further conducted in 100% CAA and rabbit serum using the same methodology discussed above. The results, shown in Table 31 below, indicate that the Chp peptides are stable in rabbit serum and 100% CAA growth medium for 2 hours at 37° C.

TABLE 31
Stability Assessment in 100% CAA and Rabbit Serum

| | | Fold change in MIC | | | Fold Change in MIC | | |
|---|---|---|---|---|---|---|---|
| | MIC | (100% CAA) | | | (rabbit serum) | | |
| Peptide | (100% | 10 | 1 | 2 | 10 | 1 | 2 |
| | CAA) | min. | hour | hours | min. | hour | hours |
| Chp1 | 0.5 | 1 | 1 | 1 | 1 | 2 | 2 |
| Chp2 | 0.25 | 1 | 0.5 | 0.5 | 1 | 2 | 2 |
| CPAR39 | 4 | 1 | 1 | 1 | 1 | 0.5 | 0.25 |
| Chp4 | 0.25 | 1 | 0.5 | 0.5 | 1 | 2 | 4 |
| Chp6 | 0.25 | 1 | 0.5 | 0.5 | 1 | 1 | 1 |
| Chp7 | 8 | 1 | 1 | 1 | 2 | 0.5 | 0.5 |
| Chp8 | 0.5 | 1 | 0.5 | 0.5 | 1 | 1 | 1 |
| Chp9 | 2 | 1 | 0.5 | 0.5 | 1 | 1 | 1 |
| Chp10 | 0.5 | 1 | 0.5 | 0.5 | 1 | 1 | 1 |
| Chp11 | 16 | 1 | 1 | 1 | 1 | 1 | 1 |
| Chp12 | 8 | 1 | 1 | 1 | 1 | 1 | 1 |
| Gkh1 | 0.5 | 1 | 0.5 | 0.5 | 1 | 0.5 | 0.5 |
| Gkh2 | 16 | 1 | 0.5 | 0.5 | 1 | 1 | 1 |
| Gkh3 | 0.5 | 1 | 0.5 | 0.5 | 1 | 1 | 1 |
| Ecp1 | 0.25 | 1 | 1 | 1 | 1 | 2 | 1 |
| Cdl2 | 8 | 1 | 0.5 | 0.5 | 1 | 1 | 1 |
| Ecp2 | 0.25 | 1 | 1 | 0.5 | 1 | 1 | 1 |
| Ecp3 | 0.5 | 1 | 1 | 1 | 1 | 1 | 2 |
| Osp1 | 4 | 2 | 1 | 0.5 | 1 | 1 | 0.5 |
| Unp1 | 4 | 1 | 1 | 0.5 | 1 | 1 | 1 |
| Unp2 | 0.25 | 1 | 1 | 1 | 1 | 2 | 2 |
| Unp3 | 4 | 1 | 1 | 0.5 | 1 | 1 | 1 |
| Unp5 | 2 | 1 | 1 | 0.5 | 1 | 1 | 0.5 |
| Unp6 | 8 | 1 | 1 | 0.5 | 1 | 1 | 1 |
| Spi1 | 1 | 1 | 1 | 0.5 | 1 | 1 | 2 |
| Spi2 | 4 | 1 | 1 | 0.5 | 1 | 1 | 1 |
| Bdp1 | 16 | 1 | 1 | 0.5 | 1 | 1 | 1 |
| Lvp1 | >64 | 1 | 0.5 | 0.5 | 1 | 1 | 1 |
| Mse1 | 4 | 1 | 1 | 0.5 | 1 | 1 | 1 |
| AVQ206 | <0.25 | 1 | 2 | 0.5 | 1 | 1 | 1 |
| (Avq1) | | | | | | | |
| AVQ244 | 64 | 1 | 2 | 1 | 1 | 1 | 1 |
| (Avq2) | | | | | | | |
| AGT915 | 2 | 1 | 2 | 0.5 | 1 | 1 | 1 |
| (Agt1) | | | | | | | |
| HH3930 | 2 | 1 | 0.5 | 0.5 | 1 | 1 | 1 |
| (Hh1) | | | | | | | |
| Fen7875 | 4 | 1 | 1 | 0.5 | 1 | 1 | 1 |
| (Fen1) | | | | | | | |
| SBR77 | >64 | 1 | 1 | 1 | 1 | 1 | 1 |
| (Sbr1 | | | | | | | |
| Chp2-M1 | 0.25 | 1 | 0.5 | 0.5 | 1 | 1 | 1 |
| Chp2-Cys | 2 | 1 | 0.5 | 0.5 | n.d. | n.d. | n.d. |
| Chp4:: | 2 | 1 | 1 | 1 | n.d. | n.d. | n.d. |
| Chp2 | | | | | | | |
| RI18 | 2 | 1 | 0.5 | 0.5 | 1 | 1 | 1 |
| WLBU2 | 16 | 1 | 1 | 1 | 1 | 0.5 | 1 |

Stability in rat serum and horse serum was also observed, as well as stability in rabbit serum and 100% CAA for incubations at 4° C. and 24° C. No precipitation was observed in any instance.

### Example 8: MIC Determination in Both Non-Tuberculosis Mycobacterium (NTM) and Mycobacterium tuberculosis Strains

MIC values for various Chp peptides were determined using the CLSI method for broth microdilution in 96-well microtiter format. Each peptide was diluted 2-fold across the x-axis and combined with a fixed concentration of the following NTM strains having approximately 1×10⁵ cells/mL in Mueller Hinton broth media: M. smegmatis, M. fortuitum, M. avium, M. scrofulaceum, and M. intracellulare. Plates were incubated at 37° C. for 45 hours, and MIC was determined. The results are shown in Table 32 below.

TABLE 32

| | 45 hr MIC (μg/mL) | | | | | | |
|---|---|---|---|---|---|---|---|
| | | | M. | | | M. | M. intra- |
| | | | fortuitum | | | scrofula- | cellulare |
| | | | subsp. | | | aceum | (Cuttino |
| | M. | M. | Fortuitum | | M. | Prissick | and |
| | smegmatis | smegmatis | da Costa | M. avium | kansasii | and | McCabe) |
| | MC² | MC² | Cruz. | Chester | Hauduroy | Masson | Runyon |
| Chp | 155, | 155, | ATCC | ATCC | ATCC | ATCC | ATCC |
| peptide | Run #1 | Run #2 | 6841 | 700898 | 12478 | 19981 | 13950 |
| Chp1 | 8 | 16 | 16 | 4 | 4 | 4 | 4 |
| Chp2 | 4 | 4 | 2 | 1 | 2 | 0.5 | 0.5 |
| CPAR39 | >128 | — | — | — | — | — | — |
| Chp4 | 4 | 4 | 4 | 1 | >0.125 | 2 | 1 |
| Chp4-M1 | | 0.25 | 2 | 1 | 1 | 1 | 1 |
| Chp6 | 4 | 4 | 4 | 1 | 1 | 2 | 0.5 |
| Chp7 | 128 | — | — | — | — | — | — |
| Chp8 | 4 | 8 | 2 | 8 | 4 | 4 | 4 |
| Chp9 | 4 | 8 | 4 | >64 | 8 | 8 | 4 |
| Chp10 | 2 | 1 | 1 | 0.5 | 4 | 0.5 | 0.5 |
| Chp10- | — | 1 | 2 | 1 | 1 | 2 | 2 |
| M1 | | | | | | | |
| Chp11 | 32 | — | — | — | — | — | — |
| Chp12 | 64 | — | — | — | — | — | — |
| Gkh1 | 4 | 4 | 1 | 4 | 2 | 8 | 4 |
| Gkh2 | 32 | — | — | — | — | — | — |
| Gkh3 | >128 | — | — | — | — | — | — |
| Ecp1 | 4 | — | — | — | — | — | — |
| Ecp2 | 4 | 8 | 4 | 2 | 2 | 4 | 8 |
| Ecp3 | 4 | 8 | 2 | 1 | 2 | 2 | 8 |
| Osp1 | 32 | — | — | — | — | — | — |
| Unp1 | 4 | 2 | 8 | 1 | 8 | 8 | 32 |
| Unp2 | 2 | 4 | 2 | 0.5 | 2 | 4 | 1 |
| Unp2-M1 | — | 1 | 4 | 1 | 2 | 4 | 2 |
| Unp3 | 32 | — | — | — | — | — | — |
| Unp3-M1 | — | 8 | 4 | 8 | 16 | 8 | >64 |
| Unp4 | — | 16 | 4 | 1 | 4 | 4 | 4 |
| Unp5 | 8 | — | — | — | — | — | — |
| Unp6 | 16 | — | — | — | — | — | — |
| Spi1 | 2 | 16 | 8 | >64 | 0.5 | 2 | 4 |
| Spi2 | 4 | 4 | 4 | >64 | 1 | 8 | >64 |
| Bdp1 | 32 | — | — | — | — | — | — |
| Lvp1 | >128 | — | — | — | — | — | — |
| ALCES1 | 1 | 2 | 4 | 8 | 2 | 2 | 2 |
| AVQ206 | 4 | 16 | 2 | 1 | 2 | 4 | 4 |
| (Avq1) | | | | | | | |
| AVQ244 | 64 | — | — | — | — | — | — |
| (Avq2) | | | | | | | |
| CDL907 | >128 | — | — | — | — | — | — |
| (Cdl2) | | | | | | | |
| AGT915 | 4 | 1 | 1 | >64 | 1 | >64 | >64 |
| (Agt1) | | | | | | | |
| HH3930 | 4 | 16 | 8 | 4 | 32 | 16 | 16 |
| (Hh1) | | | | | | | |
| Fen7875 | 4 | 16 | 8 | 2 | 32 | 16 | 8 |
| (Fen1) | | | | | | | |
| SBR77 | >64 | — | — | — | — | — | — |
| (Sbr1) | | | | | | | |
| Chp2-M1 | 0.25 | 0.5 | 4 | 1 | 2 | 4 | 0.5 |
| Ecp1-M1 | 1 | 1 | 4 | 4 | 4 | 2 | 4 |
| Chp5 | >64 | — | — | — | — | — | — |
| Chp6-M1 | 0.5 | 0.25 | 2 | 2 | 4 | 4 | 1 |
| Ecp3-M1 | 1 | 1 | 8 | 0.5 | 2 | 2 | 8 |
| Myo1 | >64 | >64 | >64 | 8 | >64 | — | >64 |
| Spi2-M1 | 1 | 1 | 4 | >64 | 4 | 16 | >64 |
| Tobramycin | — | 0.125 | 4 | 0.125 | 16 | 0.25 | 0.25 |
| (for | | | | | | | |
| quality | | | | | | | |
| control) | | | | | | | |

As shown in Table 32, the Chp peptides exhibited variable levels of activity against several NTM strains. For example, eleven Chp peptides, including ALCES1, Chp2-M1, Ecp-M1, Chp6-M1, Ecp-M1, Chp4-M1, Chp10, Chp10-M1, Unp2-M1, Agt1, and Spi2-M1, exhibited strong MIC values of less than or equal to 1 μg/mL against M. smegmatis.

Next, MIC values for various Chp peptides were determined using the CLSI method above for broth microdilution in 96-well microtiter format for two different Mycobacterium tuberculosis strains. The MIC was determined as explained above for the NTM strains. The results are shown in Table 33 below.

TABLE 33

| | MIC (μg/mL) | |
|---|---|---|
| | | Mycobacterium |
| | Mycobacterium | tuberculosis |
| | tuberculosis | (Zopf) |
| | (Zopf) Lehmann | Lehmann and |
| | and Neumann | Neumann ATCC |
| Chp peptides | ATCC 25177 | 35817 |
| AGT915 (Agt1) | 16 | 4 |
| ALCES1 | 32 | 8 |
| AVQ206 (Avq1) | 16 | 8 |
| Chp1 | 64 | 32 |
| Chp10 | 32 | 8 |
| Chp10-M1 | 8 | 8 |
| Chp2 | 16 | 16 |
| Chp2-M1 | 4 | 4 |
| Chp4 | 16 | 8 |
| Chp4-M1 | 8 | 4 |
| Chp6 | 32 | 4 |
| Chp6-M1 | 32 | 8 |
| Chp8 | 32 | 8 |
| Chp9 | 32 | 16 |
| Myo1 | — | 32 |
| Ecp1-M1 | 4 | 4 |
| Ecp2 | 32 | 8 |
| Ecp3 | 16 | 8 |
| Ecp3-M1 | >64 | 4 |
| Fen7875(Fen1) | 16 | 8 |
| Gkh1 | 32 | 4 |
| HH3930 (Hh1) | 64 | 16 |
| Spi1 | 32 | 16 |
| Spi2 | 8 | 8 |
| Spi2-M1 | 16 | 4 |
| Unpl | 32 | 8 |
| Unp2 | 16 | 4 |
| Unp2-M1 | 16 | 4 |
| Unp3-M1 | 32 | 16 |
| Unp4 | 32 | 16 |
| Tobramycin (for | 4 | 4 |
| quality control) | | |

### Example 9: Antibiofilm Activity of Chp Peptide in Explanted Hemodialysis Catheters

The activity of Chp2-M1 on human biofilms was explored in three explanted dialysis catheter samples removed from hemodialysis patients with suspected catheter-related bloodstream infections. The explanted hemodialysis catheters were cut into equal length segments and bisected to expose the lumen.

For the recovery and quantitation of biofilm bacteria, catheter segments were homogenized with a Precellys® 24 tissue homogenizer (Bertin Technologies) according to standard methodologies set forth in Jorgensen et al., A modified chronic infection model for testing treatment of Staphylococcus aureus biofilms on implants, PLoS ONE 2014; 9:e103688.

Quantitative plating was performed on TSA blood agar, for CFU counts, assessment of hemolytic phenotype, and purity of culture, and Stenotrophomonas spp. colonization was observed. Species identification was done by sequencing of 16s rRNA amplicons, and MIC was determined for Chp2-M1.

For the first of the three catheter samples studied, segments were randomized into the following groups (N=3 segments per group): (1) buffer control (i.e., treatment with Lactated Ringer's solution along); (2) treatment with 1 μg/mL CF-301, a wild-type PlySs2 lysin as disclosed for example in U.S. Pat. No. 9,034,322 to Fischetti et al., which is hereby incorporated by reference in its entirety; (3) treatment with daptomycin alone at 1 μg/mL; and (4) Chp2-M1 treatment alone at 10 μg/mL. The samples were treated for 4 hours at 37° C. before rinsing with Lactated Ringer's solution.

As show in Table 34 below, Chp2-M1 eradicated the Stenotrophomonas-containing biofilm of the first catheter sample at 10 μg/mL, while CF-301 and daptomycin at 1 μg/mL did not.

TABLE 34
Effect of CF-301, Daptomycin, and Chp2-M1 on catheter biofilm

| | | | | Log₁₀ CFU/g for |
|---|---|---|---|---|
| | | | | Catheter Sample |
| | | | | #1 (enumerated |
| | | | | after 18 hours of |
| | CF-301 | Daptomycin | Chp2-M1 | incubation at 37 |
| Study group | (μg/mL) | (μg/mL) | (μg/mL) | ° C.) |
| Buffer control | 0 | 0 | 0 | 3.37 |
| CF-301 alone | 1 | 0 | 0 | 3.66 |
| Daptomycin alone | 0 | 1 | 0 | 3.66 |
| Chp2-M1 alone | 0 | 0 | 10 | 1 |

Recovered bacterial colonies from the first catheter sample exhibited a uniform phenotype on blood agar plates, suggesting a mono-microbial biofilm. The 16s rRNA amplicon sequencing (Charles River) yielded a sequence primarily related to Pseudomonas (Stenotrophomonas) species, and the organism exhibited an MIC value of 2 μg/mL with Chp2-Ml.

Next, treatment with 1 μg/mL Chp2-M1 alone was used to evaluate the effect on biofilm of the remaining two catheter samples, which were obtained from different parts of the same catheter. A concentration of 1 μg/mL was chosen based on the aforementioned MIC value of >2 μg/mL observed against the Gram-negative clinical isolate of Pseudomonas (Stenotrophomonas) for the first catheter.

The results shown below in Table 35 indicated that Chp2-M1 showed a 3-4 log₁₀ reduction in CFU/g, as well as the ability to clear biofilms formed in a human host, containing platelets, fibrogen, and other blood components that may not be duplicated in vitro.

TABLE 35
Effect of CF-301, Daptomycin, and Chp2-M1 on two catheter
biofilm samples

| | | | | Log₁₀ | Log₁₀ |
|---|---|---|---|---|---|
| | | | | CFU/g for | CFU/g for |
| | CF- | | Chp2- | Catheter | Catheter |
| | 301 | | M1 | Sample #2 | Sample #3 |
| | (μg/ | Daptomycin | (μg/ | enumerated after 24 hours | |
| Study group | mL) | (μg/mL) | mL) | of incubation at 37 ° C. | |
| Buffer control | 0 | 0 | 0 | 4.24 | 4.22 |
| CF-301 alone | 1 | 0 | 0 | 3.77 | 4.10 |
| Daptomycin | 0 | 1 | 0 | 3.72 | 4.15 |
| alone | | | | | |
| Chp2-M1 alone | 0 | 0 | 1 | 0.7 | 0.7 |

As with the first catheter sample, for the remaining two catheter samples, bacterial colonies exhibited a uniform phenotype on blood agar plates, suggesting a mono-microbial biofilm. The 16s rRNA amplicon sequencing (Charles River) yielded a sequence primarily related to Pseudomonas (Stenotrophomonas) species, and the organism exhibited an MIC value of 1 μg/mL with Chp2-M1.

Three additional infected hemodialysis catheter samples were removed from two patients (one catheter from a first patient and two catheters from a second patient). Catheter segments were bisected and allotted into different treatment groups (n=8 segments per group) with Chp2-M1 and a Lactated Ringer's buffer control, as described above. Both 1 μg/mL and 10 μg/mL concentrations of Chp2-M1 were used, and a meropenem control treatment of 1 μg/mL was used for one of the two catheters from the second patient. The samples were incubated at 37° C. for 4 hours.

After the 4-hour treatments with Chp2-M1, buffer, or meropenem, samples were homogenized (Precellys 24 tissue homogenizer, Bertin Technologies) and enumerated by quantitative plating on TSA blood agar plates.

For the first catheter explanted from the first patient, the treatment control resulted in a log₁₀ CFU/g of 3.37, while the 10 μg/mL of Chp2-M1 had a log₁₀ CFU/g of <0.7. Surviving bacteria were enumerated after 24 hours of incubation at 37° C. The limit of detection was 0.7 log₁₀ CFU/g of catheter. For the first catheter sample from the second patient, the log₁₀ CFU/g of meropenem at 1 μg/mL was not detected, and the log₁₀ CFU/g for Chp2-M1 at 1 μg/mL was <0.7. For the second catheter sample from the second patient, the log₁₀ CFU/g of meropenem at 1 μg/mL was 3.16, and the log₁₀ CFU/g for Chp2-M1 at 1 μg/mL was <0.7. Recovered bacterial colonies exhibited a uniform phenotype on blood agar plates, suggesting a mono-microbial biofilm for each catheter, and similar colony morphologies were observed for all bacteria from all three catheter samples, suggesting the same or similar causative agent.

The eradication of biofilms by Chp2-M1 at both 1 μg/mL and 10 μg/mL is consistent with in vitro observations of minimal biofilm eradication concentrations of ≤2 μg/mL for various Gram-negative pathogens using Chp2-M1. Meropenem alone did not eliminate biofilm at 1 μg/mL. Sequence analysis revealed the uniform presence of Stenotrophomonas organisms with Chp2-M1 MIC values of ≤2 μg/mL.

After homogenization, a subset of resulting isolates (n=16) were examined to determine speciation by sequencing 16s rRNA amplicons (AccuGENX-ID, Charles River Laboratory) and to determine MIC values for Chp2-M1 and meropenem. The 16s RNA sequencing identified the presence of Stenotrophomonas spp. for all of the organisms recovered for each of the three catheter samples. For each of the three catheters sampled, the MIC values for Chp2-M1 were determined to be 2, 1, and 1, respectively.

Based on these results, it was concluded that Chp2-M1 may eradicate biofilms containing Stenotrophomonas formed on catheters in human hosts at a concentration of μg/mL.

### Example 10—Chp Peptides not Inhibited by NaCl Levels, pH, or Divalent Cations

Certain Chp peptides were evaluated over a range of physiological NaCl levels and pH values, and were further evaluated for activity in the presence of various divalent cations. MIC values were determined for the Chp peptides in the both the presence and absence of NaCl (140 mM) against P. aeruginosa strain CFS-1292 (Table 36). As shown in Table 36 below, with the exception of Unp3-M1, all of the tested Chp peptides exhibited a MIC increase of less than 4-fold in the presence of NaCl.

TABLE 36

| | CAA (no | | Fold |
|---|---|---|---|
| Chp | added | CAA + 140 | change |
| peptide | NaCI) | mM NaCl | in MIC |
| Chp1 | 1 | 1 | 1 |
| Chp2 | 0.5 | 0.5 | 1 |
| Chp4 | 0.5 | 0.5 | 1 |
| Chp6 | 0.5 | 0.5 | 1 |
| Chp10 | 0.5 | 2 | 4 |
| Ecp1 | 0.5 | 2 | 4 |
| Ecp2 | 0.25 | 2 | 8 |
| Ecp3 | 0.5 | 2 | 4 |
| Unp2 | 0.5 | 2 | 4 |
| Agt1 | 2 | 8 | 4 |
| Chp2-M1 | 0.125 | 0.5 | 4 |
| Ecp1-M1 | 0.125 | 0.5 | 4 |
| Chp4-M1 | 0.5 | 1 | 2 |
| Chp6-M1 | 0.5 | 1 | 2 |
| Chp10-M1 | 0.5 | 1 | 2 |
| Unp2-M1 | 0.5 | 2 | 4 |
| Unp3-M1 | 8 | 16 | 2 |
| Spi2-M1 | 2 | 0.5 | −4 |
| Ecp3-M1 | 0.25 | 0.125 | −2 |
| Agt1-M1 | 1 | 4 | 4 |

The Chp peptides were further evaluated at varying pH levels. The MIC values were determined at pH 6, pH 7, and pH 8 for 18 different Chp peptides against P. aeruginosa strain CFS-1292. As shown in Table 37 below, all of the tested Chp peptides with the exception of Agt1 and Spi2-M1 maintained a MIC value of 4 or less, regardless of pH, with a fold-change of less than 4.

TABLE 37

| | MIC (μg/mL) | | | |
|---|---|---|---|---|
| | CAA media | | | |
| Chp peptide | (pH 7) | pH 6 | pH 7 | pH 8 |
| Chp1 | 1 | 0.5 | 1 | 1 |
| Chp2 | 0.25 | 0.25 | 0.5 | 0.5 |
| Chp4 | 0.5 | 0.25 | 0.5 | 1 |
| Chp6 | 1 | 2 | 0.25 | 1 |
| Chp10 | 0.5 | 2 | 1 | 1 |
| Ecp1 | 0.5 | 1 | 1 | 1 |
| Ecp3 | 1 | 2 | 2 | 2 |
| Unp2 | 0.5 | 0.5 | 0.5 | 0.5 |
| Agt1 | 4 | 8 | 16 | 8 |
| Chp2-M1 | 0.125 | 0.25 | 0.25 | 0.25 |
| Ecp1-M1 | 0.25 | 1 | 0.5 | 0.5 |
| Chp4-M1 | 0.125 | 0.125 | 0.125 | 0.125 |
| Chp6-M1 | 0.25 | 0.5 | 0.5 | 0.25 |
| Chp10-M1 | 0.125 | 0.5 | 0.125 | 0.125 |
| Unp2-M1 | 0.125 | 0.25 | 0.25 | 0.5 |
| Spi2-M1 | 8 | 8 | 8 | 8 |
| Ecp3-M1 | 0.25 | 1 | 0.5 | 0.5 |
| Agt1-M1 | 1 | 4 | 4 | 4 |

Next, Chp peptides were evaluated in the presence of different divalent cations. The MICs were determined (1) in the absence of both calcium and magnesium; (2) in the presence of 2 mM calcium chloride alone; (3) in the presence of 1 mM magnesium sulfate alone; and (4) in the presence of both 2 mM calcium chloride and 1 mM magnesium sulfate. As shown in Table 38, all of the tested Chp peptides had MIC values of ≤2, indicating that none of the Chp peptides were inhibited by the presence of the divalent cations.

TABLE 38

| | MIC (μg/mL) | | | |
|---|---|---|---|---|
| | | CAA | CAA | CAA media + |
| | CAA | media + | media + | 2 mM Cal₂ + |
| | media | 2 mM | 1 mM | 1 mM |
| Chp peptide | alone | CaCl₂ | MgSO₄ | MgSO₄ |
| Chp1 | 1 | 2 | 1 | 1 |
| Chp2 | 0.5 | 0.25 | 0.5 | 0.25 |
| Chp4 | 0.5 | 1 | 0.5 | 0.125 |
| Chp6 | 1 | 0.5 | 1 | 1 |
| Chp10 | 0.5 | 1 | 1 | 2 |
| Ecp1 | 0.5 | 1 | 0.5 | 1 |
| Ecp3 | 1 | 2 | 2 | 2 |
| Unp2 | 0.25 | 0.25 | 0.25 | 0.5 |
| Chp2-M1 | 0.25 | 0.25 | 0.25 | 0.25 |
| Ecp1-M1 | 0.25 | 0.5 | 0.25 | 1 |
| Chp4-M1 | 0.25 | 0.25 | 0.25 | 0.25 |
| Chp6-M1 | 0.5 | 1 | 0.5 | 0.5 |
| Chp10-M1 | 0.125 | 0.25 | 0.125 | 0.5 |
| Unp2-M1 | 0.25 | 0.25 | 0.25 | 0.5 |
| Ecp3-M1 | 0.25 | 1 | 0.25 | 1 |
| Agt1-M1 | 2 | 2 | 2 | 2 |

### Example 11—Chp Peptides not Inhibited by Pulmonary Surfactant

MIC values against P. aeruginosa strain CFS-1292 were determined in the presence and absence of two concentrations (0.19 mg/mL and 0.78 mg/mL) of Survanta®, a synthetic pulmonary surfactant. The tested Survanta® concentrations are known to be inhibitory to daptomycin activity against S. aureus. Nonetheless, as reported in Table 39, Survanta® was not inhibitory to the Chp peptide activity against P. aeruginosa, as indicated by a <4-fold change in MIC for each Chp peptide tested.

TABLE 39

| | MIC (μg/mL) | | |
|---|---|---|---|
| | CAA | CAA media + | CAA media + |
| | media | 0.78 mg/mL | 0.19 mg/mL |
| Chp peptide | alone | Survanta ® | Survanta ® |
| Chp1 | 1 | 2 | 2 |
| Chp2 | 0.5 | 1 | 1 |
| Chp4 | 0.5 | 0.5 | 0.5 |
| Chp6 | 0.5 | 0.5 | 0.5 |
| Chp10 | 0.5 | 0.5 | 0.5 |
| Ecp1 | 0.5 | 0.5 | 0.5 |
| Ecp3 | 0.5 | 0.5 | 0.5 |
| Unp2 | 0.5 | 0.5 | 0.5 |
| Chp2-M1 | 0.125 | 0.125 | 0.125 |
| Ecp1-M1 | 0.125 | 0.125 | 0.125 |
| Chp4-M1 | 0.25 | 0.25 | 0.25 |
| Chp6-M1 | 0.5 | 0.5 | 0.5 |
| Chp10-M1 | 0.25 | 0.25 | 0.25 |
| Unp2-M1 | 0.5 | 2 | 0.25 |
| Ecp3-M1 | 0.25 | 0.25 | 0.25 |
| Agt1-M1 | 4 | 0.5 | 4 |

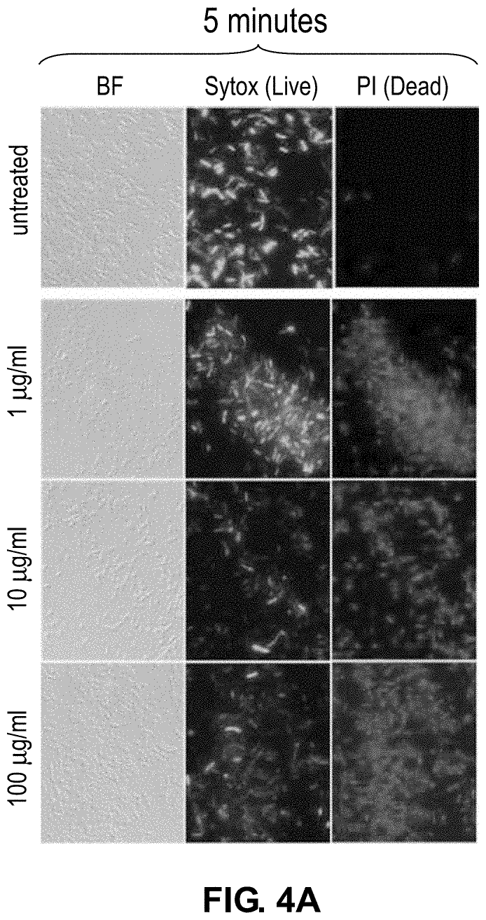

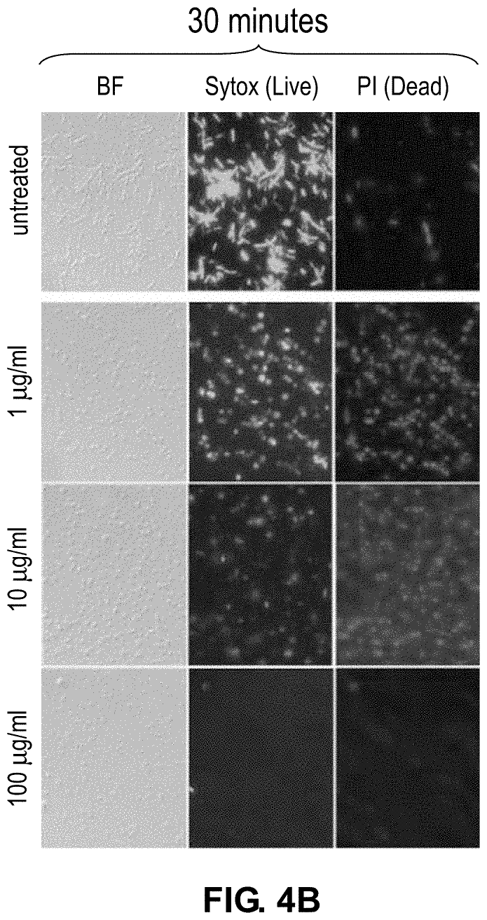

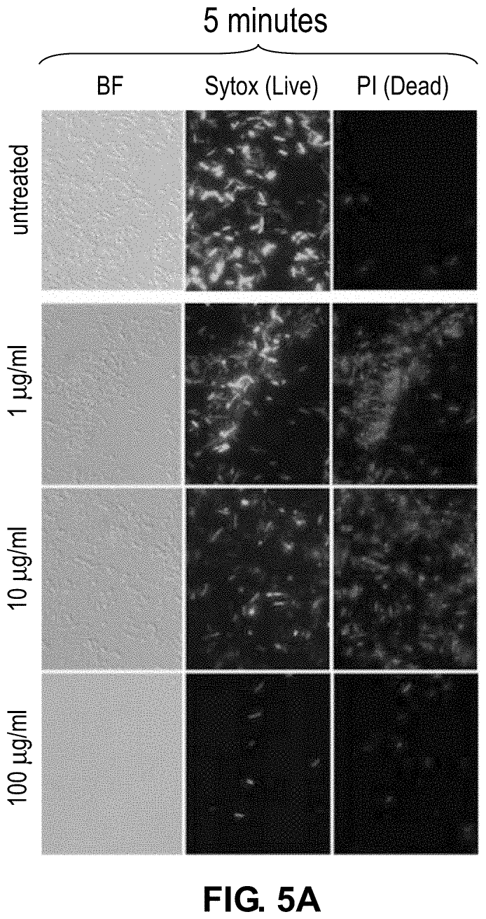

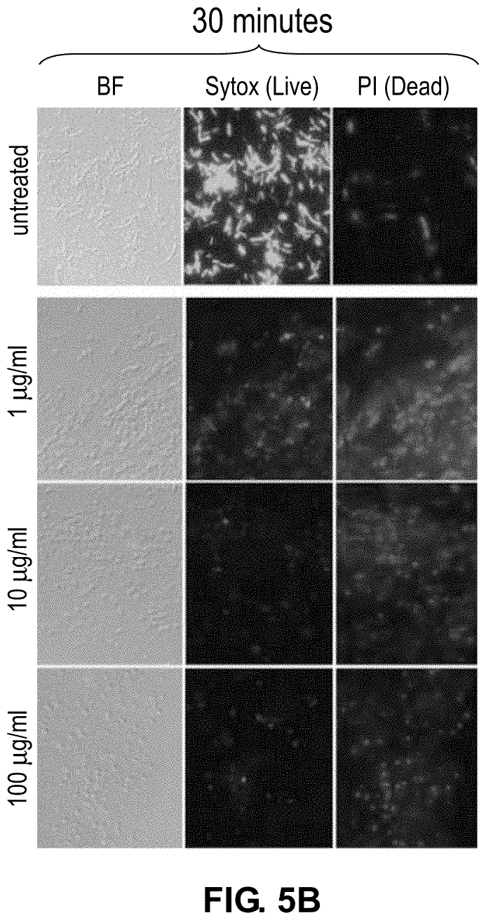

The efficacy of both Chp2-M1 and Ecp3-M1 was evaluated against P. aeruginosa strain CFS-1292 suspended in Survanta® (1.5 mg/mL) with LIVE/DEAD stain. Chp2-M1 or Ecp3-M1 in varying concentrations of 1 μg/mL, 10 μg/mL, and 100 μg/mL was added to samples of the suspension. The suspensions were evaluated 1 hour prior to treatment with Chp2-M1 or Ecp3-M1, 5 minutes after treatment with Chp2-M1 or Ecp2-M1, and 30 minutes after treatment with Chp2-M1 or Ecp3-M1. Samples were visualized by bright field (BF) and fluorescence microscopy (10 ms exposure). Sytox Green was used to label live cells, and propodium iodide (PI) was used to label damaged and dead cells. As shown in FIGS. 4A-B, Chp2-M1 induced rapid killing of P. aeruginosa after 5 minutes (FIG. 4A) and after 30 minutes (FIG. 4B) at all concentrations tested. Likewise, as shown in FIGS. 5A-B, Ecp3-M1 induced rapid killing of P. aeruginosa after 5 minutes (FIG. 5A) and after 30 minutes (FIG. 5B) at all concentrations tested.

### Example 12—Chp Peptides Active Against Broad Spectrum of Bacteria, Including ESKAPE Pathogens

In addition to the results discussed above in Example 3, further testing was conducted on several Chp peptides. MIC values were determined against different species, including P. aeruginosa, K. pneumoniae, A. baumannii, E. cloacae, E. coli, and S. maltophilia, and the results are presented below in Tables 40-45, wherein n=number of isolates tested for each species and MIC is measured as μg/mL. Good activity was determined to correspond to a MIC ranging from 0 to ≤4 (i.e., a MIC₁₀₀ of ≤4). As shown in Tables 40-45 below, several of the Chp peptides tested demonstrated good activity against ESKAPE pathogens (including P. aeruginosa, K. pneumoniae, A. baumannii, and E. cloacae), as well as E. coli and S. maltophilia.

TABLE 40
Chp peptide activity against P. aeruginosa

| Chp peptide | n | MIC₅₀ | MIC₁₀₀ | Range |
|---|---|---|---|---|
| Agt-1 | 12 | >8 | >8 | >8 |
| Agt1-M1 | 12 | >8 | >8 | 8->8 |
| ALCES1 | 12 | >8 | >8 | 8->8 |
| Avq1 | 12 | 4 | 8 | 0.25->8 |
| Chp1 | 24 | 2 | 4 | 1-4 |
| Chp2 | 25 | 1 | 1 | 0.25-1 |
| Chp2-M1 | 65 | 0.25 | 0.5 | 0.0625-1 |
| Chp4 | 24 | 1 | 1 | 0.5-1 |
| Chp4-M1 | 12 | 0.5 | 0.5 | 0.125-0.5 |
| Chp6 | 25 | 1 | 1 | 0.5-1 |
| Chp6-M1 | 65 | 0.25 | 0.5 | 0.0625-1 |
| Chp8 | 12 | 8 | >8 | 1->8 |
| Chp9 | 12 | >8 | >8 | 8->8 |
| Chp10 | 24 | 4 | 8 | 0.5-8 |
| Chp10-M1 | 24 | 0.25 | 0.5 | 0.25-0.5 |
| Ecp1 | 12 | 4 | >8 | 1->8 |
| Ecp1-M1 | 65 | 0.25 | 0.5 | 0.0625-1 |
| Ecp2 | 12 | 2 | 8 | 0.5-8 |
| Ecp3 | 25 | 2 | 4 | 0.5-4 |
| Ecp3-M1 | 25 | 0.5 | 1 | 0.125-1 |
| Gkh1 | 12 | 8 | >8 | 2->8 |
| Gkh3 | 12 | 8 | >8 | 2->8 |
| Osp1 | 12 | >8 | >8 | >8 |
| Osp1-M1 | 12 | >8 | >8 | >8 |
| Spi1 | 12 | 4 | 8 | 2-8 |
| Spi2 | 23 | >8 | >8 | >8 |
| Spi2-M1 | 23 | >8 | >8 | 2->8 |
| Unp2 | 24 | 1 | 1 | 0.5-2 |
| Unp2-M1 | 24 | 0.5 | 1 | 0.5-1 |
| Unp3 | 12 | >8 | >8 | 8->8 |
| Unp3-M1 | 12 | >8 | >8 | 4->8 |
| Unp5 | 12 | >8 | >8 | 8->8 |

TABLE 41
Chp peptide activity against K. pneumoniae

| Chp peptide | n | MIC₅₀ | MIC₁₀₀ | Range |
|---|---|---|---|---|
| Agt-1 | 12 | >8 | >8 | 8->8 |
| Agt1-M1 | 12 | >8 | >8 | 8->8 |
| ALCES1 | 12 | >8 | >8 | 8->8 |
| Avq1 | 12 | 1 | 1 | 0.5-1 |
| Chp1 | 24 | 2 | 2 | 1-4 |
| Chp2 | 25 | 0.5 | 1 | 0.125-1 |
| Chp2-M1 | 50 | 0.25 | 0.5 | 0.0625-1 |
| Chp4 | 12 | 1 | 1 | 0.5-1 |
| Chp4-M1 | 24 | 0.5 | 0.5 | 0.25-1 |
| Chp6 | 25 | 0.5 | 1 | 0.125-1 |
| Chp6-M1 | 39 | 0.125 | 0.5 | 0.0625-1 |
| Chp8 | 12 | 4 | 8 | 2-8 |
| Chp9 | 12 | >8 | >8 | >8 |
| Chp10 | 24 | 0.25 | 0.5 | 0.125-0.5 |
| Chp10-M1 | 24 | 0.25 | 1 | 0.125-1 |
| Ecp1 | 12 | 1 | 2 | 1-2 |
| Ecp1-M1 | 50 | 0.125 | 0.25 | 0.031-0.25 |
| Ecp2 | 12 | 0.5 | 1 | 0.5-1 |
| Ecp3 | 25 | 1 | 2 | 0.5-4 |
| Ecp3-M1 | 25 | 0.25 | 0.5 | 0.0625-1 |
| Gkh1 | 12 | 4 | 8 | 1-8 |
| Gkh3 | 12 | >8 | >8 | 4->8 |
| Osp1 | 12 | >8 | >8 | >8 |
| Osp1-M1 | 12 | >8 | >8 | >8 |
| Spi1 | 12 | 2 | 4 | 2-4 |
| Spi2 | 12 | >8 | >8 | >8 |
| Spi2-M1 | 15 | 8 | >8 | 0.5-8 |
| Unp2 | 24 | 0.5 | 1 | 0.25-1 |
| Unp2-M1 | 24 | 0.5 | 0.5 | 0.125-0.5 |
| Unp3 | 12 | >8 | >8 | >8 |
| Unp3-M1 | 12 | >8 | >8 | >8 |
| Unp5 | 12 | >8 | >8 | >8 |

TABLE 42
Chp peptide activity against A. baumannii

| Chp peptide | n | MIC₅₀ | MIC₁₀₀ | Range |
|---|---|---|---|---|
| Agt-1 | 12 | 4 | 8 | 2-8 |
| Agt1-M1 | 12 | 2 | 4 | 0.5-4 |
| ALCES1 | 12 | 2 | 2 | 1-2 |
| Avq1 | 12 | 0.25 | 0.5 | 0.25-0.5 |
| Chp1 | 24 | 2 | 4 | 1-4 |
| Chp2 | 24 | 0.5 | 0.5 | 0.25-1 |
| Chp2-M1 | 19 | 0.125 | 0.25 | <0.015-0.25 |
| Chp4 | 16 | 0.5 | 1 | 0.25-1 |
| Chp4-M1 | 24 | 0.5 | 1 | 0.25-1 |
| Chp6 | 24 | 0.25 | 0.5 | 0.125-1 |
| Chp6-M1 | 24 | 0.25 | 0.25 | 0.125-1 |
| Chp8 | 12 | 0.5 | 1 | 0.25-2 |
| Chp9 | 12 | 2 | 2 | 0.5-2 |
| Chp10 | 12 | 0.5 | 0.5 | 0.25-0.5 |
| Chp10-M1 | 24 | 0.5 | 0.5 | 0.06-1 |
| Ecp1 | 23 | 0.5 | 1 | 0.125-2 |
| Ecp1-M1 | 24 | 0.25 | 0.25 | 0.125-1 |
| Ecp2 | 12 | 0.25 | 0.5 | 0.125-0.5 |
| Ecp3 | 12 | 0.25 | 0.5 | 0.125-0.5 |
| Ecp3-M1 | 24 | 0.25 | 0.5 | 0.125-1 |
| Gkh1 | 12 | 0.5 | 1 | 0.25-2 |
| Gkh3 | 12 | 0.5 | 1 | 0.25-1 |
| Osp1 | 12 | 8 | 8 | 2-8 |
| Osp1-M1 | 12 | >8 | >8 | 4->8 |
| Spi1 | 12 | 1 | 1 | 1 |
| Spi2 | 12 | 2 | 2 | 2-8 |
| Spi2-M1 | 12 | 1 | 8 | 0.25-8 |
| Unp2 | 24 | 0.5 | 0.5 | 0.25-1 |
| Unp2-M1 | 24 | 0.5 | 1 | 0.25-2 |
| Unp3 | 12 | >8 | >8 | 4->8 |
| Unp3-M1 | 12 | 8 | 8 | 1-8 |
| Unp5 | 12 | 4 | 8 | 1-8 |

TABLE 43
Chp peptide activity against E. cloacae

| Chp peptide | n | MIC₅₀ | MIC₁₀₀ | Range |
|---|---|---|---|---|
| Agt-1 | 11 | 8 | >8 | 4->8 |
| Agt1-M1 | 11 | 4 | 4 | 4->8 |
| ALCES1 | n.d. | n.d. | n.d. | n.d. |
| Avq1 | 12 | 0.5 | 1 | 0.25-2 |
| Chp1 | 11 | 2 | 4 | 2-4 |
| Chp2 | 21 | 0.5 | 1 | 0.125-1 |
| Chp2-M1 | 21 | 0.125 | 0.25 | 0.06-0.5 |
| Chp4 | 21 | 0.5 | 1 | 0.25-2 |
| Chp4-M1 | 21 | 0.25 | 0.5 | 0.25-5 |
| Chp6 | 21 | 0.5 | 1 | 0.25-2 |
| Chp6-M1 | 21 | 0.25 | 0.5 | 0.125-1 |
| Chp8 | 11 | 2 | 4 | 1-8 |
| Chp9 | 11 | 4 | >8 | 2->8 |
| Chp10 | 20 | 0.25 | 1 | 0.125-2 |
| Chp10-M1 | 21 | 0.25 | 0.5 | 0.125-2 |
| Ecp1 | 11 | 0.5 | 2 | 0.25-4 |
| Ecp1-M1 | 21 | 0.0625 | 1 | 0.03-2 |
| Ecp2 | 11 | 0.25 | 0.5 | 0.125-2 |
| Ecp3 | 12 | 0.5 | 4 | 0.25-4 |
| Ecp3-M1 | 21 | 0.25 | 0.5 | 0.125-0.5 |
| Gkh1 | 11 | 1 | 2 | 0.5-8 |
| Gkh3 | 11 | 4 | 4 | 1->8 |
| Osp1 | n.d. | n.d. | n.d. | n.d. |
| Osp1-M1 | n.d. | n.d. | n.d. | n.d. |
| Spi1 | n.d. | n.d. | n.d. | n.d. |
| Spi2 | 12 | >8 | >8 | 4->8 |
| Spi2-M1 | 12 | 0.5 | 2 | 0.5-2 |
| Unp2 | 21 | 0.25 | 0.5 | 0.25-2 |
| Unp2-M1 | 21 | 0.25 | 0.5 | 0.25-1 |
| Unp3 | 11 | >8 | >8 | >8 |
| Unp3-M1 | 11 | >8 | >8 | >8 |
| Unp5 | n.d. | n.d. | n.d. | n.d. |

TABLE 44
Chp peptide activity against E. coli

| Chp peptide | n | MIC₅₀ | MIC₁₀₀ | Range |
|---|---|---|---|---|
| Agt-1 | 11 | 8 | >8 | 4->8 |
| Agt1-M1 | 11 | 0.5 | 1 | 0.125-2 |
| ALCES1 | n.d. | n.d. | n.d. | n.d. |
| Avq1 | 23 | 0.5 | 2 | 0.06-4 |
| Chp1 | 23 | 2 | 4 | 1-4 |
| Chp2 | 23 | 0.5 | 2 | 0.25-2 |
| Chp2-M1 | 23 | 0.03125 | 0.25 | <0.015-0.25 |
| Chp4 | 23 | 1 | 2 | 0.5-4 |
| Chp4-M1 | 23 | 0.25 | 0.25 | 0.06-1 |
| Chp6 | 23 | 0.5 | 1 | 0.25-2 |
| Chp6-M1 | 23 | 0.25 | 0.5 | <0.015-0.5 |
| Chp8 | 11 | 2 | 4 | 0.5-4 |
| Chp9 | 11 | 2 | 8 | 0.5-8 |
| Chp10 | 23 | 0.5 | 1 | 0.03-0.2 |
| Chp10-M1 | 23 | 0.0625 | 0.5 | 0.0312-0.5 |
| Ecp1 | 11 | 0.5 | 2 | 0.25-4 |
| Ecp1-M1 | 23 | 0.03 | 0.125 | <0.015-0.125 |
| Ecp2 | 11 | 0.5 | 1 | 0.24-4 |
| Ecp3 | 11 | 1 | 2 | 0.125-8 |
| Ecp3-M1 | 23 | 0.125 | 0.25 | 0.03-0.5 |
| Gkh1 | 11 | 2 | 2 | 0.5-8 |
| Gkh3 | 11 | 1 | 4 | 0.5-8 |
| Osp1 | n.d. | n.d. | n.d. | n.d. |
| Osp1-M1 | n.d. | n.d. | n.d. | n.d. |
| Spi1 | n.d. | n.d. | n.d. | n.d. |
| Spi2 | 11 | 4 | >8 | 0.25->8 |
| Spi2-M1 | 11 | 0.125 | 0.25 | 0.0625-1 |
| Unp2 | 23 | 0.25 | 1 | 0.125-1 |
| Unp2-M1 | 23 | 0.125 | 0.5 | 0.125-0.5 |
| Unp3 | 11 | >8 | >8 | 4->8 |
| Unp3-M1 | 11 | 1 | 4 | 0.5-8 |
| Unp5 | n.d. | n.d. | n.d. | n.d. |

TABLE 45
Chp peptide activity against S. maltophilia

| Chp peptide | n | MIC₅₀ | MIC₁₀₀ | Range |
|---|---|---|---|---|
| Agt-1 | 12 | >8 | >8 | 8->8 |
| Agt1-M1 | 12 | >8 | >8 | 8->8 |
| ALCES1 | 11 | 4 | 8 | 2->8 |
| Avq1 | 11 | 1 | 4 | 0.5->8 |
| Chp1 | 17 | 2 | 4 | 1-8 |
| Chp2 | 17 | 0.5 | 1 | 0.5-1 |
| Chp2-M1 | 17 | 0.5 | 0.5 | 0.125-2 |
| Chp4 | 17 | 0.5 | 2 | 0.5-8 |
| Chp4-M1 | 17 | 0.5 | 0.5 | 0.25-1 |
| Chp6 | 17 | 0.5 | 1 | 0.25-1 |
| Chp6-M1 | 17 | 0.5 | 0.5 | 0.125-2 |
| Chp8 | 12 | >8 | >8 | 4->8 |
| Chp9 | 12 | >8 | >8 | 8->8 |
| Chp10 | 12 | 8 | >8 | 2->8 |
| Chp10-M1 | 17 | 1 | 8 | 0.5-8 |
| Ecp1 | 12 | 8 | >8 | 2->8 |
| Ecp1-M1 | 17 | 0.5 | 2 | 0.125-2 |
| Ecp2 | 12 | 8 | >8 | 1->8 |
| Ecp3 | 17 | 4 | >8 | 1->8 |
| Ecp3-M1 | 17 | 1 | 4 | 0.5->8 |
| Gkh1 | 12 | >8 | >8 | 4->8 |
| Gkh3 | 12 | 8 | >8 | 2->8 |
| Osp1 | 11 | >8 | >8 | >8 |
| Osp1-M1 | 11 | >8 | >8 | >8 |
| Spi1 | 11 | 2 | 2 | 2->8 |
| Spi2 | 12 | >8 | >8 | >8 |
| Spi2-M1 | 12 | >8 | >8 | 4->8 |
| Unp2 | 17 | 0.5 | 1 | 0.25-2 |
| Unp2-M1 | 17 | 1 | 1 | 0.125-2 |
| Unp3 | 12 | >8 | >8 | >8 |
| Unp3-M1 | 12 | >8 | >8 | 8->8 |
| Unp5 | 11 | >8 | >8 | >8 |

Based on the data, it is noted that the following 13 Chp peptides had good activity against all of P. aeruginosa, K. pneumoniae, A. baumannii, E. cloacae, and E. coli: Chp2, Chp2-M1, Chp4, Chp4-M1, Chp6, Chp6-M1, Chp10, Chp10-M1, Ecp1-M1, Ecp3, Ecp3-M1, Unp2, and Unp2-Ml. It was further noted that of those 13 Chp peptides, Chp2, Chp2-M1, Chp4-M1, Chp6, Chp6-M1, Ecp1-M1, Ecp3-M1, Unp2, and Unp2-M1 were likewise active against S. maltophilia. Additionally, A. baumannii was sensitive to the widest range of the different Chp peptides tested.

Amongst other bacterial species tested, Chp2 (n=12), Chp4 (n=12), and Unp2 (n=12) exhibited positive activity (MICs ≤4) against Achromobacter xylosoxidans. Chp2 (n=1) and Chp4-M1 (n=1) exhibited positive activity (MICs ≤4) against Burkholderia anthina. Chp2-M1, Chp4, Chp6, Chp6-M1, Ecp1-M1, Ecp3-M1, and Spi1-M1 (n=2 for all) exhibited positive activity (MICs ≤4) against Serratia marcescens. The following Chp peptides exhibited positive activity (MICs ≤4) against Burkholderia cenocepacia: Agt1-M1, Mse1, Avq1, Chp1, Chp2, Chp2-M1, Chp4, Chp4-M1, Chp6, Chp6-M1, Chp8, Chp9, Chp10, Chp10-M1, Ecp1, Ecp1-M1, Ecp2, Ecp3, Ecp3-M1, Gkh1, Spi1, Unp2, and Unp2-Ml.

### Example 13—Chp Peptides Active Against Carbapenem-Resistance Isolates

Seven different carbapenem-resistant bacterial isolated were tested with various Chp peptides, and the MIC of the peptides measured and presented in Table 46 as μg/mL. As shown in Table 46 below, several of the Chp peptides exhibited good activity (MIC ≤4) against the carbapenem-resistant bacterial strains. The MIC values for meropenem are indicated in parenthesis for each strain analyzed in Table 46.

TABLE 46
Chp peptide activity against carbapenem-resistant isolates

| | | | | | | | WLC- |
|---|---|---|---|---|---|---|---|
| | PA19 | PA20 | PA21 | PA22 | PA23 | PA24 | 452 |
| Chp | (MIC | (MIC | (MIC | (MIC | (MIC | (MIC | (MIC |
| peptide | 32) | 16) | 32) | 16) | 8) | 32) | 16) |
| Agt1 | >8 | >8 | >8 | >8 | >8 | >8 | >8 |
| Agt- | 8 | >8 | >8 | >8 | >8 | >8 | 4 |
| M1 | | | | | | | |
| Mse1 | 8 | >8 | >8 | >8 | >8 | >8 | 8 |
| Avq1 | 0.25 | 8 | 8 | >8 | 4 | 4 | 0.5 |
| Chp1 | 2 | 4 | 4 | 4 | 4 | 4 | 2 |
| Chp2 | 0.5 | 1 | 1 | 1 | 1 | 1 | 0.5 |
| Chp2- | 0.0625 | 0.25 | 0.25 | 0.5 | 0.5 | 0.5 | 0.25 |
| M1 | | | | | | | |
| Chp4 | 0.5 | 1 | 1 | 1 | 1 | 1 | 0.5 |
| Chp4- | 0.25 | 0.5 | 0.25 | 0.5 | 0.5 | 0.5 | 0.25 |
| M1 | | | | | | | |
| Chp6 | 0.5 | 1 | 1 | 1 | 1 | 1 | 0.25 |
| Chp6- | 0.25 | 0.5 | 0.5 | 0.5 | 0.5 | 0.5 | 0.5 |
| M1 | | | | | | | |
| Chp8 | 1 | >8 | >8 | >8 | >8 | 8 | 4 |
| Chp9 | 8 | >8 | >8 | >8 | >8 | >8 | 8 |
| Chp10 | 0.5 | 8 | 8 | 8 | 4 | 4 | 1 |
| Chp10- | 0.5 | 0.5 | 0.5 | 1 | 1 | 0.5 | 0.0625 |
| M1 | | | | | | | |
| Ecp1 | 1 | >8 | >8 | 8 | 4 | 4 | 2 |
| Ecp1- | 0.0625 | 0.5 | 0.25 | 0.5 | 0.5 | 0.25 | 0.25 |
| M1 | | | | | | | |
| Ecp2 | 0.5 | 8 | 8 | 4 | 2 | 2 | 1 |
| Ecp3 | 0.5 | 4 | 4 | 4 | 2 | 2 | 1 |
| Ecp3- | 0.125 | 0.5 | 0.5 | 1 | 0.5 | 0.5 | 0.5 |
| M1 | | | | | | | |
| Gkh1 | 2 | >8 | >8 | >8 | >8 | 8 | 2 |
| Gkh3 | 2 | >8 | >8 | >8 | 8 | 8 | 2 |
| Osp1 | >8 | >8 | >8 | >8 | >8 | >8 | 8 |
| Osp1- | >8 | >8 | >8 | >8 | >8 | >8 | >8 |
| M1 | | | | | | | |
| Spi1 | 2 | 4 | 8 | 4 | 8 | 4 | 2 |
| Spi2 | >8 | >8 | >8 | >8 | >8 | >8 | >8 |
| Spi2- | >8 | >8 | 8 | >8 | >8 | 8 | 4 |
| M1 | | | | | | | |
| Unp2 | 0.5 | 1 | 2 | 1 | 1 | 1 | 0.5 |
| Unp2- | 0.5 | 0.5 | 0.5 | 1 | 1 | 0.5 | 0.5 |
| M1 | | | | | | | |
| Unp3 | 8 | >8 | >8 | >8 | >8 | >8 | >8 |
| Unp3- | 4 | >8 | >8 | >8 | >8 | >8 | >8 |
| M1 | | | | | | | |
| Unp5 | 8 | >8 | >8 | >8 | >8 | >8 | >8 |

### Example 14—Chp Peptide Activity in Animal Serum

The activity of several Chp peptides against P. aeruginosa strain CFS-1292 in animal serum was evaluated. MICs were determined in the presence of mouse, rat, rabbit, and human serum (12.5% each) and CAA, and the results are shown below in Table 47. Rat, mouse, and rabbit serum were all obtained from pooled gender samples. A MIC increase of ≤8-fold as compared to human serum indicated sufficient activity against P. aeruginosa and may aid identifying species for in vivo efficacy studies.

TABLE 47
Chp peptide activity in mouse, rat, rabbit, and human serum

| | MIC (μg/mL) | | | | |
|---|---|---|---|---|---|
| | | | New | | |
| | | | Zealand | | |
| | BALB/C | Sprague | White | | |
| | Mouse | Dawley Rat | Rabbit | | Human |
| Chp peptide | Serum | Serum | Serum | CAA | Serum |
| Chp2 | 32 | 2 | 2 | 0.5 | 0.5 |
| Chp4 | 32 | 2 | 2 | 0.5 | 0.5 |
| Chp6 | 32 | 4 | 4 | 1 | 1 |
| Chp10 | 32 | 4 | 4 | 1 | 1 |
| Ecp3 | >32 | 8 | 8 | 0.5 | 0.5 |
| Unp2 | >32 | 8 | 4 | 0.5 | 0.5 |
| Chp2-M1 | 4 | 1 | 0.5 | 0.125 | 0.125 |
| Ecpl-M1 | 4 | 1 | 1 | 0.125 | 0.125 |
| Chp4-M1 | 4 | 1 | 0.25 | 0.25 | 0.25 |
| Chp6-M1 | 8 | 1 | 1 | 0.5 | 0.5 |
| Chp10-M1 | 2 | 0.5 | 0.5 | 0.25 | 0.25 |
| Unp2-M1 | 8 | 1 | 1 | 0.25 | 0.5 |
| Ecp3-M1 | 4 | 4 | 2 | 0.25 | 0.25 |

The results indicate that rat and rabbit serum support equivalent levels of activity and are similar to human serum. The mouse serum, however, was multiple-folds higher than that observed for rat, rabbit, and human.

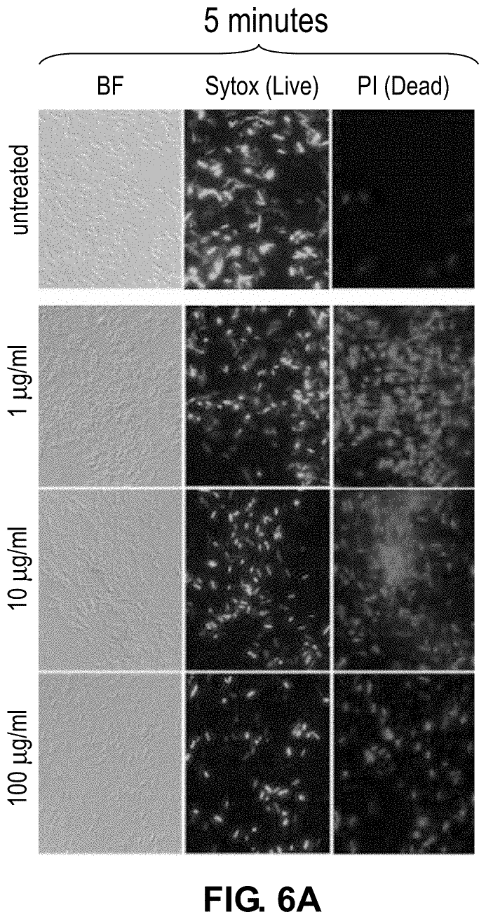

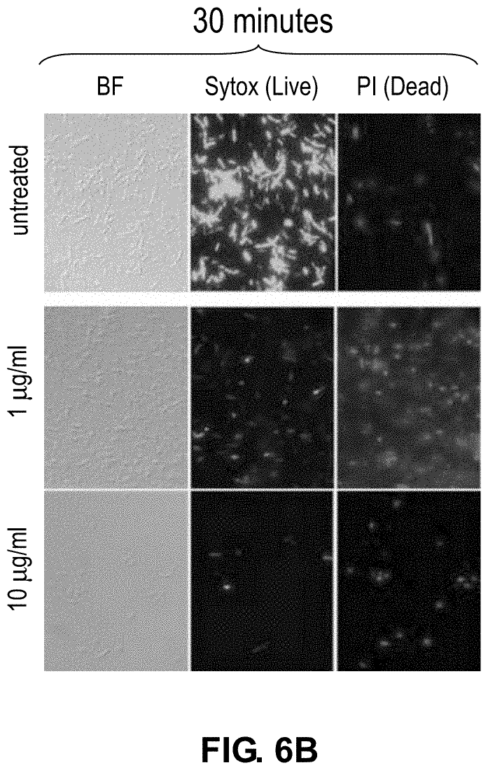

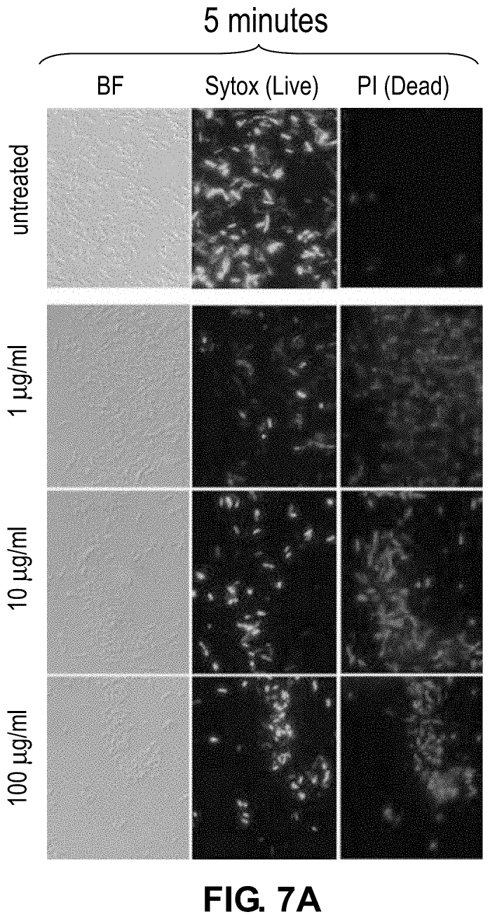

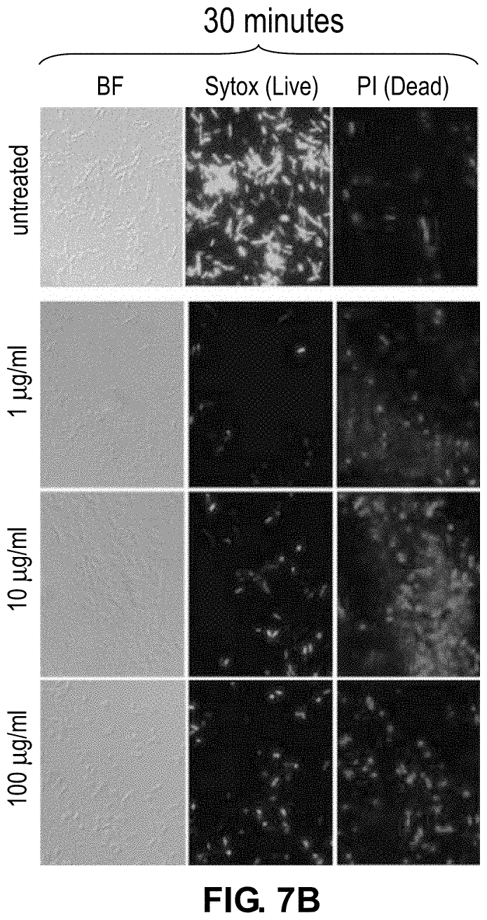

The efficacy of both Chp2-M1 and Ecp3-M1 was evaluated against P. aeruginosa strain CFS-1292 suspended in 100% human serum with LIVE/DEAD stain. Chp2-M1 or Ecp3-M1 in varying concentrations of 1 μg/mL, 10 μg/mL, and 100 μg/mL was added to samples of the suspension. The suspensions were evaluated 1 hour prior to treatment with Chp2-M1 or Ecp3-M1, 5 minutes after treatment with Chp2-M2 or Ecp2-M1, and 30 minutes after treatment with Chp2-M1 or Ecp3-M1. Samples were visualized by BF and fluorescence microscopy (10 ms exposure). Sytox Green was used to label live cells, and PI was used to label damaged and dead cells. As shown in FIGS. 6A-B, Chp2-M1 induced rapid killing of P. aeruginosa after 5 minutes (FIG. 6A) and after 30 minutes (FIG. 6B) at all concentrations tested. Likewise, as shown in FIGS. 7A-B, Ecp3-M1 induced rapid killing of P. aeruginosa after 5 minutes (FIG. 7A) and after 30 minutes (FIG. 7B) at all concentrations tested.

### Example 15—Spontaneous Resistance

Spontaneous resistance was assessed as described in Drago, et al. In vitro selection of resistance in Pseudomonas aeruginosa and Acinetobacter spp. by levofloxacin and ciprofloxacin alone and in combination with β-lactams and amikacin, J. ANTIMICROB. CHEMOTHERAPY. 2005; 56(2):353-359 and Rodriguez-Rojas et al., Frequency of Spontaneous Resistance to Fosfomycin Combined with Different Antibiotics in Pseudomonas aeruginosa, ANTIMICROBIAL AGENTS AND CHEMOTHERAPY 2010; 54(11):4948-49.

Late-log phase P. aeruginosa strain CFS-1292 was plated on CAA media supplemented with Chp peptides or antibiotics (ciprofloxacin or tobramycin) at 4×MIC. The frequency of spontaneous resistance was calculated by dividing the number of resistant colonies after 48 hours by the total number of CFUs determined by quantitative plating in the absence of selection. As shown in Table 48 below, all of the Chp peptides tested exhibited a low propensity for spontaneous resistance.

TABLE 48
Spontaneous resistance of Chp peptides

| Chp peptide | MIC | |
|---|---|---|
| or antibiotic | (μg/mL) | frequency |
| Chp2 | 0.25 | <1.25E−09 |
| Chp2-M1 | 0.125 | <1.25E−09 |
| Chp4-M1 | 0.125 | <1.25E−09 |
| Chp6-M1 | 0.25 | <1.25E−09 |
| Chp10-M1 | 0.0625 | <1.25E−09 |
| Ecp3-M1 | 0.125 | <1.25E−09 |
| Unp2-M1 | 0.125 | <1.25E−09 |
| Ciprofloxacin | 0.5 | 6.25E−08 |
| Tobramycin | 0.0625 | 3.85E−09 |

### Example 16—Serial Passage Study

A 2-fold dilution serial passage study was conducted with P. aeruginosa strain CFS 1292 against 7 Chp peptides and 2 antibiotics (in duplicate) over a 21-day time period. The MIC of subcultures was determined at daily at days 0-9 and compared to an untreated control. The results are shown below in Tables 49 and 50. As shown in Table 49, as of Day 9, no significant changes were observed in the MIC values for any of the 7 Chp peptides tested; however, ciprofloxacin exhibited a 4-fold increase in MIC. As shown in Table 50, after 21 days, Chp2, Chp2-M1, Chp4-M1, Chp6-M1, and Chp10-M1 all showed no significant change in MIC values, while Ecp3-M1 and Unp2-M1 showed a 2-fold increase in MIC. Both ciprofloxacin and tobramycin showed significant increases in the MIC values after 21 days, evidencing a relatively high propensity for spontaneous resistance in those antibiotics.

TABLE 49
Serial Passage MIC (ug/mL) of Chp Peptides over 9 Days

| Chp | | | | | | | | | | |
|---|---|---|---|---|---|---|---|---|---|---|
| peptide or | | | | | | | | | | |
| antibiotic | Day 0 | Day 1 | Day 2 | Day 3 | Day 4 | Day 5 | Day 6 | Day 7 | Day 8 | Day 9 |
| Chp2 (1) | 0.25 | 0.25 | 0.25 | 0.25 | 0.25 | 0.25 | 0.25 | 0.25 | 0.25 | 0.25 |
| Chp2 (2) | 0.25 | 0.25 | 0.25 | 0.25 | 0.25 | 0.25 | 0.25 | 0.25 | 0.25 | 0.25 |
| Chp2 | 0.25 | 0.25 | 0.25 | 0.25 | 0.25 | 0.25 | 0.25 | 0.25 | 0.25 | 0.25 |
| (untreated) | | | | | | | | | | |
| Chp2-M1 | .0625 | .0625 | .0625 | .0625 | .0625 | .0625 | .0625 | .0625 | .0625 | .0625 |
| (1) | | | | | | | | | | |
| Chp2-M1 | .0625 | .0625 | .0625 | .0625 | .0625 | .0625 | 0.125 | 0.125 | 0.125 | .0625 |
| (2) | | | | | | | | | | |
| Chp2-M1 | .0625 | .0625 | .0625 | .0625 | .0625 | .0625 | 0.125 | 0.125 | 0.125 | 0.125 |
| (untreated) | | | | | | | | | | |
| Chp4-M1 | 0.125 | 0.125 | 0.125 | 0.125 | 0.125 | 0.125 | 0.125 | 0.125 | 0.125 | 0.125 |
| (1) | | | | | | | | | | |
| Chp4-M1 | 0.125 | 0.125 | 0.125 | 0.125 | 0.125 | 0.125 | 0.125 | 0.125 | 0.125 | 0.125 |
| (2) | | | | | | | | | | |
| Chp4-M1 | 0.125 | 0.125 | 0.125 | 0.125 | 0.125 | 0.125 | 0.125 | 0.125 | 0.125 | 0.125 |
| untreated | | | | | | | | | | |
| Chp6-M1 | 0.125 | 0.125 | 0.125 | 0.125 | 0.125 | 0.125 | 0.25 | 0.25 | 0.25 | 0.125 |
| (1) | | | | | | | | | | |
| Chp6-M1 | 0.125 | 0.125 | 0.125 | 0.125 | 0.125 | 0.125 | 0.25 | 0.25 | 0.25 | 0.125 |
| (2) | | | | | | | | | | |
| Chp6-M1 | 0.125 | 0.125 | 0.125 | 0.125 | 0.125 | 0.125 | 0.25 | 0.25 | 0.25 | 0.125 |
| (untreated) | | | | | | | | | | |
| Chp10-M1 | .0625 | .0625 | .0625 | .0625 | .0625 | .0625 | .0625 | .0625 | .0625 | .0625 |
| (1) | | | | | | | | | | |
| Chp10-M1 | .0625 | .0625 | .0625 | .0625 | .0625 | .0625 | .0625 | .0625 | .0625 | .0625 |
| (2) | | | | | | | | | | |
| Chp10-M1 | .0625 | .0625 | .0625 | .0625 | .0625 | .0625 | .0625 | .0625 | .0625 | .0625 |
| (untreated) | | | | | | | | | | |
| Ecp3-M1 | 0.125 | 0.125 | 0.125 | 0.125 | 0.125 | 0.125 | 0.125 | 0.125 | 0.125 | 0.25 |
| (1) | | | | | | | | | | |
| Ecp3-M1 | 0.125 | 0.125 | 0.125 | 0.125 | 0.125 | 0.125 | 0.125 | 0.125 | 0.125 | 0.25 |
| (2) | | | | | | | | | | |
| Ecp3-M1 | 0.125 | 0.125 | 0.125 | 0.125 | 0.125 | 0.125 | 0.125 | 0.125 | 0.125 | 0.125 |
| (untreated) | | | | | | | | | | |
| Unp2-M1 | 0.125 | 0.125 | 0.125 | 0.125 | 0.125 | 0.125 | 0.125 | 0.125 | 0.125 | 0.25 |
| (1) | | | | | | | | | | |
| Unp2-M1 | 0.125 | 0.125 | 0.125 | 0.125 | 0.125 | 0.125 | 0.125 | 0.125 | 0.125 | 0.25 |
| (2) | | | | | | | | | | |
| Unp2-M1 | 0.125 | 0.125 | 0.125 | 0.125 | 0.125 | 0.125 | 0.125 | 0.125 | 0.125 | 0.25 |
| (untreated) | | | | | | | | | | |
| Imipenem | 8 | 8 | 8 | 16 | 16 | 16 | 16 | 16 | 16 | 8 |
| (1) | | | | | | | | | | |
| Imipenem | 8 | 8 | 8 | 16 | 16 | 16 | 16 | 16 | 16 | 8 |
| (2) | | | | | | | | | | |
| Imipenem | 8 | 8 | 8 | 16 | 16 | 16 | 8 | 8 | 8 | 8 |
| (untreated) | | | | | | | | | | |
| Cipro (1) | 0.25 | 0.25 | 0.25 | 1 | 1 | 1 | 1 | 1 | 1 | 2 |
| Cipro (2) | 0.25 | 0.25 | 0.25 | 1 | 1 | 1 | 2 | 2 | 2 | 2 |
| Cipro | 0.25 | 0.25 | 0.25 | 0.5 | 0.5 | 0.5 | 0.5 | 0.5 | 0.5 | 0.5 |
| (untreated) | | | | | | | | | | |

TABLE 50
Serial Passage MIC (ug/mL) of Chp
Peptides over 21 Days

| | MIC (μg/mL) | |
|---|---|---|
| Agent | Day 0 | Day 21 |
| Chp2 (l) | 0.25 | 0.25 |
| Chp2 (2) | 0.25 | 0.25 |
| Chp2-M1 (1) | 0.125 | 0.125 |
| Chp2-M1 (2) | 0.125 | 0.125 |
| Chp4-M1 (1) | 0.125 | 0.125 |
| Chp4-M1 (2) | 0.125 | 0.125 |
| Chp6-M1 (1) | 0.25 | 0.25 |
| Chp6-M1 (2) | 0.25 | 0.25 |
| Chp10-M1 (1) | 0.0625 | 0.0625 |
| Chp10-M1 (2) | 0.0625 | 0.0625 |
| Ecp3-M1 (1) | 0.125 | 0.25 |
| Ecp3-M1 (2) | 0.125 | 0.25 |
| Unp2-M1 (1) | 0.125 | 0.25 |
| Unp2-M1 (2) | 0.125 | 0.25 |
| Cipro (1) | 0.5 | 16 |
| Cipro (2) | 0.5 | 8 |
| Tobra (1) | 0.125 | 32 |
| Tobra (2) | 0.125 | 4 |

Spot dilution assays were also performed to investigate the potential for decreased susceptibility of resistance to P. aeruginosa of Chp2, Chp2-M1, Chp10-M1, ciprofloxacin, and tobramycin. P. aeruginosa (CFS 1292) was plated on CAA agar, and 25 μL of 1 mg/mL of the agent to be tested was spotted in the center of a plate. Plates were then incubated for two days at 24° C., and the clearing zone was observed. Colonies formed either within the clearing zone or at the periphery were subcultured (3×) and tested for MIC values. Resulting colonies for Chp2, Chp2-M1, and Chp10-M1 were then used as an inoculum for four additional serial passages (five passages total) to investigate the propensity for developing antibacterial resistance. Table 51 below shows the MIC of the tested agents in an untreated control, in the periphery of the clearing zone, and in the center of the clearing zone. Values in bold print indicate the colonies that were then subjected to the four serial passages, the results of which are shown in Table 52, below.

TABLE 51
MIC (μg/mL) of Agents in Spot Dilution Assays

| | MIC (μg/mL) | | |
|---|---|---|---|
| Agent | Untreated control | Periphery | Center |
| Chp2 | 0.25 | 0.25 | No growth |
| Chp2-M1 | 0.125 | 0.125 | No growth |
| Chp4-M1 | 0.125 | 0.125 | 0.125 |
| Chp6-M1 | 0.25 | 0.25 | No growth |
| Chp10-M1 | 0.0625 | 0.0625 | No growth |
| Ecp3-M1 | 0.125 | 0.125 | 0.125 |
| Unp2-M1 | 0.25 | 0.25 | 0.25 |
| Ciprofloxacin | 0.5 | 2 | 2 |
| Tobramycin | 0.125 | 0.5 | 0.5 |

TABLE 52
Serial Passage of Chp Peptides

| | Passage MIC (μg/mL) | | | | |
|---|---|---|---|---|---|
| | Passage | Passage | Passage | Passage | Passage |
| Agent | #1 | #2 | #3 | #4 | #5 |
| Chp2 (1) | 0.25 | 0.25 | 0.25 | 0.25 | 0.25 |
| Chp2 (2) | 0.25 | 0.25 | 0.25 | 0.25 | 0.25 |
| Untreated | 0.25 | 0.25 | 0.25 | 0.25 | 0.25 |
| Chp2-M1 (1) | 0.125 | 0.125 | 0.125 | 0.125 | 0.125 |
| Chp2-M1 (2) | 0.125 | 0.125 | 0.125 | 0.125 | 0.125 |
| Untreated | 0.125 | 0.125 | 0.125 | 0.125 | 0.125 |
| Chp10-M1 | 0.062 | 0.062 | 0.062 | 0.062 | 0.062 |
| (1) | | | | | |
| Chp10-M1 | 0.062 | 0.062 | 0.062 | 0.062 | 0.062 |
| (2) | | | | | |
| Untreated | 0.062 | 0.062 | 0.062 | 0.062 | 0.062 |
| Cipro (1) | 2 | 2 | 2 | 4 | 4 |
| Cipro (2) | 2 | 2 | 4 | 4 | 4 |
| Untreated | 0.5 | 0.5 | 0.5 | 0.5 | 0.5 |
| Tobra (1) | 0.5 | 0.5 | 0.5 | 0.5 | 1 |
| Tobra (2) | 0.5 | 0.5 | 0.5 | 0.5 | 1 |
| Untreated | 0.125 | 0.125 | 0.125 | 0.125 | 0.125 |

The results in Table 52 demonstrate that Chp2, Chp2-M1, and Chp10-M1 showed no propensity towards resistance.

### Example 17—Chp Peptide Activity Against Gram-Negative Bacterial Isolates from CDC Resistance Panels

MIC values were determined using the methodology described above, i.e., the standard broth microdilution reference method defined by CLSI. As used herein, MIC is the minimum concentration of peptide sufficient to suppress at least 80% of the bacterial growth compared to control, while MIC₅₀ is the minimum concentration of peptide sufficient to suppress at least 50% of the bacterial growth compared to control, and MIC₉₀ is the minimum concentration of peptide sufficient to suppress at least 50% of the bacterial growth compared to control.

Five different antibiotic-resistant isolate bank panels were chosen from the Center for Disease Control's strain lists. Specifically, two panels of 41 Acinetobacter baumannii isolates and 55 Pseudomonas aeruginosa isolates were chosen to represent a diversity of antimicrobial susceptibility results for drugs that are used to treat infections. The strains are described, for example, at wwwn.cdc.gov/ARIsolateBank/Panel/PanelDetail?ID=1 and wwwn.cdc.gov/ARIsolateBank/Panel/PanelDetail?ID=12 for the Acinetobacter baumannii isolates and the Pseudomonas aeruginosa isolates, respectively.

Additionally, a third panel of 53 carbapenamase-producing Enterobacteriaceae isolates were selected to represent a diversity of species and carbapenemases. See wwwn.cdc.gov/ARIsolateBank/Panel/PanelDetail?ID=8.

A fourth panel of 28 Gram-negative with varying susceptibility to imipenem and relebactam (see wwwn.cdc.gov/ARIsolateBank/Panel/PanelDetail?ID=1034) were also selected. Finally, a fifth panel of 11 out of 17 isolates identified as having novel antibiotic resistance were selected, where the antibiotic resistance may be based on a new resistance mechanism or phenotype. See wwwn.cdc.gov/ARIsolateBank/Panel/PanelDetail?ID=10. The data obtained are summarized in Table 53-57 below.

TABLE 53
Actinobacter baumannii Panel (n = 41)

| CDC | | | |
|---|---|---|---|
| Antibiotic- | MIC | | |
| Resistant | | Chp2- | Chp10- |
| Bank No. | Chp2 | M1 | M1 |
| 0273 | 1 | 0.5 | 0.25 |
| 0274 | 0.5 | 0.5 | 0.5 |
| 0275 | 0.5 | 0.5 | 0.25 |
| 0276 | 0.5 | 0.5 | 1 |
| 0277 | 0.5 | 0.25 | 0.125 |
| 0278 | 1 | 0.5 | 0.25 |
| 0279 | 1 | 0.5 | 0.25 |
| 0280 | 1 | 0.5 | 0.25 |
| 0281 | 1 | 0.5 | 0.25 |
| 0282 | 0.5 | 0.5 | 0.5 |
| 0283 | 1 | 0.5 | 0.25 |
| 0284 | 0.5 | 0.25 | 0.125 |
| 0285 | 0.5 | 0.5 | 0.25 |
| 0286 | 1 | 0.5 | 0.25 |
| 0287 | 0.5 | 0.5 | 0.25 |
| 0288 | 0.25 | 0.125 | 0.03125 |
| 0289 | 0.5 | 0.5 | 0.25 |
| 0290 | 0.5 | 0.5 | 0.25 |
| 0291 | 0.5 | 0.5 | 0.25 |
| 0292 | 0.5 | 0.5 | 0.25 |
| 0293 | 0.5 | 0.5 | 0.5 |
| 0294 | 0.5 | 0.5 | 0.25 |
| 0295 | 1 | 0.5 | 0.25 |
| 0296 | 0.5 | 0.5 | 0.125 |
| 0297 | 1 | 0.5 | 0.25 |
| 0298 | 0.5 | 0.5 | 0.125 |
| 0299 | 1 | 0.5 | 0.25 |
| 0300 | 2 | 1 | 0.25 |
| 0301 | 1 | 1 | 0.25 |
| 0302 | 1 | 1 | 0.25 |
| 0303 | 1 | 1 | 0.25 |
| 0304 | 0.5 | 0.5 | 0.25 |
| 0305 | 0.5 | 0.5 | 0.125 |
| 0306 | 0.5 | 1 | 0.5 |
| 0307 | 1 | 1 | 0.5 |
| 0308 | 1 | 1 | 0.5 |
| 0309 | 1 | 1 | 0.5 |
| 0310 | 0.5 | 1 | 0.25 |
| 0311 | 0.5 | 0.5 | 0.25 |
| 0312 | 0.5 | 0.5 | 0.125 |
| 0313 | 0.5 | 0.5 | 0.125 |

TABLE 54
Pseudomonas aeruginosa Panel (n = 55)

| CDC | | | |
|---|---|---|---|
| Antibiotic- | MIC | | |
| Resistant | | Chp2- | Chp10- |
| Bank No. | Chp2 | M1 | M1 |
| 0229 | 0.5 | 0.5 | 0.25 |
| 0230 | 0.5 | 0.5 | 0.5 |
| 0231 | 0.5 | 0.5 | 0.5 |
| 0232 | 0.5 | 0.5 | 0.25 |
| 0233 | 0.5 | 0.25 | 0.125 |
| 0234 | 0.5 | 0.5 | 0.25 |
| 0235 | 0.5 | 0.5 | 0.5 |
| 0236 | 0.5 | 0.5 | 0.125 |
| 0237 | 0.5 | 0.5 | 0.125 |
| 0238 | 0.5 | 1 | 0.25 |
| 0239 | 1 | 0.5 | 0.25 |
| 0240 | 1 | 0.5 | 0.25 |
| 0241 | 1 | 0.5 | 0.25 |
| 0242 | 0.5 | 1 | 0.125 |
| 0243 | 0.5 | 0.25 | 0.03125 |
| 0244 | 1 | 0.5 | 0.25 |
| 0245 | 0.5 | 0.5 | 0.25 |
| 0246 | 1 | 1 | 0.5 |
| 0247 | 1 | 1 | 0.25 |
| 0248 | 1 | 1 | 0.25 |
| 0249 | 1 | 1 | 0.25 |
| 0250 | 1 | 1 | 0.25 |
| 0251 | 1 | 1 | 0.25 |
| 0252 | 1 | 0.5 | 0.25 |
| 0253 | 1 | 0.5 | 0.125 |
| 0254 | 1 | 1 | 0.5 |
| 0255 | 1 | 1 | 0.5 |
| 0256 | 1 | 1 | 0.125 |
| 0257 | 1 | 0.5 | 0.125 |
| 0258 | 2 | 1 | 0.25 |
| 0259 | 1 | 1 | 0.25 |
| 0260 | 0.5 | 0.5 | 0.25 |
| 0261 | 1 | 1 | 0.25 |
| 0262 | 1 | 1 | 0.25 |
| 0263 | 1 | 0.25 | 0.25 |
| 0264 | 1 | 0.5 | 0.125 |
| 0265 | 1 | 1 | 0.5 |
| 0266 | 1 | 1 | 1 |
| 0267 | 1 | 1 | 0.5 |
| 0268 | 1 | 1 | 0.5 |
| 0269 | 1 | 1 | 1 |
| 0270 | 0.5 | 1 | 0.125 |
| 0271 | 1 | 1 | 0.5 |
| 0272 | 1 | 1 | 0.5 |
| 0763 | 1 | 0.5 | 0.0625 |
| 0764 | 1 | 0.5 | 0.0625 |
| 0765 | 1 | 0.5 | 0.0625 |
| 0766 | 1 | 0.5 | 0.0313 |
| 0767 | 1 | 1 | 0.5 |
| 0768 | 0.5 | 0.0313 | 0.0313 |
| 0769 | 1 | 1 | 0.5 |
| 0770 | 1 | 1 | 0.25 |
| 0771 | 1 | 1 | 0.5 |
| 0772 | 1 | 1 | 0.125 |
| 0773 | 1 | 1 | 0.5 |

TABLE 55
Carbapenamase Enterobacteriaceae Panel (n = 53)

| CPC | | | | |
|---|---|---|---|---|
| Antibiotic- | | MIC | | |
| Resistant | | | Chp2- | Chp10- |
| Bank No. | Organism | Chp2 | M1 | M1 |
| 0112 | Klebsiella pneumoniae | 1 | 0.5 | 0.5 |
| 0113 | Klebsiella pneumoniae | 1 | 0.5 | 0.25 |
| 0114 | Escherichia coli | 0.5 | 0.25 | 0.125 |
| 0115 | Klebsiella pneumoniae | 1 | 0.5 | 0.5 |
| 0116 | Citrobacter freundii | 0.5 | 0.125 | 0.125 |
| 0117 | Klebsiella pneumoniae | 0.5 | 0.25 | 0.125 |
| 0118 | Escherichia coli | 0.5 | 0.25 | 0.0625 |
| 0119 | Escherichia coli | 0.5 | 0.25 | 0.0625 |
| 0120 | Klebsiella pneumoniae | 0.25 | 0.25 | 0.5 |
| 0121 | Serratia marcescens | >8 | >8 | >8 |
| 0122 | Serratia marcescens | >8 | >8 | >8 |
| 0123 | Serratia marcescens | >8 | >8 | >8 |
| 0124 | Serratia marcescens | >8 | >8 | >8 |
| 0125 | Klebsiella pneumoniae | 1 | 1 | 0.5 |
| 0126 | Klebsiella pneumoniae | 1 | 0.5 | 0.125 |
| 0127 | Salmonella Senftenberg | 0.5 | 0.5 | 0.125 |
| 0128 | Escherichia coli | 0.5 | 0.25 | 0.0625 |
| 0129 | Klebsiella pneumoniae | 1 | 1 | 0.5 |
| 0130 | Serratia marcescens | >8 | >8 | >8 |
| 0131 | Serratia marcescens | >8 | >8 | >8 |
| 0132 | Enterobacter cloacae | 1 | 1 | 1 |
| | group | | | |
| 0133 | Morganella morganii | ng | ng | ng |
| 0134 | Raoultella ornithinolytica | 1 | 0.25 | 0.25 |
| 0135 | Klebsiella pneumoniae | 0.5 | 0.25 | 0.0625 |
| 0136 | Enterobacter cloacae | 0.5 | 0.25 | 0.25 |
| 0137 | Escherichia coli | 0.25 | 0.25 | 0.0625 |
| 0138 | Klebsiella pneumoniae | 0.5 | 0.25 | 0.125 |
| 0139 | Klebsiella pneumoniae | 1 | 0.25 | 0.125 |
| 0140 | Klebsiella pneumoniae | 0.5 | 0.25 | 0.0625 |
| 0141 | Klebsiella pneumoniae | 0.5 | 0.25 | 0.0625 |
| 0142 | Klebsiella pneumoniae | 0.5 | 0.25 | 0.0625 |
| 0143 | Klebsiella pneumoniae | 0.5 | 0.5 | 0.0625 |
| 0144 | Kluyvera ascorbata | 0.25 | 0.25 | 0.125 |
| 0145 | Klebsiella pneumoniae | 1 | 0.5 | 0.25 |
| 0146 | Klebsiella pneumoniae | 1 | 1 | 0.5 |
| 0147 | Klebsiella oxytoca | 1 | 0.25 | 0.25 |
| 0148 | Klebsiella pneumoniae | 1 | 1 | 0.5 |
| 0149 | Escherichia coli | 0.5 | 0.25 | 0.03125 |
| 0150 | Escherichia coli | 0.25 | 0.125 | 0.03125 |
| 0151 | Escherichia coli | 0.25 | 0.125 | 0.0625 |
| 0152 | Klebsiella pneumoniae | 1 | 0.5 | 0.25 |
| 0153 | Klebsiella pneumoniae | 0.5 | 0.25 | 0.0625 |
| 0154 | Enterobacter cloacae | 0.25 | 0.25 | 0.0625 |
| 0155 | Proteus mirabilis | 0.25 | 0.5 | 0.0625 |
| 0156 | Proteus mirabilis | 0.5 | 1 | 0.25 |
| 0157 | Citrobacter freundii | 0.5 | 0.25 | 0.125 |
| 0158 | Klebsiella pneumoniae | 0.5 | 0.25 | 0.0625 |
| 0159 | Proteus mirabilis | 0.25 | 1 | 0.25 |
| 0160 | Klebsiella pneumoniae | 1 | 0.5 | 0.25 |
| 0161 | Enterbacter aerogenes | 2 | 0.25 | 0.0625 |
| 0162 | Escherichia coli | 0.25 | 0.25 | 0.03125 |
| 0163 | Enterobacter cloacae | 0.25 | 0.125 | 0.0625 |
| 0164 | Enterobacter cloacae | 1 | 0.25 | 0.125 |
| | complex | | | |

TABLE 56
Imipenem/Relebactam Panel (n = 28)

| CPC | | | | |
|---|---|---|---|---|
| Antibiotic- | | MIC | | |
| Resistant | | | Chp2- | Chp10- |
| Bank No. | Organism | Chp2 | M1 | M1 |
| 0501 | Enterobacter cloacae | 0.5 | 0.25 | 0.25 |
| 0502 | Enterobacter cloacae | 0.5 | 0.25 | 0.25 |
| 0504 | Klebsiella pneumoniae | 0.5 | 0.25 | 0.25 |
| 0505 | Klebsiella pneumoniae | 1 | 0.25 | 0.5 |
| 0506 | Klebsiella pneumoniae | 1 | 0.5 | 2 |
| 0507 | Klebsiella pneumoniae | 0.5 | 0.25 | 0.25 |
| 0508 | Pseudomonas aeruginosa | 1 | 0.5 | 0.125 |
| 0509 | Pseudomonas aeruginosa | 1 | 1 | 0.5 |
| 0510 | Pseudomonas aeruginosa | 0.5 | 0.5 | 0.25 |
| 0511 | Pseudomonas aeruginosa | 0.5 | 0.5 | 0.125 |
| 0512 | Pseudomonas aeruginosa | 1 | 0.5 | 0.25 |
| 0513 | Pseudomonas aeruginosa | 0.5 | 0.5 | 0.25 |
| 0514 | Pseudomonas aeruginosa | 1 | 0.5 | 0.125 |
| 0515 | Pseudomonas aeruginosa | 0.5 | 0.5 | 0.25 |
| 0516 | Pseudomonas aeruginosa | 0.5 | 0.5 | 0.25 |
| 0517 | Serratia marcescens | >8 | 8 | >8 |
| 0518 | Pseudomonas aeruginosa | 1 | 0.5 | 0.125 |
| 0519 | Morganella morganii | ng | ng | ng |
| 0520 | Serratia marcescens | >8 | >8 | >8 |
| 0521 | Serratia marcescens | >8 | 4 | >8 |
| 0522 | Klebsiella pneumoniae | 0.5 | 0.5 | 0.25 |
| 0523 | Klebsiella pneumoniae | 0.5 | 0.25 | 0.125 |
| 0524 | Klebsiella pneumoniae | 1 | 1 | 0.5 |
| 0525 | Klebsiella pneumoniae | 1 | 1 | 0.25 |
| 0526 | Pseudomonas aeruginosa | 2 | 1 | 0.5 |
| 0527 | Pseudomonas aeruginosa | 1 | 0.5 | 0.125 |
| 0528 | Pseudomonas aeruginosa | 1 | 1 | 0.25 |
| 0529 | Pseudomonas aeruginosa | 0.5 | 0.5 | 0.125 |

TABLE 57
Novel Antibiotic Resistance Panel (n = 11)

| CPC | | | | |
|---|---|---|---|---|
| Antibiotic- | | MIC | | |
| Resistant | | | Chp2- | Chp10- |
| Bank No. | Organism | Chp2 | M1 | M1 |
| 0346 | Escherichia coli | 0.25 | 0.25 | 0.03125 |
| 0347 | Klebsiella pneumoniae | 0.5 | 0.5 | 0.25 |
| 0348 | Escherichia coli | 0.5 | 0.125 | 0.03125 |
| 0349 | Escherichia coli | 1 | 0.125 | 0.0156 |
| 0350 | Escherichia coli | 1 | 0.25 | 0.03125 |
| 0493 | Escherichia coli | 0.5 | 0.25 | 0.03125 |
| 0494 | Escherichia coli | 1 | 0.125 | 0.03125 |
| 0495 | Escherichia coli | 1 | 0.135 | 0.03125 |
| 0497 | Klebsiella pneumoniae | 0.5 | 0.25 | 0.0625 |
| 0538 | Escherichia coli | 1 | 0.25 | 0.03125 |
| 0637 | Citrobacter freundi | 1 | 0.25 | 0.125 |

For the Actinobacter baumannii panel summarized in Table 53, the MIC₅₀ for Chp2, Chp2-M1, and Chp10-M1 was 0.5, 0.5, and 0.25, and the MIC₉₀ was 1, 1, and 0.5, respectively. For the Pseudomonas aeruginosa panel summarized in Table 54, the MIC₅₀ for Chp2, Chp2-M1, and Chp10-M1 was 1, 1, and 0.25, and the MIC₉₀ was 1, 1, and 0.5, respectively. For the Carbapenamase Enterobacteriaceae panel summarized in Table 55, the MIC₅₀ for Chp2, Chp2-M1, and Chp10-M1 was 0.5, 0.25, and 0.125, and the MIC₉₀ was 1, 1, and 0.5, respectively. For the Imipenem/Relebactam panel summarized in Table 56, the MIC₅₀ for Chp2, Chp2-M1, and Chp10-M1 was 0.5, 0.5, and 0.25, and the MIC₉₀ was 1, 1, and 0.5, respectively. For the novel antibiotic resistance panel summarized in Table 57, the MIC₅₀ for Chp2, Chp2-M1, and Chp10-M1 was 0.5, 0.125, and 0.0313, and the MIC₉₀ was 1, 0.25, and 0.125, respectively.

The results establish that a variety of bacterial strains that exhibit antibiotic resistance to various standard-of-care antibiotics are nonetheless highly susceptible to Chp peptides disclosed herein, including Chp2, Chp2-M1, and Chp10-M1.

### Example 18—Endotoxin Neutralization by Chp Peptides

Lipopolysaccharides (LPS), also known as polyglycans and endotoxins, are prevalent throughout the outer membrane of Gram-negative bacteria. LPS can stimulate the expression of pro-inflammatory cytokines. Certain amurin peptides, in addition to antibacterial activity, also exhibit the ability to bind and neutralize LPS. Both LL-37 and colistin, for example, bind strongly to LPS. See, e.g., Rosenfeld et al., Endotoxin (Lipopolysaccharide) Neutralization by Innate Immunity Host-Defense Peptides, J. BIO. CHEM. 2005; 281(3):1636-1643; and Mohamed et al., A short D-enantiomeric antimicrobial peptide with potent immunomodulatory and antibiofilm activity against multidrug-resistant Pseudomonas aeruginosa and Acinetobacter baumannii, SCIENTIFIC REPORTS 2017; 6953(7):1-13. As molecules that bind LPS and neutralize its toxic effect may have clinical applications, the ability of Chp peptides as disclosed herein was evaluated to determine their binding ability to LPS.

In vitro limulus amoebocyte lysate (LAL) enzymes assays were used to examine the ability of Chp peptides Chp2, Chp2-M1, and Chp10-M1 to bind LPS and inhibit LPS-induced activation of LAL enzymes, as well as downstream cleavage of chromogenic reporters.

The protocols described in Mohamed et al., 2017 and Roberts et al., In Vitro Evaluation of the Interaction of Dextrin-Colistin Conjugates with Bacterial Lipopolysaccharide, J. MED. CHEM. 2016; 59:647-654 were used followed, and colistin, LL-37, and Chp5 were used as controls, with the exception that endotoxin neutralization was evaluated using a Pierce Chromogenic Endotoxin Quant Kit (ThermoFisher Scientific). Specifically, LPS was dissolved in pyrogen-free water (0.8 EU/mL, wherein EU indicate the endotoxin unit, and one EU equals approximately 0.1 to 0.2 ng endotoxin/mL of solution) containing the indicated concentration range (0.125 μg/mL-64 μg/mL) of Chp peptide or control peptide. The standard reference samples contained only LPS dissolved in pyrogen-free water. Solutions were mixed well and incubated at 24° C. for 1 hour, and the manufacturers' test procedure for quantitative detection of endotoxins was followed. Table 58 shows the percentage of LPS binding that was observed with increasing dosages of the Chp peptides and control peptides.

TABLE 58
Chp Peptide Binding of LPS

| Peptide | % LPS Bound | | | | | |
|---|---|---|---|---|---|---|
| concentration | | | Chp10- | | | |
| (μg/mL) | Chp2 | Chp2-M1 | M1 | Chp5 | Colistin | LL-37 |
| 0.125 | 74.4 | 60.5 | 49.8 | 2.8 | 10.5 | 61.7 |
| 0.25 | 57.9 | 50.6 | 35.1 | 6.0 | 14.8 | 88.7 |
| 0.5 | 28.7 | 27.5 | 13.3 | 0.1 | 22.5 | 90.6 |
| 1 | 28.0 | 18.3 | 7.5 | 0.1 | 51.9 | 92.2 |
| 2 | 27.0 | 16.5 | 15.6 | 0.1 | 74.2 | 92.9 |
| 4 | 29.3 | 20.4 | 11.8 | 0.1 | 88.2 | 92.3 |
| 8 | 31.0 | 25.9 | 10.3 | 5.4 | 92.4 | 92.4 |
| 16 | 40.8 | 20.4 | 7.7 | 0.1 | 92.8 | 92.1 |
| 32 | 58.1 | 17. | 0.5 | 0.1 | 92.2 | 87.3 |
| 64 | 71.0 | 31.9 | 9.1 | 0.9 | 93.4 | 75.4 |

The results indicate that both colistin and LL-37 demonstrate dose-dependent increases in LPS binding, and Chp5 does not bind LPS. Chp2, Chp2-M1, and Ch10-M1 all demonstrate binding to LPS, exhibiting a pattern of binding preferentially at both very low and very high concentrations.

### Example 19—Chp Peptide Activity Against Persister Cells

The activity of Chp peptides Chp2, Chp2-M1, and Chp10-M1 against persister cells (bacterial cell variants that are highly resistant to different classes of antibiotics) of A. baumannii and P. aeruginosa was evaluated using the methods set forth in Defraine, V. et al., Efficacy of Artilysin Art-175 against Resistant and Persistent Acinetobacter baumannii, ANTIMICROB. AGENTS AND CHEMOTHER. 2016; 60(6):3480-3488. Briefly, A. baumannii strain BAA-747 was grown overnight in 5% tryptic soy broth at 37° C. with shaking. At 18 hours, the culture was exposed to 60×MIC tobramycin for 5 hours at 37° C. to select for persister cells. Persister cells surviving antibiotic treatment were harvested; samples of ciprofloxacin, lysozyme, Chp2, Chp2-M1, Chp10-M1, Chp5, and a buffer control were then added to 100 μL volumes of isolated persister cell fractions, and samples were incubated at 37° C. for 1 hour. Cells were then harvested, plated on agar plates, and incubated at 37° C. for up to 3 days. The MICs of any surviving bacteria were then measured, wherein the limit of detection was 1.6-log₁₀ CFU/mL. The results are shown below in Table 59.

TABLE 59
MIC Log₁₀ (CFU/mL) of A. Baumanii Persister Cells

| | Run #1 | Run #2 | Run #3 | Average | Delta |
|---|---|---|---|---|---|
| | (CFU/mL) | (CFU/mL) | (CFU/mL) | (CFU/mL) | Log |
| Buffer | 4.2 | 5.6 | 4.8 | 4.9 | 0.0 |
| Ciprofloxacin | 3.5 | 4.9 | 4.5 | 4.3 | 0.6 |
| Lysozyme | 3.5 | 5.2 | 5.0 | 4.6 | 0.3 |
| Chp2 | 1.5 | 2.2 | 1.5 | 1.8 | 3.1 |
| Chp2-M1 | 1.5 | 1.6 | 1.5 | 1.6 | 3.3 |
| Chp10-M1 | 1.5 | 1.6 | 2.5 | 1.9 | 3.0 |
| Chp5 | 4.1 | 5.1 | 4.5 | 4.6 | 0.3 |

As shown in Table 59, the A. baumannii persister cells were sensitive to all of Chp2, Chp2-M1, and Chp10-M1, with a log₁₀ CFU/mL reduction as compared to buffer of 3.1, 3.3, and 3.0, respectively. Surviving persister cells of the Chp peptide treatments exhibited no change in MIC values.

The method outlined above for A. baumannii was repeated with P. aeruginosa strain PA20, wherein the limit of detection was 2-log₁₀ CFU/mL, and the results are shown below in Table 60.

TABLE 60
MIC Log₁₀ (CFU/mL) of P. Aeruginosa Persister Cells

| | Run #1 | Run #2 | Run #3 | Average | Delta |
|---|---|---|---|---|---|
| | (CFU/mL) | (CFU/mL) | (CFU/mL) | (CFU/mL) | Log |
| Buffer | 4.7 | 4.4 | 4 | 4.4 | 0.0 |
| Ciprofloxacin | 4.2 | 4.4 | 4.1 | 4.2 | 0.2 |
| Lysozyme | 4.7 | 4.4 | 4.2 | 4.4 | 0.0 |
| Chp2 | 2.7 | 2.3 | 2 | 2.3 | 2.1 |
| Chp2-M1 | 3 | 2.6 | 2.1 | 2.6 | 1.8 |
| Chp10-M1 | 2.3 | 2.0 | 2.1 | 2.1 | 2.3 |
| Chp5 | 4.4 | 4.3 | 3.1 | 3.9 | 0.5 |

As shown in Table 60, the P. aeruginosa persister cells were sensitive to all of Chp 2, Chp2-M1, and Chp1-M1, with a log₁₀ CFU/mL reduction as compared to buffer of 2.1, 1.8, and 2.3, respectively. Surviving persister cells of the Chp peptide treatments exhibited no change in MIC values.

1. A pharmaceutical composition comprising:
an effective amount of (i) an isolated Chp peptide having an amino acid sequence selected from the group consisting of SEQ ID NOs. 81-91 and 94-102 or active fragment thereof, or (ii) a modified Chp peptide having 80% sequence identity with the amino acid sequence of at least one of SEQ ID NOs. 81-91 and 94-102, wherein the modified Chp peptide inhibits the growth, reduces the population, or kills at least one species of Gram-negative bacteria or acid-fast bacteria; and
a pharmaceutically acceptable carrier.

2. The pharmaceutical composition of claim 1, wherein the Chp peptide has an amino acid sequence selected from SEQ ID NO: 81, SEQ ID NO: 89, or active fragments thereof.

3. (canceled)

4. (canceled)

5. (canceled)

6. The pharmaceutical composition according to claim 1, which is a solution, a suspension, an emulsion, an inhalable powder, an aerosol, or a spray.

7. The pharmaceutical composition according to claim 1, further comprising one or more antibiotics suitable for the treatment of Gram-negative bacteria or acid-fast bacteria.

8.-19. (canceled)

20. A method of inhibiting the growth, reducing the population, or killing of at least one species of Gram-negative bacteria, the method comprising contacting the bacteria with a composition comprising an effective amount of (i) a Chp peptide comprising an amino acid sequence selected from the group consisting of SEQ ID NOs. 81-91 and 94-102 or active fragments thereof, or (ii) a modified Chp peptide having 80% sequence identity with the amino acid sequence of at least one of SEQ ID NOs. 81-91 and 94-102, said Chp peptide or modified Chp peptide having lytic activity for a period of time sufficient to inhibit said growth, reduce said population, or kill said at least one species of Gram-negative bacteria.

21. The method of inhibiting the growth, reducing the population, or killing of at least one species of Gram-negative bacteria according to claim 20, wherein the amino acid sequence is selected from SEQ ID NO: 81, SEQ ID NO: 89, or active fragments thereof.

22. A method of inhibiting the growth, reducing the population, or killing of at least one species of acid-fast bacteria, the method comprising contacting the bacteria with a composition comprising an effective amount of (i) a Chp peptide comprising an amino acid sequence selected from the group consisting of SEQ ID NOs: 1, 2, 4, 6, 8-21, 23-26, 59-61, 63-65, 67, 81, 87-89, 91, 97, and 99-101 or active fragments thereof, or (ii) a modified Chp peptide having 80% sequence identity with the amino acid sequence of at least one of SEQ ID NOs. 1, 2, 4, 6, 8-21, 23-26, 59-61, 63-65, 67, 81, 87-89, 91, 97, and 99-101, said Chp peptide or modified Chp peptide having lytic activity for a period of time sufficient to inhibit said growth, reduce said population, or kill said at least one species of acid-fast bacteria.

23. The method of inhibiting the growth, reducing the population, or killing of at least one species of acid-fast bacteria according to claim 22, wherein the amino acid sequence is selected from the group consisting of SEQ ID NOs: 81, 87, 88, 89, 91, 97, 100, and 101 or active fragments thereof.

24. A method of preventing or treating a bacterial infection caused by at least one species of Gram-negative or acid-fast bacteria, comprising administering to a subject diagnosed with, at risk for, or exhibiting symptoms of a bacterial infection, a pharmaceutical composition according to claim 1.

25. The method of claim 20, wherein the at least one species of Gram-negative bacteria is selected from the group consisting of Acinetobacter baumannii, Pseudomonas aeruginosa, Escherichia coli, Klebsiella pneumoniae, Enterobacter cloacae, Salmonella, Neisseria gonorrhoeae, and Shigella.

26. (canceled)

27. The method of claim 20, wherein the at least one species of Gram-negative bacteria comprises persister cells.

28. The method of claim 22, wherein the at least one species of acid-fast bacteria is selected from the group consisting of Mycobacterium smegmatis, Mycobacterium tuberculosis, Mycobacterium avium, Mycobacterium kansasii, Mycobacterium scrofulaceum, Mycobacterium peregrinum, Mycobacterium marinum, Mycobacterium intracellulare, and Mycobacterium fortuitum.

29. (canceled)

30. The method of claim 20, wherein the bacterial infection is a topical or systemic bacterial infection.

31. The method of claim 24, further comprising administering to the subject an antibiotic suitable for the treatment of Gram-negative or acid-fast bacterial infection.

32. The method of claim 31, wherein the antibiotic suitable for the treatment of a Gram-negative bacterial infection is selected from one or more of azithromycin, aztreonam, fosfomycin, ceftazidime, cefepime, cefoperazone, ceftobiprole, ciprofloxacin, levofloxacin, aminoglycosides, imipenem, meropenem, doripenem, gentamicin, tobramycin, amikacin, piperacillin, ticarcillin, penicillin, rifampicin, polymyxin B, and colistin.

33. (canceled)

34. The method of claim 30 wherein the antibiotic suitable for the treatment of an acid-fast bacterial infection is selected from one or more of isoniazid, rifampin, ethambutol, and pyrazinamide.

35. The method of claim 30, wherein administering the pharmaceutical composition is more effective in inhibiting the growth, reducing the population, or killing the Gram-negative or acid-fast bacteria than administering the antibiotic alone.

36. The method of claim 30, wherein the Gram-negative or acid-fast bacteria is present in human serum and/or pulmonary surfactant.

37. A method for prevention, disruption, or eradication of a biofilm comprising at least one species of Gram-negative bacteria, the method comprising contacting a biofilm with a pharmaceutical composition comprising (i) an isolated Chp peptide having an amino acid sequence selected from the group consisting of SEQ ID NOs. 81-91 and 94-102 or active fragment thereof, or (ii) a modified Chp peptide having 80% sequence identity with the amino acid sequence of at least one of SEQ ID NOs. 81-91 and 94-102, wherein the modified Chp peptide inhibits the growth, reduces the population, or kills at least one species of Gram-negative bacteria, and wherein the biofilm is effectively prevented, dispersed, or eradicated.

38. The method of claim 37, wherein the at least one species of Gram-negative bacteria is Stenotrophomonas maltophilia.

39. The method of claim 37, wherein the isolated Chp peptide has an amino acid sequence of SEQ ID NO: 81 or active fragment thereof or a modified Chp peptide having 80% identity with SEQ ID NO: 81 wherein the modified Chp peptide inhibits the growth, reduces the population, or kills at least one species of Gram-negative bacteria.

40. The method of claim 20, wherein the at least one species of Gram-negative bacteria is resistant to at least one antibiotic suitable for the treatment of a Gram-negative bacterial infection.

41. The method of claim 22, wherein the Chp peptide comprises SEQ ID NO: 2.


SEQUENCE LISTING (124 sequences)

SEQ ID NO: 1 - protein, 36 aa, Chlamydia virus Chp1
MVRRRRLRRRISRRIFRRTVARVGRRRRSFRGGIRF

SEQ ID NO: 2 - protein, 44 aa, Unknown
  Description of Unknown Chlamydia phage 2 sequence
MRLKMARRRYRLPRRRSRRLFSRTALRMHPRNRLRRIMRGGIRF

SEQ ID NO: 3 - protein, 35 aa, Chlamydia virus CPAR39
MCKKVCKKCPKKGPKNAPKIGAFYERKTPRLKQST

SEQ ID NO: 4 - protein, 39 aa, Chlamydia phage 4
MARRYRLSRRRSRRLFSRTALRMHRRNRLRRIMRGGIRF

SEQ ID NO: 5 - protein, 39 aa, Artificial Sequence
  Description of Artificial Sequence Synthetic polypeptide
MAEQYELSQEQSEQLFSETALQMHEQNELQEIMQGGIEF

SEQ ID NO: 6 - protein, 40 aa, Unknown
  Description of Unknown Guinea pig Chlamydia phage sequence
MARRRYRLPRRRSRRLFSRTALRMHPRNRLRRIMRGGIRF

SEQ ID NO: 7 - protein, 38 aa, Chlamydia trachomatis
MKRRKMTRKGSKRLFTATADKTKSINTAPPPMRGGIRL

SEQ ID NO: 8 - protein, 39 aa, Chlamydia trachomatis
MSKKRSRMSRRRSKKLFSKTALRTKSVNTRPPMRGGFRF

SEQ ID NO: 9 - protein, 40 aa, Chlamydia trachomatis
MSLRRHKLSRKASKRIFRKGASRTKTLNTRATPMRGGFRI

SEQ ID NO: 10 - protein, 38 aa, Chlamydia trachomatis
MKRRKLSKKKSRKIFTRGAVNVKKRNLRARPMRGGFRI

SEQ ID NO: 11 - protein, 37 aa, Chlamydia trachomatis
MAKKMTKGKDRQVFRKTADRTKKLNVRPLLYRGGIRL

SEQ ID NO: 12 - protein, 39 aa, Chlamydia trachomatis
MAGKKMVSKGKDRQIFRKTADRTKKMNVRPLLYRGGIRL

SEQ ID NO: 13 - protein, 41 aa, Marine gokushovirus
MRRPRKMNYKKSKRMFSRTAARTHRKNSLRGSRPMRGGIRL

SEQ ID NO: 14 - protein, 34 aa, Gokushovirinae Fen672_31
MSKKASRKSFTKGAVKVHKKNVPTRVPMRGGIRL

SEQ ID NO: 15 - protein, 35 aa, Unknown
  Description of Unknown uncultured bacterium sequence
MKMRKRTDKRVFTRTAAKSKKVNIAPKIFRGGIRL

SEQ ID NO: 16 - protein, 39 aa, Escherichia coli
MARSRRRMSKRSSRRSFRKYAKTHKRNFKARSMRGGIRL

SEQ ID NO: 17 - protein, 47 aa, Cognatishimia maritima
MESPNSRSQLGITLYLLSTIFPDACFRYRRELPYPLVIWGVATLCLQ

SEQ ID NO: 18 - protein, 39 aa, Escherichia coli
MARSRRRMSKRSSRRSFRKYAKSHKKNFKARSMRGGIRL

SEQ ID NO: 19 - protein, 37 aa, Oscillibacter sp.
MRKRMSKRVDKKVFRRTAASAKKINIDPKIYRGGIRL

SEQ ID NO: 20 - protein, 37 aa, Unknown
  Description of Unknown uncultured bacterium sequence
MRRRRLSRRTSRRFFRKGLKVRRRNLRARPMRGGFRI

SEQ ID NO: 21 - protein, 39 aa, Unknown
  Description of Unknown uncultured bacterium sequence
MARRKKMKGKRDKRVFKQTANKTKAINISPKNMRGGTRL

SEQ ID NO: 22 - protein, 53 aa, Marine gokushovirus
MLTVWSDTPTIKRRKDMYRKRMSRKKSKKVFAKTAMKVNKRNHVKPMRGGYRI

SEQ ID NO: 23 - protein, 39 aa, Marine gokushovirus
MMKYRKKMSAKSSRKQFTKGAMKVKGKNFTKPMRGGIRL

SEQ ID NO: 24 - protein, 38 aa, Marine gokushovirus
MRRYNVNKGKSAKKFRKQVSKTKVANLRSNPMRGGWRL

SEQ ID NO: 25 - protein, 28 aa, Spiroplasma virus SpV4
MAYRGFKTSRVVKHRVRRRWFNHRRRYR

SEQ ID NO: 26 - protein, 38 aa, Spiroplasma virus SpV4
MRRKVKNTKRHQWRLTHSARSIKRANIMPSNPRGGRRF

SEQ ID NO: 27 - DNA, 111 bp, Chlamydia virus Chp1
atggttcgtagaagacgtttgagaagaagaataagtagaagaatttttagaagaacagtagctagagttggtagaaggcgaaggtcttttcgtggtggtattagattttaa

SEQ ID NO: 28 - DNA, 135 bp, Unknown
  Description of Unknown Chlamydia phage 2 sequence
atgaggttaaaaatggcacgaagaagatacagacttccgcgacgtagaagtcgaagacttttttcaagaactgcattgaggatgcatccaagaaataggcttcgaagaattatgcgtggcggcattaggttctag

SEQ ID NO: 29 - DNA, 108 bp, Chlamydia virus CPAR39
ttgtgcaaaaaagtgtgcaaaaaatgcccaaaaaaagggccaaaaaatgcccccaaaatcggagcattttacgagagaaaaacacctagacttaaacagtctacttga

SEQ ID NO: 30 - DNA, 120 bp, Chlamydia phage 4
atggcacgaagatacagactttcgcgacgcagaagtcgacgacttttttcaagaactgcattaagaatgcatcgaagaaatagacttcgaagaattatgcgtggcggcattaggttttag

SEQ ID NO: 31 - DNA, 120 bp, Artificial Sequence
  Description of Artificial Sequence Synthetic polynucleotide
atggcggaacagtatgaactgagccaggaacagagcgaacagctgtttagcgaaaccgcgctgcagatgcatgaacagaacgaactgcaggaaattatgcagggcggcattgaattttaa

SEQ ID NO: 32 - DNA, 123 bp, Unknown
  Description of Unknown Guinea pig Chlamydia phage sequence
atggcacgaagaagatacagacttccgcgacgtagaagtcgaagacttttttcaagaactgcattaaggatgcatccaagaaataggcttcgaagaattatgcgtggcggcattaggttctag

SEQ ID NO: 33 - DNA, 117 bp, Chlamydia trachomatis
atgaaacgtagaaaaatgacaagaaaaggttctaagcgtctttttactgcaactgctgataaaactaaatctatcaatactgccccgccgccaatgcgtggcggtatccggttgtaa

SEQ ID NO: 34 - DNA, 120 bp, Chlamydia trachomatis
atgtctaaaaagcgttctcgcatgtctcgccgccgttctaagaagttgttctcgaaaacggctctccgcacgaagagtgtcaacacccgtccgcctatgcgcggagggttccggttctga

SEQ ID NO: 35 - DNA, 123 bp, Chlamydia trachomatis
atgtctcttcgtcgtcataagctttctcgtaaggcgtctaagcgtatttttcgtaaaggtgcatcacgcacgaagactttgaatactcgtgctacgcctatgcgcggcggtttccgtatttaa

SEQ ID NO: 36 - DNA, 117 bp, Chlamydia trachomatis
atgaaacgtcgtaaactgtccaaaaagaaatctcgcaagattttcactcgcggtgctgtaaatgtgaaaaagcgtaaccttcgcgctcgcccaatgcgcggcggtttccggatctaa

SEQ ID NO: 37 - DNA, 114 bp, Chlamydia trachomatis
atggctaaaaaaatgactaaaggcaaggatcgtcaggtttttcgtaaaaccgctgatcgtactaagaaactcaatgttagaccgttgttatatcgaggaggtatcagattatga

SEQ ID NO: 38 - DNA, 120 bp, Chlamydia trachomatis
atggcaggaaaaaaaatggtatcaaaaggaaaagatagacagattttccgaaaaactgctgatcgcactaaaaaaatgaatgtgcgcccgctattatatcgtggaggtattagattatga

SEQ ID NO: 39 - DNA, 126 bp, Marine gokushovirus
atgagaagaccaagaaaaatgaactataaaaaatcaaaaagaatgttttcacgcacagcagcgagaacacacagaaaaaactctctaagaggtagccgacctatgagaggcggaatacgtctttaa

SEQ ID NO: 40 - DNA, 105 bp, Gokushovirinae Fen672_31
atgtcgaagaaggcgtcgaggaagagttttactaagggtgccgttaaggttcataagaaaaatgttcctactcgtgttcctatgcgtggcggtattaggctttag

SEQ ID NO: 41 - DNA, 108 bp, Unknown
  Description of Unknown uncultured bacterium sequence
atgaaaatgcgtaagcggacggacaagcgagtgtttacccgcaccgctgctaagtccaagaaagtgaacattgccccgaaaatttttagaggaggtatccgtctgtga

SEQ ID NO: 42 - DNA, 120 bp, Escherichia coli
atggctcgttctcgccgtcgtatgtccaagcgttcttcccgtcgttcgttccgtaagtacgcaaagacgcataaacgtaactttaaagcccgctctatgcgtggtggaattcgtctttga

SEQ ID NO: 43 - DNA, 144 bp, Cognatishimia maritima
atggaaagcccgaacagccgcagccagctgggcattaccctgtatctgctgagcaccatttttccggatgcgtgctttcgctatcgccgcgaactgccgtatccgctggtgatttggggcgtggcgaccctgtgcctgcagtaa

SEQ ID NO: 44 - DNA, 120 bp, Escherichia coli
atggctcgttcccgtagacgtatgtctaagcgttcttcccgccgttcgttccgcaagtatgcgaagtcgcataagaagaactttaaagcccgctcaatgcgtggcggtatccgtttataa

SEQ ID NO: 45 - DNA, 114 bp, Oscillibacter sp.
atgagaaagcgaatgtctaagcgtgttgacaagaaggtgttccgtcgtactgccgcatctgccaagaagattaacattgaccccaagatttaccgtggaggtattcgcctatga

SEQ ID NO: 46 - DNA, 114 bp, Unknown
  Description of Unknown uncultured bacterium sequence
atgagacgtcgtcgtctatcccgcagaacttcccgccgttttttccgtaaaggacttaaggttcgccgtcgtaacctccgcgcgagacccatgagaggcggattcagaatttga

SEQ ID NO: 47 - DNA, 120 bp, Unknown
  Description of Unknown uncultured bacterium sequence
atggcacgacgcaagaagatgaaaggcaagcgggataaacgggtgtttaagcagacagccaacaaaaccaaggctatcaacatcagcccaaaaaacatgagagggggtacgagactgtga

SEQ ID NO: 48 - DNA, 162 bp, Marine gokushovirus
atgttaactgtgtggagtgacacccctaccataaaaaggagaaaagacatgtatagaaagagaatgtcaagaaagaaaagtaaaaaggtttttgcaaaaaccgcaatgaaagtaaataaaagaaaccacgttaaacctatgcgtggtggatatagaatataa

SEQ ID NO: 49 - DNA, 120 bp, Marine gokushovirus
atgatgaagtacagaaaaaaaatgagcgctaaaagtagccgaaagcaatttacaaaaggcgccatgaaagtgaagggtaaaaacttcacaaaaccaatgcgcggaggcatccgtctatag

SEQ ID NO: 50 - DNA, 117 bp, Marine gokushovirus
atgcgacgttacaatgtaaataaaggtaaatctgctaagaagtttcgaaagcaggtaagtaagacgaaggttgcaaacctacgttctaatccaatgcgaggtggttggagactctaa

SEQ ID NO: 51 - DNA, 87 bp, Spiroplasma virus SpV4
atggcttatcgtggttttaaaacgagtcgtgttgtaaaacatagagtacgtagaagatggtttaatcatagaagacgttatagatag

SEQ ID NO: 52 - DNA, 114 bp, Spiroplasma virus SpV4
atgcgtcgtaaagttaaaaacaccaaacgtcaccagtggcgtctgacccactctgctcgttctatcaaacgtgctaacatcatgccgtctaacccgcgtggtggtcgtcgtttc

SEQ ID NO: 53 - DNA, 135 bp, Unknown
  Description of Unknown Chlamydia phage 3 sequence
atgaggttaaaaatggcacgaagaagatacagacttccgcgacgtagaagtcgaagacttttttcaagaactgcattaaggatgcatccaagaaataggcttcgaagaattatgcgtggcggcattaggttctag

SEQ ID NO: 54 - protein, 44 aa, Unknown; identical to SEQ ID NO: 2
  Description of Unknown Chlamydia phage 3 sequence

SEQ ID NO: 55 - protein, 39 aa, Escherichia sp.
MARSRRRMSKRSSRRSFRKYAKTHKKNFKARSMRGGIRL

SEQ ID NO: 56 - protein, 39 aa, Escherichia sp.; identical to SEQ ID NO: 18

SEQ ID NO: 57 - protein, 55 aa, Pseudomonas phage PP7
MSSTLCRWAVKALRCTRVYKEFIWKPLVALSYVTLYLLSSVFLSQLSYPIGSWAV

SEQ ID NO: 58 - protein, 35 aa, Acinetobacter phage AP205
MKKRTKALLPYAVFIILSFQLTLLTALFMYYHYTF

SEQ ID NO: 59 - protein, 38 aa, Alces alces faeces associated microvirus MP12 5423
MAKKIRNKARDRRIFTRTASRMHKANRTPRFMRGGIRL

SEQ ID NO: 60 - protein, 38 aa, Unknown
  Description of Unknown Gokushovirinae environmental samples sequence
MRRKKMSRGKSKKLFRRTAKRVHRKNLRARPMRGGIRM

SEQ ID NO: 61 - protein, 39 aa, Unknown
  Description of Unknown Gokushovirinae environmental samples sequence
MAKRHKIPQRASQHSFTRHAQKVHPKNVPRLPMRGGIRL

SEQ ID NO: 62 - protein, 37 aa, Unknown
  Description of Unknown uncultured bacterium sequence
MRKKMHKSLDKRVFNRTAKKSKKINVNPVVYRGGIRL

SEQ ID NO: 63 - protein, 38 aa, Marine gokushovirus; identical to SEQ ID NO: 24

SEQ ID NO: 64 - protein, 41 aa, Unknown
  Description of Unknown Richelia intracellularis HH01 sequence
MRPVKRSRVNKARSAGKFRKQVGKTKMANLRSNPMRGGWRL

SEQ ID NO: 65 - protein, 41 aa, Gokushovirinae Fen7875_21
MKPLKRKPVQKARSAAKFRRNVSTVKAANMAVKPMRGGWRF

SEQ ID NO: 66 - protein, 44 aa, Mycobacterium phage BabyRay
MTKRDIEYRKALGLNPSEPLPKIVGAVTRHGATLKRPRVTALAR

SEQ ID NO: 67 - protein, 38 aa, Unknown
  Description of Unknown Bdellovibrio phage phiMH2K sequence
MKRKPMSRKASQKTFKKNTGVQRMNHLNPRAMRGGIRL

SEQ ID NO: 68 - DNA, 120 bp, Escherichia sp.
atggctcgttctcgtcgtcgtatgtctaaacgttcttctcgtcgttcttttcgtaaatatgctaaaactcataaaaaaaattttaaagctcgttctatgcgtggaggaattcgtttataa

SEQ ID NO: 69 - DNA, 117 bp, Escherichia sp.
atggcgcgcagccgccgccgcatgagcaaacgcagcagccgccgcagctttcgcaaatatgcgaaaagccataaaaaaaactttaaagcgcgcagcatgcgcggcggcattcgcctg

SEQ ID NO: 70 - DNA, 165 bp, Pseudomonas phage PP7
atgtcttctaccctgtgccgttgggctgttaaagctctgcgttgcacccgtgtttacaaagaattcatctggaaaccgctggttgctctgtcttacgttaccctgtacctgctgtcttctgttttcctgtctcagctgtcttacccgatcggttcttgggctgtt

SEQ ID NO: 71 - DNA, 108 bp, Acinetobacter phage AP205
atgaagaaaaggacaaaagccttgcttccctatgcggttttcatcatactcagctttcaactaacattgttgactgccttgtttatgtattaccattataccttttag

SEQ ID NO: 72 - DNA, 117 bp, Alces alces faeces associated microvirus MP12 5423
atggcaaagaaaattagaaacaaagcacgtgatagacgtatcttcacaagaacagcttcacgcatgcacaaggcaaaccgcacaccaagatttatgagaggcggtattaggttatga

SEQ ID NO: 73 - DNA, 114 bp, Unknown
  Description of Unknown Gokushovirinae environmental samples sequence
atgcgtcgtaaaaaaatgtctcgtggtaaatctaaaaaactgttccgtcgtaccgctaaacgtgttcaccgtaaaaacctgcgtgctcgtccgatgcgtggtggtatccgtatg

SEQ ID NO: 74 - DNA, 117 bp, Unknown
  Description of Unknown Gokushovirinae environmental samples sequence
atggctaaacgtcacaaaatcccgcagcgtgcttctcagcactctttcacccgtcacgctcagaaagttcacccgaaaaacgttccgcgtctgccgatgcgtggtggtatccgtctg

SEQ ID NO: 75 - DNA, 111 bp, Unknown
  Description of Unknown uncultured bacterium sequence
atgcgtaaaaaaatgcacaaatctctggacaaacgtgttttcaaccgtaccgctaaaaaatctaaaaaaatcaacgttaacccggttgtttaccgtggtggtatccgtctg

SEQ ID NO: 76 - DNA, 117 bp, Marine gokushovirus; identical to SEQ ID NO: 50

SEQ ID NO: 77 - DNA, 126 bp, Unknown
  Description of Unknown Richelia intracellularis HH01 sequence
atgcgtccagttaaaagatcaagagtaaataaggcccgatctgcaggcaagtttcgtaagcaggtcggtaaaacaaagatggcaaatctgcgtagtaatccgatgcgcggcggatggcggctgtga

SEQ ID NO: 78 - DNA, 126 bp, Gokushovirinae Fen7875_21
atgaagccattgaagcgtaagccggttcagaaggcgcggtcagcagccaagttccgtcgaaatgtgtctaccgttaaggctgccaatatggcggtgaagccgatgcgcggcggttggcggttctga

SEQ ID NO: 79 - DNA, 135 bp, Mycobacterium phage BabyRay
atgaccaagagagacatcgagtaccggaaagctttggggctcaacccatctgagccgctcccgaagattgtgggtgccgtcacccgccacggggccactctgaaacgcccacgggtcaccgcactggcccgatag

SEQ ID NO: 80 - DNA, 117 bp, Unknown
  Description of Unknown Bdellovibrio phage phiMH2K sequence
atgaaaagaaaaccaatgagccgcaaggcctctcaaaaaaccttcaaaaagaacacaggcgttcaacgcatgaaccatctcaacccacgcgccatgcgtggtggcattagactataa

SEQ ID NO: 81 - protein, 44 aa, Artificial Sequence; identical to SEQ ID NO: 2
  Description of Artificial Sequence Synthetic polypeptide
  MOD_RES (2)..(2): D-amino acid
  MOD_RES (4)..(4): D-amino acid
  MOD_RES (7)..(9): D-amino acid
  MOD_RES (11)..(11): D-amino acid
  MOD_RES (14)..(16): D-amino acid
  MOD_RES (18)..(19): D-amino acid
  MOD_RES (23)..(23): D-amino acid
  MOD_RES (27)..(27): D-amino acid
  MOD_RES (31)..(31): D-amino acid
  MOD_RES (33)..(33): D-amino acid
  MOD_RES (35)..(36): D-amino acid
  MOD_RES (39)..(39): D-amino acid
  MOD_RES (43)..(43): D-amino acid

SEQ ID NO: 82 - protein, 45 aa, Artificial Sequence
  Description of Artificial Sequence Synthetic polypeptide
MRLKMARRRYRLPRRRSRRLFSRTALRMHPRNRLRRIMRGGIRFC

SEQ ID NO: 83 - protein, 54 aa, Artificial Sequence
  Description of Artificial Sequence Synthetic polypeptide
APKAMRLKMARRRYRLPRRRSRRLFSRTALRMHPRNRLRRIMRGGIRFLQKKGI

SEQ ID NO: 84 - protein, 60 aa, Artificial Sequence
  Description of Artificial Sequence Synthetic polypeptide
MARRYRLSRRRSRRLFSRTALRMHRRNRLRRIMRRLFSRTALRMHRRNRLRRIMRGGIRF

SEQ ID NO: 85 - protein, 44 aa, Artificial Sequence
  Description of Artificial Sequence Synthetic polypeptide
GRLYRFHRPRRRNAIGMSRMRRKMFLRRMLRLISRRTRRPRLRA

SEQ ID NO: 86 - protein, 39 aa, Artificial Sequence
  Description of Artificial Sequence Synthetic polypeptide
RTRNFRIRRAKARRKMMLSHFKYGMARKGSKSRSSRRSR

SEQ ID NO: 87 - protein, 39 aa, Artificial Sequence; identical to SEQ ID NO: 16
  Description of Artificial Sequence Synthetic polypeptide
  MOD_RES (3)..(3): D-amino acid
  MOD_RES (5)..(7): D-amino acid
  MOD_RES (10)..(11): D-amino acid
  MOD_RES (14)..(15): D-amino acid
  MOD_RES (18)..(19): D-amino acid
  MOD_RES (22)..(22): D-amino acid
  MOD_RES (25)..(26): D-amino acid
  MOD_RES (29)..(29): D-amino acid
  MOD_RES (31)..(31): D-amino acid
  MOD_RES (34)..(34): D-amino acid
  MOD_RES (38)..(38): D-amino acid

SEQ ID NO: 88 - protein, 40 aa, Artificial Sequence; identical to SEQ ID NO: 6
  Description of Artificial Sequence Synthetic polypeptide
  MOD_RES (3)..(5): D-amino acid
  MOD_RES (7)..(7): D-amino acid
  MOD_RES (10)..(12): D-amino acid
  MOD_RES (14)..(15): D-amino acid
  MOD_RES (19)..(19): D-amino acid
  MOD_RES (23)..(23): D-amino acid
  MOD_RES (27)..(27): D-amino acid
  MOD_RES (29)..(29): D-amino acid
  MOD_RES (31)..(32): D-amino acid
  MOD_RES (35)..(35): D-amino acid
  MOD_RES (39)..(39): D-amino acid

SEQ ID NO: 89 - protein, 38 aa, Artificial Sequence; identical to SEQ ID NO: 10
  Description of Artificial Sequence Synthetic polypeptide
  MOD_RES (2)..(5): D-amino acid
  MOD_RES (8)..(10): D-amino acid
  MOD_RES (12)..(13): D-amino acid
  MOD_RES (17)..(17): D-amino acid
  MOD_RES (23)..(25): D-amino acid
  MOD_RES (28)..(28): D-amino acid
  MOD_RES (30)..(30): D-amino acid
  MOD_RES (33)..(33): D-amino acid
  MOD_RES (37)..(37): D-amino acid

SEQ ID NO: 90 - protein, 38 aa, Artificial Sequence; identical to SEQ ID NO: 59
  Description of Artificial Sequence Synthetic polypeptide
  MOD_RES (3)..(4): D-amino acid
  MOD_RES (6)..(6): D-amino acid
  MOD_RES (8)..(8): D-amino acid
  MOD_RES (10)..(10): D-amino acid
  MOD_RES (12)..(13): D-amino acid
  MOD_RES (17)..(17): D-amino acid
  MOD_RES (21)..(21): D-amino acid
  MOD_RES (24)..(24): D-amino acid
  MOD_RES (27)..(27): D-amino acid
  MOD_RES (30)..(30): D-amino acid
  MOD_RES (33)..(33): D-amino acid
  MOD_RES (37)..(37): D-amino acid

SEQ ID NO: 91 - protein, 39 aa, Artificial Sequence; identical to SEQ ID NO: 4
  Description of Artificial Sequence Synthetic polypeptide
  MOD_RES (3)..(4): D-amino acid
  MOD_RES (6)..(6): D-amino acid
  MOD_RES (9)..(11): D-amino acid
  MOD_RES (13)..(14): D-amino acid
  MOD_RES (18)..(18): D-amino acid
  MOD_RES (22)..(22): D-amino acid
  MOD_RES (25)..(26): D-amino acid
  MOD_RES (28)..(28): D-amino acid
  MOD_RES (30)..(31): D-amino acid
  MOD_RES (34)..(34): D-amino acid
  MOD_RES (38)..(38): D-amino acid

SEQ ID NO: 92 - protein, 44 aa, Artificial Sequence
  Description of Artificial Sequence Synthetic polypeptide
MRLRYGHRRMTAGRIRMRSRRKFMLPRFRLLRIPRRSNRRRLRA

SEQ ID NO: 93 - protein, 44 aa, Artificial Sequence; identical to SEQ ID NO: 92
  Description of Artificial Sequence Synthetic polypeptide
  MOD_RES (2)..(2): D-amino acid
  MOD_RES (4)..(4): D-amino acid
  MOD_RES (8)..(9): D-amino acid
  MOD_RES (14)..(14): D-amino acid
  MOD_RES (16)..(16): D-amino acid
  MOD_RES (18)..(18): D-amino acid
  MOD_RES (20)..(22): D-amino acid
  MOD_RES (27)..(27): D-amino acid
  MOD_RES (29)..(29): D-amino acid
  MOD_RES (32)..(32): D-amino acid
  MOD_RES (35)..(36): D-amino acid
  MOD_RES (39)..(41): D-amino acid
  MOD_RES (43)..(43): D-amino acid

SEQ ID NO: 94 - protein, 40 aa, Marine gokushovirus
MIVRRHKMSRRRSRKLFSKTASRTRSKNLRSRPMRGGYRI

SEQ ID NO: 95 - protein, 38 aa, Artificial Sequence; identical to SEQ ID NO: 7
  Description of Artificial Sequence Synthetic polypeptide
  MOD_RES (3)..(5): D-amino acid
  MOD_RES (8)..(9): D-amino acid
  MOD_RES (12)..(13): D-amino acid
  MOD_RES (21)..(21): D-amino acid
  MOD_RES (23)..(23): D-amino acid
  MOD_RES (33)..(33): D-amino acid
  MOD_RES (37)..(37): D-amino acid

SEQ ID NO: 96 - protein, 37 aa, Artificial Sequence; identical to SEQ ID NO: 19
  Description of Artificial Sequence Synthetic polypeptide
  MOD_RES (2)..(4): D-amino acid
  MOD_RES (7)..(8): D-amino acid
  MOD_RES (11)..(12): D-amino acid
  MOD_RES (15)..(16): D-amino acid
  MOD_RES (22)..(23): D-amino acid
  MOD_RES (29)..(29): D-amino acid
  MOD_RES (32)..(32): D-amino acid
  MOD_RES (36)..(36): D-amino acid

SEQ ID NO: 97 - protein, 37 aa, Artificial Sequence; identical to SEQ ID NO: 20
  Description of Artificial Sequence Synthetic polypeptide
  MOD_RES (2)..(5): D-amino acid
  MOD_RES (8)..(9): D-amino acid
  MOD_RES (12)..(13): D-amino acid
  MOD_RES (16)..(17): D-amino acid
  MOD_RES (20)..(20): D-amino acid
  MOD_RES (22)..(24): D-amino acid
  MOD_RES (27)..(27): D-amino acid
  MOD_RES (29)..(29): D-amino acid
  MOD_RES (32)..(32): D-amino acid
  MOD_RES (36)..(36): D-amino acid

SEQ ID NO: 98 - protein, 39 aa, Artificial Sequence; identical to SEQ ID NO: 21
  Description of Artificial Sequence Synthetic polypeptide
  MOD_RES (3)..(6): D-amino acid
  MOD_RES (8)..(8): D-amino acid
  MOD_RES (10)..(11): D-amino acid
  MOD_RES (13)..(14): D-amino acid
  MOD_RES (17)..(17): D-amino acid
  MOD_RES (22)..(22): D-amino acid
  MOD_RES (24)..(24): D-amino acid
  MOD_RES (31)..(31): D-amino acid
  MOD_RES (34)..(34): D-amino acid
  MOD_RES (38)..(38): D-amino acid

SEQ ID NO: 99 - protein, 37 aa, Artificial Sequence
  Description of Artificial Sequence Synthetic polypeptide
  MOD_RES (2)..(4): D-amino acid
  MOD_RES (6)..(6): D-amino acid
  MOD_RES (9)..(10): D-amino acid
  MOD_RES (14)..(14): D-amino acid
  MOD_RES (20)..(20): D-amino acid
  MOD_RES (23)..(24): D-amino acid
  MOD_RES (33)..(33): D-amino acid
  MOD_RES (36)..(37): D-amino acid
MRRKVKNTKRHQWRLTHSARSIKRANIMPSNPRGGRR

SEQ ID NO: 100 - protein, 39 aa, Artificial Sequence; identical to SEQ ID NO: 55
  Description of Artificial Sequence Synthetic polypeptide
  MOD_RES (3)..(3): D-amino acid
  MOD_RES (5)..(7): D-amino acid
  MOD_RES (10)..(11): D-amino acid
  MOD_RES (14)..(15): D-amino acid
  MOD_RES (18)..(19): D-amino acid
  MOD_RES (22)..(22): D-amino acid
  MOD_RES (25)..(26): D-amino acid
  MOD_RES (29)..(29): D-amino acid
  MOD_RES (31)..(31): D-amino acid
  MOD_RES (34)..(34): D-amino acid
  MOD_RES (38)..(38): D-amino acid

SEQ ID NO: 101 - protein, 38 aa, Artificial Sequence; identical to SEQ ID NO: 24
  Description of Artificial Sequence Synthetic polypeptide
  MOD_RES (2)..(3): D-amino acid
  MOD_RES (8)..(8): D-amino acid
  MOD_RES (10)..(10): D-amino acid
  MOD_RES (13)..(14): D-amino acid
  MOD_RES (16)..(17): D-amino acid
  MOD_RES (21)..(21): D-amino acid
  MOD_RES (23)..(23): D-amino acid
  MOD_RES (28)..(28): D-amino acid
  MOD_RES (33)..(33): D-amino acid
  MOD_RES (37)..(37): D-amino acid

SEQ ID NO: 102 - protein, 46 aa, Mycobacterium virus Peaches
MKLTKSDIAYREALGLSTTDPLPAEIGMVTRRANRLKRPRKTARFR

SEQ ID NO: 103 - DNA, 120 bp, Marine gokushovirus
atgatcgttcgtcgtcacaaaatgtctcgtcgtcgttctcgtaaactgttctctaaaaccgcttctcgtacccgttctaaaaacctgcgttctcgtccgatgcgtggtggttaccgtatc

SEQ ID NO: 104 - DNA, 138 bp, Mycobacterium virus Peaches
atgaaactgaccaaatctgacatcgcttaccgtgaagctctgggtctgtctaccaccgacccgctgccggctgaaatcggtatggttacccgtcgtgctaaccgtctgaaacgtccgcgtaaaaccgctcgtttccgt

SEQ ID NO: 105 - DNA, 132 bp, Artificial Sequence
  Description of Artificial Sequence Synthetic polynucleotide
atgaggttaaaaatggcacgaagaagatacagacttccgcgacgtagaagtcgaagacttttttcaagaactgcattgaggatgcatccaagaaataggcttcgaagaattatgcgtggcggcattaggttc

SEQ ID NO: 106 - DNA, 135 bp, Artificial Sequence
  Description of Artificial Sequence Synthetic polynucleotide
atgaggttaaaaatggcacgaagaagatacagacttccgcgacgtagaagtcgaagacttttttcaagaactgcattgaggatgcatccaagaaataggcttcgaagaattatgcgtggcggcattaggttctgt

SEQ ID NO: 107 - DNA, 159 bp, Artificial Sequence
  Description of Artificial Sequence Synthetic polynucleotide
gctcctaaagctaggttaaaaatggcacgaagaagatacagacttccgcgacgtagaagtcgaagacttttttcaagaactgcattgaggatgcatccaagaaataggcttcgaagaattatgcgtggcggcattaggttcttacaaaaaaaaggaatt

SEQ ID NO: 108 - DNA, 180 bp, Artificial Sequence
  Description of Artificial Sequence Synthetic polynucleotide
atggctcgtcgttatcgtttatctcgtcgtcgttctcgtcgtttattttctcgtactgctttacgtatgcatcgtcgtaatcgtttacgtcgtattatgcgtcgtttattttctcgtactgctttacgtatgcatcgtcgtaatcgtttacgtcgtattatgcgtggaggaattcgtttt

SEQ ID NO: 109 - DNA, 132 bp, Artificial Sequence
  Description of Artificial Sequence Synthetic polynucleotide
ggacgtttatatcgttttcatcgtcctcgtcgtcgtaatgctattggaatgtctcgtatgcgtcgtaaaatgtttttacgtcgtatgttacgtttaatttctcgtcgtactcgtcgtcctcgtttacgtgct

SEQ ID NO: 110 - DNA, 117 bp, Artificial Sequence
  Description of Artificial Sequence Synthetic polynucleotide
cgtactcgtaattttcgtattcgtcgtgctaaagctcgtcgtaaaatgatgttatctcattttaaatatggaatggctcgtaaaggatctaaatctcgttcttctcgtcgttctcgt

SEQ ID NO: 111 - DNA, 120 bp, Artificial Sequence
  Description of Artificial Sequence Synthetic polynucleotide
atggctcgttctcgtcgtcgtatgtctaaacgttcttctcgtcgttcttttcgtaaatatgctaaaactcataaacgtaattttaaagctcgttctatgcgtggaggaattcgtttataa

SEQ ID NO: 112 - DNA, 123 bp, Artificial Sequence; identical to SEQ ID NO: 32
  Description of Artificial Sequence Synthetic polynucleotide

SEQ ID NO: 113 - DNA, 117 bp, Artificial Sequence
  Description of Artificial Sequence Synthetic polynucleotide
gtgaaacgtcgtaaactgtccaaaaagaaatctcgcaagattttcactcgcggtgctgtaaatgtgaaaaagcgtaaccttcgcgctcgcccaatgcgcggcggtttccggatctaa

SEQ ID NO: 114 - DNA, 114 bp, Artificial Sequence
  Description of Artificial Sequence Synthetic polynucleotide
atggctaaaaaaatccgtaacaaagctcgtgaccgtcgtatcttcacccgtaccgcttctcgtatgcacaaagctaaccgtaccccgcgtttcatgcgtggtggtatccgtctg

SEQ ID NO: 115 - DNA, 120 bp, Artificial Sequence; identical to SEQ ID NO: 30
  Description of Artificial Sequence Synthetic polynucleotide

SEQ ID NO: 116 - DNA, 132 bp, Artificial Sequence
  Description of Artificial Sequence Synthetic polynucleotide
atgcgtctgcgttacggtcaccgtcgtatgaccgctggtcgtatccgtatgcgttctcgtcgtaaattcatgctgccgcgtttccgtctgctgcgtatcccgcgtcgttctaaccgtcgtcgtctgcgtgct

SEQ ID NO: 117 - DNA, 132 bp, Artificial Sequence; identical to SEQ ID NO: 116
  Description of Artificial Sequence Synthetic polynucleotide

SEQ ID NO: 118 - DNA, 117 bp, Artificial Sequence; identical to SEQ ID NO: 33
  Description of Artificial Sequence Synthetic polynucleotide

SEQ ID NO: 119 - DNA, 114 bp, Artificial Sequence; identical to SEQ ID NO: 45
  Description of Artificial Sequence Synthetic polynucleotide

SEQ ID NO: 120 - DNA, 114 bp, Artificial Sequence; identical to SEQ ID NO: 46
  Description of Artificial Sequence Synthetic polynucleotide

SEQ ID NO: 121 - DNA, 120 bp, Artificial Sequence; identical to SEQ ID NO: 47
  Description of Artificial Sequence Synthetic polynucleotide

SEQ ID NO: 122 - DNA, 114 bp, Artificial Sequence; identical to SEQ ID NO: 52
  Description of Artificial Sequence Synthetic polynucleotide

SEQ ID NO: 123 - DNA, 120 bp, Artificial Sequence; identical to SEQ ID NO: 68
  Description of Artificial Sequence Synthetic polynucleotide

SEQ ID NO: 124 - DNA, 117 bp, Artificial Sequence; identical to SEQ ID NO: 50
  Description of Artificial Sequence Synthetic polynucleotide